SEQUENCE LISTING (189 sequences)

SEQ ID NO: 1 - DNA, 1053 bp, Chlamydomonas reinhardii
gacggagctggtgtccgagattgagaagaccttcggtgtggacgcctcggccgccgcccccgttgccatggctgccatcgctgcccctggcgctgctgctgctcccgctgttgaggagaagaccaccttcgatgtggtgctggaggagatccccgccgacaagaaggtcggcgtctacaaggtggtccgcaacatcgccaacatcgccgtcaaccaggttaaggacttcaccgctactctgcccaaggtcctgaaggagggcctgagcaaggaggacgctgaggccgccaaggctcagctgctggaggccggtgccaaggccaaggtcgcttaagcggcctgcgcgtaattacatcaacgtcttgtcagcagtagcgtaattggtgagcgaagagcgagctgggtcgcgctgtcggcagtgggcagtgctttcgtggtggaaatgtggtcgcggacttcaacttcgtggaccgaccgcgtcgacgccgcactgcaaagccaggtgttgtagtaatgctgggaactggtggcttgctcggagagcgatgggcttcgggtcgaggggtttcatgtggcgatgggagttggagcctggtggcagttctttgctgcgggttgggaggtcaagtcctgcatgcagtggctgaatgcgggggccatgtgcgtatgggtgcaggcaggcggggcctatgccggcaattttggactaactgtgccggccgggtcgccgctgtggggtggttaggggcgtgtgcgtcactgcgaagtgtttgctatggcggctgaccactaaccatggctatgatgcgagcctcctgatgctactcagggcccaaatggtgacgcttgtctggtgctccccaggcaacgcgtagagcaaaggcggattttcgagatagttgtggcggcgtcatgctggggtggcgcggctcacacccgcccgtagatgatgtagcgcagaagagtgtccctagcgcggaggttggaggtgtgcggtgctcaagtgccgtgaaagacagtgcgaggcttatcctggatgtaacggagaagctg

SEQ ID NO: 2 - DNA, 1635 bp, Chlamydomonas reinhardii
atattttctatgaccgcacgcgtacacgctgcgcagctaggagtttgtctaacgcgtgtgtgcctgtgcgcgtgcttggctgtggggtcgtccagtactaagtgcgcccacgtgcgtgcatagccttgcgtctcatcccgcagtgagcatatgattgtgactgacggtttggtgacaccaacaacaagacaaattgacctgaccagtgcgcgtctgtcctgcagcgtcctgttggtggttggtgcaactcgagtcggagtcagtggaaatgggatgggatacttggtgagagcgtggcaaggctcgtgcgtgccatgcgcgagtgtgtgttttgtttatcgatgatgtgcgtcgtggggctctgttaaccccccttcagattttttttgaagatgtctggacgggtggcgttgacagtcctcaggagcgagctcggcggctgggccagacgcgcttagtgcagtgtggtcatgcgaaatgcgaacataaagtccgtccggagattcacccggcgaacgaaccatggaccctggaccatgcattttacgggtagatgggtaggcattcagtactgtcgcagcgcttacaggtctagatgggagtagccccgcgatgcaaggacaggagcatctggctggacgtggaaatgacgaggtgtgttgggggatgactccataccataggttggcgcaggctggcgtgtctgtactatgtcgtgattcaagatggtgtgccgctgagcggaatgggcacaggatgcagggaaacggatagtgcggtgcacgggcagaattggtggtctgcatcacgcgttgactaggacgcgagacgcgggataggatggtcgctaagatgtgtacgagtaggcctggacgggggcacacgacggcgcaggtctctccgccgtgcgacacaatgcgcggtgggaggcgatgcggaggacattggcacacgcgccagcacatacgcacatatacggatgtgcttgcggtgcgttcaatgaggatgacagggtattgtgggggttgcagaacccaattcttttctgccgacgtatgcagctcgcgcccctaagcgctcgggtgttgtcactcaccatgtgagcgggcacgcctggacgtggcacgatggtagctgcttgacttggcattgtgtgtgtgcgattgtcgtgaagacttgaagacaggagtggagtgatgggtttgatgcgaggtatgcaagagtgaagttcgcagatggcgggcgccggatgtgggccccttggtggacagaggatggaggcaatgccccggccgaaggcttctgggacacgcagttccaccttgccgaaggttggacgattgcagggatgctgctgctggagacgaggtggctgcgaaggtagaagggcggacaagggaacaaataaatagaagttgcgacatacggtttgaatagttgcaccgcgagccgactccggtgttcaccatggtaggtacctttcgatggggtacgcatttcagtggctgaaacacctggcgctgacgcgggtgcgggctttagaggacaggtgtcgatgcctgggcctgatgcccaattgcagtagtgcaggctggcagaagcctcgcaatgttggagcctctttaatactgttgg

SEQ ID NO: 3 - DNA, 936 bp, Chlamydomonas reinhardii
tgacactgcttcccctcgcaaccatggcttcctacaagcccaagagcgtgaaggatgtgccggcggaggacttcattaagacctacgctgcgcacctgaaggccaacgataagatccagctgcccagctgggtggatgtggtgaagaccggcaagttcaaggagctggccccctacgaccccgactggtactacgtgcgcgctgcctcggtggcccgcaagctgtacatccgccagggcatgggtgtcggtctgttccgcactcagtacggtggccgcaacaagcgccgcggctccaagcccgagttccagtccaaggcttctggcggcctggttcgccacatcatgaagcagctggaggagtgcggcctgatggagaaggcccccgagaagggtcgccgcctgaccgccaacggccagcgcgacatggaccagattgccggccgcatcaccgtccagctgcaggctttcttctaagcgcgcaacagcgcttgagcggataagtccacccggcggccgcgaggcagtagcagcagctatggcagcggcgaacggagcgggagcagcagcgtgagcggcagcagtggcaggcactgcaaacgggtggatgtggcggcggcgaacgaccggggggctctccgggaggaggctggccgtgaaatggaacggcttgtattcggcgcacgtgtgattgtgtgggcagcgcaccatgcgccccgtcctcggtgcgacagctcatgcttacgggcagggctgcaaacgtgcctcctccgttgtcgatggaggagaggacgacctgccagggtgctggagggctgtggtccatgcgtgcgtgctcttggcgtgtgggcaatgcaatgtgtggccatggcagcgcggtgatgctggcccgcgtcggaagagacgttctatgaggttatgtgtaacacgcacacgc

SEQ ID NO: 4 - DNA, 531 bp, Chlamydomonas reinhardii
atgcccaaggcgaccatcgtgcagaccgtggagaagtacctgaactaagcagcctggcgcagcggcattcagccggcgggagtggggttccccgctcgcccatccgcgggcggagcgatgtgacaggggcacagtagcacacgggactagggggcaccacacccagaagggtgcggccgcattgtttcgtttcgaggggacgcttgccaccgtatgggtggcagttttgtaagcacttaaataagtaggagcacggccccccggccgtgggttcgtgtgacggaccggggccagtgtgtgtcgtgaacgttttggaccccgggccgccattcggccctgagttttggaacgcttttggatgaagcggaaaacggattgtgattgaacaattatcggggttggcccatggcaaagtgcatggagccgcgagtgagcgagtagtgacgtccggagctgtgcgcgcgggtgcggccgcggcgcagtgcgtgagttgtactgcaggcatggagaggttgtaaccgagaggctcgg

SEQ ID NO: 5 - DNA, 1441 bp, Chlamydomonas reinhardii
atgataacacctgcacaccgagtatccgctcaaagcccgcgcttgcgacccctcatacttgcgtcatccagactcatcttgcgaaatggcagctaaaccaagcggaccccgcactttggtcaacacgatcaagctacttgtggatgcgggagcagctaagccagctcctccagttggccccgccctcggccaagcagggctgaacattatggcgttctgcaaggagttcaacgcgaagaccgccaactacaaggagggcacactgctgcgcgttaaagtgcgcgtcttcagcgacaagtcttacgagtgggacctgaagacaccgcccagcacctggcttatcaagaaggccgcaggcctggcgcgcgccgcggaccggccagggcacgagctgtcgggcacagtctctctgaagcacctgtatgagatcgcgcgcgtcaagcagcgggacacacccaacctgcctctgcagtccatcgttacggcgctgctcagcacctgtcgcagcatgggtgtgcgcgtcgtggcgcgccccgaggaggcgtgagcgacggcagcctcccctttccccacctcattcgcaaggcactgcacagctttggcccgctgcaggggcgcccgctacgttggcagtccccacggcatgctgttggcagtccaggtgatggcgttcctcaacgccagaacttccgcggcttgcgcgtgccctgcctgcgcgccgcagtgtcccggtggcccgcgctcccgcctgccaccgcgtgcggccttgcctgcagcggatggcggcggctgtcctgcggacgcttctcacgcccaactttcagcaagcagtgggatgcggcttcccatacaagccagccggacgcacccgtatcacagctaagggcgccagttggtggtagtcgggcccgtcctgcacgccccctggtcgaggtcatgcgccgtgtatgcacccgcttccgtggaggtggacacctctagtagcttcgggcgttcacagcatcggcagtgggtcataggctgtatgcggatggcattgctgtccgtgctacttcattgacgccctggccgtaggggcctgggtctgctcgccctgccccgcgcggctggcaggtccgttgccgctcttgggggggcagctaggacatagatggttggacgaggcgcatgcctgtgggtgtacgcaggatgcctgatgacgattgcttacactcgactacactggtgtggttctggtgctgcgtcacccgtttagtcgcagttgcggtgcaaggggctgtcgtgagctggcgcatctgtcgtgcatcgtgagcctcgggctcccgaacatgcattgcatgtaaccacttgcaccgcttgcaccaagatgcgcttgcagtgcagcgtgcgacaatcggattggcaaactgaagtcgtccagaaaatgtaatggtgcatctag

SEQ ID NO: 6 - DNA, 900 bp, Chlamydomonas reinhardii
gtggactcatacatagtgcggggtttgttatcagatgtcgggcggccgcgcagtgtgtgtcgctggaaggtatcggcaatgtgcgaggaagtgtacactgttggtgcctgtagctagtgcgcttggtgcgtcgcgtgtgtgcaagtcatggttcctggcgggagtcagcgtgcaatggaccacttcatccgctgcccggatgttaaggtacgtgtgcgttgaggatgagagtctggttggagagccagtggcagaggggcaaggccctttgctactttgtgatcgcgtgctcatcgttgctattgttttttgccggcgtaagcggcgtggtggaggacgcaacgtgtgctgcagctgggtgttgagatcgagggacccgaagcacacggctcagaagaacgttttcatccagcctggagaggtgtgcgtgtgctgcggtcaatgagtttgcgctggcgtccagaacgactcttggggatgcgttgttgagacgtagggttagggtttggtatgaagtgcaccgaaagagcagcagtgagtggcaagtgcccctttctgcgctgttcggcccctgcaagttgaagtagttcttggatgcagtcccaacccgggcatgcggtcggtgctggtgtatcaaacaatctggagttttggtgtccggccatgggtgtcgctgtgtgtgttcatttcggggaggctgagttccaacggcccctaggccgccgcttgggggtctccgctgtgtaccattgaatcggtctgcagactgggttccgtacccaattaattttgtttcgcggtctttcataacgcgtaagaacccgcgtcggaagagtggaaatggttggtggtgagaaggagcggctcgtcagtacggaggtgttgacggagctccagtga

SEQ ID NO: 7 - DNA, 2232 bp, Chlamydomonas reinhardii
atgggtgcctggggacaaagcagcatgctacgcggggaaaccgttggtacggcacctctacaccagctgtctgcgcaaccggctgctgaaggacctgaatggggaggcggagaagctcatgtccgcagcattccagcgcgggcataccgcggccctgtccctgtatgacgaccccccaaagaccgtgcgctgcggaggccatcggaaaacaacggcgacatacacgctcacaaccagtggcggagtgtgcgtgtacaagaacggcagcgggccggagcggctgctggagttcgggtctacttgcacctgtgccgtggtgcaggggcggtccgtgtggatggccaacgtcggtgactccacagcagtgcttgggaccgacaacggtgcgtcgtacacgtccaaaacgctgacggtgcggcgcaacgggcataatgcggaggaggcgaagcggatgaaggagggcttctcgggcacagtgaacctgaaggatgctgcgcacgaggacggctacctgcaggtggtgacggggccgtggcagggttacgagctgagcgtgacgcgcgcgctggggcacaagcacatgagcgaacacggcgtgctgacggagccgtacgtggtcacatttgaggccagcaaggacgactgctgcctgatcatggcctcggacggcgtctgggacgtgatggacgggcaggaggcagtgaacagggtgatggaggtggcgagcgagggtaaaactgcggcccaagcagctaagatgctggtggaggaggcggtggagcttggcgtgaagtccccgtgtggagaggcggacaacacatcagcaattgtggtcttctttgcttagcaggaagaggacggtaacacaaggagttctgtgggtctgccctaggacgggcagcggcggatgctggcttgtgggggcgggtcctgcgttatgcgtagtctacatgatgacatgtttgtgttttcgaagtgtgttgttcttgcagaattgttgttaaagctcactcgtgtttaccttctaaccgtagtagcaacgacctgtatgccatgtgccagggtgaaagacagcgcggttacaatggcactcctggcagttatgggtgccactacgatgcggacactgaagcgctcattccaaagcgttctcaatccaagcgccagtaactcagatgcattccattgcggttgggtttggtccccacgagccccataacggggcgcattgggctgcgcagctattatgtatcgttccccgcttctgccgtcgtaaggtgaacgtggtgcctcaagctaccctgcctcagcccatactaccctgcctcagacgtggcccagaccgtggcccagagggggcggccaccttgggtgtgggcacccttgcagggcgtcgggtgatgatgtgatgagagtcgccccggttagcgcacgtgctcgagtgctgtgccgcaatagggtgaacgtggcggtgaatcggcttaggtggacattgcgccgcccaacacggcaaagattgggcgcgcattgctgaagcagagggcacggcggcgaggatagttgtctttggatgtgcataccgtattcgcattctgcggggtttggttagtgggcatatggccggtacgtcactgcgtccatttgcagtgtgccctgcgaatgtggacccttgcggacaaggcagattgcgcatgacctactcttacgcaagctcttactgcaaaaccgctgtgtattggctattggtgatttgaatttgaacattcgcgcggtcgtctcatggattgtctctgtgctacggcgctatgccaccctcagatctgattgcaacacgccagcaacggtgcgcgcacattcgcgctgcaaggtgtggaaaccgtccacgcccgcaggcttgatcgtagcatcagtagccgtggagtctatccgcacatgttttggcgcatatttggcggtatctgaagacgttttgggggttcgggggtgcgccccctagggaaaatgttccgggcgtaggggccatagacgctctgtagacgccgcgggcctgaggtcatctacaagatacgggcactggaacgcatgtcagcagtggctcagtgcctgtgccatagcattgagcattggatgcagggaagggggggggcagaaagctcaggatcttacccgatgctcgatagcgcagagcttgaggagagcactacgcacgtacttttagtgttgtcagggacg

SEQ ID NO: 8 - DNA, 1596 bp, Chlamydomonas reinhardii
atactctgcgttgataccactgcttcccccattaatactctgcgttgataccactgcttcccctagccgcggagcttgcacttgctcctctacgccacaaatggctctgcagcagacccgcgctttctcgggccgcatgtcgctgaagtcggctgcggctcccgtccgtgttagccgcgctagccgcgcccggactgtgctggtggaggctcgccagttcaggaaggccatgggtgtgctcggcaccaaggcgggcatgatgtcttacttcaccgaggacggtctgtgcgtgcctgctaccgtgattgctctggaggagggcaacgtggtgacccaggtgaagacccaggacaccgacggctacaatgccgtccaaattgggtacaaggccaccgctgagaagcgcgttaccaagcccgagctgggccacctgaagaaggcgggcgtgccgccgatgcgccacctggtggagttcaagctcaaggaccgtgctgctgtggaggcctaccagcccggccaggctctggacgtggctgctcttctgaaggagggcgagcccgtggacattgctggcatcaccgtgggcaagggcttccagggcaccatcaagcggtggcaccacaagcgcggtgccatgtcccacggttccaagtcgcaccgtgagcacggctccatcggttccgccaccaccccctcgcgcgtcttccccggcctcaagatggcgggccagatgggcaacgtccgtatgaccgtcaagaaccagtccctgctcaaggtcgacaccgagcgccacgctctggtggtcaagggctcggtgcccggcaaggttggcaacgtggtggagatcacccccgccaagctggtcggcgtcaactggtaaacaacaacaggcccagaatggcggtggttgtaacgctggcggttgacggttggtactggccgccttttgggtgcgtcggagcgctttcggcggacggagtggcaggcagcgtcgatagacgaggtgcggatgtgatttggagtgtgaaggttgtgtgatgtggggggacaggggtgctgcgtccacgggagctgcgggatggggcacttgctcaggagaagcggcggagcggctgaggcggcatgcagcagcttgcgcgctgcgcggccggcctcaaggactcgagcatgttttgtggggtcgagtagttcacaaggcccaaagatcaattatagtgaagcgtgccacgtgggtgcgggaggcggcacagagttgtcgcaagctgcggaaagtagaaacaagcacagatatagtacacacacatgtgaggtggcaggcggcgcgccggctgccagcccccggaattttcccgacttgccggtggagctggggctgagcgcgggccgcgtgagtgccgtgcttgtgtgcatttatcacacacagtgacacggctggcgaggactcgtgaatgtgtgtgtggcttgtcgtgcggctagatggcggggcacggcacatggggtggcgtgtgagtgcaacaggcggggcttgcctgtcgccacgcgccgcgcggcggggcggcgggcgtggcaatgtggaggtgtaaacaggaagaaatt

SEQ ID NO: 9 - DNA, 1413 bp, Chlamydomonas reinhardii
atgtgtaggcaccatgtcgcacaggaagttcgaacaccctcgtagcggctcgctgggcttcagcccccgcaagcgctgccgccgcggcaagggcaaggtgaagtcgttcccccgggacgatgcctccaagcctgtgcacctgactgccttcatgggctacaaggccggcatgactcacgtcgtccgcgatgtggagaagcctggctccaagctgcacaagaaggagacctgcgagccggtcaccatcatcgagtgccctcccatggtcgtggtcggtgcggtcggctacgtgaagaccccccgcggtctgcgctcgctgaacaccgtctgggctgagcacctgagcgaggaggtgaagcgcaggttctacaagaactggtacaagtccaagaagaaggccttcaccaagtacgccaagaagtacagcgacggcaagaaggccatcgaggccgagctggccgccctgaagaagcactgctgcgtgatccgtgtgctggcgcacacccaggtcaagaagctgggctttggcgtgaagaaggcccacctgatggaggtccaggtgaacggcggcactgtggcgcagaaggtcgacttcgcctacagcatgttcgagaagcaggtgtctgtggacgccgtgttccagcccaacgagatgatcgacaccatcgccatcaccaagggtcacggagtccagggtgtcgtgcagcgctggggtgtgacccgcctgccccgcaagactcaccgcggtctccgcaaggtggcttgcattggcgcgtggcaccctgcccgcgtgaagtggactgtggcccgcgccggtcagcagggcttccaccaccgcactgagatcaacaagaaggtgtacaagatcggcaagaagggcgacgccagccacctggccaccacggagttcgatgtgaccaagaaggagatcacccccatgggtggcttcccccactacggtgtggtcagcgaggactacctgatgatcaagggctgcgtgccgggcaccaagaagcgcgccatcaccctgcgccgctcgctgctgccccagaccagccgcaacgcgctggaggaggtcaagctcaagttcatcgacactgcctccaagtttggccacggccgcttccagaccaccgaggagaagctcaagaccttcggccgcaccaaggcctaagcgcttgcatggactgcttcctcgtgaacaatctagcggttgctgcaaccccctagctggcgtgctcagcctggggcgcagcctggcagggctcattgtgcgtacggtggcggactaaaggaaggatgcggggaagccccttcgtgacttttgttaattgccctacgtgttaactgcggcctcgctactttggtggcaatgggttatcaagaggaggacacacttttatgaccacttgc

SEQ ID NO: 10 - DNA, 1192 bp, Chlamydomonas reinhardii
atgcaagcagctagagcaaatgggctataacatgggtattcgcctggtggacgagttcctggcaaaggccaaaatctcacgctgttcctcctttcgcgacacggcggacgtggtggcgaagcaggcactgcccatgttcctcaacgtgacggccaacgtgaccaactggagccccgaccagaccgagtgcagcctggtgctgacggacaacccgctggctgactttgtggagctgccggacgagtaccgcgagctgcggtattgcaacgtgctggcaggcgtggtgcgcggagcgctggagatggtgaacatggaggtggagtgccgctgggtgtcggacatgctgcgcggcgacgactgctacgagctgcgcctcaagctcaaggagcaccgcgacgagaagttcccctacaaggacgacgactaagcctgtagggccggagcctgtagggccggagcctgtagggccggagcctgtagggccggagcttgtaggggtaggggctgagtctagcggagcctagcggagcctctggtggcagcgctcgcatggagagacggggagagaggggtggtaggacggagatgatgacgactggcggttctctctgggttgttgctgctcccttggtcgtggaaatctgctcagttggcgtttggcagggtaatagagcagtgtacgatacacagcaagataagtaagagcaacttggcaagaggcggtcctagcgttgactaatgttgcttcgacagcgacgcagcttgggtatgtgcggtctgggtatgcggctagctgagtggcttctgcggcgacacaggtgaccctttccgctggtacctgacgtacccttcatgcgccacaggacacgtgtgcggtgcagccgcttgtgaccacgtgtgcgttgcagtcacttgcgtgcgcggggccggcgagagcaggatggcacatgctggggagatgggcagtgccgcagtgggtcagcgtgtggccgggttctcgtctggagccctcaccctgcaacttgtcccgctctggcgtcatgtgcggcaggggttagtggtgacctagttgattgatctagtggtgaagtggggagacgtgaagatatggtagctggtgatagcacgagtgggcacatcggctggaaggggctttactggtgctatgagcaactggtgttattgtgtattg

SEQ ID NO: 11 - DNA, 1003 bp, Chlamydomonas reinhardii
atgttcacaatggcgccgaagggcaagaaggtggcccccaccccggccgcggtgaagaaggcggccgccccggccaagcagaccaaccctctctatgagaagcgggctaaggctttcggcctgggcggtgcccccaagccgaagcgcgacctgcaccgcttcgtgaagtggccccagtacgtgcgcctgcagcgccagaagcgcgtgctgtcgatgcgcctgaaggtgcccccggtcatcaaccagttcgtcacccgcgcgctggacaagaactcggcggagacctgcttcaagctgctgatgaagtaccgccccgaggacaagaagcagaaggctgagcgcctgaaggccgaggccgctgcccgcgaggccggcaaggaggctgagaagaagaagcccgtcgtggtcaagtacggcctgaaccacatcaccactctggtggagtcgggcaaggcgcagatggtggtcatcgcccacgacgtggaccccatcgagctggtgtgctggctcccagccctgtgccgcaagatgggcgtgccctacgcgattgtgaagggcaaggctcgcctgggccagattgtgcacaagaagaccgccaccgccctggccctgaccgcggtgaagaacgaggaccagcgtgagttcgccaagctggtggagagcttcaagagccagtacaacgagggcccgcgcgtccagtggggcgggcacatcctgggcatgaagtccgtgcacaagcagaagaagcgcgagcgcgccatcgccaaggagctggcgcagcgcgccggcgtgtaagcgcctcacggcggcaaccgctgtgcggcctggcgccgtcgcgcctgcttcgtgcgggaggcggcggcggacacgcatgccgctggtgggccccctgtcggaggagccagcggcgggggcggctgtggtggagcggtccgcgcagcagccaacgtcgccgtttgctagtggcgcggctctcggaagtgtaatgggttcaaagtgacct

SEQ ID NO: 12 - DNA, 791 bp, Chlamydomonas reinhardii
atactctgcgttgataccactgcttccccgcaccaagggtggcctgggcggctgcaagtaccccctggtggctgacctgaccaagcagattgccaaggactacggtgtcctgattgaggacggccccgacgccggcgtcaccctgcgcggcctgttcatcatcagccccaccggcgtcctgcgccagatcaccatcaacgacctgcccatgggccgcagcgtggacgagaccctgcgcctggtcaaggccttccagttcaccgacgagcacggcgaggtgtgcccggccaactggaaccccggcgccaagaccatgaaggccgaccccaccaagtcgctggagtacttctccacgctgtcgtaaagcaactcgccccactaggaggagtttgttgcggagggtcgatggtcgttgtgagttgatgtggacgttggcggactcggccttggcggatgagagcaggatttggtgcccaggaagcagacgggagtgcgttctgcgcacaggctgtgctcgtgtgcttcaggacggacgcctatgactgccgggtcgcaggatgggttgcatagtgcgcaatgcacttgcttaatgggtaatgcacgccgcgtaggtgggcaggtacaagcaggctccgagccagcatacccatcaggtactatcgcataaagccaaacagagcatctgcattggaagctcttgggatgcgttggaaatgtcaacgccgttgtgcacccgtgtttcaatcaagccacggtccttgggccttgtggcgctgtaacatttgacgatg

SEQ ID NO: 13 - DNA, 1755 bp, Chlamydomonas reinhardii
atgggcggccgccatgatgcagaagagcgccttcaccggcagcgccgtgtcctccaagtctggcgtccgcgccaaggctgcccgcgccgtcgtcgacgtgcgcgcggagaagaagatccgcgtggccatcaacggcttcggtcgcattggccgcaacttcctgcgctgctggcacggtcgccagaacaccctgctggacgtggttgccatcaacgacagcggcggtgtcaagcaggccagccacctgctgaagtacgactccaccctgggcacgttcgccgccgatgttaagatcgtcgacgacagccacatctcggtggacggcaagcagatcaagattgtgtccagccgcgaccccctgcagctgccctggaaggagatgaacatcgacctggtcattgagggcactggtgtcttcattgacaaggttggcactggcaagcacatccaggccggtgcctccaaggtgctgatcaccgcccccgccaaggacaaggacatccccaccttcgtggtcggtgtgaacgagggcgactacaagcacgagtaccccatcatctccaacgcctcgtgcaccaccaactgcctggcccccttcgtcaaggtgctggagcagaagttcggcattgtcaagggcacgatgaccaccacccactcctacaccggtgaccagcgcctgctggacgcgtcccaccgcgacctgcgccgcgcccgcgccgccgccctgaacattgtgcccaccaccaccggtgccgccaaggccgtgtcgctggtgctgcccagcctgaagggcaagctgaacggcattgccctgcgcgtgcccacccccaccgtgtcggtcgtcgacctggtcgtccaggttgagaagaagaccttcgccgaggaggtgaacgccgccttccgcgaggccgccaacggccccatgaagggcgtgctgcacgtcgaggacgcccccctggtgtccattgacttcaagtgcaccgaccagtcgacctccatcgaygcctccctgaccatggtcatggacgacgacatggtcaaggtcgtggcctggtgcgacaacgagtggggctactcccagcgcgtggtcgacctggctgaggtcaccgccaagaagtgggtggcgtaaatttgctgatttgtttcggcaggtttgtgctagctggttcttagttgcttgtgtctcagctacttgcggccaagtgctaggttggcgagcggcgtatggccggggcgcgtagtcgcgtgcgcgctgagcgcaggcgcgcggggccccgccggcgcccttgcgtgcctgcgggctgtggggagtgtcgggttggctcgcaaggcacccttgtattatgatctgatgcatgagaaggggccagtgcgtagtggtctggtgagacaaatagggcgggatgtcgtgccgcatctgcgatagatatggcgggcattcacgcgtgcgcacgcgcgcgcgcgcctgcgcctccctgctgggggcgtgtgggtcctggttttgatgccttgcttgctgccgcttggcgccgatttttgaatgacggcgccggaggcgcacgcgggtgagggattcggttcgtggttggtgtcgtggtttgtcggatgccctcgagatggggcgagtgtacagcaagcggcagtaggtgctccgagcaccaccggtttcggtcactgaggagtggacctgtgagtgagttcagatgtgtttggagtggagccgagagcaagcggagatgtaaagatacacaaatc

SEQ ID NO: 14 - DNA, 890 bp, Chlamydomonas reinhardii
atactctgcgttgataccactgcttccccctggacgagaaggtggctgccctgcacctgcccaagctgggcgtgaagctgaccaagctgtcggccgaccaggctgcctacatcaacgtgcccgttgacggcccctacaagcccgcccactaccgctactaagcggctcgcacttgcgcgcgtgtgtatgaggcggcatgctcccgcgcggagctggcttcggctggtctgtgtgaagcggggcgctgcgtgcggtctcggtgatgggtgaggcggtcctgatttgcttcagtggtgtgagctgcggcggttgcaggcgagcttggcgttccacgcatgctgtggagtgctctggcgcgttgcaacggctggcggttgcagcgggagcggtgatgcaggatgctcgtgagcgccgagggatcgtgtgcacatacattaaacagtacggcaatggggcagaaccctgccctgcgcagtgacatgtggatcgtcccctacatgttcgtggtggtagtgttcactagtgtcgtttggtggtgtttgttttgctggtggtgttcgatgtatgtgggtggtcagtgtatggagccggtgggttgcagctagtttgcaacactgaagtatgatgcggatagggacggctgcgccggagctgcttgccttggtcggagagcgtatgtcgtgtcgtgaccgactgggcggcgtgcgctgacggtgcgagtcaaaccgtgcggccgtatgtgataatttaggattcccctgttcaggggcaatgtagcaaagagctggtattcggacatctgtgaccgccttcgtgcggacgcgtgagggggctcgcacatgagctgtacgcagtgcgcaactgctcgtgatgtaatgccccccttggcc

SEQ ID NO: 15 - DNA, 1184 bp, Chlamydomonas reinhardii
atggcaagcggggctccagctcgagcatcgtcagcggtggcagcaccggcggtgcaggccacaccagcggtggaggcagcaccggcggtggaggcagcaccggcggtggaggcagcatcagcggggcaggcagcgccagcggtggaggcagcgccagtggcggcagcagctccagtggggacggctgcaccagcagtggcgacagtgctgggcgtgcccagcgtgccaatggtggaggcattggtggaaccgggagcagcggaaacacggagcacatggcggcaccccaggctgcccgcccgcagcgcgccggaaagggtgcgaagctgtcggcgctgctggccgcggagcacggacggaggcccaggagcggcgcatagacgtagccaggtgactgcagtcagcatcatgcgccaacgccgttgagttgtgtttggggcgcgccgcggagacctagccgcggcgcatagacgtagccaggtgattgcagttagcatcatgcgccaacgccgctgagttgtgtttggggcgcgccgcggagacctagccgcggcgcatagacgtagccaggtgattgcagttagcatcatgcgccaacgccgctgagttgtgtttggggcgcgccgcggagacctagccgcggcgcatagacgtagccaggtgattgcagttagcatcatgcgccaacgccgctgagttgtgtttggggcgcgccgcggagacctagccgcggcgcgaagacgtagccaggtgattgcagttagcatcatgcgccaacgccgctgagttgtgtttggggcgcgccgcggagacctagccgcggcgcgaagacgtagccaggtgattgcagttagcatcatgcgccaacgccgctgagttgtgtttggggcggaagcctagccgcggcgcatagacgtagccaggtgactgcagttagcatcatgcgccaacgccgctgagttgtgtttggggcgcgccgcggaagcctagccgcggcgcatagacgtagccaggtgactgcagttagcatcatgcgccaaggccgctgagttgtgtttggggcgcgccgcggagacctagccgcggcgcgaagacgtagccaggtgactgcagttagcatcaggcgccaacgccgctgagttgtgtttggggcggaagcctagccgcggcgcgaagacgtagcc

SEQ ID NO: 16 - DNA, 1706 bp, Chlamydomonas reinhardii
atgcctgcttaagcggcctcttcagctttcaaaacagctccttaaccgttttctttttcgcgggcctgaggccctgttcgaagcacaaccagcaaacatggctgccgcgccggttgagcgcaccggtttcgatgaccgcgccttcgacacgaagatgcagcagttcctgggcaacaacgaggacaagttctacaccgactgggaggagagctttgagagcttcgaccagatgaacctgcacgagaacctgctgcgcggcatttacgcgtacggtttcgagaagccctcggccattcagtcgaagggtatcgtgccgttcaccaagggcctggacgtgattcagcaggcccagtcgggcacgggcaagaccgccaccttctgcgccggcatcctgaacaacatcgattacaactcgaacgagtgccaggcgctggtgctggctcccacccgggagctggcgcagcagattgagaaggtcatgcgcgccctgggcgacttcctgcaggttaagtgccacgcctgcgtgggtggcaccagcgtgcgtgaggacgcccgcatcctgggcgccggcgtgcaggtcgtggtcggtacccccggccgtgtcttcgacatgctgcgccgccgctacctgcgcgcggactccatcaagatgttcaccctggacgaggccgatgagatgctgtctcacggtttcaaggaccagatctacgacatcttccagctgctgccccccaagctgcaggtcggtgtgttctccgccaccctgccccctgaggcgctggagattacccgcaagttcatgaacaagcccgtgcgcattctggtgaagcgcgatgagctgaccctggagggtatcaagcagttctacgtgaacgtggacaaggaggagtggaagctggacacgctgtgcgacctgtacgagacgctggccatcacccagtccgtcatcttcgccaacacccgccgcaaggtggactggctgacggacaagatgcgcgagcgcgaccacaccgtgtccgccacccacggcgacatggaccagaacacgcgcgatgtgatcatgcgcgagttccgctccggctcgtcgcgtgtgctgatcaccaccgacctgctggcccgcggcattgacgtgcagcaggtgtcgctggtcatcaactacgacctgcccacccagcccgagaactacctgcaccgcattggtcgctcgggccgcttcggacgcaagggtgttgccatcaacttcgcgaccaaggacgacgagcgcatgctccaggacatccagcggttctacaacaccgtgattgaggagctgccctcgaacgtcgctgacctcatctaagcgctctactaccaccaaatgtgcgctagcccctgggtccagccgatcccaagcacggggcgaagcctgcaggatgtgaagggcacggggcagggcgcgccgccacgcagccgccccgtgacccgacaacgcgctgtacgtgctgcgctgcgagtccttttgaacacgggccgtcgttgcgggtggtaaacgtgaacctgtgttgaggcgggtcggcgcagcttcggttgcagccatttgagagagaaggaagtttgccggtttgcaaacggcaggagttttgtcgcgaccgccatatgtttgacgccttttgagagtgccacaccgctgcccaacctattcgtatgactgtaaggacgcaagtctg

SEQ ID NO: 17 - DNA, 1370 bp, Chlamydomonas reinhardii
atgataagcttcatccgtcgtctgctcctattcagaccaaaagggtcccgatcccctgtgcgttttgctagcgtccagctcaggttcttgcaacaatggctgcaacccagcccagttcaaccgtgcccataccaggctccgttaacaagagcatcgccttccgagtcggtcggaacgcagctgccctcaaccagccctgcttcggtcagtcgcggcgaatgaatcgtgtcgtcaaggctcgcgctgtcaaggccgtcggcctgcggcagcagcggaaagctgtctcgccgcagtcgccaccctctcctaaccaggaaagcgccatgaacgcgcggacgggcaaggtggccttcggcccgtggctgggccctttcgagacgcaccccagccaggatgtggctggggtagaaagtccgatcttcagtgaccgcaagggcaacacgtacgcctcgttgggcggttcgcgcgcgcctccggtgcagaccgccgcgaccgctgcggcgctcggcgccaagggcacggccgcgtccaacgccgccaccaaggagctgtacgccatgatgaacaagatggtgcagcggcacgcggcaaacgcgccccccgtatcggggcgggcagcgcactggcagtcggggcgcagcggcaagatcatgggcaagcacggtggcggcggccgctgcggcgcgccgcgcaccattaaccagccacgccgcagttaatcgcatggccggtatctcgtagcggtgggcggctgccattaggcccccgcttcaaggccactgcggtgccgcggcatagaacgtcgggggatgcgcgctgcagcgcgctccaatgactgtaccatgatgttacatagcacccagtgtacacgatgtattgaagttgtaagaagcacatgctccagcacatgtgcatggcgtggacatatctcgtcggtccgatgcaagcgcttagcagtaggtacatttccgtctgcgctgcatggttacaataccgtcactttgtttaggcggaccaagctccttgtttttccacttggcgcctacgtgccatgtgagacgggcgggcggcctgtgttgttggggttacgttgagcaaagttgcaacgctaagcaactgtagtcgtgcgtcgtgggtgctggtaatggagctggtcgcaggttgcagagatgaaacaagcaggtagccagggcacacggaggtgctttggtagagatgacgcagtcattgacgctggatcggatacagcgaattaggtgcaaagggtttgacacgagccggtggttagaagcgagagcgactcggctttcccagcctgatagcggcacgtgccctgaatacgttagtgccatgtaaccggccgaagaacg

SEQ ID NO: 18 - DNA, 896 bp, Chlamydomonas reinhardii
atgacaaggtgttcggtaccgccaccaccgagagcgagcagaagaagggctacaagctgccccgcgcctgcatggccaacggcgacctggcccgcctgatcaacagcgacgagatccagtcggctgtccggcccgccaagagcgccggccccaagcacgcgccgctcaagaagaaccccctgcgcaacctgggcgccatgctcaagctcaacccctacgccaaggtggcccgccgcgtggagatcacccgctccaccaagaaggccgccaagcgcagcgagaagctggccaagatcgccaaaggcgagaagaccggcggccagaaggacaaggccatcaaggccatcggcaagaagttctacaagaacatgctggtggagtcggactacgccggcgacgactacgacctgttcgcccgctggatcaccgtgcagaagcagaccaagaccgcctaaacggcgtcgcccctggaaagcttgccggcgacaggccctgcgtctggcgctactgtcggagaatgatgatgggttttgtgctttcgctgccgtccaggcttttgcgtcgtgtggccgcggttggggtttgcgtctcggcgggggctggggtccgcgggctctggcgtctgcttggggcgctctgccgccgtgggcgcacggcgcgcgggctgtttgggccgatggggggctggtgccgggccgcaggctgaggcgctagcagctgctgtgttttgtttagtatgtgtgtgcacgcggttggcaacctggtcaggcgagacaatctgctcctattgcaaatgcgcgtgaggaacgcacgcgtggtgctcaaagcacggcgcctccgctgagctcgaggcgaggggtccttggccacaactttgtgtaaaaggatcttggcgt

SEQ ID NO: 19 - DNA, 593 bp, Chlamydomonas reinhardii
atggcctgcccctccgtgcgaaaatggtgctctccagtgatatcgatcttctgaaccctccagctgagctggagaagacgaagcacaagcggaagcgccttgtgcagagccccaactcgttcttcatggacgtcaagtgccagggctgcttcaacatcaccaccgtcttctctcacagccagacggtcgtcatgtgcgggagctgctcgtctgttctgtgcacccctaccggtggccgcgcccgcctgaccgagggctgctctttccgccggaagagcgactaagcgggaggatttgggcgccacctggcgccgggagccgggccttccgctgggctggcggcgggggtgtcgggcagtagaacagcgttcaccgtgagcatggcagacggtcagctggagcccagccccagccaggacaacaggagtagcgtgtaggcttctttttgaggaggggccttcgaacggacccgagctgccttggtggcaggggctccgtgtccatagccggtatgcgcgcatgttggaccatgcctggcaagccgccgtgcggtttgctgttgaccattgttgcgtgtgtaacggtcgctgcgt

SEQ ID NO: 20 - DNA, 755 bp, Chlamydomonas reinhardii
atggtcactcaacatcttaaaatggccgccgtcattgccaagtcctccgtctccgcggccgtggcccgcccggcccgctccagcgtgcgccccatggccgcgctgaagcccgccgtcaaggccgcccccgtggctgccccggctcaggccaaccagatgatggtctggaccccggtcaacaacaagatgttcgagaccttctcctacctgccccccctgagcgacgagcagatcgccgcccaggtcgactacattgtcgccaacggctggatcccctgcctggagttcgctgagtcggacaaggcctacgtgtccaacgagtcggccatccgcttcggcagcgtgtcttgcctgtactacgacaaccgctactggaccatgtggaagctgcccatgttcggctgccgcgaccccatgcaggtgctgcgcgagatcgtcgcctgcaccaaggccttccccgatgcctacgtgcgcctggtggccttcgacaaccagaagcaggtgcagatcatgggcttcctggtccagcgccccaagtctgcccgcgactggcagcccgccaacaagcgctccgtgtaaatggaggcgctcgttgatctgagccttgccccctgacgaacggcggtggatggaagatactgctctcaagtgctgaagcggtagcttagctccccgtttcgtgctgattggtctttttcaacacgtaaaaagcggaggagttttgcaattttgttggttgtaacgatcctccgttg

SEQ ID NO: 21 - DNA, 1603 bp, Chlamydomonas reinhardii
atgttcatttgctgtcttcatccaacaagtgcacgcgcgctagccgcttgctgcgtatcctcgccacgcgaactgttcttcgcgaggtcgttgagttgagtcttatcaacttgtagctctgttacagacgccaccgggagctctctgcctccacaatgctcagccagcagctcgcacggcgccaggtttccagcgcgcggccgtctacgcagagacctgtgtgctcacggccatgtgtccgcgctcgcattttccggcgagacgggcctttggctgttgaggaagtggctgtggacgagttcaatgcacctaccaagcgccagcgggagcagctaaagcgtacgcttcaggagcttggtttcactgagcaagcggctgacatccttgcattcagcaagatcaccaactcgaactgtgacctgctcatcggcgacatccgcgcctcgttcggcgtgtaccaggcgcccccgcccgtgggccccgtggagtccgtgcagaacggcgtgcaggacctgctggagcggctgcgcctgcccttcctgggcgtgttggtcgccgcgggcatctttgcagccatcagcaaccaggtaacgatgctgggctagaggagtcggaggaggagaatggagaagggcgcggaggaggagtggagcgggatacgcgggcaggcaaggagcgggggagcggcgccggcacaccgtgtgcagcccttcccgctgcctgtctgacgccgcatccgctgccgaaagtccattatgtaacgcatgcggcggctaccgctggtcaccgctgccgttgccacctccaagtcgccagggacggtatgatggttgcgtgtgagttaaggcctaggttttggacgcggtgtgctcgcttcagcactcaactgtacatcattcacgcccgcaatgcggtgtcgctgggttcaaatccgggcacgcgctgaagttacggaaggaagctggatacgcatatacgcatggcgttaggacacgcgtgaagcggttgcaaatggacatcagtaatgggtgttggatatcgtaatgtcttttcaggcgcgtatgctttggggtcgagcgcacacaagctgtacggtcccggggccggcacaacgcgaggtggataggcacaagttgaattgcggaaatctacaaggcaggtggcaggcagcagagattgctagtcgtgttggtaactgaatgctggatgttgcacatgcggcccaaggacccgacacatcgtttgttcggtgtgggtgcttgttaggggcccgggtgttagatatttgatcggcaggataggtggggagcagacacgattacggtataagtactgagagtttgtgcgagccatatgcatattgtttattacggcaagggaagcggtgatcccatacgggtgtacccttagtggaagatgggcgtggcggccggtctgtcgctgccggccgaacgcgcgcatccacacgtctggaggtgactgtcgattccttacgttgcgacgctttgctgagatgcaggggcacggggcgcaagtaattgaggccgcgctgtgccgcaattggccgagaccccaacgtgtggccattgtaacacggagagcg

SEQ ID NO: 22 - DNA, 1298 bp, Chlamydomonas reinhardii
atggtacgcatacagtattactagactgccagcaagctaggcagtaaaatggcgaccgcaatgttgagcaagactgtgcccggtgtcctgctaggtagcacctctagccgccggagtcccctagcattcagctcagggtgcacgactcgcgtgctgagggcgctggcgccccgcccagctccggctggccccacgtcgtctggccgggctgtggcgctggtagtgcgcgcgagcaacgaggagaaggaggtccagtacaacaaggagttcggatactctcgcaaggacgtcatccttatcggcgtgggcctcatcgcgctgggctatgcgctctactacgggctgcaagccgggggcatggagccgggcatggccggcaactgggtgcagctcatcatcttcatgggcatctgcgtgggctgggtgtccacctacatcttccgcgtggctaccaagcaaatgacgtacgtgaagcagctggagcagtatgaggaggcggtgatgcggaagcgcgtggaggagatgacggaggcggagctggagcagctggcgagcgaggtggacgccgacaagcagcgcaaggcagcggcgcgcgccgcggcacagcagcagcagcagtaggcgggcaagcagtgaggcaagcaatgggccgagcaggcggggggcggcacgtgcccgtacagcctcgtacatactgaacaagtgtgaggtggcgtccgaaccggttgcctggatgatgcagaatgggcatgagcaccggcgtgggcatgcgtgttgctaccgcacaagctcttgtgtgttcatggcctgagtgcggcttgacacggtagtcatgatggaaagggcaggtcatgaagcggccgctgacgctccgttgtgcgtggcttggcaccttccaagcacgtgcggggatccggcactaaagcttttagaggcttcgggcggtcatgattgtcttttgagcggcgataagcccacgggcattaggcaggtagcgccggttgtttgtagtagtcgcggcaatggccttcaggtgatgaatgaggatgctcgggtgtggccgtcgactgcaagcccaaatggaggtgggggccggaattgagtttattatgcaggcgacataatgctgaggattgcatggcagcataatggtgctacggggcagagcaagtgcgcccacgaggttagtatgagagagtcagcggctggacgcgtgtcttttgccgcttggtgcatgcctttggaatgaacggccgacggcagagaagttccccgtggcctgtaaaacaacagggcgg

SEQ ID NO: 23 - DNA, 942 bp, Chlamydomonas reinhardii
atgggctaagcccgccaagcacactgctaaggaaattgcccaaaaggttgcagcggcaacgacaaacaagggtggtggccaggcgggcctcgcggaccggttgggcggcaaggttgggcacgccaagttccagtgcaacatttgcaagcagcaggcaccggacctaaagagcatgcagatgcactttgaagcccgccaccccaaggacctgtgggagccggagaagtgcacggacctgcacgccatggtgggcggcgtcaccacacagggcgtggcggtgcgcggatctaccaagaagtgagggaactgaaaacgactcgtgatgggtacgtaaggatatgcatgggtgtatgctgaagatgtacacttcgcttccggctctgcgggtttagatgagcgtctcatgaggactcgggaccgtgctgaggacaggccgaacgtttccgtcgtgcgtcatgcactgctagcaggcgtatggggatgcgaggcgccttacgactattagtggtcccgtgtgggttgcagaaccattggacggttctgcattccttgcaaggactgcaggtgggcagaagtaaggggtttcaccaggcgctgacagggcctttttatatgtccgttggacagtgactaggattgacagcgcttggtatggtgaaagaggctagcagtttgcatgtgaggcagatgccgggccgtatgctgggccctgaccgtcgctttcacgtggacgtgtgtgatgggaagtgcgaagtgcgcaggacatggacaggcaacatgggcaacgatgatacaggctaactagagcagtatgtgtgggcgtcagtgagtgaagtggagactaacgctgaccgtcgcttgcacgtggatcagactgtggaggggctcagccccttttcccgtgaccggggaacacttgtcctatgcaaggggaacctcttg

SEQ ID NO: 24 - DNA, 1721 bp, Chlamydomonas reinhardii
atgtacttgtgcaacctcgcacaagtaaccgtcttgtcgcaccgtttcggtcgcaaacactccactcgctaccatggccctgatgatgaagtcgtcggccagcctgaaggctgtgtccgctggccgctctcgccgcgccgtcgttgtgcgcgccggcaagtacgatgaggagctgattaagaccgctggcaccgttgcctccaagggccgcggtatcctggccatggacgagtccaacgccacctgcggcaagcgcctggactccatcggcgtggagaacaccgaggagaaccgccgcgcctaccgcgagctgctggtgaccgcccccggcctgggccagtacatctccggcgctatcctgttcgaggagaccctgtatcagtccaccgcctccggcaagaagttcgtcgatgtgatgaaggagcagaacatcgtgcccggcatcaaggtcgacaagggcctggtgcccctgtccaacaccaacggtgagagctggtgcatgggcctggacggcctggacaagcgctgcgctgagtactacaaggccggcgctcgcttcgccaagtggcgctcggtcgtctcgatcccccacggcccctcgatcatcgctgcccgcgactgcgcctacggcctggcccgctacgccgccatcgcccagaacgccggtctggtgcccattgtggagcccgaggtcctgctggacggtgagcacgacatcgaccgctgcctggaggtgcaggaggccatctgggccgagaccttcaagtacatggccgacaacaaggtcatgttcgagggtatcctgctgaagcccgccatggtcacccccggcgctgactgcaagaacaaggccggccccgccaaggttgccgagtacaccctgaagatgctgcgccgccgcgtgccccccgccgtccccggcatcatgttcctgtcgggcggccagtccgagctggagtcgaccctgaacctgaacgccatgaaccagagccccaacccgtggcacgtgtcgttctcgtacgcccgcgctctgcagaacaccgttctgaagacctggcagggcaagcccgagaacgtccaggcggcccaggctgcgctgctcaagcgcgccaaggccaactcggacgctcagcagggcaagtacgacgccaccaccgagggcaaggaggctgcccagggcatgtacgagaagggctacgtctactaagcggctcgctttccatttcgcaaaacctgcgaaattgctggtcgccagctagtctgtggcgcaagccgcagcagcctgcgacgctggcgataagcgtttagctttggtaggatggcgttttgagtcgtgactcgattgtacattggcggttgcgatgtgtgtcctggccatacaactggtgaggccgcggcgccgctgggatttgttcagacgagcgggtttgcttccatgttgtttggtcgcaaggagcaggcgggagcgtgccaggagcgggtgtggtggtcctgggcgaaggcgtggcgccggcgagtagccgaggtcgcccgctgcagcccacgcccctcgcaatggcggagagcggattgtaccgaggtagagcagagacaaatgagccgggtcatccggcagttttggttggtcccgcgctgcagttttgcactgatcgtggccgggctgtgggtgtggtgttggagttccatcgtcgttacggtgctgtaaggcgctgcaaatgcat

SEQ ID NO: 25 - DNA, 1121 bp, Chlamydomonas reinhardii
atgatttccccgacctatacccttcacttcaccaactcttcaaaatggcctctctgtgcatgcgctcgtcgctggcgcccaaggttgcctcgcgcagctcggccttcctggcgtcgccggtggcccctgtgcgctccgtcgcgcccgtcaaggccgctcccaccaccgtggttgtggaggccaagctgaagacccgcaagtcggctgctaagcgcttcaaggtcactggctcgggcaaggtgactgcccgccacgccggcaagcagcacttcaacgagaagatgacccgcgaccacatccgcgactcgtccaagatgttcgtgctcagcccggccaacatctacaacgccaccaagtgcctgcccaacagcggcgttggcggcaagtaaatgcctaaacccgcagcagctaggagcgaggagcctgggcccatgcatgggcgtgcgaagcgccagccgcagagctgaatgcagctgtgcaagcagcgtcgacaggcaaacgtggcaggcgcataacgtcctggataacaggcccgaaggcagcctgacccatatcgcacgttaccatcggagggcgcggtgcatgggcgctgggtggacgttgagatgaacagtggcggcgcaggagctaagcgccagggacgcgtgccagcttcagcaggagttggcgagttcaaggcgtgcggctgttggccggaaattgggtggacatggagcacctgtcgcgggtggtatgcaagcgctgctgcgcctgcagacggggccaggtgggcgcatgtgtgctgctgcccgcggggttgtgtgttgaaggtgtcggtgtgatgaccggtgggctagattggtcagcgctggagcggtgccgtcttgaaaggcacggatgaggtgagcgcttgagccgcatgccccgcaccgtgcttgcggtggtggcctgtgccgcgccaggaggcgattttgggctgcagccgtctggtgattttgcacgggccgcccccaagttccgtgttttggcgtctgtgctcgcgacgaactttcaacagacgtccaccgttgtgagacgtcggattgcgaacttaggtaacaggtcagtcggagggcgacggcctgctgtaacaccaccaaatc

SEQ ID NO: 26 - DNA, 2234 bp, Chlamydomonas reinhardii
atgcatatgttaagttcataggtttgcgctggaagtagtcttgacgaggtgtggcgttactgcgctcactgcttgcttgcgacaaacccagcggctgctcaaacaactcgcagcgacgtggggtcatcccagcaagtaattgcatgacgggcattaagcgcccatcaaacagtgcggaaccagtgctgtcaaagtccgcgctcgcccagtcgaccctcgccgagttggcactacgcgactacatgttctacctgtaagcaaacagttgcataggcgtcgactggtgaatcgttgtcgtcgctggaacagtgctcggcagctggcgggcgggcatgagctggcggtgtcgatggttggagtatgcgtctcgcacggccacgcgccggactggcggggcgtgccggatgctgcatgggacatcgtccttgggaagctgttgtggagcgacattgcgagcgctcgcaccgcctgccgctcctgggccgtggacgtgaaccgctgcctgcagcggctgcacctaagggaccccagccccgccgccctggtgcgactgggagccgtgttcgcacacctgcaccgactgaagctcacggcgtggcagcgcggtggtgtgaggctggacggcgagggcctgcgcctgcctggggccctgggtcgcgtagcgagcctgcgcttgcgcagcaagcagcccggcggcggactgctagccgtggggcgtctggcgctgctgcggagcggctgtagcggcggggatgacggcggctgtagcagtgccggcggtagcggctgtggtggcggctgcggtggtggctgcggcagccgcggcagtagcggtggtggtgccgccgccgccgccgccgccacggtgtcaatgtcaccagcaggctttgcggcctgtgccccgttcgggacagcattgaggtggggtgcgtccagcagcgggctagccactgctattgctgcgccgccgcccttgggtggtggagctggctggctgggtgatggctgtagcagcgttggcggcagcagcagccggaaggccgcgtgcagggagtgggagcagctactgccggtggtggtgctagcgcttcgggagctgcgggggctgcgacgcgtgtccgtggagggctgcagcaagacgctggtggcggcgctgcggcgcgggctacagccggtggcggcgcggcagcagtgcagcagccgtgacgcggcgtcatggtggttggcagttgacaatggcagtagcaccggcagtggcggcagcagcagtggcagcagcagtagtagcgccgcccagggtagaagccgcagtgatgggagtggcgccagtagtagcatgtccagtccggtaacgttgtgatgatgctcgtggtggggcctttgcgggtcttcttagctgggcttagcttcaagccattgtgcgcggtgttgctgcacttttgagtgaggaggtttgcagccacactggggttagtggacctcagattgggcctcagcgagcattccatggtcgttgtatgcttgcttgcaccgtgttgtgacgtctcgagcatactatgtttcgccttgatgacgtgctgcggctccaattatgacagtattattcctgccaggttacggggcagggcaatgtacgcgcaccattagcaccagcaccagcatcagcacctcgccacccttatcctggagtctgcagcgtttgggggcttcaaagcagagcggcaggggcaggaacccaagctgtaccaagctagaccatcgtcccggcaccggtaacctggacacgtcattagtgcttgcacacttggccgcgcctatgccgctgggtagttgcggtgtggtttcttggcagtactctcgtcgtcttgagtcattgctgtggtgcttgtgttcactggggcggcccgcgggcgaaggaagtgatgcaggtgcagcggcagttcgtgacggacgtggaagaggagcatgcatgcggccaaacgggagggtgaggcgaggggcggtgacccggaggccggaggcaactgacctcaaggactcgggatggcagcaggacgcaggggtgcaaggatgcggtgggagaggaagcaagggaggcagaggcgagcgtgatgcggctgccgtggatgatggaggtcggcgtgcgtaccggtgtgtgtcttatagcgccaagctgcatggaaagtgccgtactaatccagccaggccggcgtgctgcaaatgtatttgtgttg

SEQ ID NO: 27 - DNA, 2267 bp, Chlamydomonas reinhardii
atgcggggcgctttgagcgcattcgttactcggccgtaaaccggcctttagaccactcacacaatggcgttcgctctgcgctcgcctggcgccgtgcgcgcgcctgcctgcgcccagcgcgcgtccggtgtgcgtgcggccaagcctggtttcctgcggtctgcggctgttgctcgcccccaggtgcagaccaacgccgcggctctgtccgtgccggtcaaccagctgactgatgaggagcgcgccaacctggcccgcgagctgggctacaagtccatcggccgtgagctgcccgacaacgtctcgctcaccgacattatcaagagcatgcccgctgaggtgttcaagctggaccacggcaaggcttggcgcgcgtgcctgaccaccatcgctgcgtgctcggcctgctggtacctcatctctatcagcccctggtacctgctgcccgctgcttgggccctggccggcaccgccttcaccggctgcttcgtgattggccacgactgcggccaccgctctttccacgagaacaacctgatcgaggacattgtgggccacatcttcttcgcgcccctgatctaccccttcgagccctggcggatcaagcacaaccaccaccacgcccacaccaacaagctggtggaggacactgcctggcaccccgtgactgaggcggacatggccaagtgggactccacctcggccatgctgtacaaggtgttcctgggcacccccctgaagctgtgggcctcggtgggctactggctggtgtggcacttcgacctcaacaagtacacgcccaagcagcgcactcgcgttgtcatctcgctggctgttgtgtacggcttcatggcgacggccttcccggcgctgctgtacttcggcggcccttgggccttcgtgaagtactggctgatgccgtggctgggctaccacttctggatgagcaccttcaccgtggtgcaccacaccgcgccgcacatccccttcaagaaggcggaggagtggaacgccgccaaggcccagctgtcgggcaccgtgcactgcgacttccccaactgggtggagttcctgacgcacgacatctcgtggcacgtgccccaccacgtggcccccaagatcccatggtacaacctgcgcaaggcgaccgagtcgctgcgcgagaactggggccagtacatgaccgagtgcaccttcaactggcgcgtggtgaagaacatctgcaccgagtgccacgtgtacgacgagaaggtcaactacaagccctttgactacaagaaggaggaggccctgtttgccgtgcagcgccgcgtcctgcccgactccgccgccttctaaactagctggctacaattggctatcgcgctagtgagctaatcggagctgagcaagcttctcggagccttgcgagagcttctgatgttggctttatagggctggctaaaacagctagcagctaggtagtgtggctgcgccggccgcttgcgcggccgcgcgagagagggaggggcgtcaggcatgtgctgtggaccgggctgcacgagagcagtgcagcagttgcgctactttgtgtcttgtatgaaaatggttttgaggattggttgtgtgcgtgacggcgcgagatggacggttgcgcgggcggctttgttctgtgtggtgcgtcagtgcgaggcatgccctgacatgagtaggtgaggggtgcgacgagcctgcaggaatgcgtgtgtgtctgtggagcagcggtgttcgcggcagctccgttgtcggcatgcgtttccgctcaccgccccctcgtccttcgcgtaaagtgcttggtatgcctcggcttgcccacgggacgccgaccgtgtgacggctccgcggctgtgaagaatttcatccgcattgcgcacagcctctaacagttggatcgattcttgttatgtacatgtggtgttggacgtggagtgcacagtcagaggcgtgtgtgttcgtgtgtgcgtggagcgtggcggaaagccaggaagggtgacaagagagaggacgcaacagttaccagtgtgtgagccggatgttgggggcggaagagatttactgctctccccccgtcacgtgataagtagtggtgttatgttggttgtcgtgcccagatgaacgtgcaggcagctgtggagttggccctcaattttcccaccaccccatgcaacgtgaagcgggcgagccaccggaggagcgggataccctcccgcgcctgcctgaaacgagtggtgaatgtaaagcaaattacc

SEQ ID NO: 28 - DNA, 728 bp, Chlamydomonas reinhardii
atgtcaagatgggcgcctacaagtacattgaggagctgtggcgcaagaagcagagcgatgtgctccgcttcctgctgcgtgtgcgctgctgggagtaccgccagatgcccagctgcgtgcgcctgactcgcccgtcgcgcccggacaaggcccgccgcctgggctacaaggccaagcagggctacgttgtctaccgcgtgcgcgtccgccgtggcaaccgcaagcggcctgtgcacaagggcattgtctacggcaagcccgtgaaccagggtatcacccagctgaagcccgcccgcaacctgcgcaacgtcgctgaggagcgtgctggccgcaagtgcggtggcctgcgcgtcctgaactcctactgggtgaaccaggactcgtgctacaagtacttcgaggtgatcatggtggaccccgcccacaacgccatccgcaacgatgcccgcatcaactggctgtgcaaccccgtgcacaagcaccgcgagctgcgcggtctgaccgccgcgggcaagaactaccgcggcctgcgcgccaagggccacaaggccagcaagacccgcccctcgaagagggccagctggaagcgccgcaacagcctgtcgctgcgccgcttccgctaaacagcatctgcctcatcatcagctgctgttggcgccgcctgattgcgagattacaccatcaactcttacgatcgtcattctattctctgtaacgaggagggatac

SEQ ID NO: 29 - DNA, 1639 bp, Chlamydomonas reinhardii
atactctgcgttgataccactgcttcccccagggtttcactcacagcgcaacaccctcgagcccggtcccggtgcctgcaccccgttttgcaatgtcatgctgctgctgctgtgtgtgccccgcccaggagacggtggccatcgtcgagaactgtggtaaattcagccatatcgcacacccgggcttcaactgcctgctgtgctgcttgggcgcgagtgtggccggctctttgtcgctgcgtgtacagcagctggatgtcaagtgcgagaccaagaccaaggataacgtcttcgtgagttacgagggctctgggatagggtcagggaagcagggacgtatgtggaggttaagtaggatgtcgggggcgcttcgtgtggcagcggagtccacggcgtccgcgtcacagcgggtcgactggcctgacctggaggtgacgcctgccggcggtcctatcgcctcacgtaaggtatgcacctccgcaggtgaacctggtcgtgagtgtgcagtaccaggtccagcgtgaggcggtctacgacgcctactacaggctgacggacagcaggcagcagatctcggcctacgtgtttgatgaggtgcgcgcggctgtgcccaagatgagcctggacgacacgtacgagctcaaggacgagatcgccaagggcatcaaggacgcactggccaagtccatgtccgagtacggcaacctgatcatccacgtcctggtgaatgacatcgagcccgcccacaaggtgaaggaggccatgaacgaaatcaacgcggcgcggcggatgcgcgttgcggcggcggagaaggcggaggcggagaaggtggcggtggtcaagagcgcagaggcggaggcggaggccaagttcctgcagggccagggtatcgcgcgccagcgccaggccatcatcagcgggctgcgcgattcggtgtcggacttccagaacggcgtcgtcgacatcagctccaaggaggtgctcagcctcatgctgctgacgcagtacttcgacacgctcaaggacctgggtgcgcacaaccgcgcctccacagtattcctcaaccacgcgccgggcggcgtcaacgacatcgccaaccagattcggggggcgttcatggaggccaacgccgccgggctgcctgggtcgtcgggcgcggggccgtcgctgccgcccaagaagacagcgtgagcagctgggcaacatggcaggccgctgctagagctgctgccagttgtcggatccgcgtagtgcgtggatacggcacatccgctgtccacagggttcacgcatagtgtgtgccgtacagatggcctggtggctgcgaatgacaccgtaggttcagtactggataacatgtgttattgggattgacagcagctggcgtggtgtgtgtcaagcagtgggtaagggctgccaagtatttgatacatggcgaggcttgctttcgttgtcctgctgtctttgttagtgcagagtaccgtcataggagtgcgaggtggcatagaaagccgctcacattgcgttgagtgcaatcaagctcatgcatgcccacgtgcgcaaggaacgtggcaagacttcgtcctagaacttcagcagctttcctgtgtttcatcctgcacatataatacttgccgcc

SEQ ID NO: 30 - DNA, 641 bp, Chlamydomonas reinhardii
atgtgatgcccccgcctggcatgccgcctccgggaatgcctccgccaggcatgcccccgggcatgcggccgccggggcagtaagctgcggagggcagcatgatgggcgtcaggagcagtggcagcagcttgacacggtgtgctgtgtcggctgaggcagccggagctgctgcctgactttgtgttcgggggttgtgggtgagattgttttctcgtggatgaatgcggacgtgtacgactggcgtgcgaccgggagcggcatggtttgtgcgctgctcccgtctgcagagctgcacgtgagcttggtgcgcgtcatgcttgcacgtgcagatacctgttgggaagggcggttgacggacgcacggacggccgggcacttgcccgggtggcggaggcccgggtggcggagtttgcgggtttgctccttgcgactggcgcggggcgattcacagctctgcaacgcgtgtgattggaacgtgtctacggatttcgtgtgtggattcggacgcgtcacgtgtaggaatgaaacctggatcaagtgctggtggaaggcgctccgaccatcatcgtgggtgtcggggacctgtggctgaagtgacgaaggtcgcggtcagtggagctcgatggcaaaggaacatatgt

SEQ ID NO: 31 - DNA, 986 bp, Chlamydomonas reinhardii
gacgaaatggtgtccctcaagctgcagaagcggcttgccgcttcggttctgaactgcggtctgcgcaaggtttggctggaccccaatgaggtcaacgagatttcgatggccaactcccgccagaacgtgcggaagctcatcaaggacggcctcatcttccgtaagcagcccgtcatccactcccgcgaccgcgctcgccgctcggcggaggccaaggccaagggccgccacaccggctacggtaaacgccgcggtacccgtgaggcccgcctgcccgagaaggtgatgtggatccgccgcctgcgtgtgctgcgccgcctcctgaagaagtaccgcgactcgaagaagatcgacaagcacctgtaccacgacctgtacatgaaggtcaagggtaacgtgttcaagaacaagcgcgtgctgatggaggccgtgcacaagcagaaggcggagaaggtgcgcgagaagaccatcgccgaccagttcgaggcgcgccgcgccaagaacaaggcggcgcgcgagcgcaaggccacccgtcgcgaggagaggctggccgcgggcctccacctcgaggccccggcgaccaagcccgcccccgcccagaagaagtagacggtccatctagcgggcggcacccatctgccgtctgacggtggtggtgtcaatgagagagttgtcgacggaccgctgtaactaccggggtgaccaacgacccgaatcgtaccgccgcgctgtgatggcgtggcagcgtcaactgggtgggagggcttgcgtgtatagtgcagcagtagcgacagttcggtggcgccactcacgccgcaaccagttcccggagtgcgcgcgccggccaggggcgggtccaccacccgccacggccgtgtcgtgtactgctgggggccgggcgcagcgcttggttccgccaaccacccgtcgggtgcataagaatgacttgttggtgtacgtgttggcaagtgtaacatccccgttgt

SEQ ID NO: 32 - DNA, 1441 bp, Chlamydomonas reinhardii
atgcccctctggcgcgtcgtagcgtcgttggtgcctagtgtaagcggcaggagcccagcgtctcctctccatctcggtataaccctcgcccgtcccggaaagggtcccgacaacatggccaaggaggagaagaacttcatggtggacttcctggccggcggcctctctgctgccgtgtccaagacagctgccgcgcctattgagcgtgtgaagctgctgatccagaaccaggatgagatgatcaagcagggccgcctggcttccccctacaagggcatcggcgagtgcttcgtgcgcaccgtccgcgaggagggttttggctcgctgtggcgcggcaacaccgccaacgtgattcgctacttccccacccaggccctgaacttcgctttcaaggataagttcaagcgcatgttcggcttcaacaaggacaaggagtactggaagtggttcgcgggcaacatggcctcgggcggtgctgccggcgctgtgtcgctgtcgttcgtctactcgctggactacgcccgtacccgtctggccaacgacgcgaagtcggccaagaagggtggcggagaccgccagttcaacggcctggtggacgtgtaccgcaagaccatcgcctccgacggcattgcggacctgtaccgcggcttcaacatctcgtgcgtgggcattgttgtgtaccgcggcctgtacttcggcatgtacgactcgctgaagcccgtggtgctggtcggccccctggccaacaacttcctggcggctttcctgctgggctggggcatcaccatcggcgccggcctggcgtcctaccccatcgacaccattcgtcgtcgcatgatgatgacctcgggctccgctgtcaagtacaacagctcgttccactgcttccaggagatcgtgaagaacgagggcatgaagtcgctcttcaagggcgcgggcgccaacatcctgcgtgccgtggctggcgccggtgtgctggcgggctacgaccagctgcaggtcatcctgctgggcaagaagtacggcagcggcgaggcctaaagcggccattgcatgcctggagctattgctaagaggcggcggtcggatgaggggtacaacagcttcgcaacctgggtttgctctcctaattctttaccatatgcagtgcgagtgagtgttaattaccgtggaaccgtggacactgacatggctgcggaccagcagtgacaacggaggcttgtgtgcgtgtgtggcatgtggtgatggtagcgataacgtgcactcgatgaatgctatcaacctcccaaagacacccatagcatgggtctgtggttggtgcattccgggtttagggtagattgattacggaaccatcggaagattggcatatgaaagccgaatgcggatgaggcggagcatggaccggatgtttcggacgttctgtaacacagacgcggcc

SEQ ID NO: 33 - DNA, 312 bp, Chlamydomonas reinhardii
atggtgaccggcaagggccccctgcagaacctgtccgaccacctggccaaccccggcaccaacaacgccttcgcctacgccaccaagttcaccccccagtaaatgccctggcggcacagttttgatgtaccaatagggatgcaggtctgagcggtttatttgggtcgtcttgtgtggtctggtggagcttgagttgtttgggagcggtgggttttgtgtgcggtctggccgtgcagcaggcaaggtcccgacaggcgcaggagcggctagctgcgctgggacttgtcaacgtttgtaaattttgagagaagt

SEQ ID NO: 34 - DNA, 559 bp, Chlamydomonas reinhardii
atggaccccagtgatgtggtggaggtgtgcgtgcgtgtgaccggtggtgaggtgggtgccgcctcctcgctggcccctaagattggtccgctgggtctgtcccccaagaagattggtgaggacatcgctaaggagaccctgaaggactggaagggcctgcgcatcaccgtcaagctcactgtccagaaccgtcaggcgaagatttcggtcattccctcggcggccgcgctggtcatcaaggccctgaaggagcccgcccgcgaccgcaagaaggagaagaacatcaagcacactggcagcgtgacgatgtcggacatcatcgagattgcccgcgtcatgcggccgcgcagctgcgccaaggacctggcgaacggctgcaaggaggtcctggggacagccgtgtcggtcggctgcaaggtggacggcaagggcccccgcgacgtcacctccgccatcgacgacggcgccatcgagatccccgacgcttaagcgcggggcgtgttgcagcggcagggaagagcagctgatgatgatcaagggtgtgctgtaacgtctgtgc

SEQ ID NO: 35 - DNA, 640 bp, Chlamydomonas reinhardii
atgctctcattttccaaccccaaccttaaaaatgatcgctgctcgcgtctcctcggctaagcccgtggctgccaagaacgtccaggctgcgaaacccaaggtcgcccccgccgtgctgggcgtcttcgccgctgccatgtcgtgttcgcccccattgccaacgccgccgagacggccagcgtccgccaggtgctgtgctcgtccaaccccacctccaaggtgtgcctgaaggactccgccaagaactaaattgcttggcatggcttggacaaaacttcaaggcactggacaagctggcaagccgtgatggcgggttatcctggacggttacatagcttattcccaggggccgggcgcgggcagagcgtgccgtttttcaacgctggcgcgcacgcgttgcgggtttttgagatggtctggtgagcacggtttaagcgggcggcgaagagaggttgctgcagcgagctgcggcctcgtattgccgtgtgtacactataaatgtacaacaagttttgcgtatggacggtacgcacactggtggatcatgcgttgagagcagggaaggttcctggttgagagacacaaacgccatccgacagtggcgattgcacattgtacctgtgtaaggttaaatgagctt

SEQ ID NO: 36 - DNA, 1843 bp, Chlamydomonas reinhardii
atgcgctcgtcgctgcccactgtgacacgaagcggcgtttgcccttgtcttgtttgccaagctctatctcataccaaggcataacaatgtcgcctgtcgccgagcccttcgctatggacgtcgagagccagtcctccgatggcgccaagactaaggcccagctcgtggctatggccccgattaaggacgagtgggtgcgcggcgaccctggtccgttcggcctgctctgcttcgggatgaccacttgcatgctgatgttcatcaccaccgagtggaccaccaagggcttcctgcccacggtcttctgctacgcgatgttctacggtggcctgggccagttcgtcgccggcgttctcgagctgatcaagggcaacacctttggcggcactgcctttgcctcctacggcgccttctggatgggctggttcctgctcgagtacctgacctggaccaacaaggccctgtacgccggcgtccagagcggcaagtccctgtggtgcggtctgtgggccgtcctgaccttcggcttcttcatcgtgacctgccgcaagaacggctgcctgatgaccatcttcagcaccctggtcatcacctttgcgctgctgtccggcggcgtgtgggacccccgctgcgagcaggccgccggctactttggcttcttctgcggctccagcgccatctacgccgcctttgtgtttctgtacaagattgagctgggcatctccctgcccggcgtgcgccccgtcgctttcctgtaagcagctaactaaagtagctctggagcagcgggtttgccttccatggctgtgtcagtaatgccatgctgacggcctaggcctctgatgatcgtttcggacaataactacttcgtctaacacatgctgtattaaggcgcttcgataacaacccattcggtgacagctcggtagcggcggcgccatgctgggtgtcgtgcctacctcggtttgaggctcgggaagtgcaagggacttcgacagcgcgaagatgatttgcgtggaggtggatgtataaagctcgttaagagggccgcaggagtgtttggcctgggttgcggttgtttgtcacatgcagtggcatgtatggtattgggtaccgttgggttgtcttgggcaaaagcatgtgcaatgtgtatatgtgtgcgtgccccgtgcacgcacgccatggtgcacactcatggtgcttgtgccactttccaggccttcgtgatgaggtgactgcgagaggtagagacactcgtgtggtttgtgcggctgctaatcaccaaatggtgccgccagtttgacaaatgatgacattgcgctgtgtcacactttgtatgaagcccgtcgtgcgggcaacaccccattttggcgcgtgcggctgttcagtgcggaatggcgcacgagcacgcatgcttgcggggaggtggcatagttagttgctagttgtgcgtggtgacgtgtctgctcgccccgtgcccgtgcctgcgtttacatacgttaagtggtgagcactggccgggacgagtggatgtctgtgtttgcaaactacaaagggtatatgggaatgatgtgaaatgtttggttggggttttatgtgctggggtcacccataggcgtctgcgcaaccaacgccgccacctgcgcctgcaggccctgcattctctagggctgcctctagggcgccttggcgcgcgtgttgttggcgacgccgtgacctgtgatgtatggtttctggctggatggcgtaaatggcggattcgaacgtgctttaaaacacgcatcagtaaaaccgacctcgatgagcgaaagtggttcgcactcgatttgtaacacgccaagat

SEQ ID NO: 37 - DNA, 1107 bp, Scenedesmus obliquus
atgtcaccttctgcttcgcaacatttttcttggttcgtgcgtgacaccgtctgtgcgcttgttctggttgtggagctcgggcctgcacaggaacccacatcacaaagcaacagtaccacaatgggttacgcaggctacgttcccgccaagctccctccatccaagcccacagaggtggaggccgccgtgcaggcagtcaagtctgttgagacgctggaggtgatgggcaagctgctgtacaactgcgcggtgagcccgcgcgaggacaagttcaggcgcgtgaagctcaccaacaagaaggtggcagacaccatcagcagcacacacggtgcactggaggccatggctgcgctgggctgggtggctgacgaggccaacccgcaggagctggtggtgcgcgagggcaccttcttctccatgaaggaggtgcgcatagtggaggccgccaaggagcggctgcagaaggacatgcgctccagctccagcaagaacctggcagccatggtcagcgtgcaggcgtgagcaacgccaggcagcaatgctgggctggcgcatgtacgtgcaggcacggagctaccaatgtagtgcagcgctggcgttgcggcgtgggaaagtaggtgcagtgatacacagctgcaaatgtacacttcaagaaacctgacatcaaatgcaaagtcaggcctcacagctgcggcaacagcaggctccgagcgctgactttgtgttcatggctgtcttgctgtacactggatacttgtgcatgtggcgctgtgctggcctgccttgcggcaggcagccaccagcacggggcgtgtgctgtgttggtctgagggctgactgtctaggtctcggctgcctgaaattggcagcagtgtgttgcactgaacgagtgcggggatcgcttgtgcgcgccgtaaatttacggcctgcgcatggttggtggacatctgtgcttagagggtcgaaaccttccttgagggcaagagtggtctgcgccacttggcaccaattgtggctgcagttttgagtgtcaaaactggatggtagtggctcatgagtcagctgcagcagctgcagacagggttcactctgtaacatatatgacgg

SEQ ID NO: 38 - DNA, 1199 bp, Scenedesmus obliquus
atgggagactgccaatttgctgcatctttcgctcctcgcagccacaaatcagcacaatggcactctgcgctaagtcctccacccgcgccgtgtgcgccaagcaggctgcccgccctgctgtggcccgccctgtgctggctcgccgcatgaccgtgcaggctagcgcccagaagcagcaggtgtccatgcaggtgactgctggcctggccgctgctgctgccacccagatgctgatgcccctggcagctgccgctgaggtcaccccatccctgaagaactttgtgtacagcctggtggctggtggtgtggtgctgggcggtattgccctggccatcaccgcagtgtccaacttcgacccagtgaagcgcggttaagcggtttaaaccccacgtctgtggcggtttaggcgccaaaatcccagctcgacggcggttcaggaaagaagacctaagccctgtgggcgaagggacggtgtgttgcgcaagcagcaactgtcttaggagcaggttcagtgcttttggatgttggtagtgctggcagtgtgtggtccgcagcgggactgctgcttttggtcaggtagcagcacagaagcagcaggtgtgccggtgcgttgctgcaatgcatagcttcctgctgctagcgctcgagacctagcagggccccgtgtgcggttgtgatagaggagcgtgtgtggccggaatcgtgctgccttttgtgctgctgcgcagacggtgctgtctacaatcaatatcttttggatgtagtttctgtgctggtttgcagccgagcaggcagggcagtggcggcagtttgggggctgtgtgtgcaaagcagcgcaagcgtgtcggcgggtgttttcttttcctgttgcagcagttggtaccttgctgaactgtttgcagggtgatggattgtgccatgctgtatagctggccgcagcagcgcgcacgcctgtgcagtggttggtttcgtgtttaggttttccacctaacgacgcttgcttccagcaatggaagcagagcagtgttgtatggcagttgaggcagcaccctgggagcagcagcagcacaaaattcgtttggctactgtgacagcagcgtttcctgcaagcagcaatgctgtgcagcagcaggagtctgctaggccctttgtgttgcaagagtggcagcacatttgcagacgtgtaactgttgtcaccc

SEQ ID NO: 39 - DNA, 638 bp, Scenedesmus obliquus
atggcagcatgacaccagcagctagctggatgctgttggctgcttttgcagctgacggctgcgcctgcacagggcagggtgctcagacattggcagctgggctgtgtgtcatgcgccgtgtgtagtgcacggcttttttgtatgtggctgctttgctgtgtagctgtttatgtacaatagcctgactgttcaaagcattgtgaatgacatgaactgccagctctttccaccatctccagctgtggcggagtgtgtacaggtcacacacaggacatgggggttactacttgatgcacttcagtcactgtttgtggcttctcgtgtgacgctgccaaggcagccacggcttgcaatgccgccacctgcagttgcattgcacaggtggctgttgtgatggtgtatggactcaactgtggcatatggcctgcaagcatgtacctgttcagacattgctgttgtgcacggacatgcctgggcatacctgccactcctgcttgagcaggcagggggcccaatgcaggacagactgtgcctggcactctaaatggtgactgcatcaagggtatgttgctgggtcttggcaccgctggaatgtgactgccgtagacttcagcgttttcttaacgagttctgccact

SEQ ID NO: 40 - DNA, 701 bp, Scenedesmus obliquus
atggcaggagcagcagcaacaaagccaatcaggctatggactagaagcagaaggtgacggggcagacatggttaggattgacacagacagcaaaggtggattgggaggcacaacagaaaacgtgtttggcccgttggctgtgcttgtggtcggcttcctgcctgaggagtaccaggccttcagggccatgatggtggacatagaggcagacatggtcaaggtggtgccgtgcagcaaggcgctgctggctggcagcctgcagcatgccatggagtcagagtacccgcagtatgagcagccgccgctgggccagcgccgtgcgctgttcctgtcgggcatgtacggatccgaggtggtggagctcatcgcggcttacagggaggcgggtctgccgccatgcgcgtttgcagctgccgtgcccaacaactggcaacgcaacgtgaaggagctgagtgaggcggtgtggcgggaccacgcagcaatgatggcaaagcagcagccgcagccaggcggcattgaagacagctattgagttgtaggtctcgtggctgttgaaattctgtgcttgattagcctatcatgccattcagctgagtggtgtgtgcagcaggctgaagagcagcaccagctgctcgaggtctggtagcagtggcagtgcccttagtgcccgatgacgtcagtacttgaaagttaaaactttc

SEQ ID NO: 41 - DNA, 1081 bp, Scenedesmus obliquus
atgaagctcctttactctttgcagaggtcataccccagcctttcacatatcacaatggcactcgcaatcaagtccgccgctgcccaggtggcatgcgtgaaggccgcccccgtggccaaggccaaccgcatgatggtgtggaggcccgacaacaacaagatgttcgagaccttcagctacctgccccctctcagcgatgaccagatcgcccgccaggtggactacatcgtgaacaacggctacactccctgcctggagttctccatgcccgaggacgcctatgtgtcttctggaagctccatccgcttcggcgctgtgtcttgcaactacttcgacaacaggtactggaccctgtggaagctgcccatgttcggctgctccgaccccgtgcaggtgctgcgcgagattgacaacgccaccaaggccttccccaacgcctacatccgcatggccgccttcgacgccaaccgccaggtgcaggtggccagcatgcttgtgcacaggcccagctccgccaaggagtggcgccccgtgaaccaacgccaggtgtaagtctgccctgcaacagtgtgcctgaagagcccctgctggtagccactgctgctggcgagtccccgaaggccgttgcttggtagccttgccctgtgctgaggaagggaggagctctggtgttggcgctgtgtctttgggtctggcctagccatggtctgtacgccacatgtgcaccctgcctgtccccccactgcccgcttgcttgcgagtggtgcagtgcggcggtggtggttgtggtgtggaatccggctggggtgtgtgctgccctttggcactgtgagcaacacatgtcccccagtgctgatcagctgccagttgcttgcctgagcctggcgctgtgatgcattttggcctctactgttcagcagcgtgcaacggcatcctgttggcagtctagtgtcggctgctgctccagcagcagattggtgtggagtgtgtcggttttgtagccagcattaccgcttggtactgcagttggtgctattctggttgccaattcgttaccgcgtggtaattctggtgtcatgac

SEQ ID NO: 42 - DNA, 1516 bp, Scenedesmus obliquus
atgcttcggtgttgctctgctgctgtgttcgcctctccctgagaagcaagaggtctcattgcctgcaggactgttctgctgatgtgtttttgacttgaggtgcggtgatcagggctccgggcgtggcaggtttcaggttttggggtgtagcaagcctcacgtgtttgtgcgtggcgtcgctgccatgtgttttatggtttggggttgggctgtcatgcggctgcgtcacagctgctcgatgcattcatgcagcatgcagtgcacacacaccgccccggctgcagctgcctttcagtggacttctaatcccttttttgtggagttctaatccctgttatgtgcatagtcgcgcagctgcacatcaacggggtgcatgtgtgtgctctgctatcatgcagccaatagcttgcggcagttgcagtgatgtagggtttcaggtttaggttcaaaccctaaaaccctagacccggttgcggtgatcagggctccgggcgtggcaggtttcaggttttggggtgtagcaagcctcacgtgtttgtgcgtggcgtcgctgccatgttgtgcgtcaggctgcacaccctaacctgatttaggttgaagtttcacacaggagcttgggatatgcttgcatgcatttgactagcgcgtttgtggctgcaaccacgtggtgaatagcttgcagctgcagccacgtccaagagcttgcatggttgttgtgcatcacagcttgagcttgttccgaaactgcggctggagtcaccaaatcaccagctgttccggtgttttggggatgctgcatgcgactggctagctttgtctatgttgatgttgttgttgggtagcgccgggagcgcgtaccaatgaagcagaatgcagcggcgccgggtctgttctcagcgggcccgcgtgtttgtgttataacgttatgacatgttttagggttcatgctgagctcgtttgcaaactttgcccgtgcgaaggcgtggacaggccttgaatgtgttgtggcatggcagctctgcggcttacaaactgtggcaacctgtgatcgatgaaaccgcctgcatgccagcagcgtgttgttcactggtctggcgcactttgcacaaaaaccctcttgcagaaaaccctggtggctgttgcatgtgctgcagctggtgtcactcagggtgtggggttttgggaaattgtctggctggagcaatgcacagcacttgccagtggttcagacagagttcggggacggtcgttttgcagtgtgcacatgcatgtcacatgtacctgcaagcgtgcatgcatgtatgtatgcccagtatggtgtacggcgagcaaggcgggtttcttttgtattcccgtcagtatttattacagtgtccgcatgaaaacgtttgccttgtggaagatccacatgctgtttaggcacgtgagcattccacacttccacagagactgccacacctcgtctgctggccgggctgcgagcgttacatatcaacgtcagtaactgttatgtttc

SEQ ID NO: 43 - DNA, 1516 bp, Scenedesmus obliquus; identical to SEQ ID NO: 42

SEQ ID NO: 44 - DNA, 1775 bp, Scenedesmus obliquus
atggttggctgtggccggcatggtaccttcgcttttacgcctcagcgtgcttgttcgtcagctgatgctagcagctctgggctgggtatactcttccgttgctggaagtgggacgttgcatgcaccgctatcttgcagagcatgaagggagccatttttgaccagagacagtggagtcgttgttgttgcaacctgttagtttgagggtggagcgccgtgtacctctgtttatcatgcccggcccttcctgcagagctgctgcactgctgcacttgcaccttcagcattttgtactgcttcttatcgagatgagagctgtgctgcgcttgcatggctgtgcgcgctgctttgttgtgtgcatggcacagtggaagcctgccagtcactgacactgctgccgtgttgtgtgaaacaggcacactggtatgtatgctcgtggcagcaatgccaatacagttctgagcagggaccttctcagtttgtcacagcttggcatgcgtgcaggctggcggcagttatacttcaccgctcagaggcgttatcatcagcaggtttgccagctgtgaagtgacgtagagttcattcagcatgttatgcatctgtttctggggctggtgttgacaagaagccagctacaaatgctgacacaggcccgcaatatagtatgcacccatatacacacagcagcgcgctttggatgctgctctgtgagtgttgctggaggcatgttacccactcatcatcatgcgcatatgtttgtggtatttaagcgagatggaaaggaccagatcgaccagcgggaagcagctggagatgcgaaggggagtttgtcacagcaatggtcccagcaacattccgggcgtaactgggacgtgtgcttgtggcgctactataggattgagtgctgctttgtacggtttgtatcacccttgtaccaacactctctcccgtagttgcctaacactgcaactcgtcagcctttaagctggacgtatcagctgtacagctaatgtactggcttaaaggctgaagagttgctgtggcacaacaagttgcggtgccaggcgctttcgtgcttttttgtggctgtgcgatatggtggggaaggcttttccgcgagcaaggtctgctcgtcagcttttcaatgtacatcagcactttttgtgtagtgcggcggctgcaccaaggtttgttcagcgttgcagacgagagtgtgctgtttgcctcatctctatctactactgtgtagcatggcgtgggtgcatgtgtgtagtgcgttgcctgttctttgctgggctgccagcagcagtctagtttgtaggcgtcctgtgtgagtacaccatgtgtgtgtgtgtggttagccggttgttgcgggtgtgttttgggcatgtgggtatgtactggttggctgttgtgcatggcggaggtggtgaaagtgtatgaatgtctgcagatgctactattatctggaaggtgggcattttgtgcggcgcctgtcattatgcagtgcaggcgtacatggtgtcagttctgaagtgcatatgtgctggcagcgggcctatgaggtcctgctgagctgccgtgttggctgcttgctgtgtttctgccggcgcagcagacttgagagcaccatgatgaggatgactgtcgggctgtcacgacaggattgatcattcaacacatggtgcaggcttttttgttgctggcagcgatgatgttgttgaaaaggcatgacgtttgcacgagttgctggagcgtaatatcacaagaagccg

SEQ ID NO: 45 - DNA, 769 bp, Scenedesmus obliquus
atggtgtcttgcaactacttcgacaacaggtactggaccctgtggaagctgcccatgttcggctgctccgaccccgtgcaggtgctgcgcgagattgacaacgccaccaaggccttccccaacgcctacatccgcatggccgccttcgacgccaaccgccaggtgcaggtggccagcatgcttgtgcacaggcccagctccgccaaggagtggcgccccgtgaaccaacgccaggtgtaagtctgccctgcaacagtgtgcctgaagagcccctgctggtagccactgctgctggcgagtccccgaaggccgttgcttggtagccttgccctgtgctgaggaagggaggagctctggtgttggcgctgtgtctttgggtctggcctagccatggtctgtacgccacatgtgcaccctgcctgtccccccactgcccgcttgcttgcgagtggtgcagtgcggcggtggtggttgtggtgtggaatccggctggggtgtgtgctgccctttggcactgtgagcaacacatgtcccccagtgctgatcagctgccagttgcttgcctgagcctggcgctgtgatgcattttggcctctactgttcagcagcgtgcaacggcatcctgttggcagtctagtgtcggctgctgctccagcagcagattggtgtggagtgtgtcggttttgtagccagcattaccgcttggtactgcagttggtgctattctggttgccaattcgttaccgcgtggtaattctggtgtcatgac

SEQ ID NO: 46 - DNA, 2126 bp, Scenedesmus obliquus
atggttatgtcagctgcagctgccgctggagcgtcccaccatgcctgaggtggcagcagcattagggaatttggttaaactgctgcggcaggagaagcgagtagcggcagtggctgcgctggctgctgcgtcaacagggggcggggcggcagcagcagggctgaacggcggtgcatgatgttgctgcattggctgcggcaagcagcacacagggcgaagcagcggcatgttgcagcagtatgttatgtcagctgcagctgctgttagagtgtcccaccacgccggaggtggcagcagcgctacgcaacctggtgaagctgctgcggcaggagacgcgggcagcagcagtggctgcgctggctgctgcatcggcgggtggtggcccagcagcagcagcacctgcagcagcagggctgagcagcggttcataatgttgctgcattggctgcggcaagcagcaacagagggcgcagcagcggcgtgttgcagcagcgtactttgtcagctgcagctgccgtttagagcggcccaccatgcctgaggtggcagcagcactaggccggtgcagcactacgccggtgcagcagcagcagcggcaggggtgaacagcggttcataatgttgctgcatctgctgcggcaagcagcaacagcgggcgcagcagcagcatggtgcagcagcatattttgtcagctgcagctgccgctggagcgtcccagtatgccggaggtggcagcagcgctaggcgttgcgacctggtgaagctgctgcggcaggagaagcgagcagcagcagtggctgcgccggctgctgcatcatctggtggtggcccagcagcagcagctgcagctggagcaggcactgctggcgtgtaatggtcgtgttgagggtgagctttgataggacatctatccgagttctggggcagctgcctgtagctgttgccatgctggtaattcaaggcaaagggcgtggtgatggggcacacgctttgtgtgcttttcaacaggcatatgtcagcgggcataggcctacctagatgccagcggtatgtagcgcagacagaccaaactagtttgtcaggtatgcaggggactgccatcactgctggtgctggctgctggtgtgttagctgtacataaacgcagcagtgataagcagtaggaagcagtaagaataggctgggcagtaagaatgcagagctggctagtttggagaaccctaggcagatcaactgtgtattgtcatttctgagctgtgtggtgctcgctgcggctgcatgtttcggcagctgtcaatgcgtctcagctgctgcgtgattggcagaagctccttgcgccggcaaactagaaatgtttgctggttcgttgcctgctttcattgcaagctcgtttacgttcaatttcctgttgtggtttgtggcatgtggttcatggttgaggtttatggttgtggtgctttggcgtgtacacgcacatgtgcacacgctcaacgcgcgcaaggcatctctatgcaaattctgctctgggagtccacgaatgtgtgctagatgcaacgtagcaacatgccgttatgtggtcaggatcatgtcatatttctatgtatatacgccatcattcagccagcacagcagaactatccggaccactgagcatctggcagtttcatatgactgctcacatgccagcctccgtctttcgcgcatagtctggtgcatgtatgccacggccctgaattatcctcaggctagccgctgttcgtgggtacgttaatttcgtactgaaagtggtacaaaacacatggcgggtgtcttgtgggcgttccgatgcgtgccagctgcctgctgctatttaaggtggtagtgacgtcaccatatgtgcatgtgacggcatgcattgtttcgtgcatatgccggcatgcgctgtgcatacctgcctgtgtacatctttatgttgttggttgttactaggaggccctgagtggtcaatgagtgttgctgccacagtagaactattgcatcagtttgctgcactaccaggctgatgtggtggcatgcacgatatgtgtctgtgtgtaacaccgcattacgtatgcatctggagatggtgtgtgtaacttgaagacag

SEQ ID NO: 47 - DNA, 1103 bp, Scenedesmus obliquus
atggggaaagcagtggtatcaacgcagagtattaatgaagacacagtaactctgttcagcaccgcaaactttgtgtgcggacagctgcaccttatgtgttttacgtaatgcatcctgttacctaaaatggttgaactttggagctgtgcacttctctgtttcacaccaaacgccgcatattgaagcagtgcggcctcttttcagcagagcatacttggcatccacagggccgtgtagtctttgtttagtcccataccttggcttgccaggagcaatgccgtcgctgtttagcagtgcacttccggaatgcagggttttcagcagcaagcagcgtatgcagagtctggctgaaacatatgctggaggcctcttaggattctctctctgccttgcaggttccctggaccggcacgacagtgtggatgctgcagcatgccgccacatctgggctcatctgccaggatgtttttctgccaggattgacaccatgtgcatgtgctgctgttgccatacaacattgcggtctggccacacacagtttttggcctgttgtttgtcggctgctgctgccagcctgttgttgctgagcagctgatggccttagctgacgctgtgccgcgggcgcccacgccggctgtgccgtgggctgtgcccaaaactgtgccgttggctgtgccgtgaactgtgccgtgaacggtgccgtgggttgtgccagcgtgcagtgcagactgtgcaccacacgcagtgtgctgacatggactgtgcacaaggttgcatgcaagcagtcgagcaacaattgtggtcggcgcctagcacattgcatagccttcttgctgacagctacaccgggtgcagcaatagccgcttgcagcgcctgcaggacgcaatgcttgcatggtaccgtgcagcagctgtgtggtgccgcccaatgccactatcgggcctgcatgttgctgttgctgcggtatgcaactgttgttggcccgtaactttggggcgcaagccccgatggcgatgctgctgtggcttgatgggtaatatgatggtgattattggctcgtagccagtaggatttcgtcccccctggtaacaaaaaaatatgc

SEQ ID NO: 48 - DNA, 1438 bp, Scenedesmus obliquus
atgatcactggctgcaggttgaactgcgtcggatgtgttacaggcagccaacgcgcatgcttgtgcaccacctggtcagacatgaatagtgaggaggccgcaaggggtcgccatacaccacattttggattgttgcacggtgttgcgggactgtgcacactgccgtgaactgtgcgctgcactggggccccttggtgcgtacggcgtgaccaagggaggccccttgcggaattgcacgggcagaaccagtgagttgagttccctggctgcaacttgcatttgtgactgttaagtgaacgcggcccttggccaaaacacagggccgccagcattgatacgcaatggtgaggaggtggttacgtgttggtcctggcggcatttcaaggagcaaaatggcactgggtgttgctgaatgggcgtgtgaggtgcctacagagatgttttggcggatgctaggaactgctgccggccgggggcttcaagcagtatgcattgagtgtgccacaggccacattgcatactgcccaagtgcccaacagagtgtgaattactgagcttcaacggcactgcttgggccgcaaaagttggcagctccttgtcgtgcgtcaagacgcttttgatgtgtcgcttgttcggccaactttgattgccctggttgtgcccagccacgggcctcggcggtgaagacatctgctagctgttgctgtgccttgcacgcgcagctgcgttcaggatgggaaacggtggctgtgcgatgggcaacagccgctgcagcaggtgtgggacggcttccgcttggcggcagcttttactgtccttgctgcgctgtgtggtattttctactttgattgccctggttgtgcccagccacgggcctcggcggtgaagacatctgctagctgttgctgtgccttgcacgcgcagctgcgttcaggatgggaaacggtggctgtgcgatgggcaacagccgctgcagcaggtgtgggacggcttccgcttggcggcagcttttactgtccttgctgcgctgtgtggtattttctacgttgtgcaaaccggtgttttggcagatgatggatgtgctagcattgtgcagtttactgcaatacgaggcacaaccaggaggattttagcagtctgtgtgtgttggagtgtgttgcagggtgtgtagctatactaaacaaagcagagtgtgtagctgttgtgcaggggagttgctctcgtgctgctggcatcagtgaagcgttgcaacggccaacgctggtggccacatggtgtatgcgcaggcatgtgctagctggcgttacaccgttcaagtccagccgccatttccagcaaagtgtgttgtgtgatctgcctacgggcactggccagtgagctcacaattggcgcaaaatgctgggcactctgggctgtcaggcgttgtaacatagggttc

SEQ ID NO: 49 - DNA, 1464 bp, Scenedesmus obliquus
atgggctggtgttgcgaggttgcaacgatgtgagcttgcagcgggcagcttgtagctactaaccacttgatgttggtgtgatgctagcacagtgtacagtagaggtctagtctacgagttgtcaacgagcgagcagcagcaggcaggtgttagtttgtaggtgctgacaggggtgtgcttgcaggcgtggctggtgactttgacgaatgcacctggcggttgtagctactaaccatagatgtttcaccacgcatcggtgttggtttgtagctggcggcgggtgcttgcaggcatggctggtggtgctgaggatgcagctggcagcttatagccactaaaccatacggtgttccagctctgctgtcgatgctctgcagtgcgcgtgctctactagccctgcttgttggtgctctggtgtggaacgtagcgcagctggcagctgttagtcgctaactgtgtctgctgttgcagggttggttgcctgcctgccgccgtccgtctgcctgctgctgtgtgcgctcttggccgccgtcgccacctctgccctcacaagcccttcatgcccaggcgctgctgcctgcggctgtctccacatctccccactgctgctgcatggcgcgctctccacctttcagggtgtgtgctttccagtttcagggcagggtggggtagcctagctgtagatgtacctctgctgcaattgcctatgccagacagatagataggagtggaggtgtgatgcttttgtcgcctttgccgcgccagttgccttgcagcttgccgcatgtgctgcagcatgctctgatgtattggcgtgtggaggtgcagcctgtagtcgcagctgctcgtccccgtgacagcgctcactgcctgcatacacagtcagcacgaggagcatgcacagcgcagccgcgtgcagcagcagcagcgttgcctgtgttggcttgttgtctgtgtgtgtgtgtgcgtgttggtcacgcggtggtgtgctggctggcactgcccaggaggcccaccgtggcctgctgttggtgctggtgcggtgcgcggctgcggctgacacccggccccccgcccagccctggccacggcgcggcatgccgctctgactgcggctggtgtcccccgagtgcccctgcacgaggaaaggctccgaagcatgaagcgagctggcaggcgcgccgcaacagcgccgctgaggtgcaccagggtggcagcagcaccccgggaagctcagctcagcagcacgcacaggatctgtgcatgtgcgctcagccgcaagtgtgccccaggtcggcgtgagaggacaggcctgaagtgacccgcccatgtgacagtagcttccaacaccccaagctgcagcagcggtggaggcgcaggagccgcctggggaggggtcgccttgtgtgctggctggtgcctgagcctaggtgcacgtgatatatatgtaacaaacaaaagatg

SEQ ID NO: 50 - DNA, 2810 bp, Scenedesmus obliquus
atggtctaaccatactagataacacatgcccaccaccatgggcacacctacctctcctactgctgacatcgctggtccgcagcaggagcagcaacagcagcagacaactgtaatttttacctcgaaacagtcctcacactcacctaccgtacttgctcgctaccagcgctacggccaggagcttgccctgttcctgcttcagcatggccaagactccctgcagctgtacctacgcttcctgctgagagccagcagctgcgtgaggctgtggctgcaggacaaccagcagcatctagcgctagccagcattgcaaaagcgctgcgctggctgcggctgaagctgctggcccccataggtcagctgctgtgtggcctgctgtccttgcagctgcgcatcagcacaaatgatgacttggatgatgccaccaagtgctcagcgaggctgaacagctgcctcacactgctgtttggagctatcaggctgcgcagccacagctgcctgcagctgtctctgcccagccggcagccccacttcatcctgcctgagttcagcctggtcagcccagcaacctcaccacgcctgccaggcacgccacgcagcaggcagcagcagcaggagtggccgctggcagcgcagcagcagctgcgcagcagcagcagcagcagccccatgcgttcgccgcgcaagggcgagcgcggcagcagcagcagcagtgttgtgcggcgcagcgctgctgctgaggaggaggtgcatgtgctgtgcagcagcgagctgaaggcagccatcatgtgcttcctgcagcatcacagcgccagcagcgggcaggaatttgtgctgctgcgcagtacgtcgtggggcagcgctgctgcggcaggagcagcagcagcaacattggacagcaccgcagcaggcactcgtcgctctgctgctgatggtgctgtgtcccccaggcgcaggagtggtgggcctggccaggtcatctcgccaggtgcttgtgggcgcatacagcagtcacgccagctgttccagacgctagcagacagcagcgcaggcagcgcgcgcagcagcaggagcgcaagccgtgacggcttcgcctttgcacccctgcagtcgccagccacatcagctgctgcgctgaacggcggctgcagcagcagcagcacgcagttgtggcgcagcacgagtgcagtcagcagcagcagggacgctccgcagcagcgcagcagctggcctgctgcaggcagtgcagcatcgcctggcgtgctgcagctgatgcaacacgctgcgtatgctgctgcagatgctggtgcaggtgcgaacgggtcggcagatgctgttgtggctgtcagcatagaacagccgcaggacggtgtcaccagtccacgcttgcctgacggctgcagctggcctgacaagcagcagctccagcagcagcagcagatgcagatgcagcacctgcacacacgggtgttgcagcggcagcagcagcagggaggcatcactcccaggagccttgctggctgcttttccgagccaaccaacctgagcatgtgtgtactggatgaccccagcctgacacccctgcggtccttcagtgctagcggcagctgcagccatgccctgcagcaacagcagcagcggcagcctgcctccccatctggccggctcagtggccagcagcaggtgcaggctgtgcccctgggctcgcatcgcgtgtgtgacaagggtggcggctgcaccccgccccgctccgcaagccccatcgctgcgccagcacggcatggcgtgcggaaggctgccggcaagctgccgcctgcgccgtttactgcggcccagctgcagcaacaagagcagcagcagcagcagcagcagcagcagcagcagcagctgttctagcagctgcataggtgcatggtaatagatgagcctgtggttttgcagcactgctgcaggctgttaatcaggcacagcggttgagtggcggcacactgtcctgcagccgctgattggtggcattcaacttctcgcttgccttgctacagcgcttcattggcgtgagaaaggcggcagaagcggagtcggccaactggtcaggcttgcagttttaagcgggcgagagaacagcagttggtgatggttcaggactggtacatttctcaacagcaatgccaggcctggatcctggtaaggatcgtgctctgtgttgacttggtgtgacttggtttgacatggtgggactctgagtgcacgcatgtgcagcttgttcagtgctggctggcaagccattttgaccctgtgatcttttgatgttttgagtgatgcagagatgacaactcagttgaactgggcgtagtagctgaacaagctggggcggtgttataactaatcagcctgcttggcgtggtgtgatcaacttcagtttgacaagtatcagtgctgttaagatgggttgcagcctatgcaactgcgtgcttagtattctgtgtgtacgactagttgtacatggtgcctgtcgtgttttgcagtgctgctgagcagcaggggtgtgtggcaccttgcaatcagtgcagtacttgcatgcctgcaggtgtcacttgcgacattagtggccaggccaggcctggcctgccatgcgactggcagcgaatgtgcctaatactgttgcttatatttgcaaatctgcttcgtgtatcagattgttgcagcctttaattttgtgtttcaaggggtgtagcagcacaaccgtgctacccgccaaaaaggtgtcaaagtgcagaggaccacgaggctcttgtaactcatgtttgct

SEQ ID NO: 51 - DNA, 1765 bp, Scenedesmus obliquus
atggcggactgtgagcacgtgtggtgcaaggaaccgagcagtaggaatatttccgcactgctccaatggtgcgcctgcaggcagccagcgggctcctgcgagcgctgggtccttgcattcgcagcagcctgtcgcctgagtgccagcagatagcagcagctgcagcgggcgtgcgcctgtctgagagcgtggcaccggcagtgacttacagggacatctcggacgaatggtacctcaggcagcgtagtcagatcagcttgggcaaccggctgccgcacgtggcagtcagcgcttggatctcgcccagtgcagtggtcgtgggcgatgtggacctgctggacagggtcagcgtctggaacaatgtggtgctgcggggggacctgaacaacatcaccatcggccaagtctcgaatgtgcaggaccgcaccatcattcatgcagccaggacctcacctgcgggcatttctgcaagcgtcaaggttggaaagtttgtgaccatcgaaccgaactgcacgctgcgcagctgccgtgtcggagactttgcaaaggttggtgcgcgcagtgtgttgctggagggcagcatgatggaggactacagcgtgctgcagccgggcagcgtgctgccacccactcgccgcgtgcccacaggcgaggtgtggggcggcgtgcccgcgcgcttcgtgcgggcgctcagcgaggacgagcgcgacgcgctcaaggctgaagccgatgacatccgccgcctggcctggcagcaggatgcggagcagctgccagtcggcactgcgtggcgtggtgtggaggcgtaccgtgcggctgtggttgagtctggcaaggcggtgagtgtgcccatgcggaggctcaagtatgaccagcgcaagcgccgcgaggacgaggcggcaaatgcgatcagcgtggctgcgggcgcttcgcaggccatcaagtgagcaggccatgcagtgagcgggccatcaagtgaggcggccatgcagtgagctggcgagctgatgcagtgattaggctgtccagtgagcggcgggcgctgcgcaggccagcacgtgagcggcgagcgtacctgatgcggctgcccgacaacagcgcgagcggccggctggtgcttgcgctggcacgcaggtgcgctgcgtgttgctgccaagcaaggcagcagcagctgcgggctggtggcgagtaagcgagcagctgtgtgtgctgcggcagcagtggttggtgacagtgcggcgttggcagcagcaggtggcacccgcgcagtgctgggttgacctaacgttgcaacgttgcatggttggtaggtggtgatgtgctgcagcaggcagtgatgaaagccacacaagtgcgacgtgtggcttaacttggtggcgtgagcttattttgattgtgtgctgatgctttttgcaagagcctgcgggccagtttcgtgtccaccaatcacactgtggactctgattcggaccttgttactccgcccgcaggggggcacctgccaggtgccgcggtgtgcctcctgagtgtgctgttcactgtatgcctgttgttgatagttagttgccacatgtcagacacacaagacatgctccaatggttatatttggtgttgcttcggcggattgcatcatacgctgtgcatggatctaagggctgccagcgctcgctgcagcagtgctgaggcctggctagctcaagctagtatgtgtgtactggtcagtggctgctggatcctggtactacttgaattattcggttgaag

SEQ ID NO: 52 - DNA, 909 bp, Scenedesmus obliquus
atgcaggtcacaagttcaaaacacgaaaaatggctgagactctgaccttccgcggcagcctgaagggccaccagggctgggtgactgccctggccacccccctggaccccaacagcgacatcatcctgcccgcatctcgcgacaagaccgtgatcgtgtggcacctggagcgcagcgagacccagtacggctaccccaagcgcgcactgaccggccacagccactttgtacaggatgtggtcatctccagcgacggccagtttgctctgtccggctcctgggacggcacgctgcgcctgtgggacctcaacacgggcaacaccacgcgccgcttcgtgggccacaccaaggactttgtttcactacttcttttatataaatggacgggactgcarcagcccacactctggtggtgtggccctacgtcatgtccctgtgggccctgtgggtccccatgtgctgcaggtgtgctgcaaacacgcagcgcacacacagatatgcacctggtgtttgacataattacttacaatggatgccccctgggccccgttatgttgctgcaagtgtgccgcactccttcattgttgttgtcgttgttggggctgcagggcacactggagatgttcacggctctgactggtcccttcagcagcgcccagcactttgtggacagtccaccgatgggttgagcctgtacgctgttcctgtaagtggttgtcattattggcatgtgggcacagacacagtgagtgtccgtgattgtggatctaaaagctatgtcaccactcctcacacaagagaccactgtttyggttggacggggctgcaacatgtccctgcacgcacggcgacgcaggagtaccagcactttgtttcactacttcttttatgtaaatggacgggactgcg

SEQ ID NO: 53 - DNA, 909 bp, Scenedesmus obliquus
atggccttaccccaagtcacgctactgccgtggtgtgcccgaccccaagattcgcatctatgatgtgggcatgaagaggtccgatgtggacatgttcccccactgtgtccacttggtcagttgggagaaggagaacgtgagcagtgaggcgctggaggcggcgcgcattgcgggcaacaagtacatgaccaagtttgcgggcaaggaggccttccacctgcgtgtgcgcgtgcaccccttccacgtgctgcgcatcaacaagatgctttcctgcgctggagctgataggctccagacgggcatgcgcggcgcgttcggcaagccccagggcgtgtgcgcgcgcgtgcagatcggccagatcctgctcagcatccgctgcaaggacaaccacgccgcagtggccgctgaggcgctgcgccgtgccaagttcaagttccccgggcgccagaaggtggtggccagcaggaactggggcttcaccccctactcccgcgacgactacatccagtggaagaaggagggccgcctggtcagcgctggcgtgcacgcacagctgctgggctgccgtggtgccgtggctgaccgcgaggccggcaacctgtttgccacccccgcacgcatccacaagatccccaaccacgccgagtaagcagcagctgcagcagcagcgggcggcgatcggctggcggagggaagaggttgcggcgctaccagcagagcaagcagcagtggctgcggctgcgtagcaacgcaagtggtggcagtggcaggcagcaatgggcagcagcagcagctggagtagctggtgtgctgctgaccatgcagctgcggcggggtgtgttagtgactgcttgcaggcgcccgcagcaggcaggtggtgctgcggcgcgtgtgtaactgtctatgcgc

SEQ ID NO: 54 - DNA, 1437 bp, Scenedesmus obliquus
atggcttgtgattgtcagtgaagtcaatcaccatgctggcagctcgcagcacctccgcccccagggcgttcagcagcagggccactgctgcgcccaggggcatccgcgtgattgccatggctggcaagcgcaatgacgtgtctgacagctatgccaaggcgctggtggagctggccgaggagaagaacaccctcgagtctgtgcacgccgatgtggataccctcgccggcctggtgagcgccaaccagaagctgtctgagctgctgttcaacccagtggtggaggctggcaagaagagggcagtggtggccaagatcagcaaggaggctggtttccaaaagtacaccaccaacttcctcaacctgctggtggcggaggaccgcatgaacctgctgaacgagatctgcgagtctttcgaggagcagtactgcacactcactgacacacaggtggccaccctgcgcagcgccgtgaagcttgagcaggagcagcagttcctgatcgccaagaagctgcaggagctgactggcagcaagaacatcaagctcaagcccatcatcgaccagagcctgattgccggcttcgtggttgagtacggcagcagccagattgacctgtctgtgcgtggccagatcgagaaggtggctgaggagctcacccgctcaatggccactgcctaagcagctgctgaggcatccagcatgcagtgtgcatgatgtgtttggtcaactgcgacgccgtccagcaacctgcagtgtgagggtgtgtacgctgcgcgggttttcgcttgctaggtggtgagcagacagcagcagcggtatgcttgcactagcgcgtgagctgcgcagggtatacttgcatgcaggcggcagcagcggctgctgaggctggcggttcgtagtttgtgagcagcggggaaggatcagcaggcggttgtgtgtgtgtgcgtaaggcaaggcgaagagagtgagaggcagtacagcgcagtcgagtctgtggatgctggtgcttcgggctgcggtgcccgtgcgcctgccttcacggcttttagtgtaggctgcaagggtcgcacccttttgtggccctgcgtgtcgtgccagtgtgtgcgttgcatagggcttgtggcaacagcgggtgcctgtaactgctgcagctgctttttctgtgcgtgcacagctggcggttggttaacggcgcgtgtgcgtgtccttctgagtgacagctggtgggcagttgctcttgtgccaacgtgtgcaaagtgctgctccaccaaatacaatgacttttggtggggcctgcagcataacgtatgtctgtgctgcgtggggtgtggctcagcatggggcgtgtgcatcatggttccgtcactgcttgtccaacaacagcaccccattttgatggcccgtatctcctgctaacatgtaacagctggcgcac

SEQ ID NO: 55 - DNA, 781 bp, Scenedesmus obliquus
atgggctcccgcgacaagtccataaagctgtggaacaccctgggcgagtgcaagtacaccattgccgagcccgagggccacaccgagtgggtgtcatgcgtgcgcttcagccccatgacaaacaaccccatcatcgtgtctgctggctgggacaagctggtcaaggtgttcaacctgaccaactgcaagctcaagagcaacctggtgggacactccggctacatcaacaccgtgacggtgtcacccgacggctcactgtgcgcgtccggcggcaaggacggcgtggccatgctgtgggaccttgccgagggcaagcgcctgtacagcctggacgctggtgacatcatccacgcgctggtgttcagccccaaccgctactggctgtgcgccgccacccagagctccatcaagatctgggacctggagagcaagtccgtggtggacgacctgcgccccgagttcaccaagacatacggcaagaaggccatcgtgccgtactgcgtgtccatcgcatggtccgcagacggcggcaccctgttcgccggctacaccgacggcatcatccgcgtgttcaccgtcagccacggcctgtgaagcagctgctgcaacagctgtttgctggcggggtggcgtgctgcagctgcagtgcagcccgagggctgtgctgtggtgcgtgggcagtgagcggcgctacaaaggcgctgccgctgcagcgtagcatggccaggtggttgttgggtgctgctggtggctgtgtgtctctggtaactcgatgatcatg

SEQ ID NO: 56 - DNA, 1601 bp, Scenedesmus obliquus
atgcctgtaccagacagtgctgcaggggcagcaggcggcgccagctggcgggctgctgcagctgacagcgcatgacacggtgtgggccttcctggatgggcagctgctggggcgcagctaccgctcagcgccaagcaccattgaggtgccgccatcgccattcaacagcagtgaagcacagcaagacagcagtagcagtgccgagcagcagagaaaccaagcagctgatgctggcttagctgctagcaaaaagctgctgtcccagggtagcggcagcagcaccagcagccagggccggctgctgcagctgctggtgtggccgctgggccgcaacaactttgcgctgtttggcagcagcatgaacgaccagaagggcctgctaggcaacgtcagcctggcggggcaggtgctcacagggtggaacgtgacccacctgtgcctggaggggggcgggcctgacgccttcagcgacctgcagctgccatggcggccgctggctggtgctgctgctggcgctggaggcgcgggcgcgcactgggacagtgacaacagcgcagggcagcagcaggcttccagtggtggtgaggtggcaggggatgccactgctgctgctgaagggagggtctctgggcagcacaggtcccagcaggaggctggcagcaggccgctgctggtgctgccaggcagcagcgcctggctggctgagcgtcaagctgcgcgccaacagcagcagcagcggccggcaggtgatgaagccgccagcagcaaaagcagcagcagcagcagcagcgtgcgtggcatctttgctggtgctggcaatgttactgctttcatttccactgttgctgaagcccaggcgcaagcagtgattgcagcagcagcagacgcagccaccggcaatggcccagcaactgcagcagcgcgtgcagcagccacgatccgtgctgggttggcagcagctgcagcagctgataacagcatagatgtagcacaacgcgccaagggcccgctggtcttgcgcggtacgttgcatgtgcctgctgctgctcaagctgaagccaccggcagtcagcagcaacagcagcaacgcggctcgctgctgccgcctggcaggcccgcggacaccttcctgcacctgggtgagggctggggacgcggccaggtgtgggtcaacggggtcagcctgggggcctactgggctgagcagggcccgcagatgtccctgtacctgcccggcagcttcctgcagcccgggcccaacgaggtggtgctgctggagctggatgggagggtgcctgtcaggggggatgggcctccaaccattgcgtgtgctgatgagccagattttgacgggcctgccgcagggcctgcgccactggcataggctgtcactgcaatggccatgttgggcgtggcttgggcattgtagtgctggacttgcccgatggtgggggttgtgcgcactggtttcgtagctgcaaagcattaggctgagcttgcgttgccatattctggcttctgaggtgccacaggtgcgtgtaggtggcgctggacacgctgcaccataataataatagtcttcttgtaagccataccttgtc

SEQ ID NO: 57 - DNA, 881 bp, Scenedesmus obliquus
atggcttactgtgtgctgccttttcactactgctgctgtctgctgcctggaccggccacaaacaagctcagccaccagtagcacaccgaggcccaccacttgccggattgtctgtcgacagttcgtgctcatttaacacatcaaaatcaatagcaagatgaacgccacaatgctgcgcggttcagccttcgcctccagcagtcgtgttgctgcagtgcgcccagccatcagcagctgtgcatccacagtcgtggtgcgcgcagtgcaggacctgcagggcaaggttgtcagcaagggcatgcagcagtcagtggttgtggcagtggagcgcctgtcagcacacgacaagtacttcaagcgtgtgcgcatcacaaagcgctacatcgcacacgatgatggcagcctgggcgtgggtgtgggtgattatgtgcgccttgagggctgcaggccgctcagcaaaaataagcgcttcaagctggcagaggtggtgcgcagggccgactaagccctgttctacataattggataggttggatgacgagctaaggtcactgccgccttatcgtggctttcagcatgtgcacatggctgctgtagtcggaccacacaacagcagcaggcttttgaagcctttgctgcttggcggtgtgttgcaacgaagcatgccagttacgtgcagctgcagcaccagactggcagcagccctgtgctggaggcttgcatgtcagccagcaaggtggcgcaggcagtgtgctttggcccttagacttaggttttgtggcctgattatgcaagtatgcagagatgaagggggaggggagtagacctggcatgcagctgcagccgggttgactggggttctgggagtgtaacacgtgaac

SEQ ID NO: 58 - DNA, 1280 bp, Scenedesmus obliquus
atgtgccccttttctgtgtagctgctctcttagcaggcatctctctgtgtctggcttgcgtggcaccacaccaacaaagcatgcagcagcgcagcgctggcgcatgcagctgttgcagcgcgctcgcaggcggcctggagtgtccgcctgtgctactagtaacgtacgcatatcttgcaggctctgccaccgcttgcagttaggggtgatgcgcgcttcgctgttgtttctggttgtgcgcctgctgttgtggagcaattgctgaatgcctgctgcaaccctagtttacggcacgacagagcagaggctatcacagcaggcatggtttcaggcaagaattgtcgctttggcagtgctggcagcagcagcaggtgggtgtactgtgcccgcctgctagcttggtgatcacaggcatcacgtgcacgcagtgccttgcagcatgctgatcctgttgttgccgcacgcacctcttgggtgcacagcttgtggcttgcatttacacagctccttctgaggagctgtgggctcttctgctacgtaccattcacgagcgtttgtacaaatggagtgagcagcagcgctatgtgccactgtcatcttactagtatgattcgtcgcactgcaaccagtctttaaatgcggtgtgaactgctccaggcatgtgcttcccggcgctcattcagtgtggctggcgtgcagcgccccgtgttgttgtttttgcatagtgcaaggagcgtcatggagagcatgtagagggcttgttggcaaggaacaacacctcaccccgctcatcctttttgagctagtagcatagcagcttcagcttgcagcctggagcttcctcagcagtccacttcactctggagcttgtgttgggtcctgcactgccagggtgctgtatagccacgcttgtcttgggggccgcccactgcggcatgggtgctgccgctgccacgctgccatgcgtggtgtgctggtgctgcccgcccatgtgtgcatgttgaactgtcacagtgcggtgttggcgcagtgtagcatgcctcccttttgcaggataacttgcaggataacttggaagcgtgtacatgtgcgtcgctgtcggtgctgttggtctgcaaggcatcgccttgcgcggcttcacaacggtttgtgctgaatcggaagggtgggaggagagtgaggctggcggtgtgtgcattggggtagtgtttcagggacaactgcgagtgtgcctacttctggagagcatgttgactgtacaattgtgtaactgttttatc

SEQ ID NO: 59 - DNA, 1177 bp, Scenedesmus obliquus
atgaaagcactcacgacaccgttgcgcttcaccgcacaccaaactcatcacaatggcagtctccatgaggaccagcgtcgcagctcgctctgcaacccgcgcagctcgtccctccagggccgctgtggtggtgcgtgctgaggcccgccgtgaggtgctggctggctttgtggccgccggcgctgccctgctgagcgtgggccaggcccaggccgtgacccccgtggatctgttcgatgaccgcagggtacgcaacaccggctttgacatcatctgcgaggcccgcgacctggacatcccccaggcagagcgtgacggcatgacccaggcccgcgctgacatcgaggctacaaagaagcgtgtcaaggacagcgaggcacgcatcgacaactcagtggcaaccagcatcgagaaggcctactgaactgaggcccgcgaggagctgcgccgccagatgggcactctgcgcttcgacctcaacaccctggccagcgccaagaccggcaaggaggagaagaaggcagccctggccctgcgcaaggacttcatccgctccgttgaggagctggaccttgccatgcgcaagaaggacaaggacagcgccctggccaagctggcagtggccaaggccaacctggacagcgtgatcgctaaggtgctgtaagcagccggccacagctacccaaatatcacccccaaaaacctcgtccaaggtccattgtgtagccaggcgttgatggtgacggtgtcttgtgctggctgaagaggcaagggcgggatgtggtgaggcgcaccagcggcgcagcgctttctgcgcccggcgcctgccgctcccgccgccgccagcttgtgctgatttggctgcgcagcagccgcgcatgatgagtgtactttgatgcggtcggcgcgcatgcatgcccgtttatacgtatcacccgcagcgcagcaagcagcgacgaagcagcggagctgcggggggctgcgtgaggtgcagctgctggcagagagtttatgtgtggcctttgaagccgatgctgggctgctgctcagggcagcaacagcagcgtggcgttgtgctgagggggcctgcagtgccaccttggtgcagagatgcaccatgttgtgtgtaatcgtgagatggacgcgtatgcagtccagtgtaaaggagcagaagttg

SEQ ID NO: 60 - DNA, 1543 bp, Scenedesmus obliquus
atgctgctgccagcagcggggaggatgagaaggaggcagaggagcaggcggctgcaagtgacgtgtaagcggctggtgttttggtgtttttaacagcttgtaacagcttgtaacatgtcactgacatgtggtaggtggtgtctaaggtacatggtgagggcagcagggcctgccacattgcaggcccgactgaaaggtggatggtatgtggggcgtgagctgtcagttctgtttccgcgtgcgttctgtttgagcacgctgcgtacatatgcaaactacctgtcaacagactaaacatgtgtgcagctggctctatggcggttggtgggctgatgagcagctggtacagcctgttgacagctgcagctgcagaataccagagtgtctgccccatgtgttgctgggcttgattgggctgcaaaagtaccatcagaagcagcatcacgtacttgcctttggtagtgaggtttgttgagcagcaatgctgtgcctgctgatgtggcagctgttgtgctgatgtggcagctgctttgctgatgtggcagctgtcgtgctgatgtggcagctgtgcctgctgatgtggcagctgtcgtgctgatgtggcaggctgttgtgcttaagagttgcaatgggtgagcagaggtgcagcagttgtgcggtgttcacatggagtttgcagagtttttaagcatgcaaagcaaaagttgtgatcggtaccaccaggcaggtgcatggccgatggcggtttctttatgacatagcagacacgagtgatgtggagccggctgcaatgcaatttggcgtgcgccggtgtagctgtgacacaacacgctgagcagcagcggcgtagccatgagtgcatgtgtaagtcagcgagagagtgagtttttgaaggaatcaaaagggcggctgtgagagttccttcgttgtgcggccgaggcctggtgttgccaataacattgtttgtcaacggtgcaatcagactgtggtgtgtacatgcttgttggatgtggcgagtttagtctggcacgatattcacagcacttgcagagttgcggatcggtgcttttcatgttgttgtgcgctgctggctcagcaatggacacagcaaaagattggctcgttggacagccaaggcaacttgaaatctatgtgcctgctgcagctggcgttgctatgcattggagtgtttcaggtgctgccagtggtgtgcacagagcagcgttaaactaacaatctgcagctccttaactggcagcatcgaccagagtgctgccgtctactgcaggtgtgtagcatgccgcgtgttaggtcatatctgtgatgttgtgttggctgaacagtggcctggcccgatctgatgtgcagttgtggacatgatgcatgcagctctgttcgttgatggttgttgctgcagcacttctgagagtctgaccttgtccaacgatgctgcgcagcaagactcaaacattgctcaagcatgaaggtgcttagatgttgtgctgcaccgcggctgcaggcaattgtgtaacagaatcattcc

SEQ ID NO: 61 - DNA, 1890 bp, Scenedesmus obliquus
atggggcttcatccgctccgttgaggagctggaccttgccatgcgcaagaaggacaaggacagcgccctggccaagctggcagtggccgaggccaacctggacagcgtgatcgctaaggtgctgtaagcagccggccacagctacccaaatatcacccccaaaaacctcgtccaaggtccattgtgtagccaggcgttgatggtgacggtgtcttgtgctggctgaagaggcaagggcgggatgtggtgaggcgcaccagcggcgcagcgctttctgcgcccggcgcctgccgctcccgccgccgccagcttgtgctgatttggctgcgcagcagccgcgcatgatgagggtactttgatgcggtcggcgcgcatgcatgcccgtttatacgtatcacccgcagcgcagcaagcagcgacgaagcagcggagctgcggggggctgcgtgaggtgcagctgctggcagagagtttatgtgcggcctttgaagccgatgctgggctgctgctcagggcagcaacagcagcgtggcgttgtgctgagggggcctgcagtgccaccttggtgcagagatgcaccatgttgtgtgtaatcgtgagatggacgcgtatgcagtccagtgtaaaggagcagaagccaaaaaaaaaaaaaaacattaatggagtgtttcacgtgccaacttggcaagttgccaagccaagcacacggcgcgtttgcacgggcaaagaccgtggaggtccttcaaaatcaccgttaaacagccgaacatcaatagcctgtgcaagctgaatgcagctcggctaagcatgcatgcagcagcagcacatgtcactgcagtgacatgcactacattgaagcactctctacaccagcaccgggctcagcatgcagggcagcatgcagagcagcatgcagagcagcatgcagagcagcagctggcggtcaccacgcagctcaagcaccagcagcagctccttaccacatagcgcaagcatgcagcagcagttggcagcagttgcagcacacacgggcgcagggtggcgctgcaatgctgcgcaagggcagcagcacggaacgagcagcagcaccagcagcaccacaacccccagcagcagcaatgcaagaagttgactattttgcaaccgacaagcggccgattgtcttatacgacggagtctgcaacatgtgcaatcgcggtgtaagccgcctgctgcgtagggacaagcggggagtgtaccgctttgctgcactacaaagcacacccgggcggcagctgcttgccaggtgtggcagggctccagatgacatctccagtgttgtgcttgtagaggaaaactgcttctacatcaagtctgaggcggtgctgcgcactttgctcaaaatgcggatgccgctgccactgctggctgggctggcactgctggtgcccaagttcatccgcgacatgttgtatggccagatggccaagaaccgctacacaatgtttggcaagtcagacagctgcagggtttcggatgatggctttgcagaccgcttccttggggcttgaagcagtcgctgctggcgccctgcattgccgcagcagcacatgtcagtgccactggcattggcagtgcccgtccacatttggttggcaatcccgttggttggcaatcctgttggtgtttcatccgtgtctttattcataggtgaatgcatcctcaccaatactcttcaaaaacatgatttcaaaagaatggagttgggcaagcgtggttgcctgtggtcatccaacgccaatctcttcaaagccctggcgaacgagtatgtgaagtgctttgtgagagtcaccacaccacggcttactgaatatttcagtagtgtttcaagttgaaacagtgctggaatgt

SEQ ID NO: 62 - DNA, 756 bp, Scenedesmus obliquus
atgcactgcagggtatctgttcatacatcaacaggttttggaagtgttacaagcgtgcgtgctttgggggttgaggagttgttatcgctgccttttgcagcagcacaggctctgcgcagatgctgggcagtatggtcaggtgacatcttacattcagacactgcagagtgactgtcttgcaagttctggtgatggatgtatgagtgtgctttgaagagtgaggggctttagcagtgcacttgttgtagcgcaggctctgcttaagtgctgagcggtgtgggccaggtagcacttgcaggattctgtactgcatttgctggtgatgaaaggccaggtagcacttgcaggattctgtactggcatttgctgatgacgatggatgcgttgttgtgcatagcagaatgacaggagtcctagttgcttctgcagcagcacaggcgcgcgcgttgtgtgacgcattcagagcaggctgttgagcgcgcgcaggaggggcgtgcatgttggctgctgcttgctgacatgtgcaatggatgcacagctgatgccaaagtcaaacgattcttgatgtgatgagtttggtgtaggactgtacagcgtctgtggcataacagcggttgtacatgcagggcaggccagtgtcactgcaagtgtcaccacgcctgaacccttgtgtgggtgacaactgctgtgcatgctgctgcagcaatcccgtgacagcggtcaactatggctcatactgtgtgtaagcgtcaaggc

SEQ ID NO: 63 - DNA, 1420 bp, Scenedesmus obliquus
atgattcaaaacgttttggtgttcgcttgcagctcaaacctatcaagtatcgctttggtgacctgtcaatcagttgcccaatacacccctcacaatgctgtcctcaagcctgagcacgcgggtcgcagcagcgccacgcaatcgtggcagcagatatgccttcagggcttcagcagcagaagccccagcagcgcaaaagacatgcttcattgcaatgaacgtgttcaaggtgaagccggagtgtgcagaggactttgaaaatgtgtggaagaaccgcgagtcgcacctgaaggagatgtctggctttgtgcgctttgcgctgctcaaatgctccaacgtgcccaacaagtacatctctcagtccacctgggagagcgaggaggccttccgcggctggacgcagagccagcagttcagcaaagcacacggtgaggcagataaggacaagggcggcagcaagcggcccaatgtgggggccatgcttgaggggccgcctgcacccgagttcttcagcagcgtgaccatgacagagtagtcagaggccaggacatctggccagtatgacagagtcactctgtctgactacggtagtcagaggccaggacatctggccagtatgacagagtagttgacatagtcagagggcaggacactgtccagtccaggctggccagttttggaagccaatgatgggctcatggggcagagggagcagccacaagtcaggatagaacgacagcagtgttggatgctctcatcttgtcatgacagaggagcggtggttgtcaaccagtggcttggcagcagccgcaggatgcgccacaccgactgctgcagtttgcttcgggtgccatcctggaggcgccagcggccccagactttggggaactcggagttcttcagcaatgtgtcgatgacagagtaggggatgcagatgtcttcacagtttgcgtattggagaggcagtgacaggctcatagggtacagagagcatcccagagcagaatgtactatcagcagtgatggatgacagagcagaggtgtacatgtaggtgtaccatggtagggtggggtagggtggtagggtgccgacaagtcggcgtggcagttggacgagggaagcggcacactggcagctacagagggcagtggcttcaagccgcatgcagcgagcagcaggagagagcccatgcctgatgcagcagccgagcatgtgctgtgcaaagcacgccaagcagagcatcatgttgttgtcctgtatgccaggagtcggagtgtaaaccaggggccactggttgtgtggctggatgctgtgcttgtgccatgcacgctgctgcggtagctgcgattgcaattgtctgtacattgtcattgttgcgcaggcacgtatcgtccatcatgatgtgtaacaagcagcagc

SEQ ID NO: 64 - DNA, 1767 bp, Scenedesmus obliquus
atgacccaaccagatcaggttgcccatacgtctgcacgcagtgctgaagcattttcgatgattgctgcacagctacgcaactttgcgcctgccgctagtaaatgcgcagctagacgtgtgacacttcctgggcagctgcaccagtgcatcggtgcggcatcccgcaacttggcgagcagcagcagccctagggacgtgcgtgctgaggccagcaacgcgcgattctttgtcggaggcaactggaagtgcaatggaacccacgcatcagtcgaaaagctcgtgcaggagctcaatgccggcagcgtgccttcggacatagacgtagtggtggcaccccctttcatctttctggacatggtacgcctcagcctcaagaacgagtaccaggtggcagcacagaactgctgggtgaagtcagatggggccttcactggcgagatcagcgctgagatgcttatggacaccctagtgccttgggtgatcactgggcacagcgagcgccgcagcctgtgcggcgagagcagcaagtttgtgggagagaagactggccatgcactggatgtgggactcaaggtgatcgcctgcgtgggtgagacccttgaccagcgcaacagcggcagcctgtggtacaccctggacagccagatgcaggcgctgttccccaacgacaaggactggtcacgcgtggtcatcgcatacgagccagtctgggcgatcggcacaggccaggttgccacaccagagcaagcccaggaggtgcatgcgtacctgcgcacagtgctggccaacaagctcggcgaggagactgctgcgggcgtgcgcatcatttacggaggatctgtgaatgacggcaactgcaacgagctcgcgacgatgcctgatattgatgggttcctggtgggcggcgcgtcgttcaaggcgccgtcgttcctgcagatctgcaacagcgtggcgtctcgccgcacgatggcggcgtgaacgaccatcggcatgtgctgatttctggtgcgtttacggtcgctcgaggcgccgcctttctcaaagatctgcatcagcgtgtcgttttgctgcgtgatggcggcgtgaagaatcagcatgtgctgttgtgtgtgatgctgctgtgtgtgatgctgtcgtgtgaggctgctgcgtattgggcagtgggcagctctttgtgtgctgttgtgcatctgcatgatcgcattgtgtcgttacagtgctgacagtgcggcacttgctggcggtgccacaacagagcagtgcgatcgcagggttgtgttgaccgagtcgccaaatgcatgctgttgttggtatggtaggcttgcacggtagtgtgatgatgatagcaggctgcatgctgctgcagctgtagcagccaggcgatgtgcagaacaacctttgctttgctttgcagcagcagcacgcagcagccgctctgcagagaaagccgggctgggcagcagctggtgttggatggtactacctagtagcacacattggttgtgtgattggctagcagactcttcagaaggcagcgatcagaaaagaaggatgacgtgcagtggcttagcgggggacggtttttgtgcgtgcatgtggcccacctggaatccgggattgaggggttactcgccacagtgctggttgccagcgcgctagagtgcctgtgacaactggccatacctggtgttgttgggttctctggttgcccgttgagtctgtcatttgcgaggggcggtgttctcgtcatgtaaaactcatactttc

SEQ ID NO: 65 - DNA, 1410 bp, Scenedesmus obliquus
atgctgaaccagagcctgtccacgctgcccatcacgcccacgccagcgcagctgtacgcggtgctgcagtaccacttctgccctgctccaggcggcaacgtgaagggcctgctgtacttcaagttcaaggcaaacaagttcaacccctgcctcaccctgctgggcctgacaaagcccgcccccaccctcaagctgttcttcaaccagaccctcaccgataacaaccccctcctgcccatgaagctcaccgttgactaccttgacctgggcggccagccagcgcagaccaacattgtggtgcctgacgtcaccacgcccctgaccgcgggccacgtggtggacagggtgctgattccccctctctccgcactgcccctgtaagcaagctgcacaagcccggcagcagcagaggcgcagcacaagcctggctgcatgctacgagcaaagaggctgacgtgatggctgcacacggctagcacgctcagcgcgacgatgcactgcaaccgcagacagagcggcagtgaatgacgaccgtgaatgtggatgggtagcgatgcgcgtgtatctgatgtcttggttttcgaccacttgggctggtgcagcttggcgtgccttctgctgtgtgcaggcctgtgccgcacatgcggatatcctttaagaaataaatgttgcatttcattgggcgtggccccaccgttttatctgcgtgccgcactgacacgcgggcacatgcatacatgtgtgcttccagcatctggagattgtgttttgacacggttacatttgacagttgtgtgtgatcggtgaccaaggctggctgcaggacttgtcctgagtacgcttgtaggcatcatgacgtggcacgacttgcaggagctttgggaggtgcgattactccgctgcaatcccttgttccccttcgctggggcgaggagagcggggagctgcttcaagatttcatgttgactagtaatatgatgggttttagccatggagatttggtgggaaaggagtgtgttgcatacatgccagatgtagggtggctgagagctgctgatccactcccacagcagctgcaggagcactttcctggaggctattttgagatgatgcgctctttgtgagtgagtcctttgggttgctgatacagtgcagctttgttatgcgttgtgtttggcttggatgccgccagcagtggggctatcatgggaggagtcgtgcaacagccatgtgtgattaaattgccggctggagggccccttctgtgcttgccttcactatgccagtggcagactgcatctccggtcgtgcccttgttacgtttcatagttatgtttggggcctgtgtgctgctctgctggaagcggcatcgcgcctgctgcagcatcagccgatgtaaccgtttgaacg

SEQ ID NO: 66 - DNA, 982 bp, Scenedesmus obliquus
atggagcctgctgccaaggggctctcgtcaagatgggcagacgtccagcacgttgctaccgccagagtaagggcaagccttaccccaagtcacgctactgccgtggtgtgcccgaccccaagattcgcatctatgatgtgggcatgaagaggtccgatgtggacatgttcccccactgtgtccacttggtcagttgggagaaggagaacgtgagcagtgaggcgctggaggcggcgcgcattgcgggcaacaagtacatgaccaagtttgcgggcaaggaggccttccacctgcgtgtgcgcgtgcaccccttccacgtgctgcgcatcaacaagatgctttcctgcgctggagctgataggctccagacgggcatgcgcggcgcgttcggcaagccccagggcgtgtgcgcgcgcgtgcagatcggccagatcctgctcagcatccgctgcaaggacaaccacgccgcagtggccgctgaggcgctgcgccgtgccaagttcaagttccccgggcgccagaaggtggtggccagcaggaactggggcttcaccccctactcccgcgacgactacatccagtggaagaaggagggccgcctggtcagcgctggcgtgcacgcacagctgctgggctgccgtggtgccgtggctgaccgcgaggccggcaacctgtttgccacccccgcacgcatccacaagatccccaaccacgccgagtaagcagcagctgcagcagcagcgggcggcgatcggctggcggagggaagaggttgcggcgctaccagcagagcaagcagcagtggctgcggctgcgtagcaacgcaagtggtggcagtggcaggcagcaatgggcagcagcagcagctggagtagctggtgtgctgctgaccatgcagctgcggcggggtgtgttagtgactgcttgcaggcgcccgcagcaggcaggtggtgctgcggcgcgtgtgtaactgtctatgcgc

SEQ ID NO: 67 - DNA, 857 bp, Scenedesmus obliquus
atggtttctgtcacgatcgaaggccactgccagctgctccaaggatgcagtcgcttgcgcgtgccaggacaaccccagctgctgctgccttgcggcatagaggcacgcaacgctgccaacgtaatctgctacacgttttgacgcaagcagcatcgcagcaagcagcagcgactgaagcccctgcacagacacactggttccagaataccatcacgctcccgaaccacaagcgcggttgccatgtggtgacgcgcaagctgctggcagaactgccggagctaggagaatacgaagtggggctcgcaaacctgttcatcctgcacaccagcgccagcctcaccatcaacgaaaacgccagccccgatgtgcccctagacctcaacgacgccctggacaagctggcacctgagggcaaccactatcggcacctagacgaggggctcgatgacatgccagcacacgtcaagagctccctgatgggcccctccctgaccatccctatcgccaagggccgctttgcgctgggcacatggcagggtatctacctgaatgaacacaggaactacggcggctcacgcagcatcatcgtcaccatccaaggccagaagcggccagatggcaggcgatacgggcaattcagctgatggcagcaagtttgcacacacttataattgtacattagacatgctgcggtgttagagacgtactgcatgtaatgtggtaacacttggtgcgtcttgtcgggactttaggcgctttggtttttcaagtcgctaggcatccccttcttctggtggtggccttgcactgcactttgcagatgtctatgcttgagacctgatgtaacaagcatcctt

SEQ ID NO: 68 - DNA, 1116 bp, Scenedesmus obliquus
atggcccgtttagttggttgcgagctttcaatcacctgctgccccaccactgcccataccacagccagcatggcgcgtaaaatctcttacgagaatctctttgtcattgtcgctgcggctgcgtcagcgctgctagcgtgttcggcacaagcacagatccagcaagtggcactgccgtatgcactcgacgccctggagccagaaatcgacaatgccacaatgaacttccattatggcacacactacgccacatacgtgaacaacacactggcagcgctgaagaatgctaccgacagcggagtgaggctgcctgtggctacctcaaacctgacagccctgatcagcagcatcaagaggctgccctcgcccctcaacactaccatccgcaaccagggtggaggtgcgtggaaccatgccctgtacttcaagcacctgacgcctcccggcagcccagccacgcagtccacagccatctctgctcccctgaaggatgccatcaacgccaactttggcagcgtggacaacatgacggcggcgctgagtgcagccgctggcaaggtgtttggctcgggctgggcctggctgtgctacacaggcaacagcagcgctgacctggccatcaccaccacccccaaccaggacaaccctctcatgggtcagctgcctggtgccccagcagtcagcgcggccggctgcacaccaatcctgggcatcgacgtgtgggagcatgcctactacctaaagcacggccccaagcggcccgcctacctggagtccttctggaaggcgctcaactggcagcaggtcagcaagaactacgacagcgcggtgcaagggctggagctggacctgagcgaggcagcgccccgggccctggaggcgccatccaaggcagtctcggcaggcagcagcagcagcggcagtgcaggttttggtgcaatggcagctgcagcagctgcactgctggcgctggtatgacagtgtttgtccgcagttcagtaatcgttgcaaccaactgggcacgtggtattattgtacagcctgtgcagtagttgcctgctgcaatagttctttgtaggttatacgctgtggcttgattggtaagacagcgatgat

SEQ ID NO: 69 - DNA, 1087 bp, Desmodesmus sp.
atggtgtgtgtgtcaccagcaggctggttgcccctgcctcccagcagagggcgacgtgtgggtgatggggtatgtggagccaaacagttgctgcactatgtgctttgtagggtactgtaagctgcaccaacgtgcagtgcggaccagtggcggggcgcagcagcagttgttatgccttgcacctgctatgcaggtgcttgcgcgcgagaaacacctgcaggtttttgtgttgaaagcccggccgggtagtggcctgctcaggtggatgctgtatttggactgtaggtggtgcagctagtcgatagatgcgctgccccggggtggggctgcatggccgctggttttggtgactgtgcattgcagtgcatggtgtgccgaccaagcagcaactttcatgcacagcggactgcggcgttgacaagggtgtttttttggatttggcagggccatcttgggaccgctccaggccctgctgtggtgtgatgctgttgaccaccacgcttgtctcagttttgccaatgaaggtgttgtagggttacatatatgcatggagcagcactcgccagccagggcaggcgcaccaccagactgagggctgttggttgccagctgctggctatcaatttttggctgtttgttttttgccccgcactcgctgtgtgttgtgtgagctgtctgggtggctgtgtacagggtgtgaccaactgcacaagctggggcgttgcctgtgtgtagggtactgctgttttttgccgtccaagcacagcctcataaagcccggtacgggggccaggttccctgcagctgtgctgtgggcgtaggagggcgaccaacctggtagttttttgagtgacccagggtgtgcaagcgtcgaggactgtcttgttgcctcaggttgttgtgttgaggagaccaagctcgaggagcacactgatgaggcatgttgtgtgttgagaggggggttgtgtctgcccgtttttgggggttgttgaggccgggtgacgtggtgagaacagaggaagggcgtggagtggttggggtgaggcataatgcgccgcaatcagcagttgtaacacgaaccaactgc

SEQ ID NO: 70 - DNA, 963 bp, Desmodesmus sp.
atgcaaggctgttggtgtgtcaaacttcaacaaggaccgtgtcagcaatgctgcaaaggtcctgcgtgacaggggtacatgcctctccagcaaccaggtccagtacagtctactgtataggcggccggagaccaacggtgtgtttgaggcgtgcaaggaggcaggcgtcacgatggtggcctactccccactctgccagggcttgctaacaggcaagtacacgcctgacggcccacgccccactggcccgcgaggcaacatctactccaacaagatccgtgagatccagcccctcatcagcgccatgaaggctgtgggtgaggagcggggcaagtcaccagcacaggttgccctgaactggatcatctgcaagggtgccgtgcccatccctggggccaagaacaagcgccaggtggatgagattgctggggctgtcggctggaggctcagcgagggagaggtgctggagctggagaaggcggcagacaagatcgcggcaccacttggcgcaccctttgagaactggtagagggtgcacacacaccgttgatctggctggtgctgttgagtgaatgggagagagggacggggaggtgttgcttgtattgccgtacatccggctgtacggcaggaagagttttttgtgtgagagatgtttgcgtgtcactcatcatgcggctaccccaccaaatcatgatgcacaaggggtggggcaactgagcaggacattctttttgggattatcttgtcgctcaaacgcatcctgtacattcagagcatctgtgttggtttgttgtaccagcggccaagttgtgctgcacccagttgaaagtgcactgcaacaaccgagcagctgccagccttgccgcatggcatatgtggcagctgccttgtgcacggggtggaagccaagcatcgccggtgtgcgagcttcccttgtcatgttgtaaccaggcggttgct

SEQ ID NO: 71 - DNA, 890 bp, Desmodesmus sp.
atggcccgacaacaacaagatgttcgagaccttcagctacctgccccctctgagcgacgaccagatcgctcgccaggtggactacattgtgaacaacggctggaccccctgcctggagttctctgaggctgagggcgcctatgtcagctccgccagctgcctgcgcatgggcgcagtgacctccaactacttcgacaacaggtactggacaatgtggaagctgcccatgttcggctgcaccgaccccgtgcaggtgctgcgcgagatcgacaacgccaccaaggccttccccaacgcctacatccgcatgtgcgccttcgacgcatcccgccaggtgcaggtggccagcatgctggtgcacaggcctgccgccgccaaggagtggcgcccagtgaaccaacgccaggtgtaagaagcttcatcaagttttttcactggcgagcctgtggcgaggttgtgcggccactgctgtccagcttctgctgctgcttggagtgtgtgagacgagagcgtgttgggttgacggcagccgcgcaggcagtggttgttcgctgcttgcctggtctgcgcgcaggatgtgtcgtgcagcatacgcctggaccttatagggtcgggtctgccttgtgatgcactcctggtgttggcccaatgcacctgcattgtagccagttttgctctgctgcttcctcagcgtgcaacgccgccctgttgaacagcaacttgtggccgatgcagcactgctgcaccatgttggtgtctgtttcttggcagtaaccactccttttggggtaatgggtgcttgcatgtatcataaccccagacctgggtcgcagtttttttgatgccggcttgtcaccccatagtgcgcactgggtaaaagatcttttatc

SEQ ID NO: 72 - DNA, 981 bp, Desmodesmus sp.
atgagggcgggaaaccctgctgacttgtcatcaaaccatcaacggcaacgttttggaagcggtgcaggtcacattgcttggtgtgttcaatgagaaccccgttggtagattgcgagtgtctggaatcgccctgcagccggcaagctccctcactgctgggccaggaactcagctgcagcagcagcatgactgtgctgctcgtcaggtcgcccacagcagcagcaaccagcaaggctgcaccggcgctgtttgaccaactagacaaggcgctcaagggtggcgatggcaaggaccttgtggccaaaaccaagggcctggttgtgtttgtgattgacggggacacctggaccctggacctgcgagagggtgcagggacagtgacgcagggccctccgattgacaaggccgacctgacgctcaccatcagtgacgacaactttgcaaagatggtcatgggcaagctcagcccccagcaggccttcctgttgcgcaagctgaagctgaatggcagcatgggcctggccctcaaactgcagcccatcctagatgcagcagcacctcgtgcaaagctgtaggctcctcctgtgtatgttattgtgtgttcttgtgtgttcaccagcagcgtgtgtacaacccgtggggtggtgtgaatggtggggtaggagggtaccgtagttgatctgtatactgtatgtgggtgtgggtgccaaggcatgcagccattgctgcagccttaccaggctcatgggattggttgttgcttccctgcctgctgcccttgcaattggcaggacgcactgttgtattggtagagatatccagctttggtgtccagcgggtggccttgctggccttggggtctcctgtggctgttgcttattggcaatgtgcgtgtcattttggccacgtgttcatgggttcatgccttgcacatgggcatgaccatgcacatggcagtgaatgttacacgggtgcatttgc

SEQ ID NO: 73 - DNA, 735 bp, Desmodesmus sp.
atgggttggtacatccgacataaattctggtggaatgggcggctgggtcagcaagcagcctcaggtcagcacagcagaccagcacaggctggcgcgcaagtgcaagtctctgtcccaggcctacgcccgctgccacaaggccaaccctgatgacgcagcagcctgcaacaacctgcagacatcccttgtgatgtgctacgcagcagacctatgcaaggatgctgctgctgcacatgagaagtgttacatgtccgtcataaacacaggtcgctaccagggcaagctggattgtgaggagactgtgcagcagatgaaggactgcctcagaaaatacaagctgtaccccttcagcccacagttgtagctgcaacccctattcagcatcagcagcagctctgcatacagcggttgtgaactgccgactgtctgctgccacgtgtgggtggacctctggtttccagctgctcattgttcaatgttgttgtttttgtctcagtaatcccactgcagtggttctgggctggtacactgtggatcagcatgtgtggggtggtcttgagcaagcgtttcatgtgtgttgccgtatgccctatgcatgcctgtgacagacccaggctctggggggctgcaacattgctttcccagttatgtctggtggtgcgcgttgggatcctgcgtagctatgttgggcttgtacaagggaacccgctgccattgtgtaacacgatgagattt

SEQ ID NO: 74 - DNA, 83 bp, Desmodesmus sp.
atgagcctgctgctgctggaagcagcctgcggcggagtgcctggatcagcgtgatgtgtgaatctgtaataggtgtactgatg

SEQ ID NO: 75 - DNA, 1034 bp, Desmodesmus sp.
atgactgccagcagttcttcagcaggtcccaccgtatatcacaatggctgccaccatgaagaccgcctgccaggcacgcctgaccagctctgtgaagcaggtcaaggcctcccccgtggccaaggccaaccgcatgatggtgtggaggcccgacaacaacaagatgttcgagaccttcagctacctgccccctctgagcgacgaccagatcgctcgccaggtggactacattgtgaacaacggctggaccccctgcctggagttctctgaggctgagggcgcctatgtcagctccgccagctgcctgcgcatgggcgcagtgacctccaactacttcgacaacaggtactggacaatgtggaagctgcccatgttcggctgcaccgaccccgtgcaggtgctgcgcgagatcgacaacgccaccaaggccttccccaacgcctacatccgcatgtgcgccttcgacgcatcccgccaggtgcaggtggccagcatgctggtgcacaggcctgccgccgccaaggagtggcgcccagtgaaccagcgccaggtgtaagaagcttcatcaagttttttcactggcgagcctgtggcgaggttgtgcggccactgctgtccagcttctgctgctgcttggagtgtgtgagacgagagcgtgttgggttgacggcagccgcgcaggcagtggttgttcgctgcttgcctggtctgcgcgcaggatgtgtcgtgcagcatacgcctggaccttatagggtcgggtctgccttgtgatgcactcctggtgttggcccaatgcacctgcattgtagccagttttgctctgctgcttcctcagcgtgcaacgccgccctgttgaacagcaacttgtggccgatgcagcactgctgcaccatgttggtgtctgtttcttggcagtaaccactccttttggggtaatgggtgcttgcatgtatcataaccccagacctgggtcgcagtttttttgatgccggcttgtcaccccatagtgcgcactgggtaaaagatcttttatc

SEQ ID NO: 76 - DNA, 907 bp, Desmodesmus sp.
atgggatgttgtgacgccgctctgtcgccggggcgggctttctgggtggcgggagaggcaggagtgagtggagaagatgcgtggtagagcagtatgcatgcattggaatggtgttgtcgtctgctgtatgaaaacgaaatgtggaaggcaatgaaggttgtaatttgacgggcatcacttttttcttacatgccacagcaccaggctgtcagggatgccaggtgactgtgtgtgtgtgcttcaagggtgatgcaagccgtggagagtatagcgcaggcagggtgtcttttgtctcagggctaggtgacatgctgcgcagctggccatcaggcaggtgttgtgtggctcggcatgcatgctgcctttttggctgccacacatgctgcacatgccctgtaatttttatgaggccgactgggtttgagaagattcctgctgcttccgtgctgctcggggcggcacacgtcacatatcttgcagctgttgtttttggttgttcagttgctttggcagttggtgggcacgttgtttgttgtcagggggctgcacgttgctgcagcggttggcataggctgcctggagctctggcgtgcctatgcaggtcctgtaccatgatgtcctgtatcactatccttcgggttgccagcctgactgctggcagtgatggcttgttggcttgaggtaggcagggaaagcgcagctttgcggcggatggatactccatatggctgcaagtgtgttgatatgcgcattgtcgtcaacgacaatgttgttgtatactgtttatcgctgctgttgttgtacggcaggttggactacattgggctttcctgatgggatggtctctgcttctttctttacttttctcgccgccagtttcaagcacacttggcaatgatggtgcctg

SEQ ID NO: 77 - DNA, 185 bp, Desmodesmus sp.
atggggatgtctgaacagcaggggctgcaattgcccttttatgagatagtgttcgtggaaggagatatgaggagtctcctggcatgtactgcttactccgcatgctgtccttggcgccgacatctgccttgtgctagcttctagtttgttttatgtcgcctggacactcgtaagactcttccagg

SEQ ID NO: 78 - DNA, 934 bp, Desmodesmus sp.
atgccaacactcgttgatactgcaccccgagagcccgctcgtctccaccgccactctcactccgatcgctgttgctgctgccaagggcactcgcgacacccccagacagcctcctgtcagggtagtgatcaggctcagccgcagcctgtgaacctgctactgaccccttagcgaactagcaagccgagcaccatggctgaggtccaccccaaggcataccccctggctgatgcccagctgaccaacgtcatcatggacatcgtgcagcaggcctcaaactacaagcagctgaagaagggcgccaacgaggccaccaagaccctgaaccgcggcatctcagagtttgtggtcatggcagctgacactgagcccatcgagatcctgctgcacctgccactgctggctgaggacaagaacgtgccctacgtctttgtcccttccaaggcggccctgggtcgtgcctgcggcgtgtcccgccctgtgattgctgcgtctgtgaccaccaacgagggcagccagctcaagtcccagatccagacactcaaggactccattgagaagctgctcatctaataagcagcctcataccctaagcagctgctgcatccggcagctgcccaaccaaaccaaccaagcaggtgctgcactctgctgcacccttatgctgcagccttgttggggtctgtttgcacgggtggtgcagcagtagatgtgtgtggggagttgtgcaggatgttgtgctgttggttttgttttttcgagccattcttttggttgtttactttgtatgtgagcagtgctggggaaggtgcagcctccgactgctgctgataggagcagctgtggtacaagtgctgacgcatggccgtcagtcgataagcatgtgctctggtatggctctgcacgactgatgtgttgtaacccagcacctttt

SEQ ID NO: 79 - DNA, 295 bp, Desmodesmus sp.
atggcaacatgcacttgtggtggtcaggggtgttggcaagcgcatgcacacgccaccaagcgtcaagtgcgctgaccagtgtagctcagcttctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagcca

SEQ ID NO: 80 - DNA, 1284 bp, Desmodesmus sp.
atgcagtacgctcctggtgatgctgcgctgaagaaggagctgctgaaatacatggcagagcacccatcagtggcgaggttcgcgatccccgatgacgttgtcttcctggaggagatcccccacaacgccaccggcaaggtgtccaagctgacgctgcgccagatgttcaaggactacaagccagctcagcccaagctgtaacggcaacgacaaaaaggacggcgacgttgactgctgtgtgtggacctcacacagcacagcacagcacagcacagcacacagcacagcacacagcacagcacacagcgcagctgatagggggcggcctctcaggcgggtggccagaagcttgtcagagcgcagtcttctagttcaagggggcacatgaccttttgcagccaaaagccacagggctgccatgttgtgcttgccggtgcatcactggttgtgctttgtgctgtccacaacacgtatttctggctgcactggtcatgttgtgacgcagggttttggtgctgctctgttggtgctgggcgctgactgcacagatactatgctgtcgcccttggtaaagcacagcaccagcgtgtgactgttttggcatctcctgctccatacgcatggatggggttgacatttcacacaggggttgtcttccgccgccgttgtggcttctcatcacaatttttcgagtccgcaacctggtgtcgacctcagttcgtccggagccgtaggcaagctgttgtgggagttttgacttctttcagtggccacatgcatgcatgcaagccatgggtgctgtggcaggcatgggtttgagggaagaacgtggcttggtgccggtgggtgtggctgggagtgccagtgacccaacccattggtgtgggtgggttgtgaaggaggccacaggcgggcttggaatggtgatgggttgcttttggggtgtgcagctgtgcctgggggtcacttgcactgtcgacaccttgtatatacaatagctggttgcgacctggcacatacttgttgtaccattattcgactgccatcgacaggtgttttcaggaggtgtgtatagcacaaagcgagatattcatgttggtttaccggtattttttgcgtagctgctgtagccgcagcagcaaggagtgtcaggggacaggctgtgggctgatggctgcccaaggcaaaagcttttgttttgcttggcactgggtttgatggtgtgctgggcttgttgtgttgatgtggcctggttcacatcccaagtaaattcaagctatcttc

SEQ ID NO: 81 - DNA, 959 bp, Desmodesmus sp.
atggggcactgcagaggacctggcagaggtgcagttccccagcagctgagggttttgtgtgtgtttgaatgggatgggcaggcagagaggtgttgtgcagtgtcgggtatcttcgaagagggggtaaggagcggtcacaactgctggccacgctagtggggtgttgtcgcagtgtggtttgaaggtgggagtgttgaaggagggtgccagactgctcgtgaggacagtgtcactttgttggagaggtatctgtggctgcagctgcagcgggtcaccggagcgtgaggcgcgccggttcagctgcattgaaggcataaaccccctcacatcccccactagctgcctagtgggtggattgttgtgcacacgcttgtgcatggcaactggtccactctggatggcactacctaggcaggcagtcaacctgcaggccactgctggaggcttgtgtgtgggttggcgggaaatggttgcttactaatgattgattgcttcaatgattgcttgctcatggctgcttgcccatggctgctcgccaatgattgcttgtcctaatgcagcagaatgcagtctgatgcgtgcccggcacataatggcattattgtacgtagtagttgtgattgtgcagttgatgtgtttgtttacagagctctctccacaggagtcctgccgcctgtcaccatcccccctattgttgctgaggcaagccttgaagggcgcatgttgtgtccaggccgcatgttgcagttaccttgcgggttcaagcaactgtatgcacgccattttcccgttggctgtggcctgtaattgttgggagcgcttagtacgtactgtgcggatacgttagctcaagtgggttgctgcgtggcacagaaacatgtgtaaatgtatgtacacacactgagtggctgctgttggggtgacagttagtttttcaaccgctgtaacatcgttttgag

SEQ ID NO: 82 - DNA, 879 bp, Desmodesmus sp.
atgcccaccaacaaggctggctgggcagggaggtttgggggatacggtgccctgcatgtactttacatggacgttggccccgcaagctgaatgtctggtgttccgtgcgccaatactggcaactcaggctggtttttttttcaggctgggtttggctgggttggcccagcgtagcaccccagactgttggttctcattggagccgttggtgagactggttgcacatcacttgctctgcacactgaaaccgatgtgtgtcttgtgaacatgatatgtcggcaatccgtcctgccaggcactgaaagcccaaggcgctctcaacagcttcaccggcctggctgcatgcacgcgagctggctcgtgtgacacctagaacagggcgcagttgcagcaaacttggctgatggctgcctgctgttttaggccgcaggcgtgaagcagccgcagcatgcatggtgtgccgctgcggttgtagcctgctgtgcgctcaagaaggtgtccacagctggggaagtgcttccacttcttttttccagatgggcaatgcacagcaccgacctctgcagcgcttggtgctcatggcaactcagcacgcatggcaggaccaaataatgctcatcaaatctttcgaacacacatctctgctgtgcgccccatggcctctggctacatctaagccaggctggctctcctgcatgcggctgcaccttcagcggctgcacccagaaggactcggggcaatgacagcggtgcagatgtgcatgtctggtgcaacggcacatacatacaatttggtggaggccatccgatttgaagatgctgaagcgaaaccttggagagtcgaaggtttgatgtcgagagggtattgtaaaattatat

SEQ ID NO: 83 - DNA, 811 bp, Desmodesmus sp.
atgggcggactctgagccctccactgagacagtcgaggcccccccagtcgaggcagcagcagagccctccccaacaccggagcctgagccagtggcagcagccgcagctgaaccggaagtagcacagccagcagcagaggcaccagctccggagccggagcccgaagtgaaaatgccacacaagcccaccgccgaggagctggaggctgccaaggaggagctggtgcaggagattgtggagaaggtcctcgagatccctggcgctgtcacccagacgatctccaccgccccctacgactcccgcttcccctccaccaaccagaacaggcactgcttcatccgctacaacgagtactacaagtgcgcctttgagcgtggtggcgaagacgcccgctgccagttctacaagaacgcctatgagagcctgtgcccccctgactgggttgaggagtgggaggagctgcgccagcagggcatttggttcggcaagtactgagcacagcagttccatgctcggtgctttgtgctgacggcgctttgccgtgcacgtgggccgaccaggcagcatatgcacagcgcagcaggtggagaggcacaggagcagcgacccagtttgtagttgcagcagttttgtaccatacatagggcctgcttcagggcagtggtagccgcatgttggtgtgtgtgcgtttgtttggtgacaacctcacgatatggttgatgtgtgtgtggagttggaggcctgggaagtggtggcaaagcacactgcagctgagcatccatcagataacaaggtaacaagtcccgaaac

SEQ ID NO: 84 - DNA, 1497 bp, Desmodesmus sp.
atgggagctctctcgggtcctaccgcgtatacgatgtagtctgttgggcacgctggtgcgtgtagcctactgtaaccactcctgctcattgccacataaaggccaccacgcccaagttcacgcactaggactgctagtagtagtgacattgcagctagtcagtgacagtgcaggcgttcctacccgagagagctcccttgggatggttcggcgtggctgagcagcgagtgatggcactgccagtgagggcctacagcgatcagtgcgctccggcgagcatcgaccgtcgcgatggagtcatcatttccattgtcattatctggaccgacagcgaggagagctgctactgcgacgagtgctgttcaggagcaacagcagcagggcagtcactgcgaggcccagctttgccagcagctgtggtgcaggggtcaaggccagccaggagtgctgtggggaccttctctgggctgattggaggggttgcgacatggctgtggtgctgtagcgtggtgtgatgtgcttaccgttgcggcagcaggaacggtgtagggtctggcgtgctggggtgccctgtgtacccagtggctggtctggctgcattggtggccttcagcgcatcactgggggggcacagtggggttgtctggccactatgctatccctgtgcatagaggggtgcttgtgcagcattgcagcagtatggtgcagcagtgagggcatgaggcacgctggcacaggtgtgggcttgtgcatcatggctgtgggttcgcagctgttaggacctgtgctgacatgtgtgtgcatgggtcagcaggacaggccagggttcccaggtggggacagcgtgagccagcagcgctgggctgggggggcatcgagattgccaccagcaccctgtggagacctgcagtggttgggtgctgcaggcagctgtgagagcagcttggttgatgccaagtgtggcatggctgtgggccaggcatgttgtctatgcagctgagcagctgatagttgttggttgttgttgggctcagggccccaggctgcagttcacgactggcaacggactcttgcaatggtggtgtgtgtttgcctggtttctcacgccaccgagcgctcatgtgtgcatggatggcttcccctttggtgagcaggaacgttgtgcagcatcaacactgcatgttgggtgcagatgttgagggggggaggtcggctcacgccgtaaaaacccttccgccgcaacctgacgtgacagctgtcacgctgtatgacggcgtgtgcgcagcgtggcgtggtccgccagccgcagacaggcacaaatatgtgttgtgcctgtgctggtcaagttggcagggttcaggtgttgacctgcccttgcgccgtggatttgaggcaagctaaccccgagagaggcgagctctctcgggtcctaccgcgtatacgatgtagtctgttgggcacgctggtgcgtgcagcctactgtaaccactcctgctc

SEQ ID NO: 85 - DNA, 1090 bp, Desmodesmus sp.
atggcatatcacagtccgtttgttgcccactctctcaggcgcatcgccgcacacgcacacatcttccggaacagatccaaccatgcagctctctctgtgccgtgcccccacaatgtcccgggtggtggctgcccccaccaggcgcccccggatcgtggttgtcaggagcggaaaccatccatcgatgaaggacgttgaggacatcaacgccaaggtgaaggaggccatcgaggaggtagaggacatgtgcaatggcggtgatgctgcacactgtgctgctgcctgggacaatgttgaggagctgagtgcagctgcttcccacaagaaggacgctgtgaaggaggacaaggtctcttcagatcctctcgaggctttctgcgatgacaacccagacgccgatgagtgcagggtgtatgatgactgatgtgcgcgagtgcgtctgcgccagcatgcattataacttgacttgtggagtggagcggcatgcccccaggcaaaccccacagtgtgcagtatgagggtctggcataaacggcgcagacttggggggcctagtagctttgtgtcccagcggggaggggtggcccgcaacccctgtcagcatcgccaccatctcttgagacctttgactgcagtgccctgtgcatgcagcctgtattagcgcagcgtcccagcagtgtgtcctgtttgcagcttcgtccaatgctgcagtggcagccttccgccgtggacccttgtgcaacccctttggacggtcttcactgttcgccagggtgcagttgttgcagcgggtcgcatggttgtatgtatgcagagtgtgcaggtggcgtaaggagcggtctgctgtgcgtctggagtcgtcgttctgaattttttgaagtgtttgtccccttcgctgacagtcagccagtttgtcccctttgctggctgactgtcggacaagggtgacacagtgtggtggtgtagtaacggatatggttggcgggctgtggcatgctgaggttttttgtcgtgtgcccatcgagatgtttgatggtggcgttgtggacagtttgatggtggtacgtctttgtaagtacaactgagttcg

SEQ ID NO: 86 - DNA, 1496 bp, Desmodesmus sp.
atggagctctctcgggtcctaccgcgtatacgatgtagtctgttgggcacgctggtgcgtgcagcctactgtaaccactcctgctcattgccacatgaaggccaccacgcccaagttcacgcactaggactgctagtagtagtgacactgcagctagtcagtgacagtgcaggcgttcctacccgagagagctcccttgggatggttcggcgtggctgagcagcgagtgatggcactgccagtgagggcctacagcgatcagtgcgctccggcgagcatcgaccgtcgcgatggagtcatcatttccattgtcattatctggaccgacagcgaggagagctgctactgcgacgagtgctgttcaggagcaacagcagcagggcagtcactgcgaggcccagctttgccagcagctgtggtgcaggggtcaaggccagccaggagtgctgtggggaccttctctgggctgattggaggggttgcgacatggctgtggtgctgtagcgtggtgtgatgtgcttaccgttgcggcagcaggaacggtgtagggtctggcgtgctggggtgccctgtgtacccagtggctggtctggctgcattggtggccttcagcgcatcactgggggggcacagtggggttgtctggccactatgctatccctgtgcatagaggggtgcttgtgcagcattgcagcagtatggtgcagcagtgagggcatgaggcacgctggcacaggtgtgggcttgtgcatcatggctgtgggttcgcagctgttaggacctgtgctgacatgtgtgtgcatgggtcagcaggacaggccagggttcccaggtggggacagcgtgagccagcagcgctgggctgggggggcatcgagattgccaccagcaccctgtggagacctgcagtggttgggtgctgcaggcagctgtgagagcagcttggttgatgccaagtgtggcatggctgtgggccaggcatgttgtctatgcagctgagcagctgatagttgttggttgttgttgggctcagggccccaggctgcagttcacgactggcaacggactcttgcaatggtggtgtgtgtttgcctggtttctcacgccaccgagcgctcatgtgtgcatggatggcttcccctttggtgagcaggaacgttgtgcagcatcaacactgcatgttgggtgcagatgttgagggggggaggtcggctcacgccgtaaaaacccttccgccgcaacctgacgtgacagctgtcacgctgtatgacggcgtgtgcgcagcgtggcgtggtccgccagccgcagacaggcacaaatatgtgttgtgcctgtgctggtcaagttggcagggttcaggtgttgacctgcccttgcgccgtggatttgaggcaagctaaccccgagagaggcgagctctctcgggtcctaccgcgtatacgatgtagtctgttgggcacgctggtgcgtgcagcctactgtaaccactcctgctc

SEQ ID NO: 87 - DNA, 1113 bp, Desmodesmus sp.
atggaactcctcccagctttgcctattcatccctccacacacaactacagtcaagatggcaatgctcctgaagaaggccgctgtggcccctgccaggaccagcgtgcgcagcaaggctgccatcgagtggtacggccccgaccgtcccaagttcctgggccccttcagcgagggtgacacccccgcctacctgaccggcgagttccccggtgactatggctgggacactgctggtctgtccgccgacccccagaccttcgccaagtacaaggagatcgaggtgatccacgcccgctgggccatgctcggcgctctgggctgcatcacccccgagctgctggccaagaacggcgtgcccttcggtgaggccgtgtggttcaaggccggtgcccagatcttccaggacggtggcctgaactacctgggcaacgagaacctggtccacgcccagtccatcctggcaaccctggccgtccaggtgctgctgatgggtgctgccgagagctaccgcgccaacggcggtgcccccggcggcttcggtgaggacctggacagcctgtaccccggtggtgccttcgaccccctgggcctggctgacgaccccgacaccctggctgagctcaaggtcaaggagatcaaggacggtcgcctggccatgttcagcatgttcggcttcttcgtgcaggccatcgtcaccggcaagggccccatcgccaacctggacgagcacctggctgacccctccggcaacaacgcctggaactacgccaccaagttcgtgcccggcaactaagttgttggtgccaaccttgcagctgtagcgtcctcctgcaggattgagtgtgcactgaccctccctgccgccatgtcgtgtttactggggagttccgcgctgtgactccagggtggtgctttgtataatacccattcctgtggggttgtgtgtgttcagggaccatggcagagctgaaacatgcccttcgatggtgccagagaggagagttctgtgtgtgttctgtagttgtgcagttgcacgacagccggcctgtgagccaggctgcttaacagtttgcttgatgcgccgggccgacaggagtgtaactgtgcgctgtg

SEQ ID NO: 88 - DNA, 1129 bp, Desmodesmus sp.
atgggagaagcagtggtatcaactcctcccagctttgcctattcatccctccacacacaactacagtcaagatggcaatgctcctgaagaaggccgctgtggcccctgccaggaccagcgtgcgcagcaaggctgccatcgagtggtacggccccgaccgtcccaagttcctgggccccttcagcgagggtgacacccccgcctacctgaccggcgagttccccggtgactatggctgggacactgctggtctgtccgccgacccccagaccttcgccaagtacagggagatcgaggtgatccacgcccgctgggccatgctcggcgctctgggctgcatcacccccgagctgctggccaagaacggcgtgcccttcggtgaggccgtgtggttcaaggccggtgcccagatcttccaggacggtggcctgaactacctgggcaacgagaacctggtccacgcccagtccatcctggcaaccctggccgtccaggtgctgctgatgggtgctgccgagagctaccgcgccaacggcggtgcccccggcggcttcggtgaggacctggacagcctgtaccccggtggtgccttcgaccccccgggcctggctgacgaccccgacaccctggctgagctcaaggtcaaggagatcaagaacggtcgcctggccatgttcagcatgttcggcttcttcgtgcaggccatcgtcaccggcaagggccccatcgccaacctggacgagcacctggctgacccctccggcaacaacgcctggaactacgccaccaagttcgtgcccggcaactaagttgttggtgccaaccttgcagctgtagcgtcctcctgcaggattgagtgtgcactgaccctccctgccgccatgtcgtgtttactggggagttccgcgctgtgactccagggtggtgctttgtataatacccattcctgtggggttgtgtgtgttcagggaccatggcagagctgaaacatgcccttcgatggtgccagagaggagagttctgtgtgtgttctgtagttgtgcagttgcacgacagccggcctgtgagccaggctgcttaacagtttgcttgatgcgccgggccgacaggagtgtaactgtgcgctgct

SEQ ID NO: 89 - DNA, 1129 bp, Desmodesmus sp.
atgactcctcgcaccatcacctgcttgtgagtcacctcttccccgtcactcagtcaacatggcattctcaatgatgagccgtgccacctccgtcaacgtggtcgccaagaagggcggaaaggctgcccccaagaaggtggccaagaaggccgcttctggtggctccaagggcgtggagtggtatggccccagccgtgccaagttcctgggccccttcacccaggcacccagctacctgactggcgagtttgccggtgactacggctgggacactgctggtctgtccgctgaccccgagaccttccgccgctaccgtgagctggaggtgatccacgcacgctgggccatgctgggtgccctgggctgcatcacccccgagctgctggccaagaacggcgtgaacttcggcgaggctgtgtggttcaaggccggtgcccagatcttccaggacggtggcctgaactacctgggcaacagctccctcatccatgcacagtccatcctggctaccctggctgtccaggtgatcctgatgggcctcattgagggctaccgcgtgaacggcggccccgccggtgagggcctggaccccctgtaccctggtgaggccttcgaccccctgggcctggcagacgaccccgacaccttgccgagctcaaggtcaaggagatcaagaacggtcgcctggccatgttcagcatgtttggcttcttcgtgcaggccatcgtcactggacagggccccattgccaacctggacgcccacctggcggaccctgccggcaacaacgcctggaacttcgccaccaagtttgtgcccggcaactaaaccaccactgcacactgctgtggccatgtacggccgggacccgcggggtttgtggtgttgacagctagctcccgcacagggtgcaatgcgtgcgtgcgtgcgtgtgttcgtgcgtcttgtgtcattcactgcaggcgcctgcggggcctggtctgttggaagttgcttgtagcgctgtgtaggagttgcgagcgactgtacagtagcagcgggcggtgtgtgtgtttttgttgggttgactgggtgcaggacttgctgacgcatgtgtgaggggctctgggtggtgtaactgaggagtagt

SEQ ID NO: 90 - DNA, 1430 bp, Desmodesmus sp.
atgggaagagatgcaagacaaccgctttatagatgcattgcagtcagctagtgatcctgctgctgcactccggccagacccagatttactgaactgatctacaacagatccaagcaggacaagtttgcagccgtgatggggctcaagcacacgcagctgtgcacattggcgctgctgtgcggcgtactgcagctggcagcagcctcacagtgtgcagtgggtgacgcagcgtgcttctgcaacctcatcaaaggcacctgggtggccactccctccaccatccgccctacctgtagaaagacagtggacagtcaaggagtaaagcgtatagtcgatgtttacttcccaacagacatcgggacccctggtgtcaagttcaccccctgggtgcacctccatggcgtgatgtggagcaagtggtggctggagcagaacccagagtggcagcagctgtcgaagaagtggacaccacccatgggtggcaacgcagacgcctcccgcatgaatgccctcactggcagccccgtcacaggagagggcagcaaggctgagggcgcaggcttggtgaagcagcagggctatatcctgcttgtgccacagggcacaggggatgtgacattaggccagatgtggaacagcgtcttctggccctgcatgacctccacccgctgcgtggacaagtcggtggacgacgtctccttcctggcgggtgtcatcactggcatgcagcagctgctgcctggcctgcccctcaagcctcaggtggccctgtcaggctactccaacggaggcatgatgatccagacgctgctgtgcaacaagcccgaggttgccaacagcctggctgctgtggcgctggtggggacgatgctgggcagcgactttgctgcaggcagctgcaggcagaagctgcccaagagcctccccctggtgtggctgcatggggtgaaggatccggtgctgccctacgcggctgggggcagcagcttgggggtcaaggcccttggtgcagaggctggcaccaagctctgggctgaccgcatgggctgccctggggtcagcggcacccctcagacagtcttcactgacgggggtgctggcgtgagctgtgtgaacctgtgtggaggagggggccagccgaccactgcggtgctgtgtgcagttgcagaggcaggccaccagctgtgggaccagcctggcagcgggtacagtgctggggtgatgctgtgggcctttggcggctttgcagggacacccaagccactgtgaagtgccgtgggttggctggctgccaacttgggcaacggttgggcttgtagatggtgtctgcaaattgtggtcaggtgaccgacttgattgacatagcaggtgtggttgagtgcggtttgtgctgttatggtgtacatgcgaattatgaactgcttggggcc

SEQ ID NO: 91 - DNA, 825 bp, Desmodesmus sp.
atgggcagcggcccctgaggctccccaggctgctgtggcggctctgccagaacttggaatgtgtgtgtgtttgttgtgcactgaagcacacagtagtgcatgcccggccaactggtggcggtctggcaatgggtgatgtgcctgttgtacagctgtccgtcactgttgaggcaatcatcatcatcatcatgccggtatgcggtggttggttgcagtctgcgctgtcctctgcatgtgccagctgacggaggtgataccagcaggtttgtgcctgccgctgtgtccctgtttcaagctgtgtgtttgcacttggagtgacagagagcagtacagagcatgcccgcttcccctttgtgcaccagtctgcctgagcgtctcataaatcatcggtttcagcggttggcattgtggctgtccctttgtgctcccacctactggtggtgtggggttcatgcatgaaccccataggtctggctatggcagttgggatgccgtcgtcttcgtggtttgaatgttgcaccccttgccaaggtagcgtgggctgggctgcgtggtgcaccacactgtttcaggcatgagatttggttttgtggccttggtttcaagatttgagtgattccgtcgacctggtccccagtggtcttgtctgccaggcttgctccaagcctcctcgagacgtgtgtctactttgcggtctacccagttcatctagtgtggagagagtgccgctgccaggctccgagccgagccgagcgtgtgacaccacgcatgttcatttgcattttcctttgcgttcatagccaggcattgtaacatatatgcatc

SEQ ID NO: 92 - DNA, 1003 bp, Desmodesmus sp.
atggaagtggggggtgctggtggggtgtgtggcggcgtgtggcgcctggcagcagcagctggcagcagcgccgcctgtgagcgtgggcatgatgctgtgctacggcatccaccgcctgcagcgcctgcactacgtggtgcagctgtcacggcagcagcagcagcagcagcggggggtgccgccacagcagcggcaggataggcagccgccacagcaacagcagcagcagcagcaggtagtgcttgacgcagccagcaaagctgtgggacgcagcagcaggccagcctgcaggcgacagcgacgtgctcgctcccgctcaggaacgagctcccgcgctgacagcagcagcactatcagcagcagaagcagcagcagcagcagcgacagggctttagctggccaggtagcaggtgcagcgggccagattgggactgccagtgagcaggacgtgactgaccttgtggcggtgccgtggaggtctatgatccaggaggcagacagtgtgttttgccgcaggttccctgctttcaaggcctggctagcacagcagcagcagcaggaacagcaggagcaggattgacagctgccacaggtggcagtggtgatttaggttgcttgtgctgtggtagctagcagcagcggggctcggtgttgtggtgctgttgttttgttaagaggctgttgtatggttcgctgcttgtacaatacagtggcgcctagcccatttgtactttgtggtcgaatgtatccacatctgcacttgacttgttttttgaaaggtctgtctcgcctctgcagcgctgcagcaagcgcacagcagcaggctctgtactttcaagtggtgaagttgcatgttgatgaagccattatctggagtgggctatttgcctgatgttggcggctttctgggctgttggtgttgagacaggttggctctcaactgtgtcattggctgcaggcacgatggaagtcttgtttgcaaattttttaacccaccgtgtac

SEQ ID NO: 93 - DNA, 1880 bp, Desmodesmus sp.
atggattatctgagcctacgatcaactctgcacatgctgcggctgcgatctgtcctgtagcaggggaagctcccttgctctgtctcgggcagcgacacaccccagtgcgtgcaaaccgtatcgctctattgcccacagcagcaggtgcagcagcgtccctgcctaccatggcactagcagatgcaacagggtggtctgtgctagtcgcccgatgtgggccaggggcactcccgagcccgatgacaatagtaagcctggcaggggtagtggcatggcgtcaatcatcactaaggagatcaccagctgcgacagcgtgccagagctggagtcaattttcattgagcacggcaacttcacctatctcaattcatctgcagccctcaccaagtatgccaagctacgtggaagcagcatgcgcagccctttcttcagtaagctggctgcggtgtggctgacaaggctcccagaggcaggggggcgggagtatgccaacgtcctgtgggcctgcagcaggctcggcagcagcaagcaccctgtctgggctgagacctggcaggcattccttgaccttactgaaaaggacgtcaaccgggataagccaccttcacggccgcaagacatctcaaacgtgctgtacgcagccgccaagctgcggcagcagccgcggcctgatgagctgctgctgctgctggaagccttcacacatcctgttgtgctggcagccgcaaacccccaagacaccgccaacattgtttggtcgctgggacagctcagcgttacccctggctgggaggcagaggtcagccaggagctgctgcaggggctgttggcgccgcagctgctgcagagtgtggcggcagacggcataccccagggtgtatccaacgtgctggtgggtttgggccgcatgtgcacagcccagtcaccactgctgtccacagctgcagcacaggggtatgctgggcagctgctgtcaggtgttgggctgaacaaactgtctagctggaaccctcagcacatcaccaacgtcatgtgggcactgggtgagctgcaggttaacaaggaagattttgttcgagctgctgttgcagcagccccaaagtggctgccgttgagcacgggatatgacctcactcaggcagcaagtgcctgtgcccagctgcagtacagcgatgagcacttcatgaggctgctgctgcagcggggccagcagctgctgcagcctaacaggcgcagccgcggccggcccttgtctgagccagacaaggctgccttagcaaccatatgctctgtgccggttgtgtgcttagacatgcggggtttggcagatgcagcacgcaagcttgtagcagacagcggcctcatgcagcaagcccgcacccatcctgcgcaggctatggctgttccacagctggctgttggagcaccagctgctagatgggatggggctgtcgggtttggtgacggagcagcagctgcagcagggggcaaagcaggcagcagcatggagggacaagacccggctgtgagtcgcacggcttccacagaagctgttgtattgtcatgttgctggcccgcgctggtgtctggaaacctcttccttggtttgtggtaaacttgtttgttgctggccacacgggatacctttggctgtgtcactcggtgtcattttggtgtgggaattgtccatcttaacctgtgaattcaggtgttgttgtccaacagcatatgccagtcactgtgggcctgaatctacattggtgcatatgtacttcttggccgtgaaggttgaaatgggcatgtccctgctgcttggcgtgccacttggcgtgccactcagtgcagtgcactcagtggctctgtaactgtgtcagatggtc

SEQ ID NO: 94 - DNA, 565 bp, Arthrospira maxima
atgagcaaccaaactttggattaactcaactgggggaaccacccacccaggtaataagtccgcttgagcggcgggatcgggtaaactgagcataaccaaacctttagcggttttgggatattgagcaacggttgctaaggcgattagagaaccaatagagtctcccacccagatggtcggacggcggataaaagtttgccaaaagtcatgcaccaattcggtccagagggaaacattatatggagcgatcgccttttcagaaccaccaaaccccaataagtccaaagcataaactgggtgcgaatgactgaagaccgacaaattgtgtctccagtggccgatagatgcaccaaagccgtgaaggaaaattaaaggaacaggagagttgctcccgagtgggtcttgaacctcaaaaataggcgatttagcctctttagggtgctgccaacgcagaaaagtatagcgaactctccaacccctccaaatccaatcacgctgaaagcccatctgtttgtgcaagtctggtaaatccatctaaccgtcgtcctgagtgcaacataacttg

SEQ ID NO: 95 - DNA, 1083 bp, Arthrospira maxima
atgcttcccccaaaaatgcggataatttaggcatcggtgcgacatcgcgtgaggggaaaacccgaaaagttctttaagatttcaaaaattgaagaggtattatgagccaaaaggttatacacccgatggataagtttcagcgtcaggtgcattccctagtgaagtctgacattgttaagcctgaagatagcctgtggaaaatagcactgctatttggggataagtggaagtattggaaagcagaactgattgaattcggtttttccatgcaagatccgattagtgaactgttggctgttgatgtttgggatgaagaataaaactctgtgaagcgttaacattttggtaagcatttagcgagggcatcagccaagtctgtcgcggtctgatagcgatcgcgaggggtatatttagttacccgtttaattacctcgcgtaagtccggggcaatagtgggaatgcgatcgagatcaaatccgtattcttggtcttgtttcccgatataatttaggggactctcaccagtcaataaaaaaatcaaagtcgcaccgatcgcatataaatccgactgagtgagtggctgtcccctatcctgttctggtgcgctataaccttccgccccaatacgggtttctaggggggtaccaatttccttaaccgcgccaaaatcgaggacaataatccggcgatcgcgatggcgcaccatcaggttagccggtttaatatcccggtggatgatagggggttgttgggtatggagataatccaaaacttcacaggcttggatcatgtatttaatcgccattgtcggcgttaggggtccactttgatcaacttgtttttccagatcctgaccgtgaatcatctccataactaaatactttttacccccatccacaaaaaagtcataaaacttggggactccctgatgattaagggatttaagagttctagcctcccgttcaaacaattcttgcgccttagctatcttagccatatctgcattcatctccttgagtaccaccaaaatggggattggctttccggaatgagtctgaggcgcattagccttgctattattgc

SEQ ID NO: 96 - DNA, 1097 bp, Arthrospira maxima
atgcatggttttgtgctggatgaaaagggatataaaatgagtaaatccctgggaaatgtggtagacccggctgtggttattaatgggggcaaaaatcaacaacaagaccccgcttatggtgcggatatcctgcgtttgtgggtgtcttcggtggattattcggcggatgtacctttgggtaaaaatatcctgaaacagatgtcggatgtttaccgcaaaattagaaatacggcgcggtttttgttggggaatttgcatgattttgacccagcaaaagatgcgatcgcttatgaggatttaccggagttagatcggtatatgttgcaccgcatgacggaagtgtttgcggaggtgacagacgcttttgaaacttatcagtttttccgattctttcaggtagtgcaaaatttctgtgtggtcgattatccaatttctatttggatattgcgaaagaccgactatatattagcgcggaaaatggtttccggcggcgcagttgtcagactgttttggcgatcgccctagaaaatttggcgaaagcgatcgcgcctgtcctttgtcatacggcagaggatatctggcaaaatataccatattctacgccatatttatcagtatttgaatctggttgggttcggttagatgaagcctggaaaaagcctgaattagcagagttttgggtgaaattacgggagattcgcgccgaggttaatcaggtgatggaacaggcgcgcaaagataaaatggtgggttcttctctggatgctaaaatcttgctatatgtggcagatgaaaaattgcgtcaacagttagcggcaatgaatccaaatccggcggaatttaagcagaataacggcgttgatgctttgcgatatctgttgatttgttctcaggttgagttactggaaaatccagataaactggcagatttgcagtataaaagtgagtcggaattgatggcgatcgctgtggtgaatgctgagggcgaaaaatgcgatcgctgttggaattactctgtccacgtcggtgagtcttcagaacatcccaccatctgcgaacgttgtgtttcggctttagccggagagttttaaccacat

SEQ ID NO: 97 - DNA, 109 bp, Arthrospira maxima
atggctcagtggaaccacaccgatccatcccgaactcggatgtgaaacgctgaaacggcgaagatacttggcgggtagctgcctgggaaaatagcaagatgccaggatt

SEQ ID NO: 98 - DNA, 628 bp, Arthrospira maxima
atgctactcaggaaatctttaccctagaacaggcggcgaaaaactttggttttgatagggtcaataaggcgggggcaaaatttgactgggataagctcgattggttgaatggtcagtatatccataatctgccagtgtcggaattaacagatatgttgattccctattggaaagaggcgggttatgacttcgacccacagagcgatcgcctttggttggaacagttaacaaccctcattggtcctagtcttacccgtctcaaagatgcagtagatatggccgcgatgttctttccctccagtgtcagtttagatgaagaagcccagcagcagttgcaacaagagggagcccaaaccgtcttggcagctatcaaggataaactcgaatctgagcctacactgacagcagataccgtcaaggatatgatcaaagctgttaccaaagagacgaaacttaaaaaagggctgatcatgcgatcgctcagagcagctctcactggggctgttcatggcccggatttagttgagtcctggttgcttcttcatcagcgaggaactgatctaactcgcttgcaaaacatattagatcgttaattgttattgaggctgaatgctatggcggatagccc

SEQ ID NO: 99 - DNA, 227 bp, Arthrospira maxima
atggttccctagtacaggtggaaaccacggctatcagcgaaacaggggttttagttacgggttggcaattttggcgaaaccatcagcatcagttagtttctccccatttgttagcgatcgccactttacccttaccctccctagaaaatcccctagtagccgggaccgtcgcctactacaaacagcagcgccaggactggtttcggctatatttactccccgccgcc

SEQ ID NO: 100 - protein, 88 aa, Chlamydomonas reinhardii
MAAIAAPGAAAAPAVEEKTTFDVVLEEIPADKKVGVYKVVRNIANIAVNQVKDFTATLPKVLKEGLSKEDAEAAKAQLLEAGAKAKVA

SEQ ID NO: 101 - protein, 77 aa, Chlamydomonas reinhardii
MVAKMCTSRPGRGHTTAQVSPPCDTMRGGRRCGGHWHTRQHIRTYTDVLAVRSMRMTGYCGGCRTQFFSADVCSSRP

SEQ ID NO: 102 - protein, 150 aa, Chlamydomonas reinhardii
MASYKPKSVKDVPAEDFIKTYAAHLKANDKIQLPSWVDVVKTGKFKELAPYDPDWYYVRAASVARKLYIRQGMGVGLFRTQYGGRNKRRGSKPEFQSKASGGLVRHIMKQLEECGLMEKAPEKGRRLTANGQRDMDQIAGRITVQLQAFF

SEQ ID NO: 103 - protein, 15 aa, Chlamydomonas reinhardii
MPKATIVQTVEKYLN

SEQ ID NO: 104 - protein, 153 aa, Chlamydomonas reinhardii
MAAKPSGPRTLVNTIKLLVDAGAAKPAPPVGPALGQAGLNIMAFCKEFNAKTANYKEGTLLRVKVRVFSDKSYEWDLKTPPSTWLIKKAAGLARAADRPGHELSGTVSLKHLYEIARVKQRDTPNLPLQSIVTALLSTCRSMGVRVVARPEEA

SEQ ID NO: 105 - protein, 136 aa, Chlamydomonas reinhardii
MSGGRAVCVAGRYRQCARKCTLLVPVASALGASRVCKSWFLAGVSVQWTTSSAARMLRYVCVEDESLVGEPVAEGQGPLLLCDRVLIVAIVFCRRKRRGGGRNVCCSWVLRSRDPKHTAQKNVFIQPGEVCVCCGQ

SEQ ID NO: 106 - protein, 242 aa, Chlamydomonas reinhardii
MSAAFQRGHTAALSLYDDPPKTVRCGGHRKTTATYTLTTSGGVCVYKNGSGPERLLEFGSTCTCAVVQGRSVWMANVGDSTAVLGTDNGASYTSKTLTVRRNGHNAEEAKRMKEGFSGTVNLKDAAHEDGYLQVVTGPWQGYELSVTRALGHKHMSEHGVLTEPYVVTFEASKDDCCLIMASDGVWDVMDGQEAVNRVMEVASEGKTAAQAAKMLVEEAVELGVKSPCGEADNTSAIVVFFA

SEQ ID NO: 107 - protein, 259 aa, Chlamydomonas reinhardii
MALQQTRAFSGRMSLKSAAAPVRVSRASRARTVLVEARQFRKAMGVLGTKAGMMSYFTEDGLCVPATVIALEEGNVVTQVKTQDTDGYNAVQIGYKATAEKRVTKPELGHLKKAGVPPMRHLVEFKLKDRAAVEAYQPGQALDVAALLKEGEPVDIAGITVGKGFQGTIKRWHHKRGAMSHGSKSHREHGSIGSATTPSRVFPGLKMAGQMGNVRMTVKNQSLLKVDTERHALVVKGSVPGKVGNVVEITPAKLVGVNW

SEQ ID NO: 108 - protein, 386 aa, Chlamydomonas reinhardii
MSHRKFEHPRSGSLGFSPRKRCRRGKGKVKSFPRDDASKPVHLTAFMGYKAGMTHVVRDVEKPGSKLHKKETCEPVTIIECPPMVVVGAVGYVKTPRGLRSLNTVWAEHLSEEVKRRFYKNWYKSKKKAFTKYAKKYSDGKKAIEAELAALKKHCCVIRVLAHTQVKKLGFGVKKAHLMEVQVNGGTVAQKVDFAYSMFEKQVSVDAVFQPNEMIDTIAITKGHGVQGVVQRWGVTRLPRKTHRGLRKVACIGAWHPARVKWTVARAGQQGFHHRTEINKKVYKIGKKGDASHLATTEFDVTKKEITPMGGFPHYGVVSEDYLMIKGCVPGTKKRAITLRRSLLPQTSRNALEEVKLKFIDTASKFGHGRFQTTEEKLKTFGRTKA

SEQ ID NO: 109 - protein, 135 aa, Chlamydomonas reinhardii
MGYNMGIRLVDEFLAKAKISRCSSFRDTADVVAKQALPMFLNVTANVTNWSPDQTECSLVLTDNPLADFVELPDEYRELRYCNVLAGVVRGALEMVNMEVECRWVSDMLRGDDCYELRLKLKEHRDEKFPYKDDD

SEQ ID NO: 110 - protein, 264 aa, Chlamydomonas reinhardii
MFTMAPKGKKVAPTPAAVKKAAAPAKQTNPLYEKRAKAFGLGGAPKPKRDLHRFVKWPQYVRLQRQKRVLSMRLKVPPVINQFVTRALDKNSAETCFKLLMKYRPEDKKQKAERLKAEAAAREAGKEAEKKKPVVVKYGLNHITTLVESGKAQMVVIAHDVDPIELVCWLPALCRKMGVPYAIVKGKARLGQIVHKKTATALALTAVKNEDQREFAKLVESFKSQYNEGPRVQWGGHILGMKSVHKQKKRERAIAKELAQRAGV

SEQ ID NO: 111 - protein, 51 aa, Chlamydomonas reinhardii
MGRSVDETLRLVKAFQFTDEHGEVCPANWNPGAKTMKADPTKSLEYFSTLS

SEQ ID NO: 112 - protein, 370 aa, Chlamydomonas reinhardii
MQKSAFTGSAVSSKSGVRAKAARAVVDVRAEKKIRVAINGFGRIGRNFLRCWHGRQNTLLDVVAINDSGGVKQASHLLKYDSTLGTFAADVKIVDDSHISVDGKQIKIVSSRDPLQLPWKEMNIDLVIEGTGVFIDKVGTGKHIQAGASKVLITAPAKDKDIPTFVVGVNEGDYKHEYPIISNASCTTNCLAPFVKVLEQKFGIVKGTMTTTHSYTGDQRLLDASHRDLRRARAAALNIVPTTTGAAKAVSLVLPSLKGKLNGIALRVPTPTVSVVDLVVQVEKKTFAEEVNAAFREAANGPMKGVLHVEDAPLVSIDFKCTDQSTSIDASLTMVMDDDMVKVVAWCDNEWGYSQRVVDLAEVTAKKWVA

SEQ ID NO: 113 - protein, 71 aa, Chlamydomonas reinhardii
MGEAVLICFSGVSCGGCRRAWRSTHAVECSGALQRLAVAAGAVMQDARERRGIVCTYIKQYGNGAEPCPAQ

SEQ ID NO: 114 - protein, 34 aa, Chlamydomonas reinhardii
MAAPQAARPQRAGKGAKLSALLAAEHGRRPRSGA

SEQ ID NO: 115 - protein, 413 aa, Chlamydomonas reinhardii
MAAAPVERTGFDDRAFDTKMQQFLGNNEDKFYTDWEESFESFDQMNLHENLLRGIYAYGFEKPSAIQSKGIVPFTKGLDVIQQAQSGTGKTATFCAGILNNIDYNSNECQALVLAPTRELAQQIEKVMRALGDFLQVKCHACVGGTSVREDARILGAGVQVVVGTPGRVFDMLRRRYLRADSIKMFTLDEADEMLSHGFKDQIYDIFQLLPPKLQVGVFSATLPPEALEITRKFMNKPVRILVKRDELTLEGIKQFYVNVDKEEWKLDTLCDLYETLAITQSVIFANTRRKVDWLTDKMRERDHTVSATHGDMDQNTRDVIMREFRSGSSRVLITTDLLARGIDVQQVSLVINYDLPTQPENYLHRIGRSGRFGRKGVAINFATKDDERMLQDIQRFYNTVIEELPSNVADLI

SEQ ID NO: 116 - protein, 207 aa, Chlamydomonas reinhardii
MAATQPSSTVPIPGSVNKSIAFRVGRNAAALNQPCFGQSRRMNRVVKARAVKAVGLRQQRKAVSPQSPPSPNQESAMNARTGKVAFGPWLGPFETHPSQDVAGVESPIFSDRKGNTYASLGGSRAPPVQTAATAAALGAKGTAASNAATKELYAMMNKMVQRHAANAPPVSGRAAHWQSGRSGKIMGKHGGGGRCGAPRTINQPRRS

SEQ ID NO: 117 - protein, 127 aa, Chlamydomonas reinhardii
MANGDLARLINSDEIQSAVRPAKSAGPKHAPLKKNPLRNLGAMLKLNPYAKVARRVEITRSTKKAAKRSEKLAKIAKGEKTGGQKDKAIKAIGKKFYKNMLVESDYAGDDYDLFARWITVQKQTKTA

SEQ ID NO: 118 - protein, 86 aa, Chlamydomonas reinhardii
MVLSSDIDLLNPPAELEKTKHKRKRLVQSPNSFFMDVKCQGCFNITTVFSHSQTVVMCGSCSSVLCTPTGGRARLTEGCSFRRKSD

SEQ ID NO: 119 - protein, 185 aa, Chlamydomonas reinhardii
MAAVIAKSSVSAAVARPARSSVRPMAALKPAVKAAPVAAPAQANQMMVWTPVNNKMFETFSYLPPLSDEQIAAQVDYIVANGWIPCLEFAESDKAYVSNESAIRFGSVSCLYYDNRYWTMWKLPMFGCRDPMQVLREIVACTKAFPDAYVRLVAFDNQKQVQIMGFLVQRPKSARDWQPANKRSV

SEQ ID NO: 120 - protein, 149 aa, Chlamydomonas reinhardii
MLSQQLARRQVSSARPSTQRPVCSRPCVRARIFRRDGPLAVEEVAVDEFNAPTKRQREQLKRTLQELGFTEQAADILAFSKITNSNCDLLIGDIRASFGVYQAPPPVGPVESVQNGVQDLLERLRLPFLGVLVAAGIFAAISNQVTMLG

SEQ ID NO: 121 - protein, 190 aa, Chlamydomonas reinhardii
MATAMLSKTVPGVLLGSTSSRRSPLAFSSGCTTRVLRALAPRPAPAGPTSSGRAVALVVRASNEEKEVQYNKEFGYSRKDVILIGVGLIALGYALYYGLQAGGMEPGMAGNWVQLIIFMGICVGWVSTYIFRVATKQMTYVKQLEQYEEAVMRKRVEEMTEAELEQLASEVDADKQRKAAARAAAQQQQQ

SEQ ID NO: 122 - protein, 42 aa, Chlamydomonas reinhardii
MQMHFEARHPKDLWEPEKCTDLHAMVGGVTTQGVAVRGSTKK

SEQ ID NO: 123 - protein, 377 aa, Chlamydomonas reinhardii
MALMMKSSASLKAVSAGRSRRAVVVRAGKYDEELIKTAGTVASKGRGILAMDESNATCGKRLDSIGVENTEENRRAYRELLVTAPGLGQYISGAILFEETLYQSTASGKKFVDVMKEQNIVPGIKVDKGLVPLSNTNGESWCMGLDGLDKRCAEYYKAGARFAKWRSVVSIPHGPSIIAARDCAYGLARYAAIAQNAGLVPIVEPEVLLDGEHDIDRCLEVQEAIWAETFKYMADNKVMFEGILLKPAMVTPGADCKNKAGPAKVAEYTLKMLRRRVPPAVPGIMFLSGGQSELESTLNLNAMNQSPNPWHVSFSYARALQNTVLKTWQGKPENVQAAQAALLKRAKANSDAQQGKYDATTEGKEAAQGMYEKGYVY

SEQ ID NO: 124 - protein, 114 aa, Chlamydomonas reinhardii
MASLCMRSSLAPKVASRSSAFLASPVAPVRSVAPVKAAPTTVVVEAKLKTRKSAAKRFKVTGSGKVTARHAGKQHFNEKMTRDHIRDSSKMFVLSPANIYNATKCLPNSGVGGK

SEQ ID NO: 125 - protein, 336 aa, Chlamydomonas reinhardii
MVGVCVSHGHAPDWRGVPDAAWDIVLGKLLWSDIASARTACRSWAVDVNRCLQRLHLRDPSPAALVRLGAVFAHLHRLKLTAWQRGGVRLDGEGLRLPGALGRVASLRLRSKQPGGGLLAVGRLALLRSGCSGGDDGGCSSAGGSGCGGGCGGGCGSRGSSGGGAAAAAAATVSMSPAGFAACAPFGTALRWGASSSGLATAIAAPPPLGGGAGWLGDGCSSVGGSSSRKAACREWEQLLPVVVLALRELRGLRRVSVEGCSKTLVAALRRGLQPVAARQQCSSRDAASWWLAVDNGSSTGSGGSSSGSSSSSAAQGRSRSDGSGASSSMSSPVTL

SEQ ID NO: 126 - protein, 424 aa, Chlamydomonas reinhardii
MAFALRSPGAVRAPACAQRASGVRAAKPGFLRSAAVARPQVQTNAAALSVPVNQLTDEERANLARELGYKSIGRELPDNVSLTDIIKSMPAEVFKLDHGKAWRACLTTIAACSACWYLISISPWYLLPAAWALAGTAFTGCFVIGHDCGHRSFHENNLIEDIVGHIFFAPLIYPFEPWRIKHNHHHAHTNKLVEDTAWHPVTEADMAKWDSTSAMLYKVFLGTPLKLWASVGYWLVWHFDLNKYTPKQRTRVVISLAVVYGFMATAFPALLYFGGPWAFVKYWLMPWLGYHFWMSTFTVVHHTAPHIPFKKAEEWNAAKAQLSGTVHCDFPNWVEFLTHDISWHVPHHVAPKIPWYNLRKATESLRENWGQYMTECTFNWRVVKNICTECHVYDEKVNYKPFDYKKEEALFAVQRRVLPDSAAF

SEQ ID NO: 127 - protein, 204 aa, Chlamydomonas reinhardii
MGAYKYIEELWRKKQSDVLRFLLRVRCWEYRQMPSCVRLTRPSRPDKARRLGYKAKQGYVVYRVRVRRGNRKRPVHKGIVYGKPVNQGITQLKPARNLRNVAEERAGRKCGGLRVLNSYWVNQDSCYKYFEVIMVDPAHNAIRNDARINWLCNPVHKHRELRGLTAAGKNYRGLRAKGHKASKTRPSKRASWKRRNSLSLRRFR

SEQ ID NO: 128 - protein, 191 aa, Chlamydomonas reinhardii
MSLDDTYELKDEIAKGIKDALAKSMSEYGNLIIHVLVNDIEPAHKVKEAMNEINAARRMRVAAAEKAEAEKVAVVKSAEAEAEAKFLQGQGIARQRQAIISGLRDSVSDFQNGVVDISSKEVLSLMLLTQYFDTLKDLGAHNRASTVFLNHAPGGVNDIANQIRGAFMEANAAGLPGSSGAGPSLPPKKTA

SEQ ID NO: 129 - protein, 25 aa, Chlamydomonas reinhardii
MPPPGMPPPGMPPPGMPPGMRPPGQ

SEQ ID NO: 130 - protein, 200 aa, Chlamydomonas reinhardii
MVSLKLQKRLAASVLNCGLRKVWLDPNEVNEISMANSRQNVRKLIKDGLIFRKQPVIHSRDRARRSAEAKAKGRHTGYGKRRGTREARLPEKVMWIRRLRVLRRLLKKYRDSKKIDKHLYHDLYMKVKGNVFKNKRVLMEAVHKQKAEKVREKTIADQFEARRAKNKAARERKATRREERLAAGLHLEAPATKPAPAQKK

SEQ ID NO: 131 - protein, 308 aa, Chlamydomonas reinhardii
MAKEEKNFMVDFLAGGLSAAVSKTAAAPIERVKLLIQNQDEMIKQGRLASPYKGIGECFVRTVREEGFGSLWRGNTANVIRYFPTQALNFAFKDKFKRMFGFNKDKEYWKWFAGNMASGGAAGAVSLSFVYSLDYARTRLANDAKSAKKGGGDRQFNGLVDVYRKTIASDGIADLYRGFNISCVGIVVYRGLYFGMYDSLKPVVLVGPLANNFLAAFLLGWGITIGAGLASYPIDTIRRRMMMTSGSAVKYNSSFHCFQEIVKNEGMKSLFKGAGANILRAVAGAGVLAGYDQLQVILLGKKYGSGEA

SEQ ID NO: 132 - protein, 33 aa, Chlamydomonas reinhardii
MVTGKGPLQNLSDHLANPGTNNAFAYATKFTPQ

SEQ ID NO: 133 - protein, 162 aa, Chlamydomonas reinhardii
MDPSDVVEVCVRVTGGEVGAASSLAPKIGPLGLSPKKIGEDIAKETLKDWKGLRITVKLTVQNRQAKISVIPSAAALVIKALKEPARDRKKEKNIKHTGSVTMSDIIEIARVMRPRSCAKDLANGCKEVLGTAVSVGCKVDGKGPRDVTSAIDDGAIEIPDA

SEQ ID NO: 134 - protein, 65 aa, Chlamydomonas reinhardii
MIAARVSSAKPVAAKNVQAAKPKVAPAVLGVFAAAMSCSPPLPTPPRRPASARCCARPTPPPRCA

SEQ ID NO: 135 - protein, 223 aa, Chlamydomonas reinhardii
MSPVAEPFAMDVESQSSDGAKTKAQLVAMAPIKDEWVRGDPGPFGLLCFGMTTCMLMFITTEWTTKGFLPTVFCYAMFYGGLGQFVAGVLELIKGNTFGGTAFASYGAFWMGWFLLEYLTWTNKALYAGVQSGKSLWCGLWAVLTFGFFIVTCRKNGCLMTIFSTLVITFALLSGGVWDPRCEQAAGYFGFFCGSSAIYAAFVFLYKIELGISLPGVRPVAFL

SEQ ID NO: 136 - protein, 173 aa, Scenedesmus obliquus
MSPSASQHFSWFVRDTVCALVLVVELGPAQEPTSQSNSTTMGYAGYVPAKLPPSKPTEVEAAVQAVKSVETLEVMGKLLYNCAVSPREDKFRRVKLTNKKVADTISSTHGALEAMAALGWVADEANPQELVVREGTFFSMKEVRIVEAAKERLQKDMRSSSSKNLAAMVSVQA

SEQ ID NO: 137 - protein, 105 aa, Scenedesmus obliquus
MALCAKSSTRAVCAKQAARPAVARPVLARRMTVQASAQKQQVSMQVTAGLAAAAATQMLMPLAAAAEVTPSLKNFVYSLVAGGVVLGGIALAITAVSNFDPVKRG

SEQ ID NO: 138 - protein, 74 aa, Scenedesmus obliquus
MYNSLTVQSIVNDMNCQLFPPSPAVAECVQVTHRTWGLLLDALQSLFVASRVTLPRQPRLAMPPPAVALHRWLL

SEQ ID NO: 139 - protein, 154 aa, Scenedesmus obliquus
MVRIDTDSKGGLGGTTENVFGPLAVLVVGFLPEEYQAFRAMMVDIEADMVKVVPCSKALLAGSLQHAMESEYPQYEQPPLGQRRALFLSGMYGSEVVELIAAYREAGLPPCAFAAAVPNNWQRNVKELSEAVWRDHAAMMAKQQPQPGGIEDSY

SEQ ID NO: 140 - protein, 183 aa, Scenedesmus obliquus
MKLLYSLQRSYPSLSHITMALAIKSAAAQVACVKAAPVAKANRMMVWRPDNNKMFETFSYLPPLSDDQIARQVDYIVNNGYTPCLEFSMPEDAYVSSGSSIRFGAVSCNYFDNRYWTLWKLPMFGCSDPVQVLREIDNATKAFPNAYIRMAAFDANRQVQVASMLVHRPSSAKEWRPVNQRQV

SEQ ID NO: 141 - protein, 119 aa, Scenedesmus obliquus
MHLFLGLVLTRSQLQMLTQARNIVCTHIHTAARFGCCSVSVAGGMLPTHHHAHMFVVFKRDGKDQIDQREAAGDAKGSLSQQWSQQHSGRNWDVCLWRYYRIECCFVRFVSPLYQHSLP

SEQ ID NO: 142 - protein, 79 aa, Scenedesmus obliquus
MVSCNYFDNRYWTLWKLPMFGCSDPVQVLREIDNATKAFPNAYIRMAAFDANRQVQVASMLVHRPSSAKEWRPVNQRQV

SEQ ID NO: 143 - protein, 139 aa, Scenedesmus obliquus
MFAGSLPAFIASSFTFNFLLWFVACGSWLRFMVVVLWRVHAHVHTLNARKASLCKFCSGSPRMCARCNVATCRYVVRIMSYFYVYTPSFSQHSRTIRTTEHLAVSYDCSHASLRLSRIVWCMYATALNYPQASRCSWVR

SEQ ID NO: 144 - protein, 187 aa, Scenedesmus obliquus
MLLSPLPRQLPCSLPHVLQHALMYWRVEVQPVVAAARPRDSAHCLHTQSARGACTAQPRAAAAALPVLACCLCVCVRVGHAVVCWLALPRRPTVACCWCWCGARLRLTPGPPPSPGHGAACRSDCGWCPPSAPARGKAPKHEASWQARRNSAAEVHQGGSSTPGSSAQQHAQDLCMCAQPQVCPRSA

SEQ ID NO: 145 - protein, 623 aa, Scenedesmus obliquus
MPTTMGTPTSPTADIAGPQQEQQQQQTTVIFTSKQSSHSPTVLARYQRYGQELALFLLQHGQDSLQLYLRFLLRASSCVRLWLQDNQQHLALASIAKALRWLRLKLLAPIGQLLCGLLSLQLRISTNDDLDDATKCSARLNSCLTLLFGAIRLRSHSCLQLSLPSRQPHFILPEFSLVSPATSPRLPGTPRSRQQQQEWPLAAQQQLRSSSSSSPMRSPRKGERGSSSSSVVRRSAAAEEEVHVLCSSELKAAIMCFLQHHSASSGQEFVLLRSTSWGSAAAAGAAAATLDSTAAGTRRSAADGAVSPRRRSGGPGQVISPGACGRIQQSRQLFQTLADSSAGSARSSRSASRDGFAFAPLQSPATSAAALNGGCSSSSTQLWRSTSAVSSSRDAPQQRSSWPAAGSAASPGVLQLMQHAAYAAADAGAGANGSADAVVAVSIEQPQDGVTSPRLPDGCSWPDKQQLQQQQQMQMQHLHTRVLQRQQQQGGITPRSLAGCFSEPTNLSMCVLDDPSLTPLRSFSASGSCSHALQQQQQRQPASPSGRLSGQQQVQAVPLGSHRVCDKGGGCTPPRSASPIAAPARHGVRKAAGKLPPAPFTAAQLQQQEQQQQQQQQQQQQLF

SEQ ID NO: 146 - protein, 293 aa, Scenedesmus obliquus
MVRLQAASGLLRALGPCIRSSLSPECQQIAAAAAGVRLSESVAPAVTYRDISDEWYLRQRSQISLGNRLPHVAVSAWISPSAVVVGDVDLLDRVSVWNNVVLRGDLNNITIGQVSNVQDRTIIHAARTSPAGISASVKVGKFVTIEPNCTLRSCRVGDFAKVGARSVLLEGSMMEDYSVLQPGSVLPPTRRVPTGEVWGGVPARFVRALSEDERDALKAEADDIRRLAWQQDAEQLPVGTAWRGVEAYRAAVVESGKAVSVPMRRLKYDQRKRREDEAANAISVAAGASQAIK

SEQ ID NO: 147 - protein, 219 aa, Scenedesmus obliquus
MAETLTFRGSLKGHQGWVTALATPLDPNSDIILPASRDKTVIVWHLERSETQYGYPKRALTGHSHFVQDVVISSDGQFALSGSWDGTLRLWDLNTGNTTRRFVGHTKDFVSLLLLYKWTGLQQPTLWWCGPTSCPCGPCGSPCAAGVLQTRSAHTDMHLVFDIITYNGCPLGPVMLLQVCRTPSLLLSLLGLQGTLEMFTALTGPFSSAQHFVDSPPMG

SEQ ID NO: 148 - protein, 192 aa, Scenedesmus obliquus
MKRSDVDMFPHCVHLVSWEKENVSSEALEAARIAGNKYMTKFAGKEAFHLRVRVHPFHVLRINKMLSCAGADRLQTGMRGAFGKPQGVCARVQIGQILLSIRCKDNHAAVAAEALRRAKFKFPGRQKVVASRNWGFTPYSRDDYIQWKKEGRLVSAGVHAQLLGCRGAVADREAGNLFATPARIHKIPNHAE

SEQ ID NO: 149 - protein, 215 aa, Scenedesmus obliquus
MLAARSTSAPRAFSSRATAAPRGIRVIAMAGKRNDVSDSYAKALVELAEEKNTLESVHADVDTLAGLVSANQKLSELLFNPVVEAGKKRAVVAKISKEAGFQKYTTNFLNLLVAEDRMNLLNEICESFEEQYCTLTDTQVATLRSAVKLEQEQQFLIAKKLQELTGSKNIKLKPIIDQSLIAGFVVEYGSSQIDLSVRGQIEKVAEELTRSMATA

SEQ ID NO: 150 - protein, 197 aa, Scenedesmus obliquus
MGSRDKSIKLWNTLGECKYTIAEPEGHTEWVSCVRFSPMTNNPIIVSAGWDKLVKVFNLTNCKLKSNLVGHSGYINTVTVSPDGSLCASGGKDGVAMLWDLAEGKRLYSLDAGDIIHALVFSPNRYWLCAATQSSIKIWDLESKSVVDDLRPEFTKTYGKKAIVPYCVSIAWSADGGTLFAGYTDGIIRVFTVSHGL

SEQ ID NO: 151 - protein, 341 aa, Scenedesmus obliquus
MNDQKGLLGNVSLAGQVLTGWNVTHLCLEGGGPDAFSDLQLPWRPLAGAAAGAGGAGAHWDSDNSAGQQQASSGGEVAGDATAAAEGRVSGQHRSQQEAGSRPLLVLPGSSAWLAERQAARQQQQQRPAGDEAASSKSSSSSSSVRGIFAGAGNVTAFISTVAEAQAQAVIAAAADAATGNGPATAAARAAATIRAGLAAAAAADNSIDVAQRAKGPLVLRGTLHVPAAAQAEATGSQQQQQQRGSLLPPGRPADTFLHLGEGWGRGQVWVNGVSLGAYWAEQGPQMSLYLPGSFLQPGPNEVVLLELDGRVPVRGDGPPTIACADEPDFDGPAAGPAPLA

SEQ ID NO: 152 - protein, 115 aa, Scenedesmus obliquus
MNATMLRGSAFASSSRVAAVRPAISSCASTVVVRAVQDLQGKVVSKGMQQSVVVAVERLSAHDKYFKRVRITKRYIAHDDGSLGVGVGDYVRLEGCRPLSKNKRFKLAEVVRRAD

SEQ ID NO: 153 - protein, 123 aa, Scenedesmus obliquus
MAVSMRTSVAARSATRAARPSRAAVVVRAEARREVLAGFVAAGAALLSVGQAQAVTPVDLFDDRRVRNTGFDIICEARDLDIPQAERDGMTQARADIEATKKRVKDSEARIDNSVATSIEKAY

SEQ ID NO: 154 - protein, 78 aa, Scenedesmus obliquus
MLVGCGEFSLARYSQHLQSCGSVLFMLLCAAGSAMDTAKDWLVGQPRQLEIYVPAAAGVAMHWSVSGAASGVHRAALN

SEQ ID NO: 155 - protein, 28 aa, Scenedesmus obliquus
MRKKDKDSALAKLAVAKANLDSVIAKVL

SEQ ID NO: 156 - protein, 56 aa, Scenedesmus obliquus
MHCRVSVHTSTGFGSVTSVRALGVEELLSLPFAAAQALRRCWAVWSGDILHSDTAE

SEQ ID NO: 157 - protein, 146 aa, Scenedesmus obliquus
MLSSSLSTRVAAAPRNRGSRYAFRASAAEAPAAQKTCFIAMNVFKVKPECAEDFENVWKNRESHLKEMSGFVRFALLKCSNVPNKYISQSTWESEEAFRGWTQSQQFSKAHGEADKDKGGSKRPNVGAMLEGPPAPEFFSSVTMTE

SEQ ID NO: 158 - protein, 325 aa, Scenedesmus obliquus
MTQPDQVAHTSARSAEAFSMIAAQLRNFAPAASKCAARRVTLPGQLHQCIGAASRNLASSSSPRDVRAEASNARFFVGGNWKCNGTHASVEKLVQELNAGSVPSDIDVVVAPPFIFLDMVRLSLKNEYQVAAQNCWVKSDGAFTGEISAEMLMDTLVPWVITGHSERRSLCGESSKFVGEKTGHALDVGLKVIACVGETLDQRNSGSLWYTLDSQMQALFPNDKDWSRVVIAYEPVWAIGTGQVATPEQAQEVHAYLRTVLANKLGEETAAGVRIIYGGSVNDGNCNELATMPDIDGFLVGGASFKAPSFLQICNSVASRRTMAA

SEQ ID NO: 159 - protein, 126 aa, Scenedesmus obliquus
MLNQSLSTLPITPTPAQLYAVLQYHFCPAPGGNVKGLLYFKFKANKFNPCLTLLGLTKPAPTLKLFFNQTLTDNNPLLPMKLTVDYLDLGGQPAQTNIVVPDVTTPLTAGHVVDRVLIPPLSALPL

SEQ ID NO: 160 - protein, 229 aa, Scenedesmus obliquus
MGRRPARCYRQSKGKPYPKSRYCRGVPDPKIRIYDVGMKRSDVDMFPHCVHLVSWEKENVSSEALEAARIAGNKYMTKFAGKEAFHLRVRVHPFHVLRINKMLSCAGADRLQTGMRGAFGKPQGVCARVQIGQILLSIRCKDNHAAVAAEALRRAKFKFPGRQKVVASRNWGFTPYSRDDYIQWKKEGRLVSAGVHAQLLGCRGAVADREAGNLFATPARIHKIPNHAE

SEQ ID NO: 161 - protein, 199 aa, Scenedesmus obliquus
MQSLARARTTPAAAALRHRGTQRCQRNLLHVLTQAASQQAAATEAPAQTHWFQNTITLPNHKRGCHVVTRKLLAELPELGEYEVGLANLFILHTSASLTINENASPDVPLDLNDALDKLAPEGNHYRHLDEGLDDMPAHVKSSLMGPSLTIPIAKGRFALGTWQGIYLNEHRNYGGSRSIIVTIQGQKRPDGRRYGQFS

SEQ ID NO: 162 - protein, 325 aa, Scenedesmus obliquus
MARLVGCELSITCCPTTAHTTASMARKISYENLFVIVAAAASALLACSAQAQIQQVALPYALDALEPEIDNATMNFHYGTHYATYVNNTLAALKNATDSGVRLPVATSNLTALISSIKRLPSPLNTTIRNQGGGAWNHALYFKHLTPPGSPATQSTAISAPLKDAINANFGSVDNMTAALSAAAGKVFGSGWAWLCYTGNSSADLAITTTPNQDNPLMGQLPGAPAVSAAGCTPILGIDVWEHAYYLKHGPKRPAYLESFWKALNWQQVSKNYDSAVQGLELDLSEAAPRALEAPSKAVSAGSSSSGSAGFGAMAAAAAALLALV

SEQ ID NO: 163 - protein, 77 aa, Desmodesmus sp.
MGYVEPNSCCTMCFVGYCKLHQRAVRTSGGAQQQLLCLAPAMQVLAREKHLQVFVLKARPGSGLLRWMLYLDCRWCS

SEQ ID NO: 164 - protein, 118 aa, Desmodesmus sp.
MVAYSPLCQGLLTGKYTPDGPRPTGPRGNIYSNKIREIQPLISAMKAVGEERGKSPAQVALNWIICKGAVPIPGAKNKRQVDEIAGAVGWRLSEGEVLELEKAADKIAAPLGAPFENW

SEQ ID NO: 165 - protein, 130 aa, Desmodesmus sp.
MFETFSYLPPLSDDQIARQVDYIVNNGWTPCLEFSEAEGAYVSSASCLRMGAVTSNYFDNRYWTMWKLPMFGCTDPVQVLREIDNATKAFPNAYIRMCAFDASRQVQVASMLVHRPAAAKEWRPVNQRQV

SEQ ID NO: 166 - protein, 161 aa, Desmodesmus sp.
MRTPLVDCECLESPCSRQAPSLLGQELSCSSSMTVLLVRSPTAAATSKAAPALFDQLDKALKGGDGKDLVAKTKGLVVFVIDGDTWTLDLREGAGTVTQGPPIDKADLTLTISDDNFAKMVMGKLSPQQAFLLRKLKLNGSMGLALKLQPILDAAAPRAKL

SEQ ID NO: 167 - protein, 109 aa, Desmodesmus sp.
MGGWVSKQPQVSTADQHRLARKCKSLSQAYARCHKANPDDAAACNNLQTSLVMCYAADLCKDAAAAHEKCYMSVINTGRYQGKLDCEETVQQMKDCLRKYKLYPFSPQL

SEQ ID NO: 168 - protein, 170 aa, Desmodesmus sp.
MAATMKTACQARLTSSVKQVKASPVAKANRMMVWRPDNNKMFETFSYLPPLSDDQIARQVDYIVNNGWTPCLEFSEAEGAYVSSASCLRMGAVTSNYFDNRYWTMWKLPMFGCTDPVQVLREIDNATKAFPNAYIRMCAFDASRQVQVASMLVHRPAAAKEWRPVNQRQV

SEQ ID NO: 169 - protein, 123 aa, Desmodesmus sp.
MLRSWPSGRCCVARHACCLFGCHTCCTCPVIFMRPTGFEKIPAASVLLGAAHVTYLAAVVFGCSVALAVGGHVVCCQGAARCCSGWHRLPGALACLCRSCTMMSCITILRVASLTAGSDGLLA

SEQ ID NO: 170 - protein, 47 aa, Desmodesmus sp.
MGMSEQQGLQLPFYEIVFVEGDMRSLLACTAYSACCPWRRHLPCASF

SEQ ID NO: 171 - protein, 126 aa, Desmodesmus sp.
MAEVHPKAYPLADAQLTNVIMDIVQQASNYKQLKKGANEATKTLNRGISEFVVMAADTEPIEILLHLPLLAEDKNVPYVFVPSKAALGRACGVSRPVIAASVTTNEGSQLKSQIQTLKDSIEKLLI

SEQ ID NO: 172 - protein, 54 aa, Desmodesmus sp.
MHLWWSGVLASACTRHQASSALTSVAQLLSCAMTLPAKGRAGCETASRRCMQHR

SEQ ID NO: 173 - protein, 66 aa, Desmodesmus sp.
MQYAPGDAALKKELLKYMAEHPSVARFAIPDDVVFLEEIPHNATGKVSKLTLRQMFKDYKPAQPKL

SEQ ID NO: 174 - protein, 116 aa, Desmodesmus sp.
MIACSWLLAHGCSPMIACPNAAECSLMRARHIMALLYVVVVIVQLMCLFTELSPQESCRLSPSPLLLLRQALKGACCVQAACCSYLAGSSNCMHAIFPLAVACNCWERLVRTVRIR

SEQ ID NO: 175 - protein, 142 aa, Desmodesmus sp.
MVCRCGCSLLCAQEGVHSWGSASTSFFQMGNAQHRPLQRLVLMATQHAWQDQIMLIKSFEHTSLLCAPWPLATSKPGWLSCMRLHLQRLHPEGLGAMTAVQMCMSGATAHTYNLVEAIRFEDAEAKPWRVEGLMSRGYCKII

SEQ ID NO: 176 - protein, 107 aa, Desmodesmus sp.
MPHKPTAEELEAAKEELVQEIVEKVLEIPGAVTQTISTAPYDSRFPSTNQNRHCFIRYNEYYKCAFERGGEDARCQFYKNAYESLCPPDWVEEWEELRQQGIWFGKY

SEQ ID NO: 177 - protein, 161 aa, Desmodesmus sp.
MCLPLRQQERCRVWRAGVPCVPSGWSGCIGGLQRITGGAQWGCLATMLSLCIEGCLCSIAAVWCSSEGMRHAGTGVGLCIMAVGSQLLGPVLTCVCMGQQDRPGFPGGDSVSQQRWAGGASRLPPAPCGDLQWLGAAGSCESSLVDAKCGMAVGQACCLCS

SEQ ID NO: 178 - protein, 113 aa, Desmodesmus sp.
MQLSLCRAPTMSRVVAAPTRRPRIVVVRSGNHPSMKDVEDINAKVKEAIEEVEDMCNGGDAAHCAAAWDNVEELSAAASHKKDAVKEDKVSSDPLEAFCDDNPDADECRVYDD

SEQ ID NO: 179 - protein, 161 aa, Desmodesmus sp.; identical to SEQ ID NO: 177

SEQ ID NO: 180 - protein, 245 aa, Desmodesmus sp.
MAMLLKKAAVAPARTSVRSKAAIEWYGPDRPKFLGPFSEGDTPAYLTGEFPGDYGWDTAGLSADPQTFAKYKEIEVIHARWAMLGALGCITPELLAKNGVPFGEAVWFKAGAQIFQDGGLNYLGNENLVHAQSILATLAVQVLLMGAAESYRANGGAPGGFGEDLDSLYPGGAFDPLGLADDPDTLAELKVKEIKDGRLAMFSMFGFFVQAIVTGKGPIANLDEHLADPSGNNAWNYATKFVPGN

SEQ ID NO: 181 - protein, 245 aa, Desmodesmus sp.
MAMLLKKAAVAPARTSVRSKAAIEWYGPDRPKFLGPFSEGDTPAYLTGEFPGDYGWDTAGLSADPQTFAKYREIEVIHARWAMLGALGCITPELLAKNGVPFGEAVWFKAGAQIFQDGGLNYLGNENLVHAQSILATLAVQVLLMGAAESYRANGGAPGGFGEDLDSLYPGGAFDPPGLADDPDTLAELKVKEIKNGRLAMFSMFGFFVQAIVTGKGPIANLDEHLADPSGNNAWNYATKFVPGN

SEQ ID NO: 182 - protein, 276 aa, Desmodesmus sp.
MAFSMMSRATSVNVVAKKGGKAAPKKVAKKAASGGSKGVEWYGPSRAKFLGPFTQAPSYLTGEFAGDYGWDTAGLSADPETFRRYRELEVIHARWAMLGALGCITPELLAKNGVNFGEAVWFKAGAQIFQDGGLNYLGNSSLIHAQSILATLAVQVILMGLIEGYRVNGGPAGEGLDPLYPGEAFDPLGLADDPDTLPSSRSRRSRTVAWPCSACLASSCRPSSLDRAPLPTWTPTWRTLPATTPGTSPPSLCPATKPPLHTAVAMYGRDPRGLWC

SEQ ID NO: 183 - protein, 377 aa, Desmodesmus sp.
MGLKHTQLCTLALLCGVLQLAAASQCAVGDAACFCNLIKGTWVATPSTIRPTCRKTVDSQGVKRIVDVYFPTDIGTPGVKFTPWVHLHGVMWSKWWLEQNPEWQQLSKKWTPPMGGNADASRMNALTGSPVTGEGSKAEGAGLVKQQGYILLVPQGTGDVTLGQMWNSVFWPCMTSTRCVDKSVDDVSFLAGVITGMQQLLPGLPLKPQVALSGYSNGGMMIQTLLCNKPEVANSLAAVALVGTMLGSDFAAGSCRQKLPKSLPLVWLHGVKDPVLPYAAGGSSLGVKALGAEAGTKLWADRMGCPGVSGTPQTVFTDGGAGVSCVNLCGGGGQPTTAVLCAVAEAGHQLWDQPGSGYSAGVMLWAFGGFAGTPKPL

SEQ ID NO: 184 - protein, 72 aa, Desmodesmus sp.
MPGQLVAVWQWVMCLLYSCPSLLRQSSSSSCRYAVVGCSLRCPLHVPADGGDTSRFVPAAVSLFQAVCLHLE

SEQ ID NO: 185 - protein, 163 aa, Desmodesmus sp.
MMLCYGIHRLQRLHYVVQLSRQQQQQQRGVPPQQRQDRQPPQQQQQQQQVVLDAASKAVGRSSRPACRRQRRARSRSGTSSRADSSSTISSRSSSSSSDRALAGQVAGAAGQIGTASEQDVTDLVAVPWRSMIQEADSVFCRRFPAFKAWLAQQQQQEQQEQD

SEQ ID NO: 186 - protein, 467 aa, Desmodesmus sp.
MWARGTPEPDDNSKPGRGSGMASIITKEITSCDSVPELESIFIEHGNFTYLNSSAALTKYAKLRGSSMRSPFFSKLAAVWLTRLPEAGGREYANVLWACSRLGSSKHPVWAETWQAFLDLTEKDVNRDKPPSRPQDISNVLYAAAKLRQQPRPDELLLLLEAFTHPVVLAAANPQDTANIVWSLGQLSVTPGWEAEVSQELLQGLLAPQLLQSVAADGIPQGVSNVLVGLGRMCTAQSPLLSTAAAQGYAGQLLSGVGLNKLSSWNPQHITNVMWALGELQVNKEDFVRAAVAAAPKWLPLSTGYDLTQAASACAQLQYSDEHFMRLLLQRGQQLLQPNRRSRGRPLSEPDKAALATICSVPVVCLDMRGLADAARKLVADSGLMQQARTHPAQAMAVPQLAVGAPAARWDGAVGFGDGAAAAAGGKAGSSMEGQDPAVSRTASTEAVVLSCCWPALVSGNLFLGLW

SEQ ID NO: 187 - protein, 72 aa, Arthrospira maxima
MSQKVIHPMDKFQRQVHSLVKSDIVKPEDSLWKIALLFGDKWKYWKAELIEFGFSMQDPISELLAVDVWDEE

SEQ ID NO: 188 - protein, 174 aa, Arthrospira maxima
MHGFVLDEKGYKMSKSLGNVVDPAVVINGGKNQQQDPAYGADILRLWVSSVDYSADVPLGKNILKQMSDVYRKIRNTARFLLGNLHDFDPAKDAIAYEDLPELDRYMLHRMTEVFAEVTDAFETYQFFRFFQVVQNFCVVDYPISIWILRKTDYILARKMVSGGAVVRLFWRSP

SEQ ID NO: 189 - protein, 146 aa, Arthrospira maxima
MLIPYWKEAGYDFDPQSDRLWLEQLTTLIGPSLTRLKDAVDMAAMFFPSSVSLDEEAQQQLQQEGAQTVLAAIKDKLESEPTLTADTVKDMIKAVTKETKLKKGLIMRSLRAALTGAVHGPDLVESWLLLHQRGTDLTRLQNILDR

(19) United States
(12) Patent Application Publication
(10) Pub. No.: US 2019/0112616 A1
(43) Pub. Date: Apr. 18, 2019

(54) BIOMASS GENES

(71) Applicant: RENEW BIOPHARMA, INC., San Diego, CA (US)

(72) Inventors: Christopher YOHN, San Diego, CA (US); Eric HAMPTON, San Diego, CA (US); Yan POON, Novato, CA (US)

(21) Appl. No.: 16/090,186

(22) PCT Filed: Mar. 29, 2017
(86) PCT No.: PCT/US2017/024860

§ 371 Date: Sep. 28, 2018

Related U.S. Application Data
(60) Provisional application No. 62/314,855, filed on Mar. 29, 2016.

(51) Int. Cl. C12N 15/82; C12N 1/12; C12N 1/20; C07K 14/195; C12N 1/04

(52) U.S. Cl. CPC C12N 15/8261; C12N 1/12; C12N 1/04; C07K 14/195; C12N 1/20

(57) ABSTRACT

Disclosed herein are polynucleotides and the polypeptides encoded thereby and their use to increase biomass production by photosynthetic organisms. Also provided are photosynthetic organisms transformed by such polynucleotides and expressing such polypeptides.

### BACKGROUND

As the Earth's population continues to grow, there is an increasing demand for sources of food. Photosynthetic organisms are especially useful for meeting this increasing demand, because in addition to producing high quality food for humans and animals, they also fix carbon dioxide which has been implicated in climate change. Photosynthetic organisms suitable for producing food products range from conventional agricultural crops to micro algae.

While in some instances only parts of a plant are consumed, such as seeds, in many instances the entire plant is consumed. Thus, much of the growing need for food may be able to be met by increasing the amount of biomass produced by photosynthetic organisms. Traditional plant breeding techniques have made substantial increases in biomass production in the past, but that increase is plateauing. The introduction of genetic engineering techniques has greatly increased the speed at which progress in increasing biomass production can be made. In order to achieve this increase, however, it is necessary to identify genes associated with production of biomass. The relatively slow generation interval of many traditional agricultural plants slows the speed at which new growth associated genes can be identified. Algae with their rapid generation interval provide a means to quickly identify and validate genes associated with increases in biomass productivity. Also, because terrestrial plants and algae share the same basic biochemical processes, discoveries made in algae are readily applicable to terrestrial plants.

Provided herein are polynucleotides, which when overexpressed in photosynthetic organisms, result in increased biomass production. These genes can be readily applied to increase biomass production to help alleviate the increasing need for food, feed, nutritional supplements and energy while working to decrease the amount of atmospheric carbon.

### SUMMARY

The present disclosure provides: (1) A photosynthetic organism transformed with at least one polynucleotide comprising (a) a nucleic acid sequence of SEQ ID NO: 1 to 99 or (b) a nucleotide sequence with at least 80%, at least 85%, at least 90%, at least 95%, at least 98%, or at least 99% sequence identity to the nucleic acid sequence of SEQ ID NO: 1 to 99; wherein the transformed photosynthetic organism's biomass is increased as compared to a biomass of an untransformed photosynthetic organism of the same species. (2) The transformed photosynthetic organism of 1, wherein the increase is measured by a competition assay, growth rate, carrying capacity, productivity, cell proliferation, seed yield, organ growth, or polysome accumulation. (3) The transformed photosynthetic organism of 2, wherein the increase is measured by a competition assay. (4) The transformed photosynthetic organism of 3, wherein the competition assay is performed in a turbidostat. (5) The transformed photosynthetic organism of 1, wherein the increase is shown by the transformed photosynthetic organism having a positive selection coefficient as compared an untransformed photosynthetic organism of the same species. (6) The transformed photosynthetic organism of 5, wherein the selection coefficient is from 0.05 to 0.10, from 0.10 to 0.5, from 0.5 to 0.75, from 0.75 to 1.0, from 1.0 to 1.5, from 1.5 to 2.0, or 2.0 to 3.0. (7) The transformed photosynthetic organism of 1, wherein the increase is measured by growth rate. (8) The transformed photosynthetic organism of 7, wherein the transformed photosynthetic organism has an increase in growth rate as compared to an untransformed photosynthetic organism of the same species of from 5% to 10%, from 10% to 15%, from 15% to 25%, from 25% to 50%, from 50% to 75%, from 75% to 100%, from 100% to 150%, from 150% to 200%, from 200% to 300%, or from 300% to 400%. (9) The transformed photosynthetic organism of 1, wherein the increase is measured by an increase in carrying capacity. (10) The transformed photosynthetic organism of 9, wherein the units of carrying capacity are mass per unit of volume or area. (11) The transformed photosynthetic organism of 1, wherein the increase is measured by an increase in productivity. (12) The transformed photosynthetic organism of 11, wherein the units of productivity are grams per meter squared per day or mass per acre, mass per unit area such as tons per acre/hectare, or volume per unit area such as bushels per acre/hectare. (13) The transformed photosynthetic organism of 12, wherein the transformed photosynthetic organism has an increase in productivity as measured in grams per meter squared per day, as compared to an untransformed photosynthetic organism of the same species of from 5% to 10%, from 10% to 15%, from 15% to 25%, from 25% to 50%, from 50% to 75%, from 75% to 100%, from 100% to 150%, from 150% to 200%, from 200% to 300%, or from 300% to 400%. (14) The transformed photosynthetic organism of 1, wherein the transformed photosynthetic organism is grown in an aqueous environment. (15) The transformed photosynthetic organism of 1, wherein the transformed photosynthetic organism is a bacterium. (16) The transformed photosynthetic organism of 15, wherein the bacterium is a cyanobacterium. (17) The transformed photosynthetic organism of 1, wherein the transformed photosynthetic organism is an alga. (18) The transformed photosynthetic organism of 17, wherein the alga is a microalga. (19) The transformed photosynthetic organism of 18, wherein the microalga is at least one of a Chlamydomonas sp., Volvacales sp., Desmid sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmus sp. (20) The transformed photosynthetic organism of 18, wherein the microalga is at least one of Chlamydomonas reinhardtii, N. oceanica, N. salina, Dunaliella salina, H. pluvalis, S. dimorphus, Dunaliella viridis, N. oculata, Dunaliella tertiolecta, S. Maximus, or A. Fusiformus. (21) The transformed photosynthetic organism of 1, wherein the transformed photosynthetic organism is a vascular plant. (22) The transformed photosynthetic organism of 21, wherein the transformed photosynthetic organism is Brassica (e.g., Brassica nigra, Brassica napus, Brassica hirta, Brassica rapa, Brassica campestris, Brassica carinata, and Brassica juncea), soybean (Glycine max), castor bean (Ricinus communis), cotton, safflower (Carthamus tinctorius), sunflower (Helianthus annuus), flax (Linum usitatissimum), corn (Zea mays), coconut (Cocos nucifera), palm (Elaeis guineensis), oil nut trees such as olive (Olea europaea), sesame, and peanut (Arachis hypogaea), as well as Arabidopsis, tobacco, wheat, sugarcane, sugar beet, barley, oats, amaranth, potato, rice, tomato, legumes (e.g., peas, beans, lentils, alfalfa, etc.), grasses (e.g. Miscanthus, switchgrass, energy cane), vegetable crops and fruits.

Also provided is: (23) A transformed photosynthetic organism comprising at least one exogenous polynucleotide encoding a polypeptide comprising (a) at least one amino acid sequence of SEQ ID NO: 100 to 189 or (b) an amino acid sequence with at least 80%, at least 85%, at least 90%, at least 95%, at least 98%, or at least 99% sequence identity to at least one of SEQ ID NO: 100 to 189; wherein the transformed photosynthetic organism expresses the at least one exogenous polynucleotide; and wherein the transformed photosynthetic organism's biomass is increased as compared to a biomass of an untransformed photosynthetic organism of the same species. (24) The transformed photosynthetic organism of 23, wherein the increase is measured by a competition assay, growth rate, carrying capacity, productivity, cell proliferation, seed yield, organ growth, or polysome accumulation. (25) The transformed photosynthetic organism of 24, wherein the increase is measured by a competition assay. (26) The transformed photosynthetic organism of 25, wherein the competition assay is performed in a turbidostat. (27) The transformed photosynthetic organism of 23, wherein the increase is shown by the transformed photosynthetic organism having a positive selection coefficient as compared to an untransformed photosynthetic organism of the same species. (28) The transformed photosynthetic organism of 27, wherein the selection coefficient is from 0.05 to 0.10, from 0.10 to 0.5, from 0.5 to 0.75, from 0.75 to 1.0, from 1.0 to 1.5, from 1.5 to 2.0, or 2.0 to 3.0. (29) The transformed photosynthetic organism of 23, wherein the increase is measured by growth rate. (30) The transformed photosynthetic organism of 29, wherein the transformed photosynthetic organism has an increase in growth rate as compared to an untransformed photosynthetic organism of the same species of from 5% to 10%, from 10% to 15%, from 15% to 25%, from 25% to 50%, from 50% to 75%, from 75% to 100%, from 100% to 150%, from 150% to 200%, from 200% to 300%, or from 300% to 400%. (31) The transformed photosynthetic organism of 23, wherein the increase is measured by an increase in carrying capacity. (32) The transformed photosynthetic organism of 31, wherein the units of carrying capacity are mass per unit of volume or area. (33) The transformed photosynthetic organism of 23, wherein the increase is measured by an increase in productivity. (34) The transformed photosynthetic organism of 33, wherein the units of culture productivity are grams per meter squared per day or mass per acre, mass per unit area such as tons per acre/hectare, or volume per unit area such as bushels per acre/hectare. (35) The transformed photosynthetic organism of 34, wherein the transformed photosynthetic organism has an increase in productivity as measured in grams per meter squared per day, as compared to an untransformed photosynthetic organism of the same species of from 5% to 10%, from 10% to 15%, from 15% to 25%, from 25% to 50%, from 50% to 75%, from 75% to 100%, from 100% to 150%, from 150% to 200%, from 200% to 300%, or from 300% to 400%. (36) The transformed photosynthetic organism of 23, wherein the transformed photosynthetic organism is grown in an aqueous environment. (37) The transformed photosynthetic organism of 23, wherein the transformed photosynthetic organism is a bacterium. (38) The transformed photosynthetic organism of 37, wherein the bacterium is a cyanobacterium. (39) The transformed photosynthetic organism of 23, wherein the transformed photosynthetic organism is an alga. (40) The transformed photosynthetic organism of 39, wherein the alga is a microalga. (41) The transformed photosynthetic organism of 40, wherein the microalga is at least one of a Chlamydomonas sp., Volvacales sp., Desmid sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmus sp. (42) The transformed photosynthetic organism of 40, wherein the microalga is at least one of Chlamydomonas reinhardtii, N. oceanica, N. salina, Dunaliella salina, H. pluvalis, S. dimorphus, Dunaliella viridis, N. oculata, Dunaliella tertiolecta, S. Maximus, or A. Fusiformus. (43) The transformed photosynthetic organism of 23, wherein the transformed photosynthetic organism is a vascular plant. (44) The transformed photosynthetic organism of 43, wherein the transformed photosynthetic organism is Brassica (e.g., Brassica nigra, Brassica napus, Brassica hirta, Brassica rapa, Brassica campestris, Brassica carinata, and Brassica juncea), soybean (Glycine max), castor bean (Ricinus communis), cotton, safflower (Carthamus tinctorius), sunflower (Helianthus annuus), flax (Linum usitatissimum), corn (Zea mays), coconut (Cocos nucifera), palm (Elaeis guineensis), oil nut trees such as olive (Olea europaea), sesame, and peanut (Arachis hypogaea), as well as Arabidopsis, tobacco, wheat, sugarcane, sugar beet, barley, oats, amaranth, potato, rice, tomato, legumes (e.g., peas, beans, lentils, alfalfa, etc.), grasses (e.g. Miscanthus, switchgrass, energy cane), vegetable crops and fruits.

Also provided herein is: (45) A method of increasing biomass of a photosynthetic organism, comprising (a) transforming the photosynthetic organism with at least one polynucleotide to produce a transformed photosynthetic organism, wherein the polynucleotide comprises: (i) a nucleic acid sequence of SEQ ID NO: 1 to 99; or (ii) a nucleotide sequence with at least 80%, at least 85%, at least 90%, at least 95%, at least 98%, or at least 99% sequence identity to the nucleic acid sequence of SEQ ID NO: 1-99; wherein the transformed photosynthetic organism expresses said polynucleotide; and wherein the transformed photosynthetic organism produces an increase in biomass as compared to an untransformed photosynthetic organism of the same species. (46) The method of 45, wherein the increase is measured by a competition assay, growth rate, carrying capacity, productivity, cell proliferation, seed yield, organ growth, or polysome accumulation. (47) The method of 46, wherein the increase is measured by a competition assay. (48) The method of 47, wherein the competition assay is performed in a turbidostat. (49) The method of 45, wherein the increase is shown by the transformed photosynthetic organism having a positive selection coefficient as compared to an untransformed photosynthetic organism of the same species. (50) The method of 49, wherein the selection coefficient is from 0.05 to 0.10, from 0.10 to 0.5, from 0.5 to 0.75, from 0.75 to 1.0, from 1.0 to 1.5, from 1.5 to 2.0, or 2.0 to 3.0. (51) The method of 45, wherein the increase is measured by growth rate. (52) The method of 51, wherein the transformed photosynthetic organism has an increase in growth rate as compared to an untransformed photosynthetic organism of the same species of from 5% to 10%, from 10% to 15%, from 15% to 25%, from 25% to 50%, from 50% to 75%, from 75% to 100%, from 100% to 150%, from 150% to 200%, from 200% to 300%, or from 300% to 400%. (53) The method of 45, wherein the increase is measured by an increase in carrying capacity. (54) The method of 53, wherein the units of carrying capacity are mass per unit of volume or area. (55) The method of 45, wherein the increase is measured by an increase in culture productivity. (56) The method of 55, wherein the units of productivity are grams per meter squared per day, mass per unit area such as tons per acre/hectare, or volume per unit area such as bushels per acre/hectare. (57) The method of 45, wherein the transformed photosynthetic organism has an increase in productivity as measured in grams per meter squared per day, as compared to an untransformed photosynthetic organism of the same species of from 5% to 10%, from 10% to 15%, from 15% to 25%, from 25% to 50%, from 50% to 75%, from 75% to 100%, from 100% to 150%, from 150% to 200%, from 200% to 300%, or from 300% to 400%. (58) The method of 45, wherein the transformed photosynthetic organism is grown in an aqueous environment. (59) The method of 45, wherein the transformed photosynthetic organism is a bacterium. (60) The method of 59, wherein the bacterium is a cyanobacterium. (61) The method of 45, wherein the transformed photosynthetic organism is an alga. (62) The method of 61, wherein the alga is a microalga. (63) The method of 62, wherein the microalga is at least one of a Chlamydomonas sp., Volvacales sp., Desmid sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmus sp. (64) The method of 62, wherein the microalga is at least one of Chlamydomonas reinhardtii, N. oceanica, N. salina, Dunaliella salina, H. pluvalis, S. dimorphus, Dunaliella viridis, N. oculata, Dunaliella tertiolecta, S. Maximus, or A. Fusiformus. (65) The method of 45, wherein the transformed photosynthetic organism is a vascular plant. (66) The method of 65, wherein the transformed photosynthetic organism is Brassica (e.g., Brassica nigra, Brassica napus, Brassica hirta, Brassica rapa, Brassica campestris, Brassica carinata, and Brassica juncea), soybean (Glycine max), castor bean (Ricinus communis), cotton, safflower (Carthamus tinctorius), sunflower (Helianthus annuus), flax (Linum usitatissimum), corn (Zea mays), coconut (Cocos nucifera), palm (Elaeis guineensis), oil nut trees such as olive (Olea europaea), sesame, and peanut (Arachis hypogaea), as well as Arabidopsis, tobacco, wheat, sugarcane, sugar beet, barley, oats, amaranth, potato, rice, tomato, legumes (e.g., peas, beans, lentils, alfalfa, etc.), grasses (e.g. Miscanthus, switchgrass, energy cane), vegetable crops and fruits.

In addition is provided: (67) A method of increasing biomass of a photosynthetic organism, comprising (a) transforming the photosynthetic organism with at least one polynucleotide to produce a transformed photosynthetic organism, wherein the polynucleotide comprises (i) a nucleic acid sequence encodes a polypeptide with an amino acid sequence of SEQ ID NO: 100 to 189; or (ii) a polypeptide with an amino acid sequence with at least 80%, at least 85%, at least 90%, at least 95%, at least 98%, or at least 99% sequence identity to the nucleic acid sequence of SEQ ID NO: 100 to 189; wherein the transformed photosynthetic organism expresses the at least one polynucleotide to produce the polypeptide; and wherein the transformed photosynthetic organism produces an increase in biomass as compared to an untransformed photosynthetic organism of the same species. (68) The method of 67, wherein the increase is measured by a competition assay, growth rate, carrying capacity, productivity, cell proliferation, seed yield, organ growth, or polysome accumulation. (69) The method of 68, wherein the increase is measured by a competition assay. (70) The method of 69, wherein the competition assay is performed in a turbidostat. (71) The method of 67, wherein the increase is shown by the transformed photosynthetic organism having a positive selection coefficient as compared to an untransformed photosynthetic organism of the same species. (72) The method of 71, wherein the selection coefficient is from 0.05 to 0.10, from 0.10 to 0.5, from 0.5 to 0.75, from 0.75 to 1.0, from 1.0 to 1.5, from 1.5 to 2.0, or 2.0 to 3.0. (73) The method of 67, wherein the increase is measured by growth rate. (74) The method of 73, wherein the transformed photosynthetic organism has an increase in growth rate as compared to an untransformed photosynthetic organism of from 5% to 10%, from 10% to 15%, from 15% to 25%, from 25% to 50%, from 50% to 75%, from 75% to 100%, from 100% to 150%, from 150% to 200%, from 200% to 300%, or from 300% to 400%. (75) The method of 67, wherein the increase is measured by an increase in carrying capacity. (76) The method of 75, wherein the units of carrying capacity are mass per unit of volume or area. (77) The method of 67, wherein the increase is measured by an increase in productivity. (78) The method of 77, wherein the units of productivity are grams per meter squared per day, mass per unit area such as tons per acre/hectare, or volume per unit area such as bushels per acre/hectare. (79) The method of 67, wherein the transformed photosynthetic organism has an increase in productivity as measured in grams per meter squared per day, as compared to an untransformed photosynthetic organism of from 5% to 10%, from 10% to 15%, from 15% to 25%, from 25% to 50%, from 50% to 75%, from 75% to 100%, from 100% to 150%, from 150% to 200%, from 200% to 300%, or from 300% to 400%. (80) The method of 67, wherein the transformed photosynthetic organism is grown in an aqueous environment. (81) The method of 67, wherein the transformed photosynthetic organism is a bacterium. (82) The method of 81, wherein the bacterium is a cyanobacterium. (83) The method of 67, wherein the transformed photosynthetic organism is an alga. (84) The method of 83, wherein the alga is a microalga. (85) The method of 84, wherein the microalga is at least one of a Chlamydomonas sp., Volvacales sp., Desmid sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmus sp. (86) The method of 85, wherein the microalga is at least one of Chlamydomonas reinhardtii, N. oceanica, N. salina, Dunaliella salina, H. pluvalis, S. dimorphus, Dunaliella viridis, N. oculata, Dunaliella tertiolecta, S. Maximus, or A. Fusiformus. (87) The method of 67, wherein the transformed photosynthetic organism is a vascular plant. (88) The method of 87, wherein the transformed photosynthetic organism is Brassica (e.g., Brassica nigra, Brassica napus, Brassica hirta, Brassica rapa, Brassica campestris, Brassica carinata, and Brassica juncea), soybean (Glycine max), castor bean (Ricinus communis), cotton, safflower (Carthamus tinctorius), sunflower (Helianthus annuus), flax (Linum usitatissimum), corn (Zea mays), coconut (Cocos nucifera), palm (Elaeis guineensis), oil nut trees such as olive (Olea europaea), sesame, and peanut (Arachis hypogaea), as well as Arabidopsis, tobacco, wheat, sugarcane, sugar beet, barley, oats, amaranth, potato, rice, tomato, legumes (e.g., peas, beans, lentils, alfalfa, etc.), grasses (e.g. Miscanthus, switchgrass, energy cane), vegetable crops and fruits.

### BRIEF DESCRIPTION OF THE DRAWINGS

FIG. 1 shows plate reactor growth conditions used to mimic conditions in Las Cruces, N. Mex.

FIG. 2A shows expression vector pSENuc2643

FIG. 2B shows expression vector SENuc 1060

FIG. 3 shows a cDNA shuttle vector used in the experiments

FIG. 4 shows an exemplary validation process

### DETAILED DESCRIPTION

The following detailed description is provided to aid those skilled in the art in practicing the present disclosure. Even so, this detailed description should not be construed to unduly limit the present disclosure as modifications and variations in the embodiments discussed herein can be made by those of ordinary skill in the art without departing from the spirit or scope of the present inventive discovery.

As used in this specification and the appended claims, the singular forms “a”, “an” and “the” include plural reference unless the context clearly dictates otherwise.

An endogenous nucleic acid, nucleotide, polypeptide, or protein as described herein is defined in relationship to the host organism. An endogenous nucleic acid, nucleotide, polypeptide, or protein is one that naturally occurs in the host organism.

An exogenous nucleic acid, nucleotide, polypeptide, or protein as described herein is defined in relationship to the host organism. An exogenous nucleic acid, nucleotide, polypeptide, or protein is one that does not naturally occur in the host organism or is a different location in the host organism.

If an initial start codon (Met) is not present in any of the amino acid sequences disclosed herein, including sequences contained in the sequence listing, one of skill in the art would be able to include, at the nucleotide level, an initial ATG, so that the translated polypeptide would have the initial Met. If a start and/or stop codon is not present at the beginning and/or end of a coding sequence, one of skill in the art would know to insert an “ATG” at the beginning of the coding sequence and nucleotides encoding for a stop codon (any one of TM, TAG, or TGA) at the end of the coding sequence. Any of the disclosed nucleotide sequences can be, if desired, fused to another nucleotide sequence that when operably linked to a “control element” results in the proper translation of the encoded amino acids (for example, a fusion protein). In addition, two or more nucleotide sequences can be linked by a short peptide, for example, a viral peptide.

Increased yield in higher plants can be manifested in phenotypes such as increased cell proliferation, increased organ or cell size and increased total plant mass. The phrases “an increase in biomass yield” and “an increase in biomass” are used interchangeably throughout the specification.

An increase in biomass yield can be defined by a number of growth measures, including, for example, a selective advantage during competitive growth, increased growth rate, increased carrying capacity, and/or increased culture productivity (as measured on a per volume or per area basis). For example, a competition assay can be between a transgenic strain and a wild-type strain, between several transgenic strains, or between several transgenic strains and a wild-type strain.

Disclosed herein are methods for increasing biomass of an organism by transforming a host cell or host organism with one or more of the nucleotides sequences disclosed herein. In some embodiments, a host cell is part of a multicellular organism. In other embodiments, a host cell is cultured as a unicellular organism. Host organisms can include any suitable host, for example, a microorganism. Microorganisms which are useful for the methods described herein include, for example, photosynthetic bacteria (e.g., cyanobacteria), non-photosynthetic bacteria (e.g., E. coli), yeast (e.g., Saccharomyces cerevisiae), and algae.

Examples of host organisms that can be transformed with one or more of the polynucleotides disclosed herein include vascular and non-vascular organisms. The organism can be prokaryotic or eukaryotic. The organism can be unicellular or multicellular. A host organism is an organism comprising a host cell. In other embodiments, the host organism is photosynthetic. A photosynthetic organism is one that naturally photosynthesizes (e.g., an alga) or that is genetically engineered or otherwise modified to be photosynthetic. In some instances, a photosynthetic organism may be transformed with a construct or vector of the disclosure which renders all or part of the photosynthetic apparatus inoperable. By way of example and not limitation, a non-vascular photosynthetic microalga species include C. reinhardtii, Nannochloropsis oceania, N. salina, D. salina, H. pluvalis, S. dimorphus, D. viridis, Chlorella sp., and D. tertiolecta.

In other embodiments the host organism is a vascular plant. Non-limiting examples of such plants include various monocots and dicots, including high oil seed plants such as high oil seed Brassica (e.g., Brassica nigra, Brassica napus, Brassica hirta, Brassica rapa, Brassica campestris, Brassica carinata, and Brassica juncea), soybean (Glycine max), castor bean (Ricinus communis), cotton, safflower (Carthamus tinctorius), sunflower (Helianthus annuus), flax (Linum usitatissimum), corn (Zea mays), coconut (Cocos nucifera), palm (Elaeis guineensis), oil nut trees such as olive (Olea europaea), sesame, and peanut (Arachis hypogaea), as well as Arabidopsis, tobacco, wheat, sugarcane, sugar beet, barley, oats, amaranth, potato, rice, tomato, legumes (e.g., peas, beans, lentils, alfalfa, etc.), grasses (e.g. Miscanthus, switchgrass, energy cane), vegetable crops and fruits.

The host cell can be prokaryotic. Examples of some prokaryotic organisms useful in the practice of the present disclosure include, but are not limited to, cyanobacteria (e.g., Synechococcus, Synechocystis, Athrospira, Gleocapsa, Oscillatoria, and, Pseudoanabaena). Suitable prokaryotic cells include, but are not limited to, any of a variety of laboratory strains of Escherichia coli, Lactobacillus sp., Salmonella sp., and Shigella sp. (for example, as described in Carrier et al. (1992) J. Immunol. 148:1176-1181; U.S. Pat. No. 6,447,784; and Sizemore et al. (1995) Science 270:299-302). Examples of Salmonella strains which can be employed in the present disclosure include, but are not limited to, Salmonella typhi and S. typhimurium. Suitable Shigella strains include, but are not limited to, Shigella flexneri, Shigella sonnei, and Shigella disenteriae. Typically, the laboratory strain is one that is non-pathogenic. Non-limiting examples of other suitable bacteria include, but are not limited to, Pseudomonas pudita, Pseudomonas aeruginosa, Pseudomonas mevalonii, Rhodobacter sphaeroides, Rhodobacter capsulatus, Rhodospirillum rubrum, and Rhodococcus sp.

In some embodiments, the host organism is eukaryotic (e.g. green algae, red algae, brown algae). In some embodiments, the algae is a green algae, for example, a Chlorophycean. The algae can be unicellular or multicellular. Suitable eukaryotic host cells include, but are not limited to, yeast cells, insect cells, plant cells, fungal cells, and algal cells. Suitable eukaryotic host cells include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, and Chlamydomonas reinhardtii.

In some embodiments, eukaryotic microalgae, such as for example, a Chlamydomonas, Volvacales, Dunaliella, Nannochloropsis, Desmodesmus, Scenedesmus, Chlorella, or Hematococcus species, can be used in the disclosed methods. In more specific embodiments, the host cell is Chlamydomonas reinhardtii, Dunaliella salina, Haematococcus pluvialis, Nannochloropsis oceania, Nannochloropsis salina, Scenedesmus dimorphus, a Chlorella species, a Spirulina species, a Desmid species, Spirulina maximus, Arthrospira fusiformis, Dunaliella viridis, or Dunaliella tertiolecta.

In some instances the organism is a rhodophyte, chlorophyte, heterokontophyte, tribophyte, glaucophyte, chlorarachniophyte, euglenoid, haptophyte, cryptomonad, dinoflagellum, or phytoplankton.

In some instances a host organism is vascular and photosynthetic. Examples of vascular plants include, but are not limited to, angiosperms, gymnosperms, rhyniophytes, or other tracheophytes. In other instances a host organism is non-vascular and photosynthetic. As used herein, the term “non-vascular photosynthetic organism,” refers to any macroscopic or microscopic organism, including, but not limited to, algae, cyanobacteria and photosynthetic bacteria, which does not have a vascular system such as that found in vascular plants. Examples of non-vascular photosynthetic organisms include bryophtyes, such as marchantiophytes or anthocerotophytes. In some instances the organism is a cyanobacteria. In some instances, the organism is algae (e.g., macroalgae or microalgae). The algae can be unicellular or multicellular algae.

In certain embodiments, the host cell is a plant. The term “plant” is used broadly herein to refer to a eukaryotic organism containing plastids, such as chloroplasts, and includes any such organism at any stage of development, or to part of a plant; including a plant cutting, a plant cell, a plant cell culture, a plant organ, a plant seed, and a plantlet. A plant cell is the structural and physiological unit of the plant, comprising a protoplast and a cell wall. A plant cell can be in the form of an isolated single cell or a cultured cell, or can be part of higher organized unit, for example, a plant tissue, plant organ, or plant. Thus, a plant cell can be a protoplast, a gamete producing cell, or a cell or collection of cells that can regenerate into a whole plant. As such, a seed, which comprises multiple plant cells and is capable of regenerating into a whole plant, is considered plant cell for purposes of this disclosure. A plant tissue or plant organ can be a seed, protoplast, callus, or any other groups of plant cells that is organized into a structural or functional unit. Particularly useful parts of a plant include harvestable parts and parts useful for propagation of progeny plants. A harvestable part of a plant can be any useful part of a plant, for example, flowers, pollen, seedlings, tubers, leaves, stems, fruit, seeds, and roots. A part of a plant useful for propagation includes, for example, seeds, fruits, cuttings, seedlings, tubers, and rootstocks.

Some of the host organisms useful in the disclosed embodiments are, for example, are extremophiles, such as hyperthermophiles, psychrophiles, psychrotrophs, halophiles, barophiles and acidophiles. Some of the host organisms which may be used to practice the present disclosure are halophilic (e.g., Dunaliella salina, D. viridis, or D. tertiolecta). For example, D. salina can grow in ocean water and salt lakes (for example, salinity from 30-300 parts per thousand) and high salinity media (e.g., artificial seawater medium, seawater nutrient agar, brackish water medium, and seawater medium). In some embodiments of the disclosure, a host cell expressing a protein of the present disclosure can be grown in a liquid environment which is, for example, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 31., 3.2, 3.3, 3.4, 3.5, 3.6, 3.7, 3.8, 3.9, 4.0, 4.1, 4.2, 4.3 molar or higher concentrations of sodium chloride. One of skill in the art will recognize that other salts (sodium salts, calcium salts, potassium salts, or other salts) may also be present in the liquid environments.

An organism may be grown under conditions which permit photosynthesis, however, this is not a requirement (e.g., a host organism may be grown in the absence of light). In some instances, the host organism may be genetically modified in such a way that its photosynthetic capability is diminished or destroyed. In growth conditions where a host organism is not capable of photosynthesis (e.g., because of the absence of light and/or genetic modification), typically, the organism will be provided with the necessary nutrients to support growth in the absence of photosynthesis. For example, a culture medium in (or on) which an organism is grown, may be supplemented with any required nutrient, including an organic carbon source, nitrogen source, phosphorous source, vitamins, metals, lipids, nucleic acids, micronutrients, and/or an organism-specific requirement. Organic carbon sources include any source of carbon which the host organism is able to metabolize including, but not limited to, acetate, simple carbohydrates (e.g., glucose, sucrose, and lactose), complex carbohydrates (e.g., starch and glycogen), proteins, and lipids. One of skill in the art will recognize that not all organisms will be able to sufficiently metabolize a particular nutrient and that nutrient mixtures may need to be modified from one organism to another in order to provide the appropriate nutrient mix.

Optimal growth of algal organisms occurs usually at a temperature of about 20° C. to about 25° C., although some organisms can still grow at a temperature of up to about 35° C. Active growth is typically performed in liquid culture. If the organisms are grown in a liquid medium and are shaken or mixed, the density of the cells can be anywhere from about 1 to 5×10⁸ cells/ml at the stationary phase. For example, the density of the cells at the stationary phase for Chlamydomonas sp. can be about 1 to 5×10⁷ cells/ml; the density of the cells at the stationary phase for Nannochloropsis sp. can be about 1 to 5×10⁸ cells/ml; the density of the cells at the stationary phase for Scenedesmus sp. can be about 1 to 5×10⁷ cells/ml; and the density of the cells at the stationary phase for Chlorella sp. can be about 1 to 5×10⁸ cells/ml. Exemplary cell densities at the stationary phase are as follows: Chlamydomonas sp. can be about 1×10⁷ cells/ml; Nannochloropsis sp. can be about 1×10⁸ cells/ml; Scenedesmus sp. can be about 1×10⁷ cells/ml; and Chlorella sp. can be about 1×10⁸ cells/ml. An exemplary growth rate may yield, for example, a two to twenty fold increase in cells per day, depending on the growth conditions. In addition, doubling times for organisms can be, for example, 5 hours to 30 hours. The organism can also be grown on solid media, for example, media containing about 1.5% agar, in plates or in slants.

One source of energy is fluorescent light that can be placed, for example, at a distance of about 1 inch to about two feet from the algae. Examples of types of fluorescent lights includes, for example, cool white and daylight. Bubbling with air or CO₂ improves the growth rate of the organism. Bubbling with CO₂ can be, for example, at 1% to 5% CO₂. If the lights are turned on and off at regular intervals (for example, 12:12 or 14:10 hours of light:dark) the cells of some organisms will become synchronized.

Long term storage of algae can be achieved by streaking them onto plates, sealing the plates with, for example, PARAFILM™, and placing them in dim light at about 10° C. to about 18° C. Alternatively, algae may be grown as streaks or stabs into agar tubes, capped, and stored at about 10° C. to about 18° C. Both methods allow for the storage of the organisms for several months.

For longer storage, the algae can be grown in liquid culture to mid to late log phase and then supplemented with a penetrating cryoprotective agent like DMSO or MeOH, and stored at less than −130° C. An exemplary range of DMSO concentrations that can be used is 5 to 8%. An exemplary range of MeOH concentrations that can be used is 3 to 9%.

Organisms can be grown on a defined minimal medium (for example, high salt medium (HSM), modified artificial sea water medium (MASM), or F/2 medium) with light as the sole energy source. In other instances, the organism can be grown in a medium (for example, tris acetate phosphate (TAP) medium), and supplemented with an organic carbon source.

Organisms, such as algae, can grow naturally in fresh water or marine water. Culture media for freshwater algae can be, for example, synthetic media, enriched media, soil water media, and solidified media, such as agar. Various culture media have been developed and used for the isolation and cultivation of fresh water algae and are described in Watanabe, M. W. (2005). Freshwater Culture Media. In R. A. Andersen (Ed.), Algal Culturing Techniques (pp. 13-20). Elsevier Academic Press. Culture media for marine algae can be, for example, artificial seawater media or natural seawater media. Guidelines for the preparation of media are described in Harrison, P. J. and Berges, J. A. (2005). Marine Culture Media. In R. A. Andersen (Ed.), Algal Culturing Techniques (pp. 21-33). Elsevier Academic Press.

Organisms may be grown in outdoor open water, such as ponds, the ocean, seas, rivers, waterbeds, marshes, shallow pools, lakes, aqueducts, and reservoirs. When grown in water, the organism can be contained in a halo-like object comprised of lego-like particles. The halo-like object encircles the organism and allows it to retain nutrients from the water beneath while keeping it in open sunlight.

In some instances, organisms can be grown in containers wherein each container comprises one or two organisms, or a plurality of organisms. The containers can be configured to float on water. For example, a container can be filled by a combination of air and water to make the container and the organism(s) in it buoyant. An organism that is adapted to grow in fresh water can thus be grown in salt water (i.e., the ocean) and vice versa. This mechanism allows for automatic death of the organism if there is any damage to the container. Culturing techniques for algae are well known to one of skill in the art and are described, for example, in Freshwater Culture Media. In R. A. Andersen (Ed.), Algal Culturing Techniques. Elsevier Academic Press.

Because photosynthetic organisms, for example, algae, require sunlight, CO₂ and water for growth, they can be cultivated in, for example, open ponds and lakes. However, these open systems are more vulnerable to contamination than a closed system. One challenge with using an open system is that the organism of interest may not grow as quickly as a potential invader. This becomes a problem when another organism invades the liquid environment in which the organism of interest is growing, and the invading organism has a faster growth rate and takes over the system. In addition, in open systems there is less control over water temperature, CO₂ concentration, and lighting conditions. The growing season of the organism is largely dependent on location and, aside from tropical areas, is limited to the warmer months of the year. In addition, in an open system, the number of different organisms that can be grown is limited to those that are able to survive in the chosen location. An open system, however, is cheaper to set up and/or maintain than a closed system.

Another approach to growing an organism is to use a semi-closed system, such as covering the pond or pool with a structure, for example, a “greenhouse-type” structure. While this can result in a smaller system, it addresses many of the problems associated with an open system. The advantages of a semi-closed system are that it can allow for a greater number of different organisms to be grown, it can allow for an organism to be dominant over an invading organism by allowing the organism of interest to out compete the invading organism for nutrients required for its growth, and it can extend the growing season for the organism. For example, if the system is heated, the organism can grow year round.

A variation of the pond system is an artificial pond, for example, a raceway pond. In these ponds, the organism, water, and nutrients circulate around a “racetrack.” Paddlewheels provide constant motion to the liquid in the racetrack, allowing for the organism to be circulated back to the surface of the liquid at a chosen frequency. Paddlewheels also provide a source of agitation and oxygenate the system. These raceway ponds can be enclosed, for example, in a building or a greenhouse, or can be located outdoors. Raceway ponds are usually kept shallow because the organism needs to be exposed to sunlight, and sunlight can only penetrate the pond water to a limited depth. The depth of a raceway pond can be, for example, about 4 to about 12 inches. In addition, the volume of liquid that can be contained in a raceway pond can be, for example, about 200 liters to about 600,000 liters.

If the raceway pond is placed outdoors, there are several different ways to address the invasion of an unwanted organism. For example, the pH or salinity of the liquid in which the desired organism is in can be such that the invading organism either slows down its growth or dies. Also, chemicals can be added to the liquid, such as bleach, or a pesticide can be added to the liquid, such as glyphosate. In addition, the organism of interest can be genetically modified such that it is better suited to survive in the liquid environment. Any one or more of the above strategies can be used to address the invasion of an unwanted organism.

Alternatively, organisms, such as algae, can be grown in closed structures such as photobioreactors, where the environment is under stricter control than in open systems or semi-closed systems. A photobioreactor is a bioreactor which incorporates some type of light source to provide photonic energy input into the reactor. The term photobioreactor can refer to a system closed to the environment and having no direct exchange of gases and contaminants with the environment. A photobioreactor can be described as an enclosed, illuminated culture vessel designed for controlled biomass production of phototrophic liquid cell suspension cultures. Examples of photobioreactors include, for example, glass containers, plastic tubes, tanks, plastic sleeves, and bags. Examples of light sources that can be used to provide the energy required to sustain photosynthesis include, for example, fluorescent bulbs, LEDs, and natural sunlight. Because these systems are closed everything that the organism needs to grow (for example, carbon dioxide, nutrients, water, and light) must be introduced into the bioreactor.

Photobioreactors, despite the costs to set up and maintain them, have several advantages over open systems, they can, for example, prevent or minimize contamination, permit axenic organism cultivation of monocultures (a culture consisting of only one species of organism), offer better control over the culture conditions (for example, pH, light, carbon dioxide, and temperature), prevent water evaporation, lower carbon dioxide losses due to out gassing, and permit higher cell concentrations. On the other hand, certain requirements of photobioreactors, such as cooling, mixing, control of oxygen accumulation and biofouling, make these systems more expensive to build and operate than open systems or semi-closed systems.

Photobioreactors can be set up to be continually harvested (as is with the majority of the larger volume cultivation systems), or harvested one batch at a time (for example, as with polyethlyene bag cultivation). A batch photobioreactor is set up with, for example, nutrients, an organism (for example, algae), and water, and the organism is allowed to grow until the batch is harvested. A continuous photobioreactor can be harvested, for example, either continually, daily, or at fixed time intervals.

High density photobioreactors are described in, for example, Lee, et al., Biotech. Bioengineering 44:1161-1167, 1994. Other types of bioreactors, such as those for sewage and waste water treatments, are described in, Sawayama, et al., Appl. Micro. Biotech., 41:729-731, 1994. Additional examples of photobioreactors are described in, U.S. Appl. Publ. No. 2005/0260553, U.S. Pat. Nos. 5,958,761, and 6,083,740. Also, organisms, such as algae may be mass-cultured for the removal of heavy metals (for example, as described in Wilkinson, Biotech. Letters, 11:861-864, 1989), hydrogen (for example, as described in U.S. Patent Application Publication No. 2003/0162273), and pharmaceutical compounds from a water, soil, or other source or sample. Organisms can also be cultured in conventional fermentation bioreactors, which include, but are not limited to, batch, fed-batch, cell recycle, and continuous fermentors. Additional methods of culturing organisms and variations of the methods described herein are known to one of skill in the art.

CO₂ can be delivered to any of the systems described herein, for example, by bubbling in CO₂ from under the surface of the liquid containing the organism. Also, sparges can be used to inject CO₂ into the liquid. Spargers are, for example, porous disc or tube assemblies that are also referred to as Bubblers, Carbonators, Aerators, Porous Stones and Diffusers. Nutrients that can be used in the systems described herein include, for example, nitrogen (in the form of NO₃⁻ or NH₄⁺), phosphorus, and trace metals (Fe, Mg, K, Ca, Co, Cu, Mn, Mo, Zn, V, and B). The nutrients can come, for example, in a solid form or in a liquid form. If the nutrients are in a solid form they can be mixed with, for example, fresh or salt water prior to being delivered to the liquid containing the organism, or prior to being delivered to a photobioreactor.

Algae can be grown in large scale cultures, where large scale cultures refers to growth of cultures in volumes of greater than about 6 liters, or greater than about 10 liters, or greater than about 20 liters. Large scale growth can also be growth of cultures in volumes of 50 liters or more, 100 liters or more, or 200 liters or more. Large scale growth can be growth of cultures in, for example, ponds, containers, vessels, or other areas, where the pond, container, vessel, or area that contains the culture is for example, at lease 5 square meters, at least 10 square meters, at least 200 square meters, at least 500 square meters, at least 1,500 square meters, at least 2,500 square meters, in area, or greater.

It should be recognized that the present disclosure is not limited to transgenic cells, organisms, and plastids containing polynucleotides disclosed herein, but also encompasses such cells, organisms, and plastids transformed with additional nucleotide sequences encoding enzymes involved in fatty acid synthesis. Thus, some embodiments involve the introduction of one or more sequences encoding proteins involved in fatty acid synthesis in addition to a protein disclosed herein. For example, several enzymes in a fatty acid production pathway may be linked, either directly or indirectly, such that products produced by one enzyme in the pathway, once produced, are in close proximity to the next enzyme in the pathway. These additional sequences may be contained in a single vector either operatively linked to a single promoter or linked to multiple promoters, e.g. one promoter for each sequence. Alternatively, the additional coding sequences may be contained in a plurality of additional vectors. When a plurality of vectors are used, they can be introduced into the host cell or organism simultaneously or sequentially.

Additional embodiments provide a plastid, and in particular a chloroplast, transformed with a polynucleotide of the present disclosure. The polynucleotide may be introduced into the genome of the plastid using any of the methods described herein or otherwise known in the art. The plastid may be contained in the organism in which it naturally occurs. Alternatively, the plastid may be an isolated plastid, that is, a plastid that has been removed from the cell in which it normally occurs. Methods for the isolation of plastids are known in the art and can be found, for example, in Maliga et al., Methods in Plant Molecular Biology, Cold Spring Harbor Laboratory Press, 1995; Gupta and Singh, J. Biosci., 21:819 (1996); and Camara et al., Plant Physiol., 73:94 (1983). The isolated plastid transformed with a protein of the present disclosure can be introduced into a host cell. The host cell can be one that naturally contains the plastid or one in which the plastid is not naturally found.

Also within the scope of the present disclosure are artificial plastid genomes, for example chloroplast genomes, that contain nucleotide sequences encoding any one or more of the proteins of the present disclosure. Methods for the assembly of artificial plastid genomes can be found in U.S. patent application Ser. No. 12/287,230 filed Oct. 6, 2008, published as U.S. Publication No. 2009/0123977 on May 14, 2009, and U.S. patent application Ser. No. 12/384,893 filed Apr. 8, 2009, published as U.S. Publication No. 2009/0269816 on Oct. 29, 2009, each of which is incorporated by reference in its entirety.

One or more polynucleotides of the present disclosure can also be modified such that the resulting amino acid is “substantially identical” to the unmodified or reference amino acid. A “substantially identical” amino acid sequence is a sequence that differs from a reference sequence by one or more conservative or non-conservative amino acid substitutions, deletions, or insertions, particularly when such a substitution occurs at a site that is not the active site (catalytic domains (CDs)) of the molecule and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine). Conservative substitutions are those that substitute a given amino acid in a polypeptide by another amino acid of like characteristics. Examples of conservative substitutions are the following replacements: replacements of an aliphatic amino acid such as Alanine, Valine, Leucine and Isoleucine with another aliphatic amino acid; replacement of a Serine with a Threonine or vice versa; replacement of an acidic residue such as Aspartic acid and Glutamic acid with another acidic residue; replacement of a residue bearing an amide group, such as Asparagine and Glutamine, with another residue bearing an amide group; exchange of a basic residue such as Lysine and Arginine with another basic residue; and replacement of an aromatic residue such as Phenylalanine, Tyrosine with another aromatic residue. In alternative aspects, these conservative substitutions can also be synthetic equivalents of these amino acids.

To generate a genetically modified host cell or organism, a polynucleotide, or a polynucleotide cloned into a vector, is introduced stably or transiently into a host cell, using established techniques, including, but not limited to, electroporation, calcium phosphate precipitation, DEAE-dextran mediated transfection, and liposome-mediated transfection. For transformation, a polynucleotide of the present disclosure will generally further include a selectable marker, e.g., any of several well-known selectable markers such as neomycin resistance, ampicillin resistance, tetracycline resistance, chloramphenicol resistance, and kanamycin resistance.

A polynucleotide or recombinant nucleic acid molecule described herein, can be introduced into a cell (e.g., alga cell) using any method known in the art. A polynucleotide can be introduced into a cell by a variety of methods, which are well known in the art and selected, in part, based on the particular host cell. For example, the polynucleotide can be introduced into a cell using a direct gene transfer method such as electroporation or microprojectile mediated (biolistic) transformation using a particle gun, or the “glass bead method,” or by pollen-mediated transformation, liposome-mediated transformation, transformation using wounded or enzyme-degraded immature embryos, or wounded or enzyme-degraded embryogenic callus (for example, as described in Potrykus, Ann. Rev, Plant. Physiol. Plant Mol. Biol. 42:205-225, 1991).

As discussed above, microprojectile mediated transformation can be used to introduce a polynucleotide into a cell (for example, as described in Klein et al., Nature 327:70-73, 1987). This method utilizes microprojectiles such as gold or tungsten, which are coated with the desired polynucleotide by precipitation with calcium chloride, spermidine or polyethylene glycol. The microprojectile particles are accelerated at high speed into a cell using a device such as the BIOLISTIC PD-1000 particle gun (BioRad; Hercules Calif.). Methods for the transformation using biolistic methods are well known in the art (for example, as described in Christou, Trends in Plant Science 1:423-431, 1996). Microprojectile mediated transformation has been used, for example, to generate a variety of transgenic plant species, including cotton, soybean, tobacco, corn, hybrid poplar and papaya. Important cereal crops such as wheat, oat, barley, sorghum and rice also have been transformed using microprojectile mediated delivery (for example, as described in Duan et al., Nature Biotech. 14:494-498, 1996; and Shimamoto, Curr. Opin. Biotech. 5:158-162, 1994). The transformation of most dicotyledonous plants is possible with the methods described above. Transformation of monocotyledonous and dicotyledonous plants can be transformed using, for example, biolistic methods as described above, bacterially mediated or Agrobacterium-mediated transformation, protoplast transformation, electroporation of partially permeabilized cells, introduction of DNA using glass fibers, glass bead agitation method, etc., as known in the art. Methods for biolistic transformation of algae are known in the art.

The basic techniques used for transformation and expression in photosynthetic microorganisms are similar to those commonly used for E. coli, Saccharomyces cerevisiae and other species. Transformation methods customized for a photosynthetic microorganisms, e.g., the chloroplast of a strain of algae, are known in the art. These methods have been described in a number of texts for standard molecular biological manipulation (see Packer & Glaser, 1988, “Cyanobacteria”, Meth. Enzymol., Vol. 167; Weissbach & Weissbach, 1988, “Methods for plant molecular biology,” Academic Press, New York, Sambrook, Fritsch & Maniatis, 1989, “Molecular Cloning: A laboratory manual,” 2nd edition Cold Spring Harbor Laboratory Press, Cold Spring Harbor, N.Y.; and Clark M S, 1997, Plant Molecular Biology, Springer, N.Y.). These methods include, for example, biolistic devices (See, for example, Sanford, Trends In Biotech. (1988) 6: 299-302, U.S. Pat. No. 4,945,050; electroporation (Fromm et al., Proc. Nat'l. Acad. Sci. (USA) (1985) 82: 5824-5828); use of a laser beam, electroporation, microinjection or any other method capable of introducing DNA into a host cell.

Plastid transformation is a routine and well known method for introducing a polynucleotide into a plant cell chloroplast (see U.S. Pat. Nos. 5,451,513, 5,545,817, and 5,545,818; WO 95/16783; McBride et al., Proc. Natl. Acad. Sci., USA 91:7301-7305, 1994). In some embodiments, chloroplast transformation involves introducing regions of chloroplast DNA flanking a desired nucleotide sequence, allowing for homologous recombination of the exogenous DNA into the target chloroplast genome. In some instances one to 1.5 kb flanking nucleotide sequences of chloroplast genomic DNA may be used. Using this method, point mutations in the chloroplast 16S rRNA and rps12 genes, which confer resistance to spectinomycin and streptomycin, can be utilized as selectable markers for transformation (Svab et al., Proc. Natl. Acad. Sci., USA 87:8526-8530, 1990), and can result in stable homoplasmic transformants, at a frequency of approximately one per 100 bombardments of target leaves. Methods for the transformation of algal chloroplasts can be found in U.S. Patent Application Publication 2012/0252054 which is incorporated by reference in its entirety.

A further refinement in chloroplast transformation/expression technology that facilitates control over the timing and tissue pattern of expression of introduced DNA coding sequences in plant plastid genomes has been described in PCT International Publication WO 95/16783 and U.S. Pat. No. 5,576,198. This method involves the introduction into plant cells of constructs for nuclear transformation that provide for the expression of a viral single subunit RNA polymerase and targeting of this polymerase into the plastids via fusion to a plastid transit peptide. Transformation of plastids with DNA constructs comprising a viral single subunit RNA polymerase-specific promoter specific to the RNA polymerase expressed from the nuclear expression constructs operably linked to DNA coding sequences of interest permits control of the plastid expression constructs in a tissue and/or developmental specific manner in plants comprising both the nuclear polymerase construct and the plastid expression constructs. Expression of the nuclear RNA polymerase coding sequence can be placed under the control of either a constitutive promoter, or a tissue- or developmental stage-specific promoter, thereby extending this control to the plastid expression construct responsive to the plastid-targeted, nuclear-encoded viral RNA polymerase.

When nuclear transformation is utilized, the protein can be modified for plastid targeting by employing plant cell nuclear transformation constructs wherein DNA coding sequences of interest are fused to any of the available transit peptide sequences capable of facilitating transport of the encoded enzymes into plant plastids, and driving expression by employing an appropriate promoter. Targeting of the protein can be achieved by fusing DNA encoding plastid, e.g., chloroplast, leucoplast, amyloplast, etc., transit peptide sequences to the 5′ end of DNAs encoding the enzymes. The sequences that encode a transit peptide region can be obtained, for example, from plant nuclear-encoded plastid proteins, such as the small subunit (SSU) of ribulose bisphosphate carboxylase, EPSP synthase, plant fatty acid biosynthesis related genes including fatty acyl-ACP thioesterases, acyl carrier protein (ACP), stearoyl-ACP desaturase, β-ketoacyl-ACP synthase and acyl-ACP thioesterase, or LHCPII genes, etc. Plastid transit peptide sequences can also be obtained from nucleic acid sequences encoding carotenoid biosynthetic enzymes, such as GGPP synthase, phytoene synthase, and phytoene desaturase. Other transit peptide sequences are disclosed in Von Heijne et al. (1991) Plant Mol. Biol. Rep. 9: 104; Clark et al. (1989) J. Biol. Chem. 264: 17544; della-Cioppa et al. (1987) Plant Physiol. 84: 965; Romer et al. (1993) Biochem. Biophys. Res. Commun. 196: 1414; and Shah et al. (1986) Science 233: 478. Another transit peptide sequence is that of the intact ACCase from Chlamydomonas (genbank EDO96563, amino acids 1-33). The encoding sequence for a transit peptide effective in transport to plastids can include all or a portion of the encoding sequence for a particular transit peptide, and may also contain portions of the mature protein encoding sequence associated with a particular transit peptide. Numerous examples of transit peptides that can be used to deliver target proteins into plastids exist, and the particular transit peptide encoding sequences useful in the present disclosure are not critical as long as delivery into a plastid is obtained. Proteolytic processing within the plastid then produces the mature enzyme. This technique has proven successful with enzymes involved in polyhydroxyalkanoate biosynthesis (Nawrath et al. (1994) Proc. Natl. Acad. Sci. USA 91: 12760), and neomycin phosphotransferase II (NPT-II) and CP4 EPSPS (Padgette et al. (1995) Crop Sci. 35: 1451), for example.

Of interest are transit peptide sequences derived from enzymes known to be imported into the leucoplasts of seeds. Examples of enzymes containing useful transit peptides include those related to lipid biosynthesis (e.g., subunits of the plastid-targeted dicot acetyl-CoA carboxylase, biotin carboxylase, biotin carboxyl carrier protein, α-carboxy-transferase, and plastid-targeted monocot multifunctional acetyl-CoA carboxylase (Mw, 220,000); plastidic subunits of the fatty acid synthase complex (e.g., acyl carrier protein (ACP), malonyl-ACP synthase, KASI, KASII, and KASIII); steroyl-ACP desaturase; thioesterases (specific for short, medium, and long chain acyl ACP); plastid-targeted acyl transferases (e.g., glycerol-3-phosphate and acyl transferase); enzymes involved in the biosynthesis of aspartate family amino acids; phytoene synthase; gibberellic acid biosynthesis (e.g., ent-kaurene synthases 1 and 2); and carotenoid biosynthesis (e.g., lycopene synthase).

In one embodiment, a transformation may introduce a nucleic acid into a plastid genome of the host cell (e.g., chloroplast). In another embodiment, a transformation may introduce a nucleic acid into the nuclear genome of the host cell. In still another embodiment, a transformation may introduce nucleic acids into both the nuclear genome and into a plastid genome.

Transformed cells can be plated on selective media following introduction of exogenous nucleic acids. This method may also comprise several steps for screening. A screen of primary transformants can be conducted to determine which clones have proper insertion of the exogenous nucleic acids. Clones which show the proper integration may be propagated and re-screened to ensure genetic stability. Such methodology ensures that the transformants contain the genes of interest. In many instances, such screening is performed by polymerase chain reaction (PCR); however, any other appropriate technique known in the art may be utilized. Many different methods of PCR are known in the art (e.g., nested PCR, real time PCR). For any given screen, one of skill in the art will recognize that PCR components may be varied to achieve optimal screening results. For example, magnesium concentration may need to be adjusted upwards when PCR is performed on disrupted alga cells to which (which chelates magnesium) is added to chelate toxic metals. Following the screening for clones with the proper integration of exogenous nucleic acids, clones can be screened for the presence of the encoded protein(s), products and/or phenotypes. Protein expression screening can be performed by Western blot analysis and/or enzyme activity assays. Transporter and/or product screening may be performed by any method known in the art, for example ATP turnover assay, substrate transport assay, HPLC or gas chromatography.

The expression of the polynucleotide can be accomplished by inserting a polynucleotide sequence (gene) encoding the protein or enzyme into the chloroplast or nuclear genome of a microalgae. The modified cell can be made homoplasmic to ensure that the polynucleotide will be stably maintained in the chloroplast genome of all descendents. A cell is homoplasmic for a gene when the inserted gene is present in all copies of the chloroplast genome, for example. It is apparent to one of skill in the art that a chloroplast may contain multiple copies of its genome, and therefore, the term “homoplasmic” or “homoplasmy” refers to the state where all copies of a particular locus of interest are substantially identical. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% or more of the total soluble plant protein.

Construct, vector and plasmid are used interchangeably throughout the disclosure. Nucleic acids described herein, can be contained in vectors, including cloning and expression vectors. A cloning vector is a self-replicating DNA molecule that serves to transfer a DNA segment into a host cell. Three common types of cloning vectors are bacterial plasmids, phages, and other viruses. An expression vector is a cloning vector designed so that a coding sequence inserted at a particular site will be transcribed and translated into a protein. Both cloning and expression vectors can contain nucleotide sequences that allow the vectors to replicate in one or more suitable host cells. In cloning vectors, this sequence is generally one that enables the vector to replicate independently of the host cell chromosomes, and also includes either origins of replication or autonomously replicating sequences.

In some embodiments, a polynucleotide of the present disclosure is cloned or inserted into an expression vector using cloning techniques known to one of skill in the art. The nucleotide sequences may be inserted into a vector by a variety of methods. In the most common method the sequences are inserted into an appropriate restriction endonuclease site(s) using procedures commonly known to those skilled in the art and detailed in, for example, Sambrook et al., Molecular Cloning, A Laboratory Manual, 2nd Ed., Cold Spring Harbor Press, (1989) and Ausubel et al., Short Protocols in Molecular Biology, 2nd Ed., John Wiley & Sons (1992). Vectors for plant transformation have been reviewed in Rodriguez et al. (1988) Vectors: A Survey of Molecular Cloning Vectors and Their Uses, Butterworths, Boston; Glick et al. (1993) Methods in Plant Molecular Biology and Biotechnology CRC Press, Boca Raton, Fla.; and Croy (1993) In Plant Molecular Biology Labfax, Hames and Rickwood, Eds., BIOS Scientific Publishers Limited, Oxford, UK.

Suitable expression vectors include, but are not limited to, baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, and herpes simplex virus), PI-based artificial chromosomes, yeast plasmids, yeast artificial chromosomes, and any other vectors specific for specific hosts of interest (such as E. coli and yeast). Such vectors can include, for example, chromosomal, nonchromosomal and synthetic DNA sequences.

Numerous suitable expression vectors are known to those of skill in the art. The following vectors are provided by way of example; for bacterial host cells: pQE vectors (Qiagen), pBluescript plasmids, pNH vectors, lambda-ZAP vectors (Stratagene), pTrc99a, pKK223-3, pDR540, and pRIT2T (Pharmacia); for eukaryotic host cells: pXT1, pSG5 (Stratagene), pSVK3, pBPV, pMSG, pET21a-d(+) vectors (Novagen), and pSVLSV40 (Pharmacia). However, any other plasmid or other vector may be used so long as it is compatible with the host cell.

In some embodiments, the vector may comprise nucleotide sequences that are codon-biased for expression in the organism being transformed. In another embodiment, a gene of interest, for example, a biomass yield gene, may comprise nucleotide sequences that are codon-biased for expression in the organism being transformed. In addition, the nucleotide sequence of a tag may be codon-biased or codon-optimized for expression in the organism being transformed. A polynucleotide sequence may comprise nucleotide sequences that are codon biased for expression in the organism being transformed. The skilled artisan is well aware of the “codon-bias” exhibited by a specific host cell in usage of nucleotide codons to specify a given amino acid. Without being bound by theory, by using a host cell's preferred codons, the rate of translation may be greater. Therefore, when synthesizing a gene for improved expression in a host cell, it may be desirable to design the gene such that its frequency of codon usage approaches the frequency of preferred codon usage of the host cell. In some organisms, codon bias differs between the nuclear genome and organelle genomes, thus, codon optimization or biasing may be performed for the target genome (e.g., nuclear codon biased or chloroplast codon biased). In some embodiments, codon biasing occurs before mutagenesis to generate a polypeptide. In other embodiments, codon biasing occurs after mutagenesis to generate a polynucleotide. In yet other embodiments, codon biasing occurs before mutagenesis as well as after mutagenesis.

In some embodiments, a vector comprises a polynucleotide operably linked to one or more control elements, such as a promoter and/or a transcription terminator. Such polynucleotide may be heterologous with respect to the one or more control elements. The operably linked control element(s) and polynucleotide sequence are heterologous if not operably linked to each other in nature. A nucleic acid sequence is operably linked when it is placed into a functional relationship with another nucleic acid sequence. For example, DNA for a presequence or secretory leader is operatively linked to DNA for a polypeptide if it is expressed as a preprotein which participates in the secretion of the polypeptide; a promoter is operably linked to a coding sequence if it affects the transcription of the sequence; or a ribosome binding site is operably linked to a coding sequence if it is positioned so as to facilitate translation. Generally, operably linked sequences are contiguous and, in the case of a secretory leader, contiguous and in reading phase. Linking is achieved by ligation at restriction enzyme sites. If suitable restriction sites are not available, then synthetic oligonucleotide adapters or linkers can be used as is known to those skilled in the art. Sambrook et al., Molecular Cloning, A Laboratory Manual, 2^{nd} Ed., Cold Spring Harbor Press, (1989) and Ausubel et al., Short Protocols in Molecular Biology, 2^{nd} Ed., John Wiley & Sons (1992).

A regulatory or control element, as the term is used herein, broadly refers to a nucleotide sequence that regulates the transcription or translation of a polynucleotide or the localization of a polypeptide to which it is operatively linked. Examples include, but are not limited to, an RBS, a promoter, enhancer, transcription terminator, an initiation (start) codon, a splicing signal for intron excision and maintenance of a correct reading frame, a STOP codon, an amber or ochre codon, and an IRES. A regulatory element can include a promoter and transcriptional and translational stop signals. Elements may be provided with linkers for the purpose of introducing specific restriction sites facilitating ligation of the control sequences with the coding region of a nucleotide sequence encoding a polypeptide. Additionally, a sequence comprising a cell compartmentalization signal (i.e., a sequence that targets a polypeptide to the cytosol, nucleus, chloroplast membrane or cell membrane) can be attached to the polynucleotide encoding a protein of interest. Such signals are well known in the art and have been widely reported (see, e.g., U.S. Pat. No. 5,776,689).

In a vector, a nucleotide sequence of interest is operably linked to a promoter recognized by the host cell to direct mRNA synthesis. Promoters are untranslated sequences located generally 100 to 1000 base pairs (bp) upstream from the start codon of a structural gene that regulate the transcription and translation of nucleic acid sequences under their control.

Promoters useful for the present disclosure may come from any source (e.g., viral, bacterial, fungal, protist, and animal) and may further include homologous, engineered or synthetic promoter sequences. The promoters contemplated herein can be specific to photosynthetic organisms, non-vascular photosynthetic organisms, and vascular photosynthetic organisms (e.g., algae, plants) and capable of driving expression of a sequence operably linked to such promoter in those organisms. In some instances, the nucleic acids above are inserted into a vector that comprises a promoter of a photosynthetic organism, e.g., algae. The promoter can be a constitutive promoter, tissue-specific promoter, developmental stage specific promoter, or an inducible promoter. A promoter typically includes necessary nucleic acid sequences near the start site of transcription, (e.g., a TATA element). Common promoters used in expression vectors include, but are not limited to, LTR or SV40 promoter, the E. coli lac or trp promoters, and the phage lambda PL promoter. Non-limiting examples of promoters are endogenous promoters such as the psbA and atpA promoter. Other promoters known to control the expression of genes in prokaryotic or eukaryotic cells can be used and are known to those skilled in the art. Expression vectors may also contain a ribosome binding site for translation initiation, and a transcription terminator. The vector may also contain sequences useful for the amplification of gene expression. Useful algal chloroplast promoters include, but are not limited to, the atpA, psbA, psbB, psbC, psbD, rbcL, 165 and psaA promoters. Useful algal nuclear promoters include, but are not limited to, arg7, nit1, tubulin, PsaD, Hsp70A, rbcS2 and Hsp70A/rbcS2 fusion (see Rasala, B. A., Lee, P. A., Shen, Z., Briggs, S. P., Mendez, M., & Mayfield, S. P. (2012). Robust Expression and Secretion of Xylanasel in Chlamydomonas reinhardtii by Fusion to a Selection Gene and Processing with the FMDV 2A Peptide. PLoS ONE, 7(8), e43349. http://doi.org/10.1371/journal.pone.0043349).

A “constitutive” promoter is, for example, a promoter that is active under most environmental and developmental conditions. Constitutive promoters can, for example, maintain a relatively constant level of transcription.

An “inducible” promoter is a promoter that is active under controllable environmental or developmental conditions. For example, inducible promoters are promoters that initiate increased levels of transcription from DNA under their control in response to some change in the environment, e.g. the presence or absence of a nutrient or a change in temperature. Examples of inducible promoters/regulatory elements include, for example, a nitrate-inducible promoter (for example, as described in Bock et al, Plant Mol. Biol. 17:9 (1991)), or a light-inducible promoter, (for example, as described in Feinbaum et al, Mol Gen. Genet. 226:449 (1991); and Lam and Chua, Science 248:471 (1990)), or a heat responsive promoter (for example, as described in Muller et al., Gene 111: 165-73 (1992)).

In many embodiments, a polynucleotide of the present disclosure includes a nucleotide sequence, where the nucleotide sequence encoding the polypeptide is operably linked to an inducible promoter. Inducible promoters are well known in the art. Suitable inducible promoters include, but are not limited to, the pL of bacteriophage λ; Placo; Ptrp; Ptac (Ptrp-lac hybrid promoter); an isopropyl-beta-D-thiogalactopyranoside (IPTG)-inducible promoter, e.g., a IacZ promoter; a tetracycline-inducible promoter; an arabinose inducible promoter, e.g., P_{BAD} (for example, as described in Guzman et al. (1995) J. Bacteriol. 177:4121-4130); a xylose-inducible promoter, e.g., Pxyl (for example, as described in Kim et al. (1996) Gene 181:71-76); a GAL1 promoter; a tryptophan promoter; a lac promoter; an alcohol-inducible promoter, e.g., a methanol-inducible promoter, an ethanol-inducible promoter; a raffinose-inducible promoter; and a heat-inducible promoter, e.g., heat inducible lambda P_{L} promoter and a promoter controlled by a heat-sensitive repressor (e.g., C1857-repressed lambda-based expression vectors; for example, as described in Hoffmann et al. (1999) FEMS Microbiol Lett. 177(2):327-34).

Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter; an araBAD promoter; in vivo regulated promoters, such as an ssaG promoter or a related promoter (for example, as described in U.S. Patent Publication No. 20040131637), a pagC promoter (for example, as described in Pulkkinen and Miller, J. Bacteriol., 1991: 173(1): 86-93; and Alpuche-Aranda et al., PNAS, 1992; 89(21): 10079-83), a nirB promoter (for example, as described in Harborne et al. (1992) Mol. Micro. 6:2805-2813; Dunstan et al. (1999) Infect. Immun. 67:5133-5141; McKelvie et al. (2004) Vaccine 22:3243-3255; and Chatfield et al. (1992) Biotechnol. 10:888-892); a sigma70 promoter, e.g., a consensus sigma70 promoter (for example, GenBank Accession Nos. AX798980, AX798961, and AX798183); a stationary phase promoter, e.g., a dps promoter, an spy promoter; a promoter derived from the pathogenicity island SPI-2 (for example, as described in WO96/17951); an actA promoter (for example, as described in Shetron-Rama et al. (2002) Infect. Immun. 70:1087-1096); an rpsM promoter (for example, as described in Valdivia and Falkow (1996). Mol. Microbiol. 22:367-378); a tet promoter (for example, as described in Hillen, W. and Wissmann, A. (1989) In Saenger, W. and Heinemann, U. (eds), Topics in Molecular and Structural Biology, Protein-Nucleic Acid Interaction. Macmillan, London, UK, Vol. 10, pp. 143-162); and an SP6 promoter (for example, as described in Melton et al. (1984) Nucl. Acids Res. 12:7035-7056).

In yeast, a number of vectors containing constitutive or inducible promoters may be used. For a review of such vectors see, Current Protocols in Molecular Biology, Vol. 2, 1988, Ed. Ausubel, et al., Greene Publish. Assoc. & Wiley Interscience, Ch. 13; Grant, et al., 1987, Expression and Secretion Vectors for Yeast, in Methods in Enzymology, Eds. Wu & Grossman, 31987, Acad. Press, N.Y., Vol. 153, pp. 516-544; Glover, 1986, DNA Cloning, Vol. II, IRL Press, Wash., D.C., Ch. 3; Bitter, 1987, Heterologous Gene Expression in Yeast, Methods in Enzymology, Eds. Berger & Kimmel, Acad. Press, N.Y., Vol. 152, pp. 673-684; and The Molecular Biology of the Yeast Saccharomyces, 1982, Eds. Strathern et al., Cold Spring Harbor Press, Vols. I and II. A constitutive yeast promoter such as ADH or LEU2 or an inducible promoter such as GAL may be used (for example, as described in Cloning in Yeast, Ch. 3, R. Rothstein In: DNA Cloning Vol. 11, A Practical Approach, Ed. DM Glover, 1986, IRL Press, Wash., D.C.). Alternatively, vectors may be used which promote integration of foreign DNA sequences into the yeast chromosome.

Non-limiting examples of suitable eukaryotic promoters include CMV immediate early, HSV thymidine kinase, early and late SV40, LTRs from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

A vector utilized in the practice of the disclosure also can contain one or more additional nucleotide sequences that confer desirable characteristics on the vector, including, for example, sequences such as cloning sites that facilitate manipulation of the vector, regulatory elements that direct replication of the vector or transcription of nucleotide sequences contain therein, and sequences that encode a selectable marker. As such, the vector can contain, for example, one or more cloning sites such as a multiple cloning site, which can, but need not, be positioned such that a exogenous or endogenous polynucleotide can be inserted into the vector and operatively linked to a desired element.

The vector also can contain a prokaryote origin of replication (ori), for example, an E. coli ori or a cosmid ori, thus allowing passage of the vector into a prokaryote host cell, as well as into a plant chloroplast. Various bacterial and viral origins of replication are well known to those skilled in the art and include, but are not limited to the pBR322 plasmid origin, the 2u plasmid origin, and the SV40, polyoma, adenovirus, VSV, and BPV viral origins.

A vector, or a linearized portion thereof, may include a nucleotide sequence encoding a reporter polypeptide or other selectable marker. The term “reporter” or “selectable marker” refers to a polynucleotide (or encoded polypeptide) that confers a detectable phenotype. A reporter generally encodes a detectable polypeptide, for example, a green fluorescent protein or an enzyme such as luciferase, which, when contacted with an appropriate agent (a particular wavelength of light or luciferin, respectively) generates a signal that can be detected by eye or using appropriate instrumentation (for example, as described in Giacomin, Plant Sci. 116:59-72, 1996; Scikantha, J. Bacteriol. 178:121, 1996; Gerdes, FEBS Lett. 389:44-47, 1996; and Jefferson, EMBO J. 6:3901-3907, 1997, fl-glucuronidase).

A selectable marker (or selectable gene) generally is a molecule that, when present or expressed in a cell, provides a selective advantage (or disadvantage) to the cell containing the marker, for example, the ability to grow in the presence of an agent that otherwise would kill the cell. The selection gene can encode for a protein necessary for the survival or growth of the host cell transformed with the vector. A selectable marker can provide a means to obtain, for example, prokaryotic cells, eukaryotic cells, and/or plant cells that express the marker and, therefore, can be useful as a component of a vector of the disclosure. The selection gene or marker can encode for a protein necessary for the survival or growth of the host cell transformed with the vector. One class of selectable markers are native or modified genes which restore a biological or physiological function to a host cell (e.g., restores photosynthetic capability or restores a metabolic pathway). Other examples of selectable markers include, but are not limited to, those that confer antimetabolite resistance, for example, dihydrofolate reductase, which confers resistance to methotrexate (for example, as described in Reiss, Plant Physiol. (Life Sci. Adv.) 13:143-149, 1994); neomycin phosphotransferase, which confers resistance to the aminoglycosides neomycin, kanamycin and paromycin (for example, as described in Herrera-Estrella, EMBO J. 2:987-995, 1983), hygro, which confers resistance to hygromycin (for example, as described in Marsh, Gene 32:481-485, 1984), trpB, which allows cells to utilize indole in place of tryptophan; hisD, which allows cells to utilize histinol in place of histidine (for example, as described in Hartman, Proc. Natl. Acad. Sci., USA 85:8047, 1988); mannose-6-phosphate isomerase which allows cells to utilize mannose (for example, as described in PCT Publication Application No. WO 94/20627); ornithine decarboxylase, which confers resistance to the ornithine decarboxylase inhibitor, 2-(difluoromethyl)-DL-ornithine (DFMO; for example, as described in McConlogue, 1987, In: Current Communications in Molecular Biology, Cold Spring Harbor Laboratory ed.); and deaminase from Aspergillus terreus, which confers resistance to Blasticidin S (for example, as described in Tamura, Biosci. Biotechnol. Biochem. 59:2336-2338, 1995). Additional selectable markers include those that confer herbicide resistance, for example, phosphinothricin acetyltransferase gene, which confers resistance to phosphinothricin (for example, as described in White et al., Nucl. Acids Res. 18:1062, 1990; and Spencer et al., Theor. Appl. Genet. 79:625-631, 1990), a mutant EPSPV-synthase, which confers glyphosate resistance (for example, as described in Hinchee et al., BioTechnology 91:915-922, 1998), a mutant acetolactate synthase, which confers imidazolione or sulfonylurea resistance (for example, as described in Lee et al., EMBO J. 7:1241-1248, 1988), a mutant psbA, which confers resistance to atrazine (for example, as described in Smeda et al., Plant Physiol. 103:911-917, 1993), or a mutant protoporphyrinogen oxidase (for example, as described in U.S. Pat. No. 5,767,373), or other markers conferring resistance to an herbicide such as glufosinate. Selectable markers include polynucleotides that confer dihydrofolate reductase (DHFR) or neomycin resistance for eukaryotic cells; tetramycin or ampicillin resistance for prokaryotes such as E. coli; and bleomycin, gentamycin, glyphosate, hygromycin, kanamycin, methotrexate, phleomycin, phosphinotricin, spectinomycin, dtreptomycin, streptomycin, sulfonamide and sulfonylurea resistance in plants (for example, as described in Maliga et al., Methods in Plant Molecular Biology, Cold Spring Harbor Laboratory Press, 1995, page 39). The selection marker can have its own promoter or its expression can be driven by a promoter driving the expression of a polypeptide of interest. The promoter driving expression of the selection marker can be a constitutive or an inducible promoter.

Reporter genes greatly enhance the ability to monitor gene expression in a number of biological organisms. Reporter genes have been successfully used in chloroplasts of higher plants, and high levels of recombinant protein expression have been reported. In addition, reporter genes have been used in the chloroplast of C. reinhardtii. In chloroplasts of higher plants, β-glucuronidase (uidA, for example, as described in Staub and Maliga, EMBO J. 12:601-606, 1993), neomycin phosphotransferase (nptII, for example, as described in Carrer et al., Mol. Gen. Genet. 241:49-56, 1993), adenosyl-3-adenyltransf-erase (aadA, for example, as described in Svab and Maliga, Proc. Natl. Acad. Sci., USA 90:913-917, 1993), and the Aequorea victoria GFP (for example, as described in Sidorov et al., Plant J. 19:209-216, 1999) have been used as reporter genes (for example, as described in Heifetz, Biochemie 82:655-666, 2000). Each of these genes has attributes that make them useful reporters of chloroplast gene expression, such as ease of analysis, sensitivity, or the ability to examine expression in situ. Based upon these studies, other exogenous proteins have been expressed in the chloroplasts of higher plants such as Bacillus thuringiensis Cry toxins, conferring resistance to insect herbivores (for example, as described in Kota et al., Proc. Natl. Acad. Sci., USA 96:1840-1845, 1999), or human somatotropin (for example, as described in Staub et al., Nat. Biotechnol. 18:333-338, 2000), a potential biopharmaceutical. Several reporter genes have been expressed in the chloroplast of the eukaryotic green alga, C. reinhardtii, including aadA (for example, as described in Goldschmidt-Clermont, Nucl. Acids Res. 19:4083-4089 1991; and Zerges and Rochaix, Mol. Cell Biol. 14:5268-5277, 1994), uidA (for example, as described in Sakamoto et al., Proc. Natl. Acad. Sci., USA 90:477-501, 1993; and Ishikura et al., J. Biosci. Bioeng. 87:307-314 1999), Renilla luciferase (for example, as described in Minko et al., Mol. Gen. Genet. 262:421-425, 1999) and the amino glycoside phosphotransferase from Acinetobacter baumanii, aphA6 (for example, as described in Bateman and Purton, Mol. Gen. Genet 263:404-410, 2000).

In some instances, the vectors of the present disclosure will contain elements such as an E. coli or S. cerevisiae origin of replication. Such features, combined with appropriate selectable markers, allows for the vector to be “shuttled” between the target host cell and a bacterial and/or yeast cell. The ability to passage a shuttle vector of the disclosure in a secondary host may allow for more convenient manipulation of the features of the vector. For example, a reaction mixture containing the vector and inserted polynucleotide(s) of interest can be transformed into prokaryote host cells such as E. coli, amplified and collected using routine methods, and examined to identify vectors containing an insert or construct of interest. If desired, the vector can be further manipulated, for example, by performing site directed mutagenesis of the inserted polynucleotide, then again amplifying and selecting vectors having a mutated polynucleotide of interest. A shuttle vector then can be introduced into plant cell chloroplasts, wherein a polypeptide of interest can be expressed and, if desired, isolated according to a method of the disclosure.

Knowledge of the chloroplast or nuclear genome of the host organism, for example, C. reinhardtii, is useful in the construction of vectors for use in the disclosed embodiments. Chloroplast vectors and methods for selecting regions of a chloroplast genome for use as a vector are well known (see, for example, Bock, J. Mol. Biol. 312:425-438, 2001; Staub and Maliga, Plant Cell 4:39-45, 1992; and Kavanagh et al., Genetics 152:1111-1122, 1999, each of which is incorporated herein by reference). The entire chloroplast genome of C. reinhardtii is available to the public on the world wide web, at the URL “biology.duke.edu/chlamy_genome/—chloro.html” (see “view complete genome as text file” link and “maps of the chloroplast genome” link; J. Maul, J. W. Lilly, and D. B. Stern, unpublished results; revised Jan. 28, 2002; to be published as GenBank Acc. No. AF396929; and Maul, J. E., et al. (2002) The Plant Cell, Vol. 14 (2659-2679)). Generally, the nucleotide sequence of the chloroplast genomic DNA that is selected for use is not a portion of a gene, including a regulatory sequence or coding sequence. For example, the selected sequence is not a gene that if disrupted, due to the homologous recombination event, would produce a deleterious effect with respect to the chloroplast. For example, a deleterious effect on the replication of the chloroplast genome or to a plant cell containing the chloroplast. In this respect, the website containing the C. reinhardtii chloroplast genome sequence also provides maps showing coding and non-coding regions of the chloroplast genome, thus facilitating selection of a sequence useful for constructing a vector (also described in Maul, J. E., et al. (2002) The Plant Cell, Vol. 14 (2659-2679)). For example, the chloroplast vector, p322, is a clone extending from the Eco (Eco RI) site at about position 143.1 kb to the Xho (Xho I) site at about position 148.5 kb (see, world wide web, at the URL “biology.duke.edu/chlamy_genome/chloro.html”, and clicking on “maps of the chloroplast genome” link, and “140-150 kb” link; also accessible directly on world wide web at URL “biology.duke.edu/chlam-y/chloro/chlorol40.html”). In addition, the entire nuclear genome of C. reinhardtii is described in Merchant, S. S., et al., Science (2007), 318(5848):245-250, thus facilitating one of skill in the art to select a sequence or sequences useful for constructing a vector.

For expression of the polypeptide in a host, an expression cassette or vector may be employed. The expression vector will comprise a transcriptional and translational initiation region, which may be inducible or constitutive, where the coding region is operably linked under the transcriptional control of the transcriptional initiation region, and a transcriptional and translational termination region. These control regions may be native to the gene, or may be derived from an exogenous source. Expression vectors generally have convenient restriction sites located near the promoter sequence to provide for the insertion of nucleic acid sequences encoding exogenous or endogenous proteins. A selectable marker operative in the expression host may be present.

The nucleotide sequences may be inserted into a vector by a variety of methods. In the most common method the sequences are inserted into an appropriate restriction endonuclease site(s) using procedures commonly known to those skilled in the art and detailed in, for example, Sambrook et al., Molecular Cloning, A Laboratory Manual, 2^{nd} Ed., Cold Spring Harbor Press, (1989) and Ausubel et al., Short Protocols in Molecular Biology, 2^{nd} Ed., John Wiley & Sons (1992).

The description herein provides that host cells may be transformed with vectors. One of skill in the art will recognize that such transformation includes transformation with circular vectors, linearized vectors, linearized portions of a vector, or any combination of the above. Thus, a host cell comprising a vector may contain the entire vector in the cell (in either circular or linear form), or may contain a linearized portion of a vector of the present disclosure.

Certain embodiments include the use of nucleotide sequences having a given percent sequence identity to a reference sequence such as those contained in the sequence listing that is part of this disclosure. One example of an algorithm that is suitable for determining percent sequence identity or sequence similarity between nucleic acid or polypeptide sequences is the BLAST algorithm, which is described, e.g., in Altschul et al., J. Mol. Biol. 215:403-410 (1990). Software for performing BLAST analysis is publicly available through the National Center for Biotechnology Information. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (as described, for example, in Henikoff & Henikoff (1989) Proc. Natl. Acad. Sci. USA, 89:10915). In addition to calculating percent sequence identity, the BLAST algorithm also can perform a statistical analysis of the similarity between two sequences (for example, as described in Karlin & Altschul, Proc. Nat'l. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, less than about 0.01, or less than about 0.001.

The following examples are intended to provide illustrations of the application of the present invention. The following examples are not intended to completely define or otherwise limit the scope of the invention.

### EXAMPLES

### Media

The following media were used in the experiments

TABLE 1

| Component | TAP | HSM | mHSM | MASM(F) | 00S | 10AC3-101 |
|---|---|---|---|---|---|---|
| Tris | 20 mM | — | — | 8.25 nM | | |
| NaHCO₃ | | | | | 195 mM | 43.8 mM |
| NH₄Cl | 7.5 mM | 7.5 mM | — | — | | |
| NaNO₃ | — | — | — | 12.3 mM | 29.4 mM | |
| KNO₃ | — | — | 7.42 mM | — | | |
| NH₄NO₃ | | | | | | 0.625 mM |
| Urea | | | | | | 1.5 mM |
| NaCl | | | | | 17.1 mM | 18.7 mM |
| Na₂SO₄ | | | | | | 33.6 mM |
| CaCl₂ | 0.35 mM | 0.35 mM | 0.35 mM | 2.04 mM | 0.4 mM | |
| MgSO₄ | 0.4 mM | 0.4 mM | 0.4 mM | 10.1 mM | 0.8 mM | 2.1 mM |
| Potassium | 1.35 mM | 1.35 mM | 1.35 mM | 0.37 mM | | |
| Phosphate solution | | | | | | |
| K₂HPO₄ | | | | | 2.9 mM | 1 mM |
| K₂SO₄ | | | | | 5.7 mM | |
| KCl | | | | | | 6.6 mM |
| Acetate | 17.4 mM | — | — | — | | |
| NaF | | | | | | 3.5 mM |
| NaEDTA | | | | | 0.2 mM | |

| | |
|---|---|
| Trace elements | <<1 mM Zn, B, Mn, Fe, Co, Cu, Mo, V, Cr, Ni, W, Co, Ti |

### Library Construction

A total of 10 cDNA libraries were used for screening. Three cDNA libraries were obtained from Chlamydomonas reinhardii wild type strain CC-1690 mt+21 gr (Sager, 1955, Genetics, 40(4): 476-89), three from Scenedesmus dimorphus (UTEX 1237), two from Desmodesmus sp. (SE60239), and two from Arthrospira maxima (SE0017).

The first C. reinhardii library was obtained from a photoautotrophically grown shake-flask culture (grown in HSM) under constant light (^{˜}100 μEinstein) in a 5% CO₂ in air environment. Cells were harvested at mid-log phase to represent normal lab-based growth. The other two libraries were derived from cultures grown under stress conditions in order to sample a larger set of genes for screening.

The second library was derived from C. reinhardtii grown photoautotrophically in HSM under constant light in a shake-flask. 5% CO₂ was bubbled in the culture, then switched to air (0.04% CO₂) followed by harvest 2H later. C. reinhardtii cultures grown under relatively high levels of CO₂ that are then switched to a low CO₂ environment undergo a number of changes to adapt to the lower levels of CO₂ and continue to fix carbon and produce biomass. Many of these changes can be seen at the molecular level within hours. This adaptation to low CO₂ levels may induce genes that can increase growth or yield under non-limiting conditions.

The third library was derived from C. reinhardtii grown photoautotrophically in HSM in a shake-flask in a 5% CO₂ in air environment with light that was shifted from ^{˜}100 μEinstein to ^{˜}1200 μEinstein followed by harvest 1H, 2H and 4H later. RNA and cDNA was prepped and synthesized individually from the three timepoints, but mixed for library transformation in E. coli. C. reinhardtii is not typically grown under high light conditions and will photobleach if left in high-intensity light for long periods. When cultures encounter high light, the photoadaptation they undergo includes a number of molecular changes. These changes may provide an additional source of expressed RNAs that could impact yield in our screens.

The fourth library was obtained from a photoautotrophic shake-flask culture of S. dimorphus grown in HSM with 12-hour light-dark cycle in a 5% CO₂ in air environment. The culture was acclimated to the light-dark cycle for 24 hours prior to the first timepoint being sampled. Samples were collected following 6H of constant light, 6H of constant darkness, and 30 minutes after the light-to-dark or dark-to-light transition (red arrows in figure at right). RNA and cDNA was prepped and synthesized individually from the four timepoints, but mixed prior to library normalization.

The fifth library was obtained from S. dimorphus grown photoautotrophically in HSM under constant light (^{˜}100 μE) in a 5% CO₂ air environment at 25° C. A 1 L culture was seeded at a density of 3.5×10⁶ cells/ml and the temperature was shifted to 33° C. Samples were harvested at 30 minutes, 1H, 2H, 6H, 12H, 24H, and 48H after the temperature change. RNA and cDNA was prepped and synthesized individually from the seven timepoints, but mixed prior to library normalization.

The sixth library was derived from S. dimorphus grown photoautotrophically in HSM under constant light (^{˜}100 μE) with 1% CO₂ bubbled directly into the culture at 25° C. Once the culture reached a density of 3.5×10⁶ cells/ml, the light level was increased to 1600 μE. Samples were collected at 1H, 2H, and 4H later. RNA and cDNA was prepped and synthesized individually from the three timepoints, but mixed prior to library normalization.

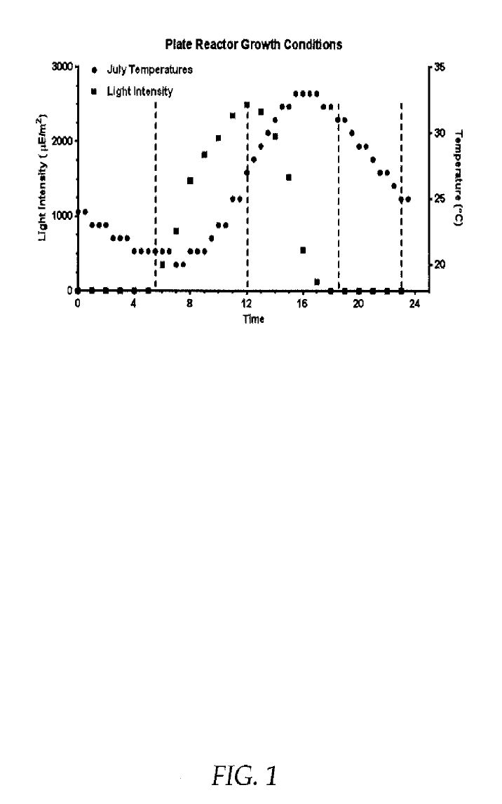

In the seventh library, Desmodesmus inoculum was grown to mid log phase in IABR-10AC3-101 media under 1% CO₂ and 65 μE/m² constant light at 25° C. Plate reactors were inoculated to a starting density of 0.3 g/L, at a volume of 1.6 L each. Reactors were run at a pH set point of 9.5, with diurnal light and temperature cycling based on peak summer weather station data from Las Cruces, N. Mex. depicted in the graph shown in FIG. 1. Quantum yield and absorbance measurements were taken daily to confirm cultures were healthy and growing as expected. Phosphate levels were monitored daily and nitrogen levels measured on day 4 of the experiment to ensure no starvation occurred. After five days of growth in the reactors, samples were taken at set intervals over the course of the light cycle as indicated by the vertical dashed lines in FIG. 1.

In the eighth library, Desmodesmus inoculum was grown under sustained high light and temperature conditions in IABR-10AC3-101 for creation of the second library. The culture was inoculated at 0.115 g/L into 1 L airlift columns. Cultures were grown under 600-700 μE/m² light over a temperature range of 28.9° C. to 35° C. Columns were sampled daily for dry weights, quantum yield, and nitrate and phosphate levels. Observation and data analysis identified a range between 31.7° C. and 32.2° C. where the cultures showed visible signs of stress, but remained viable. RNA source cultures were grown in sterile vessels in an incubator with precise control over temperature and CO₂ levels. Replicate 30 ml cultures in T175 flasks (Corning Inc, Corning, N.Y.) were seeded at a density of 1.0×10⁶ cells/ml in IABR-10AC3-101 media and grown under 1% CO₂ and ^{˜}600 μE/m² light at 32° C. Cultures were harvested when quantum yield readings reached 0.500.

The ninth library was obtained from a photoautotrophic shake-flask A. maxima culture grown in 005 media with 12-hour light-dark cycling in a temperature controlled, 5% CO₂ in air environment. The culture was acclimated to the light-dark cycle at 35° C. for 24 hours prior to the first timepoint being sampled. Samples were collected following 6H of constant light, 6H of constant darkness, and 15 minutes after the light-to-dark or dark-to-light transition. RNA and cDNA was prepped and synthesized individually from the four timepoints, but mixed prior to library normalization.

The tenth library was from a heat stressed A. maxima culture obtained as follows. A. maxima was grown photoautotrophically in 005 media under constant light (^{˜}100 μE/m²) in a temperature controlled, 5% CO₂ air environment. A 1 L culture was seeded at a density of 3.5×10⁶ cells/ml and the temperature was shifted from 35° C. to 40° C. Samples were harvested at 1H, 2H, 6H, 12H, 24H, and 48H after the temperature change. RNA and cDNA was prepped and synthesized individually from the six timepoints, but mixed prior to library normalization.

RNA prepared from these 10 cultures was used to construct independent libraries. For libraries 1-8, mRNA was isolated using oligo(dT) cellulose columns. Two methods were used to synthesize the libraries. For the first, reverse transcription with a dT primer containing a unique sequence (including a restriction site for cloning) was followed by second strand synthesis using RNase H and DNA Polymerase. The double stranded cDNA was treated with Pfu polymerase to produce blunt ends followed by ligation of an adapter to the 5′ end. The second method incorporated a step to increase the number of full length transcripts in the library. Reverse transcription with a dT primer containing a unique sequence (including a restriction site for cloning) was followed by digestion of the cDNA/RNA hybrid with RNase I. A 7-methylguanosine mRNA cap-specific antibody (Life Technologies, Carlsbad, Calif.) was used to enrich for full length cDNA. An adapter was ligated to the 5′ end and the second strand was synthesized by primer extension.

For libraries 9 and 10, 16s and 23s rRNA was removed using the MICROBExpress Kit (Ambion, Austin, Tex.) and the enriched mRNA was synthetically polyadenylated with E. coli Poly(A) Polymerase enzyme (Ambion, Austin, Tex.). Reverse transcription with a dT primer containing a unique sequence (including a SbfI restriction site for cloning) was followed by second strand synthesis using RNase H and DNA Polymerase. The double stranded cDNA was treated with T4 polymerase to produce blunt ends followed by ligation of an adapter to the 5′ end.

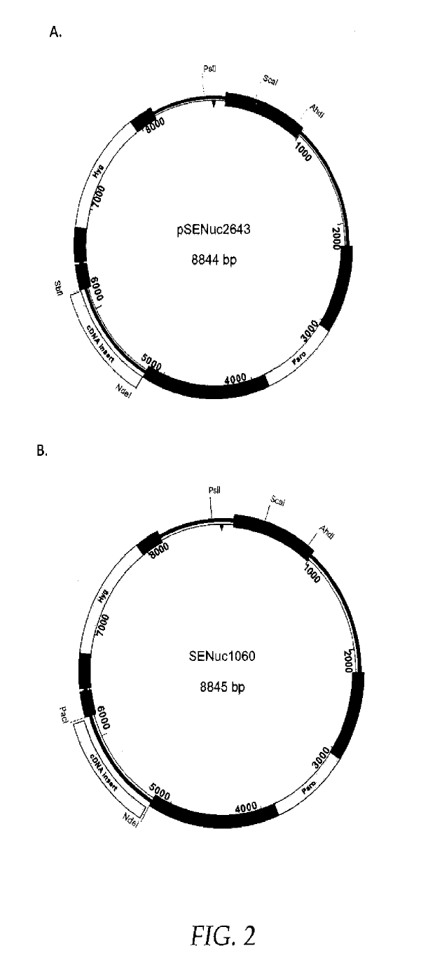

Normalization of the libraries was accomplished with a kit from Evrogen (Moscow, Russia) that utilized a double stranded DNA nuclease after dissociation and re-annealing of the cDNA. For the A. maxima library, PCR amplification and restriction enzyme digestion (NdeI/SbfI) produced cDNA that was then ligated into a cDNA overexpression vector, SENuc2643 (NdeI/SbfI—FIG. 2A). The NdeI sequence at the 5′ end of the cDNA transcript creates an ATG at the beginning of the cloned cDNA so that any truncated cDNAs can be translated in frame in one of three cases. For the remaining libraries, PCR amplification and restriction enzyme digestion (AseI/PacI) produced cDNA that was then ligated into our cDNA overexpression vector, SENuc1060 (NdeI/PacI—FIG. 2B). The sequence at the NdeI/AseI site also creates an ATG at the beginning of the cloned cDNA so that any truncated cDNAs can be translated in frame in one of three cases. The vectors contain a constitutive hybrid promoter (AR1) derived from C. reinhardtii rbcs2, hsp70A, and the first intron from the rbcS2 gene as well as the 3′ UTR and terminator from rbcS2. The cDNA overexpression cassette is flanked by hygromycin and paromomycin resistance cassettes for C. reinhardtii transformation.

Once the libraries were ligated into the vector, they were transformed into E. coli for amplification and QC. A number of individual clones were selected and the cDNA insert was PCR amplified and sequenced. (Note that the sequence was usually only derived from the 5′ end of the cDNA because vector specific primers that sequence from the 3′ end encounter the polyA tail after the 3′ cloning site and the Sanger sequence fails on the homopolymer). Sequences were considered full length if they contained the endogenous ATG as annotated in the C. reinhardtii genome, since the 5′ UTR is not necessary for expression from the platform vector. Additionally, the vector ATG at the cloning site allowed for ⅓ of truncated coding regions to still be translated in frame. Those sequences that did not match a predicted gene model were classified as scaffold hits and identified by their genome coordinates. The 10 libraries used for screening are detailed in Table 1

TABLE 2

| Library | Complexity | Quality |
|---|---|---|
| C. reinhardtii photoautotrophic, | 3.3 × 10⁵ clones | 54% full-length |
| core library | | 61% in-frame CDS |
| C. reinhardtii low CO₂ inducdtion | 1.03 × 10⁵ clones | 42% full-length |
| | | 46% in-frame CDS |
| C. reinhardtii 1500 microE light | 2.1 × 10⁴ clones | 43% full-length |
| stress | | 50% in-frame CDS |
| S. dimorphus photosutotrophic | 2.4 × 10⁵ clones | 50% full-length |
| 12H light/dark cycling | | 66% in-frame CDS |
| S. dimorphus 1600 microE light | 2.8 × 10⁵ clones | 30% full-length |
| stress | | 50% in-frame CDS |
| S. dimorphus 25° C. to 33° C. | 2.0 × 10⁵ clones | 50% full-length |
| temperature shift | | 70% in-frame CDS |
| Desmodesmus sp. New Mexico | 8 × 10⁵ clones | 29.2% full-length |
| peak summer months | | 62.5% in-frame CDS |
| | | 42.2% scaffold hits |
| Desmodesmus sp. constant high | 1.3 × 10⁶ clones | 30.0% full-length |
| light/temperature | | 64.5% in-frame CDS |
| | | 34.0% scaffold hits |
| A. maxima | 6 × 10⁵ clones | 20.5% full-length |
| | | 86.1% in-frame CDS |
| A. maxima | 1.1 × 10⁶ | 21.0% full-length |
| | | 56.7% in-frame CDS |

The S. dimorphus genome was sequenced, assembled and annotated to facilitate identification of cDNA clones. Four genomic DNA libraries with different insert sizes (300 bp, 500 bp, 2 kbp, 5 kbp) were constructed and sequenced with 2×100 chemistry on an Illumina HiSeq instrument. The sequencing, assembly and BLASTX against the published C. reinhardtii and A. thaliana genomes was completed by Cofactor Genomics (St. Louis, Mo.). Additionally, the augustus algorithm (Stanke et al., 2006, BMC Bioinformatics, 7, 62. doi:10.1186/1471-2105-7-62) was run on the assembly to predict gene models for the genome (C. reinhardtii used as a training set). 451 contigs with N50 of 763 kbp were derived. Total sequence length was 110.5 Mbp and 14.83% of the assembly was unknown (N's). 18,408 gene models were predicted by augustus. This size is very similar to the C. reinhardtii genome (111 Mbp with 17,737 gene loci).

The Desmodesmus genome was sequenced, assembled and annotated to facilitate identification of cDNA clones. Four genomic DNA libraries with different insert sizes (300 bp, 500 bp, 2 kbp, 5 kbp) were constructed and sequenced with 2×100 chemistry on an Illumina HiSeq instrument. The sequencing, assembly and BLASTX against the published C. reinhardtii and A. thaliana genomes was completed by Cofactor Genomics (St. Louis, Mo.). Additionally, the augustus algorithm was run on the assembly to predict gene models for the genome (C. reinhardtii used as a training set). 990 contigs with N50 of 334 kbp were derived. Total sequence length is 126.9 Mbp and 8.31% of the assembly was unknown (N's). 11,118 gene models were predicted by augustus.

### Primary Turbidostat Screening

DNA from the libraries was independently transformed into wild type C. reinhardtii cells. Transformation of the C. reinhardtii nuclear genome often results in the insertion of digested DNA due to exonucleases and/or endonucleases. Dual antibiotic selection for transformants minimizes the representation of these insertions in the cDNA strain library. After selection on plates containing both hygromycin and paromomycin, transformed algal colonies were scraped in 1000 colony sets into flasks containing TAP media (20 mM Tris, 7.5 mM NH₄Cl, 0.35 mM CaCl₂, 0.4 mM MgSO₄, 1.35 mM potassium Phosphate sol'n., 17.4 mM Acetate, trace elements). Each of these sets is referred to as a Pool. The next day, cells were passaged to a new flask, and then inoculated into turbidostats the following day.

For the C. reinhardtii libraries, turbidostats were filled with HSM media (7.5 mM NH₄Cl, 0.35 mM CaCl₂, 0.4 mM MgSO₄, 1.35 mM potassium phosphate sol'n., trace elements) and set to an OD₇₅₀ of approximately 0.3, which represents an early- to mid-log growth phase. Constant light of ^{˜}150 μEinstein was provided, with a constant stream of 1% CO₂ bubbling into the culture. Growth rates were monitored by media consumption via solenoid click rate on the turbidostat. Cultures were monitored at least daily for media replenishment, CO₂ delivery, culture settling, cell sticking, mechanical failure or any other issues. The cultures were grown under these optimal photoautotrophic conditions for up to six weeks. Samples were taken at weekly intervals and single cells were sorted by fluorescence-activated cell sorting (FACS) into 96-well plates containing TAP media. Weekly sorts were a risk-mitigation strategy, as some turbidostats were expected to fail prior to the six-week endpoint. In the cases where turbidostat failure occurred, the cultures sorted on an earlier week were used as an alternative endpoint. After a week or more of growth, sorted strains were replicated onto solid media for longer-term recovery and isolation of transformed lines.

For S. dimorphus libraries, turbidostats were filled with HSM media and set to an OD₇₅₀ of approximately 0.3, which represents an early- to mid-log growth phase. Constant light of ^{˜}150 ME was provided, with a constant stream of 0.2% CO₂ bubbling into the culture. Cultures were monitored at least daily for media replenishment, CO₂ delivery, culture settling, cell sticking, mechanical failure or any other issues. The cultures were grown under these optimal photoautotrophic conditions for up to five weeks. Samples were taken at weekly intervals and single cells were sorted by fluorescence-activated cell sorting (FACS) into 96-well plates containing TAP media. Weekly sorts were a risk-mitigation strategy. In the cases where turbidostat failure occurred, the cultures sorted on an earlier week were used as an alternative endpoint. After a week or more of growth, sorted strains were replicated onto solid media for longer-term recovery and isolation of transformed lines.

Turbidostat growth conditions for the four Desmodesmus and A maxima cDNA library screening involved diurnal cycling. Prior to running the library screen, the cycling parameters for selection in turbidostats were validated. Wild type C. reinhardtii was grown under three different light regimes in high replication—constant light, 16H light-8H dark cycle, and 14H light-10H dark cycle. Previous cDNA library screens conducted under constant light would average 3.14 generations per day based on this experiment. Over a five week screen, this results in ^{˜}110 generations. To achieve the same number of generations a 16H/8H diurnal cycle was chosen. At 2.58 generations per day, cultures achieve 110 generations after 42.6 days or 6 weeks.

The turbidostats were filled with HSM media and set to an OD₇₅₀ of approximately 0.3, which represents an early- to mid-log growth phase. Cultures were grown under a constant stream of 0.2% CO₂ and a 16H/8H light-dark diurnal cycle. A light intensity of ^{˜}150 μE/m² was provided during the 16H phase of the cycle. Cultures were monitored at least daily for media replenishment, CO₂ delivery, culture settling, cell sticking, mechanical failure or any other issues. The cultures were grown under these conditions for up to six weeks. Samples were taken at weekly intervals and single cells were sorted by fluorescence-activated cell sorting (FACS) into 96-well plates containing TAP media. Weekly sorts were a risk-mitigation strategy, in the event some turbidostats failed prior to the six-week endpoint. In the cases where turbidostat failure occurred, the cultures sorted on an earlier week were used as an alternative endpoint. After a week or more of growth, sorted strains were replicated onto solid media for longer-term recovery and isolation of transformed lines.

### Sequencing and Analysis Form Primary Turbidostat Screening

After 5-7 days of growth in 96-well plates, the individual strains were used as template in a PCR reaction that amplified the cDNA insert based on common vector primers. After ascertaining success in producing a single product from the reactions, the PCR products were treated for sequencing with Exonuclease I/Shrimp Alkaline Phosphatase (ExoSAP). These products were then sequenced via Sanger chemistry (by outside vendors) using a common vector primer that reads into the 5′ end of the cDNA insert.

Sequences were analyzed in sets derived from each turbidostat replicate at each timepoint, with the exception being baseline (time 0) datasets, which were analyzed per pool and then used as the starting point for each turbidostat replicate of that pool. Sanger reads were processed using CLC bio's Genomics Workbench software and a custom plugin. The plugin imports the data into the Genomic Workbench, trimming each sequence for quality and vector. The sequences are then compared to the Chlamydomonas reinhardtii genome using blastn. The gene locus for the top hit was determined and the relation of the BLAST hit and gene CDS was determined. A final result table was generated containing primarily the gene locus and how many times it was hit by a sequence within the dataset.

Hit counts and total sequences were used to calculate the frequency of each gene present in a given timepoint. These numbers can then be used to calculate a selection coefficient using the formula below (Lenski, 1991, Biotechnology 15:173-92). Note that the selection coefficients used in this analysis do not conform strictly to some of the assumptions upon which the formula is based, in that this was not a single clone compared against a uniform population. Each clone was compared to the rest of the pool, which itself was made up of many other clones. However, within the experiment, the calculated selection coefficients provided a valid way to compare and rank potentially winning clones.

In(rₜ)=In(r₀)+s·t

where r₀ is the ratio of hits for a given clone to hits for the remainder of the population at a starting time, rₜ is this ratio at time t and s is the selection coefficient (expressed in units of t⁻¹).

In many cases, a given sequence/gene was identified at one time point but not detected in another time point (most commonly, a potential winner that was not seen in the early or baseline sample). As the natural log of zero produces an error, assumptions were necessary in such a case. For the primary screen, 1000 clones per pool were targeted. As not sequence enough clones were sequenced to fully determine the population at early stages, it was assumed that any sequence not detected initially was present at ^{˜}0.1% ( 1/1000).

The formula was used to estimate the length of time required for competition and the number of clones to analyze in order to reach a desired level of sensitivity. Assuming a 1/1000 starting ratio, approximately 200 sequences at the endpoint and a sensitivity of 5% (i.e. 10 sequences out of 200), it is possible to calculate the time necessary to identify a clone with a selection coefficient of 0.1000 as follows:

In(10/190)=In( 1/1000)+0.1000d⁻¹·t days; t=39.6 days

Thus in the primary screen, an s value of approximately 0.1 should be detectable within 6 weeks of growth by sequencing approximately 200 clones. These calculated selection coefficients were then used to rank and select potential winning clones.

### Secondary Turbidostat Screening.

Potential winners from the primary screening were recombined and subjected to a secondary screen. Selected lines were clonally isolated from the replicated solid media plates corresponding to the FACS sorted plate from which the final data was derived. Multiple isolates (usually 4) of each of these lines were inoculated into 4-5 mL liquid TAP media in 24-well blocks (i.e. 4 lines each for 6 independent winners/genes per block). After growth to near saturation, cell density was determined by OD₇₅₀ for normalization during the re-rack into pools. A sequence confirmed isolate of each potential winner was inoculated into 5 mL liquid TAP media in 24-well blocks. After growth to near saturation, cell density was determined by OD₇₅₀ for normalization during the re-rack into pools. Potential winners were randomized to generate fifty pools of 50-52 genes each.

For the C. reinhardtii libraries, 24 well blocks were arbitrarily paired so each pair contained lines from 12 potential winners/genes. Four of these paired sets (i.e. 48 potential winners) were combined into one pool that was then inoculated into replicate turbidostats. A sliding window of four sets of paired blocks, moving down one set at a time, was used to make up the remaining pools for inoculation into replicate turbidostats. This resulted in each potential winner residing in 4 separate pools; and in each of these four pools a given potential winner was always in combination with the eleven other clones in the set of 12. Twelve additional pools were then created, each pool containing a single winner from each set of 12 potential winners. In this way, each potential winner was separated from every other potential winner in at least one pool. This would avoid a situation where an especially dominant line masks a slightly lesser (but still interesting) line if they happened to always be screened together. In total, each potential winner was combined into five distinct pools of 37 to 48 clones each.

These pools were normalized by OD₇₅₀. An average across the blocks was calculated, and then the volume of each well was adjusted up or down based on +/−50% variation from that average. This normalization was applied on the pairs of blocks to create an initial culture of 12 potential winners that was then combined based on the window strategy described above with three other cultures of 12 clones. Pooled cultures were inoculated into quadruplicate turbidostats. Additionally, single cells were sorted by FACS from each pool into 96-well plates for a baseline data point. The turbidostats were filled with HSM media and set to an OD₇₅₀ of approximately 0.3, which represents an early- to mid-log phase. Constant light of ^{˜}150 μEinstein was provided, with a constant stream of 1% CO₂ bubbling into the culture. Growth rates were monitored by media consumption via solenoid click rate. Cultures were monitored at least daily for media replenishment, CO₂ delivery, culture settling, cell sticking, mechanical failure or any other issues. Samples were taken at 7 days and at 10 or 12 days, and single cells were sorted by FACS into 96-well plates. After a week or more of growth, sorted strains were replicated onto solid media for longer term recovery and isolation of transformed lines.

Again, the selection coefficient calculation was used to estimate the length of time required for competition and the number of clones to analyze in order to reach a desired level of sensitivity. Assuming a 1/47 starting ratio, an average of 220 sequences at the endpoint and a sensitivity of about twice the starting ratio (i.e. 9 sequences out of 220), the detectable s was calculated as follows:

In(9/211)=In(1/47)+s·12 days; s=0.0580 d⁻¹

Thus in this secondary screen, an s value of approximately 0.05 should be detectable within 12 days of growth by sequencing approximately 220 clones.

Over 400 winners were combined into 37 sets of approximately 12 potential winners. Some sets did not have 12 winners in order to accommodate operational efficiencies or because certain lines were not successfully recovered and grown from the primary screen. This resulted in 37 pools from the sliding window strategy plus an additional 12 pools from combining one winner from each of the sets for a total of 49 pools and 196 turbidostats. Because of the shorter time frame necessary for screening (due to lower complexity in secondary screening as compared to primary), only a few turbidostats failed prior to providing an endpoint sample. In all, 165 out of 198 turbidostats reached their endpoint. In only six cases did less than three replicates from a pool produce final data.

For S. dimorphus libraries, each potential winner was represented in 5 separate pools. The randomization process ensured that no two potential winners occurred together in all 5 pools. This avoided a situation where an especially dominant line masks a slightly lesser (but still interesting) line if they happened to always be screened together. Pools were inoculated into quadruplicate turbidostats. Additionally, single cells were sorted by FACS from each pool into 96-well plates for a baseline data point. The turbidostats were filled with HSM media and set to an OD₇₅₀ of approximately 0.3, which represents an early- to mid-log phase. Constant light of ^{˜}150 μE was provided, with a constant stream of 0.2% CO₂ bubbling into the culture. Cultures were monitored at least daily for media replenishment, CO₂ delivery, culture settling, cell sticking, mechanical failure or any other issues. Samples were taken at day 0, day 9 or 10, and day 14 or 15, and single cells were sorted by FACS into 96-well plates. Endpoint samples were collected on multiple days due to the size of the secondary screen and time constraints for FACS. Two hundred turbidostats were sampled over a 2 day period; 100 turbidostats were sorted on day 9 and the remaining 100 were sorted on day 10. The 100 turbidostats that were sorted on day 9 were then subsequently sorted on day 14. Those 100 turbidostats from day 10 likewise were sorted on day 15.

For the Desmodesmus and A. maxima libraries, potential winners were randomized to generate sixty-five pools of 32 winners for Desmodesmus sp. and twenty-five pools of 20 winners for A. maxima. Each potential winner was represented in 5 separate pools. The randomization process ensured that no two potential winners occurred together in all 5 pools.

Pools were inoculated into quadruplicate turbidostats. Additionally, single cells were sorted by FACS from each pool into 96-well plates for a baseline, day 0, data point. The turbidostats were filled with HSM media and set to an OD₇₅₀ of approximately 0.3, which represents an early- to mid-log phase. Cultures were grown under a constant stream of 0.2% CO₂ and a 16H/8H light-dark diurnal cycle. A light intensity of ^{˜}150 μE/m² was provided during the 16H light phase of the cycle. Cultures were monitored at least daily for media replenishment, CO₂ delivery, culture settling, cell sticking, mechanical failure or any other issues. Turbidostats were sampled at day 13 for A. maxima and day 18 for Desmodesmus and single cells were sorted by FACS into 96-well plates.

Sequencing and Analysis from Secondary Turbidostat Screening.

### Overall

Samples were processed, sequenced, and analyzed as described for Primary Turbidostat Screening, with only two exceptions. First, if a clone was not detected in the baseline dataset, it was assumed that the clone was actually sequenced one time, thereby producing a starting frequency of 1/(# of sequences screened). Second, if a particular sequence was not seen in the final set but was prevalent at the baseline, a negative selection coefficient would be produced. While this type of data would not lead to selection of this candidate as a winner, it is still relevant data that could inform the overall selection process. In this case, a non-zero frequency was assumed even if there are no final hits, so that the sequence was assumed to be detected at a 0.1% frequency at the endpoint. During the analysis, these assumptions were monitored to avoid consideration of artifactual data. As an example, if a clone was sequenced once in one timepoint and zero times in the other (therefore an assumed single hit), this could produce a rather large s value, negative or positive, depending on which timepoint had more total sequences. However, winners were not based on this type of data as a single sequence is not sufficient for accurate results. The calculated selection coefficient was then used to rank and select potential winning clones.

Four independent transformation waves provided the transgenic lines of C. reinhardtii used for the primary screen. After colonies had grown on transformation plates, they were counted and grouped into sets of 1000 colonies. Each set of 1000 colonies represented the overexpressed cDNA clones that made up the pools for turbidostat screening.

Based on our experience with operating turbidostats, attrition is expected over the course of a multi-week experiment due to occasional equipment failure or culture crash. Therefore excess pools and replicates were set up for screening. 171, 100 and 105 pools were initially set up for the C. reinhardtii, S. dimorphus and combined Desmodesmus and A. maxium libraries, respectively. For each pool of approximately 1000 colonies, four replicate turbidostats were established. The target screening time for the cultures was 4-6 weeks.

In those C. reinhardtii cases where a 3-week sample was the final time point (due to turbidostat failure before week 4), the 3-week set was used for final data based on an analysis showing that selection can be measured even at this early time point. All pools were set up in 6 rounds of approximately 30 pools (120 turbidostats) for operational efficiency. 119 of the 171 pools had, on average, 2.74 replicates at the 4-week mark (this excludes pools with only single replicates). This exceeded the target of 100 pools of replicates (or 100,000 clones) established at the outset.

All S dimorphus pools were set up in 4 rounds of 25 pools (100 turbidostats) for operational efficiency. The first round consisted of transformants from the photoautotrophic light-cycled cDNA library. The second round was the high light stress cDNA library and the third round contained the high temperature cDNA library. The fourth round was a mixture of all three cDNA libraries.

All Desmodesmus and A. maxima pools were set up in 4 staggered rounds for operational efficiency—three rounds of Desmodesmus pools (^{˜}81,000 clones) and one round of A. maxima pools (^{˜}24,000 clones). The first two rounds consisted of transformants from the Desmodesmus plate reactor cDNA libraries. The third round was the sustained high light and temperature Desmodesmus cDNA library and the fourth round was a mixture of the two A. maxima cDNA libraries.

For each turbidostat, the latest sample taken was used as the final timepoint. For example, if a specific turbidostat did not reach the 6-week mark, then the 5-week sample was used as the endpoint. In a few cases, this endpoint did not produce adequate data and the previous week's sample was used. The earliest timepoint used as an endpoint was a 3-week sample and most winner were selected on a full endpoint. In all cases, analysis took these different durations into account. The distribution of endpoints sequenced is shown in Table 2, showing the number of pools with differing numbers of endpoint replicates.

TABLE 3

| Library | Round | Quadruplicate | Triplicate | Duplicate | Single | Total |
|---|---|---|---|---|---|---|
| C reinhardtii | 1 | 0 | 7 | 9 | 8 | 24 |
| | 2 | 0 | 4 | 7 | 4 | 15 |
| | 3 | 0 | 1 | 6 | 2 | 9 |
| | 4 | 5 | 7 | 9 | 7 | 28 |
| | 5 | 3 | 3 | 7 | 13 | 26 |
| | 6 | 2 | 5 | 13 | 4 | 24 |
| | Total | 10 | 27 | 51 | 38 | 126 |
| S. dimorphus | 1 | 25 | 0 | 0 | 0 | 25 |
| | 2 | 20 | 4 | 1 | 0 | 25 |
| | 3 | 22 | 3 | 0 | 0 | 25 |
| | 4 | 24 | 1 | 0 | 0 | 25 |
| | Total | 91 | 8 | 1 | 0 | 100 |
| Desmodesmus | 1 | 17 | 6 | 4 | 0 | 27 |
| A. maxima | 2 | 20 | 6 | 1 | 0 | 27 |
| | 3 | 14 | 13 | 0 | 0 | 27 |
| | 4 | 8 | 9 | 7 | 0 | 24 |
| | Total | 59 | 36 | 12 | 0 | 105 |

The majority of data from the primary screen consisted of clones that were positively selected. This is inherent in the nature of the screening and output, as the signal for a given clone was, by design, low at the beginning of the experiment and only positively selected clones would have a signal at the final timepoint. Thus most clones that are neutral or negatively selected were never detected.

C. reinhardtii

All potential winners from the primary screen with a positive selection coefficient were nominated to be taken forward to secondary screening. As the selection of a given clone depended on both the genetics/physiology of the clone in addition to the environment, even a clone that showed only a slight advantage in the primary screen could become a dominant winner in another competition (and vice versa). 544 winners were identified in the primary screen and assigned numeric identifiers (W0001-W0546, W0199 and W0200 were skipped). Candidates with negative s values were excluded from secondary screening.

The sequences derived from the PCR amplified cDNAs gave the number of hits for each clone/gene, but also some information about the nature of the cDNA insert. From the hit frequencies, potential winners were selected, with initially no regard for the cloned cDNA insert. From this 5′ end read, information about the relative position of the cDNA end to the annotated gene and the presence of an open reading frame (ORF) could be ascertained. In the cases where no ORF was present and/or the insert consisted of only cDNA cloning artifacts (e.g. linker/adapter sequences), it was assumed that any selective phenotype would be due to an insertional event, i.e. gene disruption in the Chlamydomonas host. These insertional events are always a possibility for every potential winner, even in the case of insertion of a full-length cDNA, but those without a translatable protein are more likely.

Any clone that was identified in a replicate of a turbidostat was given a winner number and initially treated as independent from all other potential winners. Given that the same set of approximately 1000 clones went into each set of replicate turbidostats, some clones may be identified more than once. Additionally, in these cases and also in the case where a given gene was identified in distinct pools, it is possible that the two clones are distinct events and are not clonal duplicates.

Only 34 of the 171 pools produced winning clones that hit the same gene in multiple replicates, with most of these repeating in two replicates and only one showing the same clone in all four replicates. Additionally, 64 genes were identified as potential winners in more than one distinct pool. A significant possibility is that there is clonal interference. This occurs when the majority of the clones have a similar fitness, where stochasticity (drift) could play a large role in driving shifts in the population. If this were occurring, the replicates would vary. Despite the low levels of replication within a set, identification of a given clone in multiple pools can only occur if independent transformation events produced winners expressing the same gene.

Once potential winners were identified, algae clones representing each were identified and isolated. The liquid culture FACS plates were transferred to solid media at the time of sequencing. The colonies grown up on these plates were used to recover the strains for each potential winner. The strains were struck out for single colonies to ensure clonal isolation, then the cDNA insert was PCR amplified and sequenced to confirm the identity of each clone. These individual clones were also used to determine the full length sequence of the insert rather than relying on the Chlamydomonas gene annotations for that part of the cDNA not reached by the single 5′ sequencing read used for sequencing.

S. dimorphus

All potential winners from the primary screen with a selection coefficient greater than 0.1 were nominated to be taken forward to secondary screening. Clones that were likely insertional events were not included (based on short blast hits and/or cDNA cloning artefacts). As the selection of a given clone depends on both the genetics/physiology of the clone in addition to the environment, even a clone that shows only a slight advantage in the primary screen could become a dominant winner in another competition (and vice versa). 637 winners were identified in the primary screen and assigned numeric identifiers (W0601-W1237).

The sequences derived from the PCR amplified cDNAs provided the number of hits for each clone/gene, but also some information about the nature of the cDNA insert. From the hit frequencies, potential winners were selected, with initially no regard for the cloned cDNA insert. From this 5′ end read, information about the relative position of the cDNA end to the annotated gene and the presence of an open reading frame (ORF) could be ascertained. In the cases where the blastn hit against the genome was only a few nucleotides long and/or the insert consists of only cDNA cloning artifacts (e.g. linker/adapter sequences), it was assumed that any selective phenotype would be due to an insertional event, i.e. gene disruption in the Chlamydomonas reinhardtii host. These insertional events are always a possibility for every potential winner, even in the case of insertion of a full-length cDNA, but those without a translatable protein are more likely.

Any clone that was identified in a replicate of a turbidostat was not assigned a winner number unless the predicted coding sequence percentage was different for both gene hits. Given that the same set of approximately 1000 clones went into each set of replicate turbidostats, some clones may be identified more than once. Additionally, in the cases where a given gene was identified in distinct pools, it is probable that the two clones are distinct transformation events and are not clonal duplicates. This led to treatment of these isolated candidates as a separate winner from those with an identical gene locus.

Once potential winners were identified, algae clones representing each were identified and isolated. The liquid culture FACS plates were transferred to solid media at the time of sequencing. The colonies grown up on these plates were used to recover the strains for each potential winner. The strains were struck out for single colonies to ensure clonal isolation and the cDNA insert was subsequently PCR amplified and sequenced to confirm the identity of each clone.

Desmodesmus sp./A. maxima

All potential winners from the Desmodesmus primary screen with a selection coefficient greater than 0.09 were nominated to be taken forward to secondary screening. All potential winners from the A. maxima primary screen with a selection coefficient greater than 0.08 were also nominated for secondary screening. Clones that were likely insertional events were not included (based on short blast hits and/or cDNA cloning artifacts). As the selection of a given clone depends on both the genetics/physiology of the clone in addition to the environment, even a clone that shows only a slight advantage in the primary screen could become a dominant winner in another competition (and vice versa). 441 winners were identified in the Desmosdesmus primary screen and assigned numeric identifiers (W1301-W1740). 124 winners were identified in the A maxima primary screen and assigned numeric identifiers (W1741-W1863).

The sequences derived from the PCR amplified cDNAs provided the number of hits for each clone/gene, but also some information about the nature of the cDNA insert. From the hit frequencies, potential winners were selected, with initially no regard for the cloned cDNA insert. From this 5′ end read, information about the relative position of the cDNA end to the annotated gene and the presence of an open reading frame (ORF) could be ascertained. In the cases where the blastn hit against the genome was only a few nucleotides long and/or the insert consists of only cDNA cloning artifacts (e.g. linker/adapter sequences), it was assumed that any selective phenotype would be due to an insertional event, i.e. gene disruption in the Chlamydomonas reinhardtii host. These insertional events are always a possibility for every potential winner, even in the case of insertion of a full-length cDNA, but those without a translatable protein are more likely.

Any clone identified in a replicate of a turbidostat was not assigned a winner number unless the predicted coding sequence percentage was different for both gene hits. Given that the same set of approximately 1,000 clones went into each set of replicate turbidostats, some clones may be identified more than once. Additionally, in the cases where a given gene was identified in distinct pools, it is probable that the two clones are distinct transformation events and are not clonal duplicates. This led to treatment of these isolated candidates as a separate winner from those with an identical gene locus.

Once potential winners were identified, algae clones representing each were identified and isolated. The liquid culture FACS plates were transferred to solid media at the time of sequencing. The colonies grown up on these plates were used to recover the strains for each potential winner. The strains were struck out for single colonies to ensure clonal isolation and the cDNA insert was subsequently PCR amplified and sequenced to confirm the identity of each clone. These individual clones were also used to determine the full length sequence of the insert.

### Secondary Screening Results

C. reinhardtii

Potential winner clones to be carried into secondary screening were grown in 4-5 mL cultures of TAP in 24-well blocks. Where possible, more than one clonal isolate of each potential winner was inoculated to ensure cultures were ready for combination and inoculation into turbidostats. After growth of the cultures for 4-6 days, OD₇₅₀ was measured for each well. Cultures that deviated outside 0.5× to 2× the block average OD were normalized by adding more or less of the given culture when combining. The potential winners were grouped into sets of 12 (based on two 24-well blocks with 4 replicates of each potential winner), resulting in 37 sets. Clones that were likely insertional events were excluded. 113 potential winners made up this excluded set. Some additional attrition occurred as clones with only a few representative winning clones were sometimes not recovered, and some cultures did not grow. A few lines were not confirmed as sequence positive for the cDNA insert. In all, 38 genes that were identified in primary screening were not successfully entered into secondary screening.

These 37 sets were combined in pools of up to 48 winning clones, resulting in 37 pools. An additional 12 pools were derived by taking a single clone from each of the 37 sets, thus separating each set of 12 clones screened together in the first 37 pools from each other. These 49 pools were then each inoculated into four replicate turbidostats and run for 10-12 days as described above. The first 17 pools were set up in one round with the remaining 32 pools set up a few days later. Each potential winner ended up in 5 distinct pools and 20 turbidostats, to allow for some turbidostat attrition, and to put each winner in 5 different environments to elicit any possible selective advantage. In all, 33 of the 198 turbidostats did not make an endpoint of 10 or 12 days, with only 2 pools ending up with less than 2 replicates.

For each potential winner in a pool, the number of hits at baseline and at the final data point were determined. Using the total number of sequences derived for each pool at the baseline and final timepoints, hit frequencies were calculated. As expected, the baseline frequencies were very low, centered around a median of 0.022 (the expected value was 1/47, or 0.21). Final frequencies ranged up to approximately 10.0 (for example, 303 hits out of 334 total sequences equates to 303/(334-303) or 9.77), though most were 2.0 or below and almost 90% were below 0.2. Many of these low values were due to the large number of potential winners that were not detected in the final timepoint and thus were assumed to have a single hit.

Selection coefficients were calculated for each replicate turbidostat, using the common baseline hit frequency for the pool and the final hit frequency for each replicate (column sᵣₑₚ below). The average of these replicate sᵣₑₚ values was calculated as s_{avg}. Additionally, a third selection coefficient was calculated for the entire pool by summing all the final hits and the sum of total sequences for all replicates and using that as the final frequency for s calculation (column sₛᵤₘ). In the example given below, time is 10 days. As a demonstration, sᵣₑₚ for the first replicate in the table below is calculated as follows:

In(rₜ)=In(r₀)+s·t

In(52/(206−52))=In(8/(249−8))+s·10

In(0.3377)=In(0.0332)+s·10

s=0.2320

TABLE 4

| | | | | | | | | Final | Final | |
|---|---|---|---|---|---|---|---|---|---|---|
| Baseline | Baseline | Final | Final | | | | S_{avg} | hits | total | |
| hits | total | hits | total | Days | Sᵣₑₚ | S_{avg} | stdev | sum | sum | Sₛᵤₘ |
| 8 | 249 | 52 | 206 | 10 | 0.2320 | 0.2445 | 0.1045 | 247 | 794 | 0.2610 |
| 8 | 249 | 15 | 144 | 10 | 0.1254 | | | | | |
| 8 | 249 | 110 | 184 | 10 | 0.3802 | | | | | |
| 8 | 249 | 70 | 260 | 10 | 0.2407 | | | | | |

Note that the s_{avg} for the replicates and the sₛᵤₘ of the summed replicates are within 10% of each other in this example. Comparing all of the s_{avg} values for the replicates with the sₛᵤₘ value on the summed replicates gives an r² of 0.86 suggesting that either measure would be useful for selecting winners. Given that they are not perfectly correlated, both were used to ensure all winners were identified. An s value of 0.0500 was used as the initial cutoff for winner selection.

As a first pass for selecting winners from this data, those candidates whose s values were consistently high across all five pools were examined. By taking the average of all the pool sₛᵤₘ values (calculated from the summed hit values), those potential winners that had a selective advantage no matter the environment in which they were screened were identified. From the same averaged sₛᵤₘ values, candidates with strong negative selection across pools were also identified. The average sₛᵤₘ across pools provided the first set of winners. Forty winners (representing 31 genes or genomic regions) had an average sₛᵤₘ across all five pools of 0.0500 or greater.

Because the concept of selection is a function of both genetics and the environment, winners were not selected based solely on a competitive advantage across the board in all experiments. In fact, a winner could show that advantage in a single pool and not in any of the other four in which it was screened. Using the criteria that at least a single pool had an s value of at least 0.0500 (either from the average of replicates—s_{avg}—or via summed hits—sₛᵤₘ), additional winners were selected. Of course, this list was inclusive of the first winners selected based on average sₛᵤₘ value across all five pools. 126 winners comprising 94 unique genes or genomic regions make up this list. This set of genes also includes strong winners and these make up the second tier of candidates. Interestingly, these winners also encompassed all of the lines with a positive average sₛᵤₘ across all pools (this criterion was used above for the first set of genes, though with a 0.500 cutoff rather than 0).

A few genes showed strong selection in the primary screen, often in multiple replicates or different pools, but did not demonstrate a strong competitive advantage in secondary screening. As the secondary screening involved competition against other lines that were selected for growth advantage, it is possible that a line from the primary screen would be obscured by other competitors in all five pools it participated in during secondary screening. Because of this, some additional genes that showed higher s values in primary screening were selected as potential winners.

S. dimorphus

517 successfully isolated and sequence confirmed potential winner clones that were carried into secondary screening were grown in 4-5 mL cultures of TAP in 24-well blocks. Failure to isolate all 637 potential winners was a result of clone death and/or relatively few sorted isolates to choose from. After growth of the cultures for 4-6 days, OD₇₅₀ was measured for each well. Cultures that deviated outside the block average OD were normalized by adding more or less of the given culture when combining into secondary pools. Potential winners were selectively randomized to generate fifty pools of 50-52 genes each.

These 50 pools were each inoculated into four replicate turbidostats and run for 14-15 days as described above. All 50 pools were set up in one round. Each potential winner ended up in 5 distinct pools and 20 turbidostats, so that each winner was placed in 5 different environments to elicit any possible selective advantage. In all, 2 of the 200 turbidostats did not make an endpoint and 3 replicates did not generate any data due to chronic PCR failures.

For each potential winner in a pool, the number of hits at baseline and at the final data point was determined as described previously. Using the total number of sequences derived for each pool at the baseline and final timepoints, hit frequencies were calculated. As expected, the baseline frequencies were very low, centered around a median of 0.0167 (the expected value was 1/50, or 0.02). Final frequencies ranged up to approximately 13.0 (for example, 231 hits out of 248 total sequences equates to 231/(248−231) or 13.59), though most were 1.0 or below and almost 98% were below 0.2. Many of these low values were due to the large number of potential winners that were not detected in the final timepoint and thus were assumed to have a final frequency of 1/1000.

Selection coefficients were calculated for each replicate turbidostat, using the common baseline hit frequency for the pool and the final hit frequency for each replicate (column sᵣₑₚ below) as previously described. The results of the calculations are in as follows.

TABLE 5

| | | | | | | | | Final | Final | |
|---|---|---|---|---|---|---|---|---|---|---|
| Baseline | Baseline | Final | Final | | | | S_{avg} | hits | total | |
| hits | total | hits | total | Days | Sᵣₑₚ | S_{avg} | stdev | sum | sum | Sₛᵤₘ |
| 4 | 344 | 147 | 212 | 14 | 0.3756 | 0.4036 | 0.0508 | 662 | 878 | 0.3973 |
| 4 | 344 | 203 | 226 | 14 | 0.4729 | | | | | |
| 4 | 344 | 172 | 220 | 14 | 0.4085 | | | | | |
| 4 | 344 | 140 | 220 | 14 | 0.3573 | | | | | |

The process of selecting winners from this data applied specific criteria to classify each candidate. Those candidates whose s values were consistently high across all five pools were initially reviewed. If the average of the sₛᵤₘ across all five pools was greater than 0.05 and was statistically different from zero using a 95% confidence interval (one-sample, one-sided t test, p<0.05), those candidates were assigned to Category 1. If the average of the sₛᵤₘ across all pools was greater than 0.1, but not statistically different compared to zero (using a 95% confidence interval)—those candidates were assigned to Category 2. The third category focused on clones that showed good performance in only one (or few) of the five pools. If the s_{avg} for a pool was statistically different from zero using a 95% confidence interval (one-sample, one-sided t test, p<0.05), then those candidates were included in Category 3. All of these had an s_{avg} value greater than 0.12. The final set (Category 4), selected using secondary screen data, included candidates with good performance in a single pool that did not meet the statistical test of being outside the 95% confidence interval (compared to zero). One final source of genes for the Proposed Gene list was considered. A few genes showed strong selection in the primary screen, often in multiple replicates or different pools, but did not demonstrate a strong competitive advantage in secondary screening. As the secondary screening involved competition against other lines that were selected for growth advantage, it was possible that a line from the primary screen would be obscured by other competitors in all five pools it participated in during secondary screening. Because of this, some additional genes that showed higher s values in primary screening were included as Category 5 genes.

Desmodesmus sp./A. maxima

405 Desmodesmus sp. and 97 A. maxima successfully isolated and sequence confirmed potential winner clones for secondary screening were grown in 5 mL cultures of TAP in 24-well blocks. Failure to isolate all 565 potential winners was a result of clone death and/or relatively few sorted isolates to choose from. After growth of the cultures for 4-6 days, cultures were split back into HSM. Following two days of growth in HSM, OD₇₅₀ was measured for each well and cultures were normalized to an OD₇₅₀=0.2. Potential winners were randomized to generate sixty-five pools of 32 winners for Desmodesmus sp. and twenty-five pools of 20 winners for A maxima.

These ninety pools were each inoculated into four replicate turbidostats and run for 13 or 18 days as described above. Each potential winner ended up in 5 distinct pools and 20 turbidostats, replication that puts each winner in 5 different environments to elicit any possible selective advantage.

For each potential winner in a pool, the number of hits at baseline and at the final data point was determined as described previously. Selection coefficients were calculated for the replicate turbidostats, using the common baseline hit frequency for the pool and the final hit frequency for each replicate as described previously. The results are shown in Table 5.

TABLE 6

| Baseline | Baseline | Final | Final | | | | S_{avg} | Final hits | Final | |
|---|---|---|---|---|---|---|---|---|---|---|
| hits | total | hits | total | Days | Sᵣₑₚ | S_{avg} | stdev | sum | total sum | Sₛᵤₘ |
| 9 | 221 | 135 | 176 | 18 | 0.2417 | 0.2495 | 0.0434 | 400 | 516 | 0.2443 |
| 9 | 221 | 158 | 176 | 18 | 0.2962 | | | | | |
| 9 | 221 | 107 | 164 | 18 | 0.2105 | | | | | |

The process of selecting winners from the Desmodesmus and A. maxima data was performed independently. Each analysis applied specific criteria to classify each candidate. For Desmodesmus winners, those candidates whose s values were consistently high across all five pools were selected. If the average of the sₛᵤₘ across all five pools was greater than 0.1 and was statistically different from zero using a 95% confidence interval (one-sample, one-sided t test, p<0.05), those candidates were assigned to Category 1. If the average of the sₛᵤₘ across all pools was greater than 0.1, but not statistically different compared to zero (using a 95% confidence interval)—those candidates were assigned to Category 2. The third category focused on clones that showed good performance in only one (or few) of the five pools. If the s_{avg} was statistically different from zero using a 95% confidence interval (one-sample, one-sided t test, p<0.05), then those candidates were included in Category 3. All of these had an s_{avg} value greater than 0.1. Category 4 included those candidates with good performance in a single pool that did not meet the statistical test of being outside the 95% confidence interval (compared to zero). However, all of these clones had an s_{avg} value greater than 0.1 and should be considered as potential winners. A few genes showed strong selection in the primary screen, often in multiple replicates or different pools, but did not demonstrate a strong competitive advantage in secondary screening. As the secondary screening involved competition against other lines that were selected for growth advantage, it is possible that a line from the primary screen would be obscured by other competitors in all five pools it participated in during secondary screening. Because of this, some additional genes that showed higher s values in primary screening were included as Category 5 genes.

A similar approach was used to classify each candidate from the SE0017 secondary screen. Selection criteria are found in the Table 6.

TABLE 7

| Category | A. maxima Selection Criteria |
|---|---|
| 1 | sₛᵤₘ average across all pools >0.05 and significantly different |
| | than 0 |
| 2 | sₛᵤₘ average across all pools >0.06 |
| 3 | s_{avg} across a single pool >0.1 and significantly different than 0 |
| 4 | s_{avg} across a single pool >0.05 |
| 5 | S_{primary} >0.1, 2+ pools |

For all organisms (C. reinhardtii, S. dimorphus, Desmodesmus and A. maxima), the nature of the cDNA cloned into the overexpression vector for each potential winner may influence whether it made the list. Mainly, if there was no significant ORF anywhere in the sequence, it was not included. These were assumed to be insertional gene disruption events. The ORF that qualifies a gene for the list could be one of several types. The clearest cut was the full annotated CDS of the gene hit by the cDNA, where the 5′ end of the cloned cDNA encompasses at least the ATG and some 5′ UTR. Partial translation of the CDS could occur if the cloned cDNA was not full length, either from the ATG built into the vector or from an internal ATG in the annotated CDS. There could also be an unannotated ORF, perhaps in the 3′ UTR. Finally, in some cases an unannotated ORF may be present within the CDS but in a different frame than the genomic annotation. Any of these could qualify a potential winner for the proposed gene list. While most obvious insertional events were left out of the re-rack, the sequence analysis done at the primary screen level did not catch all such events. Additionally, the predicted Desmodesmus sp. gene models are only algorithmically generated and as such, could have significant differences from the cDNAs expressed in vivo and present in the candidate genes.

### Gene Validation

### General Procedures

Validation of selected genes will consisted of three independent approaches. Selected genes that fail to confirm for a given approach were not advanced to further validation assays. In the first approach, selected genes isolated from turbidostats were competed against 1) wild type and 2) one another en masse to both confirm the phenotype and rank which phenotypes are stronger than others and better than wild-type using the same conditions as in the library screen (numerical and statistical comparisons will be provided). In the second approach, selected genes were regenerated to confirm that the observed phenotype was indeed due to the underlying cDNA or mutation. The phenotype was determined as in the first approach by competitive growth against wild type. A selected gene must have confirmed in both approaches one and two to be designated a validated gene. In the third approach, selected genes were analyzed individually for potential physiologic and/or biochemical properties that gave rise to the observed growth advantage. In the case of improved photosynthesis as a function of cDNA expression, clones were analyzed for phenotypes such as growth under different light and carbon regimes, photosynthetic health (chlorophyll fluorescence) and chlorophyll accumulation. In the case of improved nitrogen utilization as a function of cDNA expression, clones were analyzed for phenotypes such as growth under limiting nitrogen, chlorophyll breakdown, and lipid accumulation.

C. reinhardtti

For each of the 90 selected genes, one primary transgenic line (winner line) was advanced to validation. If a gene was identified more than once in the primary screen (and therefore had more than one winner line), the primary line was the transgenic line containing the longest CDS of the gene. If other winner lines contained different percentages of the CDS (i.e. they are assumed to be non-identical) then another winner line for that gene also entered the validation process. In all, 110 winner lines representing the 90 selected genes entered the validation process.

Turbidostat Competitions with Primary Lines

Starter cultures (5 ml) were grown in TAP media to saturation in deep-well blocks. Three days prior to inoculation of turbidostats, 25 ml cultures in HSM media in flasks were inoculated with 1 ml starter culture. The wild type/parental strain was treated in the same manner though at larger scale. For inoculation into turbidostats, OD₇₅₀ readings of wild type and winner cultures were taken and used to generate a solution containing wild type and winner line at a ratio of 10:1 at a final OD₇₅₀ of approximately 0.5. 10 ml of this mixture was used to inoculate turbidostats with a final volume of 30 ml. Four replicate turbidostats were inoculated from each winner line. The turbidostats were filled with HSM media and set to an OD₇₅₀ of approximately 0.3, which represents an early- to mid-log growth phase. Constant light of ^{˜}150 μEinstein (μE) was provided, with a constant stream of 1% CO₂ bubbling into the culture.

A sample of the mixture used for turbidostat inoculation (time=0) was sorted using FACS onto both TAP media and TAP media containing 20 μg/ml paromomycin (to select for the transgenic line). 384 events were sorted onto each media type. After one week of turbidostat growth, a sample was taken and used for the same sorting procedure.

After approximately one week of growth, photographs of sorted plates were taken by digital camera. Colony numbers on each plate were calculated using the colony counter plugin for ImageJ software(http://imagej.nih.gov/ij/). These colony numbers were then used to calculate a selection coefficient using the formula below (Lenski, 1991, Biotechnology, 15:173-92), as before.

In(rₜ)=In(r₀)+s·t 1.

where r₀ is the ratio of colonies that are paromomycin resistant to colonies that are wild type at the baseline sort, rₜ is this ratio at time t and s is the selection coefficient (expressed in units of t⁻¹).

For en masse experiments, selected lines were grown in 5 ml cultures in TAP media. Cultures were normalized by OD₇₅₀ and pooled. This pooled mixture was sorted by FACS into 96-well liquid cultures for a baseline reading of the distribution of genes. 12 plates were sorted for baseline analysis at the time of entering turbidostats. 12 replicate turbidostats were inoculated from this pool and cultured as before in HSM for two weeks. At 1 week and 2 week time points, samples were taken from turbidostats and sorted into 96-well liquid cultures (4 plates per turbidostat). After approximately one week of growth in 96-well plates, cultures were amplified by PCR and submitted for sequencing. Sanger reads were processed using CLC bio's Genomics Workbench software and a custom plugin. The plugin imports the data into the Genomic Workbench, trimming each sequence for quality and vector. The sequences are then compared to the Chlamydomonas reinhardtii genome using blastn. The gene locus for the top hit was determined and the relation of the BLAST hit and gene CDS was determined. A final result table was generated containing primarily the gene locus and how many times it was hit by a sequence within the dataset. These were compared to the gene loci identified in primary screening and winner numbers were assigned. The distribution of these genes can be compared between the baseline and later time points.

### Regeneration of Lines

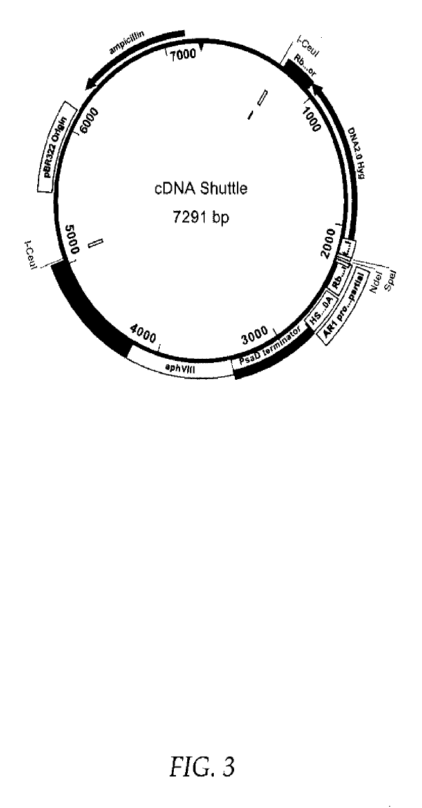

Cold Fusion technology (System Biosciences Inc, USA) was used to re-clone all the selected lines. This method allows cloning of PCR fragments via homology regions at each end of the PCR product and the linearized destination vector. The screening primers used earlier for detection of cloned cDNA were used for this purpose. A vector was built that contains all the regions of the cDNA expression vector except the region between the sites homologous to the screening primers. This region was replaced with the restriction sites NdeI and SpeI (see FIG. 3). A further modification was also made to the expression vector by the addition of I-CeuI sites flanking the entire cassette. These homing endonuclease sites facilitate linearization for transformation since the recognition site is 29 base pairs in length it is unlikely to be found in any cDNA fragment cloned into the library.

Cell lysate of the original selected lines was used as PCR template for cloning. In a few cases where the original line was no longer available, the cDNA insert was PCR amplified from the plasmid cDNA library originally used for primary screening. The cDNA shuttle vector was digested with NdeI and SpeI and purified by gel extraction. PCR product and linearized vector were used for the Cold Fusion reaction as per the manufacturer's guidelines. Cloning in this manner creates an expression cassette identical to the one found in the original lines. Cloned constructs were confirmed by DNA sequencing.

Re-cloned genes were transformed into Chlamydomonas reinhardtii CC-1690 (wild type) and selected for resistance to both hygromycin and paromomycin (each at 10 μg/ml). For each gene, 36 transgenic lines were selected by PCR-based screening. At least 10 PCR positive lines per gene were selected to enter turbidostats in competition with wild type. In three cases (W0143, W0167, W0355), less than 10 lines were PCR positive from the original 36 selected. In these cases, all PCR positive lines (minimum 6) were advanced.

Turbidostat Competitions with Regenerated Lines

Selected lines were grown in TAP media in deep-well 96-well blocks with constant shaking. This starter culture was used to inoculate 1 ml cultures in HSM media three days prior to turbidostat inoculation at a dilution of 1:25. The wild type/parental strain was also grown in this manner except at larger volumes in shake flasks. The 12 transgenic lines were normalized by OD₇₅₀ and pooled. This pooled sample for one gene was then mixed at a ratio of 1:10 (calculated by OD₇₅₀) with the wild type strain and inoculated into quadruplicate turbidostats. A sample of the mixture used for turbidostat inoculation was sorted using FACS onto both TAP media and TAP media containing 20 m/ml paromomycin (to select for the transgenic line). 384 events were sorted onto each media type. Samples were also taken for sorting after one and two weeks of growth in turbidostats.

After approximately one week of growth, photographs of sorted plates were taken by digital camera. Colony numbers on each plate were calculated using the colony counter plugin for ImageJ software. Selection coefficients were calculated as described above.

An additional en masse experiment using regenerated lines was completed. Selected lines were grown in 1 ml cultures in TAP media. Cultures were normalized by OD₇₅₀ and pooled. This pooled mixture was sorted by FACS into 96-well liquid cultures for a baseline reading of the distribution of genes. 12 plates were sorted for baseline analysis prior to entering turbidostats. 12 replicate turbidostats were inoculated from this pool and cultured as before in HSM for two weeks. At 1 week and 2 week time points, samples were taken from turbidostats and sorted into 96-well liquid cultures (4 plates per turbidostat). After approximately one week of growth in 96-well plates, cultures were amplified by PCR and submitted for sequencing. Analysis proceeded as described above.

### Growth and Photosynthesis Assays

Selected Genes were analyzed by a high-throughput 96-well plate-based assay. Briefly, cultures were grown to stationary phase in TAP, MASM, or HSM media. Cultures were diluted to OD₇₅₀=0.1 and grown overnight. Overnight growth was followed by a second dilution to OD₇₅₀=0.02. These initial culture densities put the cells in lag or early log phase. At this point, 200 μl of each culture was added to a 96-well microtiter plate in randomized replicates. 96-well microtiter plates used in this assay contain opaque sides and a transparent base so that light exposure is equal across the entire plate. Plates were sealed using a silicone lid in order to allow for gas exchange but minimize culture volume loss to evaporation. Sealed plates were then set onto a shaker within a growth chamber supplied with 5% CO₂ (except where indicated). Intermittent shaking was set to occur for 5 s/min at 1700 rpm. Light incidence upon each plate lid was set to 130 μE/m². OD₇₅₀ was read every 6 hours for a maximum of 120 hours (until the cultures clearly enter stationary phase as evidenced by the leveling of the curve). The resulting OD₇₅₀ readings, which reflect culture growth, were plotted vs. time. The data are entered into a curve-fitting software package where a 3 parameter logistic function of the form

N(t)=K/(1+(K/Nₒ−1)·e^{(-r·t)})

is fit to the data. The 3 parameters are system specific and represent the carrying capacity (K), the maximal growth rate (r), and the initial density (Nₒ). Differentiating the logistic function yields a rate function; this function can be optimized and solved analytically. This solution for this optimization is equivalent to Kr/4, which is thus the peak theoretical productivity.

Selected Genes were also assessed for photosynthetic quantum yield using a MINI-PAM photosynthesis Yield analyzer (Walz, Germany). The MINI-PAM works by pulsing cultures with saturating light, which briefly suppresses photochemical yield and induces maximal fluorescence yield. The Photosynthesis Yield Analyzer MINI-PAM specializes in the quick and reliable assessment of the effective quantum yield of photochemical energy conversion in photosynthesis. The fluorescence yield (F) and the maximal yield (Fm) are measured and the photosynthesis yield (Y=ΔF/Fm) is calculated. Samples were grown to an OD₇₅₀=0.3 in either HSM or MASM prior to measurement.

### Biochemical Assays

Selected genes were analyzed for increased lipid content by lipid dye staining. Briefly, cultures were grown to an OD₇₅₀=0.5-0.8 in MASM, TAP, or HSM media. 200 μl of each culture was stained with one of three dyes: Nile Red, Bodipy or LipidTox Green (all of which stain neutral lipids). Stained samples were incubated at room temperature for 30 minutes and then processed by the Guava EasyCyte for fluorescent characteristics. Median fluorescence of each sample was used in calculations to determine fold change fluorescence in comparison to wild-type cultures.

Selected genes were processed by Fourier transform infrared spectroscopy (FT-IR) to analyze fatty acid methyl ester (FAME) content. Briefly, samples were grown in a 96 deep-well block format (1 ml total culture volume) in MASM or HSM media. Cultures were harvested by centrifugation in mid-log phase (OD₇₅₀=0.3-0.8). Cell pellets were washed once with distilled water and resuspended in 200 μl of distilled water. 50 μl of the resuspended cells were spotted on to an aluminum 96-well IR plate, dried for 1 hr in a vacuum oven (80° C.), and cooled in a desiccator. Spectra were collected using a vortex 70 FT-IR equipped with an HTS-XT (Bruker Optics). Total relative lipid content (TRLC) was predicted for each spectrum using a PLS (partial least squares) chemometric model created in Opus Quant. Based upon this analysis alone, the transgenic lines appeared to contain more TAGs than the WT line. FT-IR can be used as a high-throughput screening tool to identify potential “high lipid” candidates that are then processed using lower throughput methods, such as microextraction and HPLC analysis.

Selected genes were analyzed for lipid content using HPLC. Briefly, 800 ml cultures grown in HSM media were harvested in late-log phase and extracted using an MTBE/methanol/water solvent mixture. Extracted samples were then injected on to a C18 reverse phase HPLC column equipped with ELSD and DAD detectors. Percent extractables was calculated using standard curves and response factors for multiple compounds. Compounds were chosen to cover general classes of molecules known to be found in algae: monoacylglycerols (MAGs), diacylglycerols (DAGs), triacylglycerols (TAGs), β-carotene, chlorophyll, and other pigments. The general lipid profile was integrated to provide the percent extractable lipid fraction (% ELF) and values were normalized to ash free dry weight (AFDW).

Selected genes that HPLC analysis determined to have high lipid or chlorophyll content were further analyzed by LC/MS to provide a more detailed compound analysis. A C18 reverse phase column was used for separation and a Bruker maXis Q-TOF mass spectrometer was used to record the mass spectra. Mobile phase A is MeOH:H₂O:formic acid:1M NH₄Ac at a 360:40:0.4:4 ratio and mobile phase B is MTBE:MeOH:formic acid:1M NH₄Ac at a 340:60:0.4:4 ratio. A gradient was used in the analysis (from 5% B to 95% B in 18 minutes).

### Validation Results

### Primary Line Competitions

Of the 110 selected lines, 104 were successfully competed against wild type in turbidostats. Failed turbidostats or non-recoverable strain stocks accounted for the remaining 5—these lines advanced directly into the cloning and regeneration steps. One line (W0420) was not successfully regenerated and no data was collected for this line. The majority of lines had an average positive s value in this experiment (85 lines). 72 lines had an average s value of above 0.2. 15 lines representing 14 selected genes showed an s value of 0 or below for all replicates and were considered to have failed validation (W0054, W0074, W0085, W0136, W0143, W0215, W0288, W0297, W0484, W0489, W0496, W0518, W0521, W0526, W0535). While these lines would normally not be carried forward to additional experiments, in some cases additional data was generated. A few lines had negative mean s values but had individual replicates with positive values—these were advanced to the next stage of validation. W0430 also showed a negative coefficient after competition of the original line with wild type but since data from only one turbidostat was obtained it was considered for further validation.

In some cases the number of paromomycin resistant colonies in the sorted samples was higher than the number of colonies on TAP plates containing no antibiotic. In this situation accurate s values were unable to be determined. It is likely in these cases that the population in the turbidostat consisted almost entirely of the selected line and our sample size was not large enough to detect the relatively small number of wild type cells left. In the experiment described here this would result in an s value of around 1 or higher. To allow calculation of s in cases where the number of colonies was higher on the paromomycin plates, the colony number was manually adjusted to one below that of the colony number on the TAP only plate. This allowed a calculation of s that represented the minimum positive correct value. It was also not possible to calculate an accurate s value if there were no colonies present on the plates containing paromomycin (i.e. no transgenic lines found in the sample size taken). In this situation the number of colonies was manually adjusted from 0 to 1 to allow a calculation of s. The s value calculated in this manner would be the minimum negative correct value.

A number of selected lines had s values of close to or above 1 for all replicas and thus almost completely outcompeted wild type in seven days (for example W0018, W0165, W0212, W0159, W0273).

A few control strains were run in wild type competitions as well. A line overexpressing the luciferase gene (Lux) was used and showed a negative selection coefficient relative to wild type, likely due to the increased burden on the cell caused by high expression of this enzyme. A transgenic line overexpressing a cDNA that confers fungicide resistance (FG1) also showed slightly decreased competitive advantage vs. wild type. A bleach tolerant cDNA overexpression line (BT10) had a significant competitive advantage relative to wild type. The line BT10 was originally selected for bleach tolerance using turbidostats under similar conditions as the cDNA screening experiments and therefore has a growth advantage in the conditions of this experiment.

The primary lines representing the selected genes were also run in an en masse competition experiment. All lines were combined in approximately equal amounts and allowed to grow and compete in replicate turbidostats. This experiment was completed twice, each time samples were taken and analyzed at one week after setup. The first run (EM1-12) was also sampled at two weeks. 38 lines showed a level of competitive advantage (relative to the population of all transgenic lines) in at least one of the replicates in the en masse pools. 17 of these lines (W0018, W0032, W0033, W0038, W0040, W0048, W0091, W0109, W0156, W0177, W0273, W0280, W0323, W0365, W0371, W0430, W0512) repeated in both en masse experiments. W0091 and W0177 were two of the most consistent winners from the en masse pools.

### Regenerated Line Competitions

Regenerated lines for 108 of the original winner lines representing 88 selected genes were created. Cloning and regeneration of W0104 was unsuccessful, so only original line data was available for this gene. Line W0240 was also unsuccessful and no data was collected for this line. Of the remaining lines, 4 were regenerated but not screened due to poor performance in the competition with wild type of the original line (W0054, W0074, W0215, W0518). All other lines were regenerated and entered into competitions with wild type in turbidostats.

The samples that entered turbidostat competition contained a pool of 12 transgenic lines. It is likely that only some of these lines were expressing the selected gene to a level sufficient to cause the phenotype of increased selection coefficient. The other lines within the pool could thus have had no selective advantage over wild type in turbidostat growth or could have been at a disadvantage. For this reason, the competition was continued for 2 weeks with a sample also taken after one week (W1). An s value was calculated for week 1 (W0-W1), week 2 (W1-W2), and for the entire two weeks (W0-W2).

The table below incorporates the selection coefficients calculated from the original lines (mean and standard deviation) as well as the s calculations (mean and standard deviation) from the regenerated lines—calculated for three time periods based on two sampling times, week 0-1 (baseline to week 1), week 1-2 (from week 1 to week 2), and week 0-2 (baseline to week 2). If no standard deviation is shown, then the mean value is from a single replicate.

TABLE 8

| | Original | | Regenerated lines | | | | | |
|---|---|---|---|---|---|---|---|---|
| Lin | week 0-1 | | week 0-1 | | week 1-2 | | week 0-2 | |
| Line | Mean | STDEV | Mean | STDEV | Mean | STDEV | Mean | STDEV |
| W0006 | 0.5019 | 0.0933 | −0.2499 | 0.1169 | −0.0460 | 0.2946 | −0.1708 | 0.0899 |
| W0012 | 0.7545 | 0.1586 | | | | | −0.1104 | 0.1230 |
| W0013 | 0.6476 | 0.0402 | −0.0845 | 0.2089 | −0.2590 | | −0.1136 | |
| W0018 | 1.1660 | 0.1802 | −0.0545 | 0.0877 | 0.1239 | 0.0159 | 0.0597 | 0.0018 |
| W0024 | 0.8902 | 0.0659 | 0.1977 | 0.1268 | −0.2549 | 0.4168 | −0.0089 | 0.2407 |
| W0027 | 0.1982 | 0.0490 | −0.2520 | 0.2036 | −0.0017 | 0.2251 | −0.0706 | 0.0707 |
| W0032 | 0.8916 | 0.2395 | −0.0334 | 0.0769 | −0.0520 | | −0.0537 | |
| W0033 | 0.7297 | 0.3064 | 0.2213 | 0.1351 | 0.1605 | | 0.1825 | |
| W0038 | 0.7616 | 0.2701 | 0.2917 | 0.0491 | 0.1514 | 0.6533 | 0.2218 | 0.2913 |
| W0040 | 0.7057 | 0.0619 | −0.3183 | 0.0303 | −0.3133 | 0.0744 | −0.3142 | 0.0532 |
| W0046 | 0.9011 | 0.2430 | −0.3917 | 0.2010 | 0.0004 | | −0.3148 | |
| W0048 | 0.8596 | 0.2708 | 0.1696 | 0.0820 | 0.0191 | 0.3578 | 0.0943 | 0.2036 |
| W0049 | 0.2314 | 0.1146 | 0.1293 | 0.1985 | −0.2799 | 0.2599 | −0.0753 | 0.0854 |
| W0054 | −0.0761 | 0.0580 | | | | | | |
| W0057 | 0.5468 | 0.0607 | 0.1632 | 0.2002 | −0.2958 | 0.2982 | −0.0663 | 0.1788 |
| W0058 | 0.6181 | 0.0310 | 0.2689 | 0.0476 | 0.0832 | 0.0741 | 0.1698 | 0.0208 |
| W0062 | 0.5945 | 0.1681 | 0.1250 | 0.0841 | 0.1087 | | 0.1365 | |
| W0065 | 0.2238 | 0.0612 | 0.4249 | 0.0575 | 0.0713 | 0.1154 | 0.2481 | 0.0796 |
| W0074 | −0.2356 | 0.1961 | | | | | | |
| W0085 | −0.0834 | 0.0735 | −0.4315 | 0.1468 | −0.0296 | 0.2055 | −0.2238 | 0.0003 |
| W0087 | 0.8396 | 0.1173 | −0.3702 | 0.1603 | −0.3379 | | −0.2684 | |
| W0091 | 0.3608 | 0.2165 | −0.4164 | 0.1663 | 0.7177 | 0.4036 | 0.1507 | 0.1836 |
| W0104 | 0.5331 | 0.0748 | | | | | | |
| W0106 | 0.7930 | 0.1531 | −0.2778 | 0.1485 | 0.1480 | 0.4219 | −0.0257 | 0.1686 |
| W0109 | 0.5602 | 0.0764 | −0.3316 | 0.1500 | −0.2170 | 0.0317 | −0.2488 | 0.0202 |
| W0110 | 0.6154 | 0.0496 | −0.1454 | 0.1485 | | | | |
| W0127 | 0.8235 | 0.1530 | −0.2936 | 0.0851 | −0.3542 | | −0.2890 | |
| W0134 | | | 0.4749 | 0.0691 | 0.0484 | | 0.2252 | |
| W0136 | −0.2588 | 0.1539 | −0.2404 | 0.0330 | | | | |
| W0138 | 0.1162 | 0.0307 | −0.5530 | 0.0937 | 0.0231 | 0.2471 | −0.2610 | 0.1260 |
| W0139 | 0.4989 | 0.0659 | −0.1870 | 0.0962 | −0.1831 | 0.1324 | −0.1713 | 0.0200 |
| W0143 | −0.3119 | 0.0955 | −0.0161 | 0.1973 | 0.0783 | 0.2638 | 0.0311 | 0.0528 |
| W0149 | 0.0290 | 0.1642 | 0.2717 | 0.1251 | 0.3268 | 0.4727 | 0.4046 | 0.3983 |
| W0150 | 0.4411 | 0.1030 | 0.4575 | 0.0299 | | | | |
| W0156 | 0.8265 | 0.2528 | −0.1748 | 0.1075 | −0.2477 | 0.2864 | −0.2277 | 0.1687 |
| W0159 | 1.0250 | 0.2210 | 0.1411 | 0.1775 | −0.2933 | 0.2142 | −0.0761 | 0.0212 |
| W0160 | 0.2095 | 0.0287 | −0.0676 | 0.0731 | −0.1013 | 0.1150 | −0.1056 | 0.0581 |
| W0162 | 0.3435 | 0.0453 | 0.2229 | 0.0814 | 0.1301 | 0.2655 | 0.1765 | 0.1170 |
| W0163 | 0.3586 | 0.0980 | −0.2644 | 0.1901 | −0.0900 | | −0.2576 | |
| W0165 | 1.1950 | 0.1706 | −0.1984 | 0.0799 | −0.0045 | 0.2406 | −0.0841 | 0.1114 |
| W0167 | 0.6544 | 0.0280 | 0.2413 | 0.1026 | 0.4146 | 0.4966 | 0.4408 | 0.4104 |
| W0172 | | | 0.2492 | 0.0762 | −0.3235 | 0.3221 | −0.0371 | 0.1992 |
| W0177 | 0.3187 | 0.0252 | −0.4516 | 0.0684 | | | −0.2534 | |
| W0184 | 0.6075 | 0.0300 | −0.0280 | 0.3633 | | | 0.0912 | |
| W0190 | 0.4162 | 0.0391 | 0.1203 | 0.0946 | 0.1316 | 0.2844 | 0.1260 | 0.1657 |
| W0193 | 0.1833 | 0.0724 | −0.4998 | 0.0790 | −0.1084 | | −0.2761 | |
| W0194 | | | 0.2970 | 0.1495 | 0.0812 | 0.3374 | 0.1891 | 0.1943 |
| W0201 | 0.5667 | 0.0314 | 0.4264 | 0.0479 | 0.1963 | 0.0027 | 0.2726 | 0.0689 |
| W0210 | 0.6493 | 0.0491 | −0.2024 | 0.0852 | −0.1988 | 0.0011 | −0.1742 | 0.0467 |
| W0211 | 0.4464 | 0.0903 | 0.4456 | 0.2030 | −0.0618 | 0.3117 | 0.2260 | 0.0459 |
| W0212 | 1.0600 | 0.1860 | −0.3445 | 0.1642 | −0.2449 | 0.1622 | −0.2617 | 0.0020 |
| W0215 | −0.2648 | 0.2441 | | | | | | |
| W0219 | 0.2684 | 0.0724 | −0.3176 | 0.0051 | | | | |
| W0227 | 0.8363 | 0.1931 | 0.3910 | 0.0948 | 0.0997 | 0.2271 | 0.2453 | 0.0871 |
| W0229 | | | −0.3116 | 0.0855 | −0.0201 | 0.1178 | −0.1575 | 0.0020 |
| W0242 | −0.0214 | 0.2844 | −0.0439 | 0.1905 | −0.8152 | | −0.3092 | |
| W0255 | 0.1376 | 0.4177 | 0.0883 | 0.0337 | 0.2495 | 0.2246 | 0.1689 | 0.1100 |
| W0267 | 0.1774 | 0.0598 | −0.2476 | 0.0649 | −0.2149 | | −0.2547 | |
| W0268 | 0.5076 | 0.0908 | −0.1154 | 0.1460 | −0.2014 | | −0.0895 | |
| W0273 | 0.9723 | 0.2102 | −0.0106 | 0.0509 | −0.4317 | 0.3377 | −0.2212 | 0.1661 |
| W0280 | 0.7112 | 0.0613 | −0.5226 | 0.0980 | −0.0881 | | −0.2557 | |
| W0282 | 0.5717 | 0.1696 | 0.3008 | 0.0500 | 0.0604 | | 0.1874 | |
| W0288 | −0.0968 | 0.0640 | −0.2741 | 0.1653 | | | | |
| W0293 | 0.3711 | 0.1146 | −0.4214 | 0.1668 | −0.0416 | 0.2814 | −0.2186 | 0.0032 |
| W0297 | −0.1260 | 0.1324 | −0.2031 | 0.0640 | | | | |
| W0312 | 0.5393 | 0.1768 | −0.2885 | 0.0645 | −0.0274 | 0.0958 | −0.1511 | 0.0126 |
| W0318 | 0.4273 | 0.1214 | 0.3399 | 0.0434 | −0.1653 | 0.1409 | 0.0955 | 0.0718 |
| W0319 | 0.7158 | 0.1131 | −0.4211 | 0.1140 | −0.1595 | 0.0609 | −0.2757 | 0.0440 |
| W0320 | −0.0136 | 0.2599 | −0.2510 | 0.0586 | | | | |
| W0322 | 0.6741 | 0.2891 | −0.3407 | 0.0821 | | | | |
| W0323 | 0.0798 | 0.1126 | 0.3545 | 0.1060 | −0.1107 | 0.0932 | 0.1219 | 0.0272 |
| W0325 | 0.7530 | 0.0720 | 0.3164 | 0.0142 | −0.0714 | 0.1077 | 0.1225 | 0.0469 |
| W0331 | 0.1865 | 0.1019 | −0.5009 | 0.0616 | −0.2087 | 0.0695 | −0.3457 | 0.0440 |
| W0335 | 0.2834 | 0.0178 | 0.2466 | 0.0632 | 0.5074 | 0.0249 | 0.3598 | 0.0022 |
| W0339 | 0.5907 | 0.0758 | −0.3693 | 0.1172 | 0.0205 | 0.1340 | −0.1877 | 0.0183 |
| W0343 | 0.2161 | 0.2706 | −0.3510 | 0.0615 | −0.1672 | 0.0228 | −0.2591 | 0.0196 |
| W0351 | 0.5151 | 0.2962 | 0.3811 | 0.1200 | 0.1835 | 0.2671 | 0.2823 | 0.0903 |
| W0354 | 0.6190 | 0.2689 | −0.1716 | 0.0998 | | | | |
| W0355 | 0.2177 | 0.2451 | 0.2890 | 0.3470 | −0.1215 | 0.1083 | 0.0837 | 0.1249 |
| W0363 | 0.7865 | 0.0651 | −0.2637 | 0.0893 | −0.2312 | 0.2185 | −0.2282 | 0.1513 |
| W0365 | 0.5895 | 0.1670 | −0.2426 | 0.0829 | −0.2229 | 0.1807 | −0.2336 | 0.1090 |
| W0371 | 0.8270 | 0.5240 | 0.2126 | 0.6172 | | | | |
| W0417 | 0.1503 | 0.0983 | −0.5146 | 0.1483 | −0.1831 | | −0.3648 | |
| W0422 | 0.6721 | 0.3283 | −0.2439 | 0.1240 | 0.2372 | 0.0004 | 0.0212 | 0.0120 |
| W0425 | 0.3132 | 0.1481 | −0.1231 | 0.0235 | −0.2850 | | −0.2112 | |
| W0428 | 0.3485 | 0.2347 | −0.4461 | 0.0900 | −0.2664 | | −0.3244 | |
| W0430 | −0.1292 | | 0.1635 | 0.0872 | 0.0415 | 0.1161 | 0.1082 | 0.0110 |
| W0436 | 0.2722 | | −0.3462 | 0.1982 | −0.3352 | 0.0914 | −0.2565 | 0.0786 |
| W0445 | 0.4832 | 0.1040 | 0.5077 | 0.1486 | 0.1623 | 0.4254 | 0.3350 | 0.1450 |
| W0461 | 0.3221 | 0.1432 | 0.0987 | 0.0062 | −0.3370 | 0.2877 | −0.1192 | 0.1460 |
| W0462 | 0.1875 | 0.1160 | −0.1895 | 0.1046 | 0.3805 | | 0.2325 | |
| W0463 | 0.7943 | 0.1762 | −0.1534 | 0.0484 | −0.0201 | 0.0656 | −0.0995 | 0.0466 |
| W0475 | 0.8714 | 0.1741 | | | | | | |
| W0481 | 0.0668 | 0.1014 | 0.0477 | 0.1992 | 0.3048 | | 0.1371 | |
| W0484 | −0.1387 | 0.0820 | −0.4574 | 0.0706 | 0.1571 | 0.4664 | −0.1502 | 0.2175 |
| W0488 | 0.0976 | 0.2730 | 0.3197 | 0.0827 | −0.1515 | 0.0432 | 0.0926 | 0.0619 |
| W0489 | −0.3813 | 0.0594 | −0.3295 | 0.1130 | 0.0549 | 0.2986 | −0.1612 | 0.1816 |
| W0490 | 0.4160 | | 0.2662 | 0.1501 | −0.2025 | | −0.0212 | |
| W0492 | | | −0.1889 | 0.1417 | −0.0138 | 0.0788 | −0.0679 | 0.0415 |
| W0496 | −0.2028 | 0.2321 | −0.2171 | 0.0507 | −0.3395 | | −0.3044 | |
| W0502 | 0.3212 | 0.2321 | 0.0190 | 0.2131 | −0.1423 | 0.1816 | −0.0138 | 0.1452 |
| W0512 | 0.0094 | 0.1100 | −0.2021 | 0.0006 | −0.1123 | 0.2416 | −0.1135 | 0.0842 |
| W0518 | −0.2276 | 0.0276 | | | | | | |
| W0521 | −0.1087 | 0.3676 | −0.1335 | 0.1549 | −0.1826 | 0.1782 | −0.1557 | 0.0632 |
| W0523 | 0.2932 | 0.0814 | −0.1268 | 0.2417 | −0.0770 | 0.2007 | −0.0582 | 0.0468 |
| W0526 | −0.6405 | 0.0016 | −0.2330 | 0.0962 | −0.0517 | 0.0443 | −0.1423 | 0.0549 |
| W0532 | −0.1714 | 0.1775 | −0.1587 | 0.0442 | −0.2801 | | −0.2492 | |
| W0535 | −0.2181 | 0.2658 | −0.3204 | 0.0866 | −0.1364 | 0.1862 | −0.2185 | 0.0460 |
| W0546 | 0.5609 | 0.1858 | −0.3871 | 0.2266 | −0.0064 | | −0.2351 | 0.0672 |

The regenerated lines were also run in an en masse competition experiment. All lines were combined in approximately equal amounts and allowed to grow and compete in replicate turbidostats. Samples were taken at one week and two weeks after setup. 14 lines showed a level of competitive advantage (relative to the population of all transgenic lines) in at least one of the replicates in the en masse pools. W0033 was the most consistent winner from the regenerated en masse pools. Only the week 1 samples were analyzed, as the dominance of W0033 at this time point made analysis after another week of growth likely uninformative.

### Validated Genes

The data for the selection coefficients divided the winner lines into five classes. Class 1 includes those lines that gave positive s values for all calculations of s in all wild type competition replicates (for which data was available) using both the original line and regenerated lines. This class contains 9 lines (W0033, W0058, W0062, W0134, W0150, W0201, W0255, W0282, W0335) representing 9 Selected Genes that are considered validated with very high confidence. Of note in this group is W0033, which is the line that ranked top in the en masse competition of regenerated lines, though the s values in wild type competitions were not among the highest.

Class 2 includes lines that had positive average s values for all calculations of s. Some replicates had a negative value, but all means were positive. This class contains 13 lines, one of which represents a selected gene already present in Class 1. The other 12 selected genes represented by Class 2 are considered validated with a high degree of confidence.

A further 26 lines representing 25 selected genes had variable s values. These lines form Class 3. Of these winner lines, 17 (representing 16 selected genes) have an average s value greater than 0.1 in the original line competition as well as in at least one of the regenerated line competition time points. Three of these genes (W0057, W0211, W0462), are already represented in Class 1 or 2. The remaining 13 Selected Genes were also considered validated, bringing the total to 34 validated genes.

Class 4 includes lines that had a negative average s value for all calculations of s. Some replicates had a positive value, but all means were negative. This group contains 19 lines representing 19 selected genes. One of these (W0268) represents a validated gene from Class 1, but the Class 4 winner line has only 11% of the CDS while the Class 1 winner line for this gene contains 100% CDS.

Class 5 includes 36 lines representing 35 selected genes that have a negative s values for all calculations and replicates. Interestingly, four of the genes represented by Class 5 winner lines (W0087, W0343, W0363, W0496) are considered validated because other winner lines containing these genes are Validated from Class 1, 2 or 3. In all of these cases, the Class 5 line has 100% of the CDS and the Class 1, 2 or 3 line has less than 100% CDS, suggesting either a dominant negative or gene regulation mechanism, as opposed to a simple overexpression of the full length protein. Several lines that gave a negative s value using the original lines were carried forward and re-generated prior to the data analysis indicating they could be dropped. With the exception of W0430 (which had only one replicate for the original line), these lines are found within the lower Classes, confirming that these genes should generally not be considered validated.

The table below lists all 90 selected genes and the winner lines representing them, along with the Class to which they are assigned. Winner lines that contain the same gene are listed together. 34 of these selected genes are considered validated, and are indicated by bold text in the Locus ID column.

TABLE 9

| Gene | | | Description (best arabidopsis TAIR10 | | |
|---|---|---|---|---|---|
| # | Winner | Locus ID | hit defline) | % CDS | Class |
| 1 | W0512 | chromosome_16:206 | | 0 | 4 |
| | | 0033-2061262 | | | |
| 2 | W0318 | Cre01 · g000850 | | 100 | 3 |
| 3 | W0273 | Cre01 · g011000 | Ribosomal protein L6 family protein | 100 | 4 |
| 4 | W0323 | Cre01 · g046300 | | 100 | 3 |
| 5 | W0417 | Cre01 · g051900 | Ubiquinol-cytochrome C reductase | 7 | 5 |
| | | | iron-sulfur subunit | | |
| 6 | W0091 | Cre01 · g059600 | Transport protein particle (TRAPP) | 75 | 3 |
| | | | component | | |
| 7 | W0110 | Cre02 · g077800 | | 4 | 5 |
| 8 | W0422 | Cre02 · g091100 | Ribosomal protein L23/L15e family | 100 | 3 |
| | | | protein | | |
| 9 | W0033 | Cre02 · g106600 | Ribosomal protein S19e family | 100 | 1 |
| | | | protein | | |
| 10 | W0106 | Cre02 · g114600 | 2-cysteine peroxiredoxin B | 56 | 3 |
| 11 | W0057 | Cre02 · g120150 | ribulose bisphosphate carboxylase | 52 | 3 |
| | | | small chain 1A | | |
| 11 | W0255 | Cre02 · g120150 | ribulose bisphosphate carboxylase | 100 | 1 |
| | | | small chain 1A | | |
| 12 | W0488 | Cre03 · g162750 | RNA-binding protein-defense related 1 | 0 | 3 |
| 13 | W0065 | Cre05 · g234550 | fructose-bisphosphate aldolase 2 | 92 | 2 |
| 13 | W0335 | Cre05 · g234550 | fructose-bisphosphate aldolase 2 | 100 | 1 |
| 14 | W0162 | Cre06 · g298650 | eukaryotic translation initiation | 95 | 2 |
| | | | factor 4A1 | | |
| 15 | W0523 | Cre06 · g302900 | ArfGap/RecO-like zinc finger domain- | | 4 |
| | | | containing protein | | |
| 16 | W0085 | Cre11 · g475250 | photosystem II reaction center W | 12 | 4 |
| 16 | W0219 | Cre11 · g475250 | photosystem II reaction center W | 100 | 5 |
| 17 | W0267 | Cre11 · g479500 | ribosomal protein L4 | 0 | 5 |
| 18 | W0280 | Cre11 · g480150 | Ribosomal protein S11 family protein | 28 | 5 |
| 19 | W0032 | Cre12 · g494750 | chloroplast 30S ribosomal protein | 33 | 4 |
| | | | S20, putative | | |
| 20 | W0461 | Cre12 · g501550 | | 100 | 3 |
| 21 | W0177 | Cre12 · g515200 | F-box family protein | 100 | 5 |
| 22 | W0165 | Cre12 · g549300 | gamma tonoplast intrinsic protein | 100 | 4 |
| 23 | W0012 | Cre13 · g580850 | ribosomal protein L22 | 100 | 4 |
| 24 | W0018 | Cre13 · g581650 | ribosomal protein L12-A | 67 | 3 |
| 25 | W0363 | Cre13 · g590500 | fatty acid desaturase 6 | 100 | 5 |
| 25 | W0371 | Cre13 · g590500 | fatty acid desaturase 6 | 57 | 3 |
| 26 | W0038 | Cre14 · g621550 | thioredoxin M-type 4 | 11 | 2 |
| 27 | W0521 | Cre16 · g665650 | GTP-binding protein, HfIX | 43 | 4 |
| 28 | W0339 | Cre19 · g753000 | | 35 | 3 |
| 29 | W0365 | chromosome_14:410 | | | 5 |
| | | 8464-4109141 | | | |
| 30 | W0322 | chromosome_16:239 | | 0 | 5 |
| | | 6473-2397244 | | | |
| 31 | W0320 | Cre01 · g005150 | alanine:glyoxylate aminotransferase | 58 | 5 |
| 32 | W0134 | Cre01 · g010900 | glyceraldehyde-3-phosphate | 100 | 1 |
| | | | dehydrogenase B subunit | | |
| 32 | W0268 | Cre01 · g010900 | glyceraldehyde-3-phosphate | 11 | 4 |
| | | | dehydrogenase B subunit | | |
| 33 | W0046 | Cre01 · g032300 | poly(A) binding protein 7 | 53 | 5 |
| 34 | W0049 | Cre01 · g043350 | Pheophorbide a oxygenase family | 0 | 3 |
| | | | protein with Rieske [2Fe—2S] domain | | |
| 35 | W0062 | Cre01 · g050308 | Ribosomal protein L3 family protein | 70 | 1 |
| 36 | W0430 | Cre01 · g072350 | SPFH/Band 7/PHB domain-containing | 100 | 2 |
| | | | membrane-associated protein family | | |
| 37 | W0190 | Cre02 · g075700 | Ribosomal protein L19e family | 98 | 2 |
| | | | protein | | |
| 37 | W0462 | Cre02 · g075700 | Ribosomal protein L19e family | 100 | 3 |
| | | | protein | | |
| 38 | W0532 | Cre02 · g076250 | Translation elongation factor | 44 | 5 |
| | | | EFG/EF2 protein | | |
| 39 | W0156 | Cre02 · g080200 | Transketolase | 31 | 4 |
| 39 | W0535 | Cre02 · g080200 | Transketolase | 34 | 5 |
| 40 | W0425 | Cre02 · g097900 | aspartate aminotransferase 5 | 24 | 5 |
| 41 | W0013 | Cre02 · g115200 | Ribosomal protein L18e/L15 | 97 | 4 |
| | | | superfamily protein | | |
| 42 | W0193 | Cre02 · g143050 | 60S acidic ribosomal protein family | 100 | 5 |
| 42 | W0502 | Cre02 · g143050 | 60S acidic ribosomal protein family | 70 | 3 |
| 43 | W0319 | Cre03 · g174850 | Polyketide cyclase/dehydrase and | 0 | 5 |
| | | | lipid transport superfamily protein | | |
| 44 | W0312 | Cre03 · g195000 | | 100 | 4 |
| 45 | W0058 | Cre03 · g198000 | Protein phosphatase 2C family | 84 | 1 |
| | | | protein | | |
| 46 | W0149 | Cre03 · g204250 | S-adenosyl-L-homocysteine hydrolase | 9 | 2 |
| 47 | W0139 | Cre05 · g239500 | | 0 | 5 |
| 48 | W0484 | Cre07 · g314150 | zeta-carotene desaturase | 22 | 3 |
| 49 | W0160 | Cre07 · g315300 | | 33 | 4 |
| 50 | W0463 | Cre08 · g377550 | Yippee family putative zinc-binding | 100 | 5 |
| | | | protein | | |
| 51 | W0325 | Cre09 · g416500 | zinc finger (C2H2 type) family protein | 97 | 3 |
| 52 | W0027 | Cre10 · g441950 | Small nuclear ribonucleoprotein | 0 | 4 |
| | | | family protein | | |
| 53 | W0167 | Cre10 · g447950 | | 100 | 2 |
| 54 | W0210 | Cre10 · g448250 | Leucine-rich repeat protein kinase | 10 | 5 |
| | | | family protein | | |
| 55 | W0354 | Cre12 · g485150 | glyceraldehyde-3-phosphate | 8 | 5 |
| | | | dehydrogenase of plastid 1 | | |
| 56 | W0040 | Cre12 · g498600 | GTP binding Elongation factor Tu | 67 | 5 |
| | | | family protein | | |
| 56 | W0143 | Cre12 · g498600 | GTP binding Elongation factor Tu | 100 | 3 |
| | | | family protein | | |
| 57 | W0104 | Cre12 · g529650 | Ribosomal protein | 86 | only |
| | | | L7Ae/L30e/S12e/Gadd45 family | | primary |
| | | | protein | | data |
| 58 | W0212 | Cre12 · g533650 | TRAM, LAG1 and CLN8 (TLC) | 100 | 5 |
| | | | lipid-sensing domain containing | | |
| | | | protein | | |
| 59 | W0024 | Cre12 · g551451 | | 0 | 3 |
| 60 | W0150 | Cre13 · g572300 | | 23 | 1 |
| 61 | W0163 | Cre13 · g574300 | Protein kinase superfamily protein | 31 | 5 |
| 62 | W0445 | Cre14 · g611150 | Small nuclear ribonucleoprotein | 10 | 2 |
| | | | family protein | | |
| 63 | W0282 | Cre14 · g612800 | | 100 | 1 |
| 64 | W0351 | Cre14 · g624000 | F-box/RNI-like superfamily protein | 100 | 2 |
| 65 | W0546 | Cre15 · g635850 | gamma subunit of Mt ATP synthase | 31 | 5 |
| 66 | W0048 | Cre17 · g722200 | mitochondrial ribosomal protein L11 | 100 | 2 |
| 67 | W0428 | Cre22 · g764100 | | 97 | 5 |
| 68 | W0481 | Cre23 · g766250 | photosystem II light harvesting | 12 | 2 |
| | | | complex gene 2.2 | | |
| 69 | W0242 | Cre01 · g052100 | Ribosomal L18p/L5e family protein | 83 | 4 |
| 69 | W0297 | Cre01 · g052100 | Ribosomal L18p/L5e family protein | 78 | 5 |
| 70 | W0138 | Cre02 · g108450 | multiprotein bridging factor 1A | 100 | 3 |
| 71 | W0074 | Cre02 · g124150 | Peroxisomal membrane 22 kDa | 21 | dropped |
| | | | (Mpv17/PMP22) family protein | | |
| 71 | W0288 | Cre02 · g124150 | Peroxisomal membrane 22 kDa | 100 | 5 |
| | | | (Mpv17/PMP22) family protein | | |
| 72 | W0492 | Cre02 · g126650 | Protein kinase superfamily protein | 0 | 4 |
| 73 | W0172 | Cre02 · g134700 | Ribosomal protein L4/L1 family | 36 | 3 |
| 74 | W0490 | Cre02 · g139950 | | 100 | 3 |
| 75 | W0227 | Cre03 · g210050 | Ribosomal protein L35 | 71 | 2 |
| 75 | W0343 | Cre03 · g210050 | Ribosomal protein L35 | 100 | 5 |
| 76 | W0184 | Cre06 · g261000 | photosystem 11 subunit R | 100 | 3 |
| 77 | W0215 | Cre06 · g290950 | ribosomal protein 5B | 93 | dropped |
| 78 | W0229 | Cre06 · g309000 | | 99 | 4 |
| 79 | W0109 | Cre07 · g349250 | | 100 | 5 |
| 80 | W0054 | Cre07 · g353450 | acetyl-CoA synthetase | 10 | dropped |
| 80 | W0293 | Cre07 · g353450 | acetyl-CoA synthetase | 2 | 4 |
| 80 | W0436 | Cre07 · g353450 | acetyl-CoA synthetase | 22 | 5 |
| 81 | W0136 | Cre08 · g380250 | CP12 domain-containing protein 1 | 97 | 5 |
| 82 | W0194 | Cre09 · g386650 | ADP/ATP carrier 3 | 29 | 2 |
| 82 | W0475 | Cre09 · g386650 | ADP/ATP carrier 3 | 100 | only |
| | | | | | primary |
| | | | | | data |
| 83 | W0087 | Cre10 · g417700 | ribosomal protein 1 | 100 | 5 |
| 83 | W0355 | Cre10 · g417700 | ribosomal protein 1 | 99 | 3 |
| 84 | W0331 | Cre10 · g434750 | ketol-acid reductoisomerase | 50 | 5 |
| 84 | W0526 | Cre10 · g434750 | ketol-acid reductoisomerase | 43 | 5 |
| 85 | W0006 | Cre10 · g459250 | Ribosomal protein L35Ae family | 100 | 4 |
| | | | protein | | |
| 86 | W0159 | Cre12 · g528750 | Ribosomal protein L11 family protein | 100 | 3 |
| 86 | W0489 | Cre12 · g528750 | Ribosomal protein L11 family protein | 96 | 3 |
| 87 | W0518 | Cre16 · g693700 | ubiquitin-conjugating enzyme 28 | 48 | dropped |
| 88 | W0201 | Cre17 · g700750 | | 24 | 1 |
| 88 | W0211 | Cre17 · g700750 | | 0 | 3 |
| 88 | W0496 | Cre17 · g700750 | | 100 | 5 |
| 89 | W0240 | Cre12 · g529400 | ribosomal protein S27 | | no data |
| 90 | W0127 | chromosome_14:403 | | | 5 |
| | | 2130-4032881 | | | |

### Growth and Biochemical Characteristics

Winner lines that were carried forward after initial turbidostat competitions (95 lines) were tested in microtiter plate growth assays using three different media: HSM, MASM, and TAP. HSM and MASM are both minimal medias with different nitrogen sources (NH₄ for HSM, NO₃ for MASM) while TAP contains an organic carbon source (acetate) and supports mixotrophic growth. While testing growth in HSM media, it was noticed that the pH dropped significantly as the culture approached late log phase, which resulted in cell death and failure to obtain a full growth curve. Therefore, for the HSM experiments, only growth rate (r) was calculated. Of the 95 strains, 9 displayed a significant increase in r when compared to WT (see table below). In MASM media, full growth curves were obtained. 8 of the 95 samples did show a significant increase in growth rate. Only one line (W0318) showed a significant increase in growth rate in both media. Despite the fact that full growth curves were obtained, none of the samples showed a significant increase in carrying capacity when compared to WT. Microtiter plate assays ran in TAP media grew well and provided full growth curves. However, growth in this replete media (containing an organic carbon source) was so rapid that distinction between WT and transgenic lines was not possible.

Below are summary tables for the initial microtiter plate experiments. An ANOVA with Dunnett's statistic test (p<0.05) was applied to the samples to determine which were significantly different than WT. In the tables below, samples that are highlighted in bold text are samples that are significantly higher than WT samples. Samples that are highlighted by underlining are samples that are significantly lower than WT. If no standard deviation is listed, only a single replicate was available.

TABLE 9
HSM media-Growth rate (r)

| | Mean | STDEV |
|---|---|---|
| Wild Type | 0.104 | 0.007 |
| W0006 | 0.101 | 0.003 |
| W0012 | 0.113 | 0.051 |
| W0013 | 0.081 | 0.003 |
| W0018 | 0.097 | 0.003 |
| W0024 | 0.073 | 0.001 |
| W0027 | 0.126 | 0.017 |
| W0032 | 0.119 | 0.067 |
| W0038 | 0.076 | 0.003 |
| W0040 | 0.108 | 0.008 |
| W0046 | 0.081 | 0.027 |
| W0048 | 0.065 | 0.005 |
| W0049 | 0.100 | 0.012 |
| W0054 | 0.103 | 0.007 |
| W0057 | 0.164 | 0.029 |
| W0058 | 0.110 | 0.031 |
| W0062 | 0.114 | 0.010 |
| W0065 | 0.112 | 0.005 |
| W0074 | 0.101 | 0.008 |
| W0087 | 0.069 | |
| W0091 | 0.073 | 0.004 |
| W0104 | 0.107 | 0.011 |
| W0106 | 0.112 | 0.041 |
| W0109 | 0.107 | 0.011 |
| W0110 | 0.095 | 0.006 |
| W0127 | 0.138 | 0.037 |
| W0136 | 0.099 | 0.013 |
| W0138 | 0.113 | 0.006 |
| W0139 | 0.092 | 0.013 |
| W0149 | 0.094 | 0.011 |
| W0150 | 0.109 | 0.010 |
| W0156 | 0.115 | 0.003 |
| W0159 | 0.100 | 0.008 |
| W0160 | 0.199 | 0.028 |
| W0162 | 0.085 | 0.007 |
| W0163 | 0.093 | 0.007 |
| W0165 | 0.077 | 0.004 |
| W0177 | 0.087 | 0.003 |
| W0184 | 0.125 | 0.023 |
| W0190 | 0.096 | 0.003 |
| W0201 | 0.109 | 0.011 |
| W0210 | 0.131 | 0.067 |
| W0211 | 0.108 | 0.011 |
| W0212 | 0.093 | 0.012 |
| W0215 | 0.080 | 0.002 |
| W0219 | 0.084 | 0.009 |
| W0227 | 0.133 | 0.018 |
| W0242 | 0.095 | 0.006 |
| W0255 | 0.087 | 0.008 |
| W0267 | 0.123 | 0.007 |
| W0268 | 0.110 | 0.007 |
| W0273 | 0.098 | 0.010 |
| W0280 | 0.150 | 0.030 |
| W0282 | 0.165 | 0.021 |
| W0288 | 0.094 | |
| W0293 | 0.103 | 0.002 |
| W0297 | 0.094 | 0.014 |
| W0312 | 0.097 | 0.004 |
| W0318 | 0.186 | 0.012 |
| W0320 | 0.114 | |
| W0322 | 0.105 | 0.012 |
| W0323 | 0.070 | 0.007 |
| W0325 | 0.098 | |
| W0331 | 0.073 | 0.004 |
| W0297 | −0.126 | 0.132 |
| W0312 | 0.539 | 0.177 |
| W0318 | 0.427 | 0.121 |
| W0319 | 0.716 | 0.113 |
| W0320 | −0.014 | 0.260 |
| W0322 | 0.674 | 0.289 |
| W0323 | 0.080 | 0.113 |
| W0325 | 0.753 | 0.072 |
| W0331 | 0.187 | 0.102 |
| W0335 | 0.094 | 0.012 |
| W0339 | 0.108 | 0.007 |
| W0343 | 0.085 | 0.007 |
| W0351 | 0.129 | 0.017 |
| W0354 | 0.067 | 0.013 |
| W0355 | 0.088 | 0.009 |
| W0363 | 0.202 | 0.031 |
| W0365 | 0.130 | 0.019 |
| W0417 | 0.111 | 0.004 |
| W0422 | 0.163 | 0.046 |
| W0425 | 0.107 | 0.013 |
| W0428 | 0.192 | 0.061 |
| W0430 | 0.118 | 0.008 |
| W0436 | 0.101 | 0.004 |
| W0445 | 0.094 | 0.004 |
| W0461 | 0.137 | 0.017 |
| W0462 | 0.091 | 0.011 |
| W0463 | 0.096 | 0.006 |
| W0481 | 0.125 | |
| W0484 | 0.142 | 0.017 |
| W0489 | 0.075 | |
| W0490 | 0.083 | 0.004 |
| W0496 | 0.111 | 0.019 |
| W0502 | 0.097 | 0.009 |
| W0512 | 0.109 | 0.007 |
| W0521 | 0.101 | 0.007 |
| W0523 | 0.125 | 0.024 |
| W0526 | 0.113 | 0.010 |
| W0532 | 0.087 | 0.005 |
| W0535 | 0.129 | 0.045 |
| W0546 | 0.165 | 0.030 |

TABLE 10
MASM media-Carrying capacity, K and growth rate, r

| | K mean | STDEV | r mean | STDEV |
|---|---|---|---|---|
| WT | 0.930 | 0.093 | 0.090 | 0.008 |
| W0006 | 0.814 | 0.040 | 0.089 | 0.009 |
| W0012 | 0.533 | 0.033 | 0.114 | 0.015 |
| W0013 | 0.541 | 0.078 | 0.110 | 0.024 |
| W0018 | 0.646 | 0.106 | 0.095 | 0.018 |
| W0024 | 0.629 | 0.109 | 0.100 | 0.030 |
| W0027 | 0.872 | 0.024 | 0.099 | 0.012 |
| W0032 | 0.566 | 0.098 | 0.090 | 0.019 |
| W0033 | 0.737 | 0.047 | 0.071 | 0.005 |
| W0038 | 0.686 | 0.144 | 0.062 | 0.006 |
| W0040 | 0.811 | 0.096 | 0.048 | 0.005 |
| W0046 | 0.681 | 0.059 | 0.071 | 0.010 |
| W0048 | 0.521 | 0.121 | 0.104 | 0.040 |
| W0049 | 0.806 | 0.117 | 0.092 | 0.009 |
| W0054 | 0.668 | 0.070 | 0.073 | 0.016 |
| W0057 | 0.891 | 0.087 | 0.083 | 0.006 |
| W0058 | 0.703 | 0.112 | 0.096 | 0.037 |
| W0062 | 0.553 | 0.050 | 0.105 | 0.027 |
| W0065 | 0.796 | 0.093 | 0.084 | 0.026 |
| W0085 | 0.236 | 0.103 | 0.059 | 0.013 |
| W0087 | 0.545 | 0.043 | 0.148 | 0.029 |
| W0091 | 0.912 | 0.144 | 0.056 | 0.003 |
| W0104 | 0.790 | 0.071 | 0.048 | 0.004 |
| W0106 | 0.702 | 0.152 | 0.099 | 0.025 |
| W0109 | 0.930 | 0.093 | 0.058 | 0.010 |
| W0110 | 0.891 | 0.078 | 0.048 | 0.005 |
| W0127 | 0.428 | 0.060 | 0.218 | 0.026 |
| W0138 | 0.769 | 0.064 | 0.083 | 0.010 |
| W0139 | 0.449 | 0.043 | 0.187 | 0.047 |
| W0143 | 0.908 | 0.110 | 0.048 | 0.005 |
| W0149 | 0.611 | 0.124 | 0.188 | 0.065 |
| W0150 | 0.646 | 0.125 | 0.121 | 0.063 |
| W0156 | 0.464 | 0.058 | 0.235 | 0.110 |
| W0159 | 0.987 | 0.102 | 0.071 | 0.004 |
| W0160 | 0.526 | 0.080 | 0.136 | 0.057 |
| W0162 | 0.196 | 0.077 | 0.072 | 0.016 |
| W0163 | 0.814 | 0.080 | 0.106 | 0.011 |
| W0165 | 0.467 | 0.064 | 0.049 | 0.007 |
| W0167 | 0.533 | 0.064 | 0.114 | 0.005 |
| W0177 | 0.677 | 0.105 | 0.090 | 0.012 |
| W0184 | 0.680 | 0.091 | 0.113 | 0.027 |
| W0190 | 0.765 | 0.097 | 0.080 | 0.020 |
| W0193 | 0.716 | 0.201 | 0.092 | 0.065 |
| W0201 | 0.485 | 0.071 | 0.189 | 0.035 |
| W0210 | 0.510 | 0.059 | 0.128 | 0.035 |
| W0211 | 0.804 | 0.032 | 0.069 | 0.005 |
| W0212 | 0.609 | 0.247 | 0.085 | 0.032 |
| W0219 | 0.998 | 0.050 | 0.076 | 0.004 |
| W0227 | 0.665 | 0.073 | 0.099 | 0.020 |
| W0242 | 0.654 | 0.162 | 0.161 | 0.101 |
| W0255 | 0.177 | 0.140 | 0.161 | 0.096 |
| W0267 | 0.849 | 0.044 | 0.067 | 0.003 |
| W0268 | 0.637 | 0.052 | 0.083 | 0.011 |
| W0273 | 0.789 | 0.092 | 0.065 | 0.006 |
| W0280 | 0.810 | 0.145 | 0.051 | 0.008 |
| W0282 | 0.550 | 0.098 | 0.071 | 0.028 |
| W0293 | 0.554 | 0.132 | 0.099 | 0.134 |
| W0312 | 0.637 | 0.266 | 0.158 | 0.136 |
| W0318 | 0.490 | 0.225 | 0.204 | 0.114 |
| W0319 | 0.619 | 0.108 | 0.105 | 0.027 |
| W0322 | 0.919 | 0.084 | 0.077 | 0.008 |
| W0323 | 0.707 | 0.095 | 0.055 | 0.006 |
| W0325 | 0.507 | 0.054 | 0.202 | 0.024 |
| W0331 | 0.439 | 0.145 | 0.121 | 0.015 |
| W0335 | 0.827 | 0.209 | 0.071 | 0.035 |
| W0339 | 0.859 | 0.134 | 0.059 | 0.007 |
| W0343 | 0.524 | 0.142 | 0.123 | 0.073 |
| W0351 | 0.605 | 0.119 | 0.104 | 0.024 |
| W0354 | 0.619 | 0.144 | 0.149 | 0.058 |
| W0355 | 1.024 | 0.073 | 0.065 | 0.004 |
| W0363 | 0.455 | 0.044 | 0.117 | 0.024 |
| W0365 | 0.691 | 0.098 | 0.093 | 0.010 |
| W0371 | 0.840 | 0.100 | 0.069 | 0.013 |
| W0417 | 0.562 | 0.130 | 0.105 | 0.044 |
| W0422 | 0.574 | 0.192 | 0.087 | 0.017 |
| W0425 | 0.468 | 0.083 | 0.208 | 0.064 |
| W0428 | 0.792 | 0.164 | 0.076 | 0.016 |
| W0436 | 0.965 | 0.088 | 0.063 | 0.022 |
| W0445 | 0.897 | 0.043 | 0.049 | 0.005 |
| W0461 | 0.479 | 0.040 | 0.160 | 0.027 |
| W0462 | 0.892 | 0.138 | 0.051 | 0.006 |
| W0463 | 0.263 | 0.169 | 0.070 | 0.035 |
| W0475 | 0.651 | 0.151 | 0.140 | 0.037 |
| W0481 | 0.598 | 0.028 | 0.092 | 0.016 |
| W0484 | 0.415 | 0.051 | 0.192 | 0.062 |
| W0488 | 0.546 | 0.168 | 0.091 | 0.031 |
| W0489 | 0.733 | 0.031 | 0.077 | 0.005 |
| W0490 | 0.865 | 0.061 | 0.079 | 0.007 |
| W0496 | 0.831 | 0.061 | 0.081 | 0.012 |
| W0502 | 0.885 | 0.162 | 0.055 | 0.007 |
| W0512 | 0.673 | 0.118 | 0.050 | 0.003 |
| W0521 | 0.892 | 0.132 | 0.057 | 0.017 |
| W0523 | 0.950 | 0.056 | 0.056 | 0.002 |
| W0526 | 0.836 | 0.091 | 0.091 | 0.011 |
| W0532 | 0.855 | 0.085 | 0.080 | 0.005 |
| W0546 | 0.545 | 0.091 | 0.125 | 0.049 |

Using data from the first round of HSM, TAP and MASM microplate experiments, 23 strains were selected for further analysis. Samples were selected based upon increases (though not always significant) in growth rate and/or carrying capacity. Additionally, some samples were selected as negative control samples for these experiments. This experiment was set up such that different media, carbon sources, and light sources were tested for each of the 23 strains. Each condition was replicated multiple times for each strain. The variables for this experiment were: media (TAP or MASM), CO₂ (low or 5%), and light intensity (70 [symbol] E or 130 [symbol] E). Using these variables, six different conditions were set up:

1) TAP, high light, low CO₂
2) TAP, high light, high CO₂
3) TAP, low light, high CO2
4) MASM, high light, low CO₂
5) MASM, high light, high CO₂
6) MASM, low light, high CO₂

Plates were grown for a maximum of 120 hours. Data was analyzed for carrying capacity (K), growth rate (r), and productivity (Kr/4). Data is summarized for each of the 6 conditions in the table below. The header indicates the condition, with red indicating low levels (of organic carbon, light or CO₂) and green indicating higher levels. Any strain that shows a significant increase over wild type in one of the three growth parameters (K, r or Kr/4) is indicated with a black box. Following the summary table are numerical tables that support the summary. Based upon ANOVA with Dunnett's statistic test (p<0.05), samples that are highlighted in green are samples that are significantly higher than WT samples. Samples that are highlighted in brown are samples that are significantly lower than WT.

TABLE 12

| | TAP media-High light (130 μE), Low CO₂ | | | | | |
|---|---|---|---|---|---|---|
| | K mean | STDEV | r mean | STDEV | Kr/4 mean | STDEV |
| WT | 1.050 | 0.090 | 0.200 | 0.040 | 0.050 | 0.010 |
| W0085 | 0.670 | 0.110 | 0.130 | 0.040 | 0.020 | 0.010 |
| W0109 | 1.080 | 0.020 | 0.190 | 0.010 | 0.050 | 0.000 |
| W0127 | 1.040 | 0.080 | 0.150 | 0.020 | 0.040 | 0.000 |
| W0149 | 1.100 | 0.030 | 0.190 | 0.000 | 0.050 | 0.000 |
| W0156 | 1.020 | 0.040 | 0.150 | 0.010 | 0.040 | 0.000 |
| W0159 | 1.050 | 0.040 | 0.150 | 0.010 | 0.040 | 0.000 |
| W0160 | 1.090 | 0.010 | 0.160 | 0.020 | 0.040 | 0.010 |
| W0184 | 1.060 | 0.040 | 0.200 | 0.030 | 0.050 | 0.010 |
| W0219 | 1.190 | 0.030 | 0.160 | 0.010 | 0.050 | 0.000 |
| W0282 | 1.070 | 0.060 | 0.150 | 0.000 | 0.040 | 0.000 |
| W0318 | 0.940 | 0.060 | 0.130 | 0.000 | 0.030 | 0.000 |
| W0325 | 1.140 | 0.050 | 0.160 | 0.020 | 0.050 | 0.000 |
| W0355 | 1.160 | 0.020 | 0.170 | 0.030 | 0.050 | 0.010 |
| W0363 | 1.010 | 0.030 | 0.210 | 0.010 | 0.050 | 0.000 |
| W0417 | 1.090 | 0.040 | 0.220 | 0.020 | 0.060 | 0.000 |
| W0425 | 1.100 | 0.080 | 0.190 | 0.030 | 0.050 | 0.010 |
| W0428 | 0.930 | 0.070 | 0.150 | 0.020 | 0.030 | 0.010 |
| W0436 | 1.080 | 0.050 | 0.170 | 0.030 | 0.050 | 0.010 |
| W0484 | 1.070 | 0.030 | 0.180 | 0.030 | 0.050 | 0.010 |
| W0489 | 0.730 | 0.050 | 0.240 | 0.010 | 0.040 | 0.000 |
| W0523 | 1.130 | 0.050 | 0.140 | 0.010 | 0.040 | 0.000 |
| W0526 | 1.050 | 0.030 | 0.170 | 0.030 | 0.050 | 0.010 |
| W0546 | 1.050 | 0.020 | 0.180 | 0.000 | 0.050 | 0.000 |

TABLE 13

| | TAP Media-High light (130 μE), High CO₂ | | | | | |
|---|---|---|---|---|---|---|
| | K mean | STDEV | r mean | STDEV | Kr/4 mean | STDEV |
| WT | 1.020 | 0.110 | 0.210 | 0.030 | 0.050 | 0.010 |
| W0085 | 0.690 | 0.150 | 0.150 | 0.050 | 0.030 | 0.010 |
| W0109 | 1.100 | 0.050 | 0.230 | 0.020 | 0.060 | 0.010 |
| W0127 | 1.040 | 0.040 | 0.210 | 0.020 | 0.050 | 0.000 |
| W0149 | 1.110 | 0.020 | 0.210 | 0.010 | 0.060 | 0.000 |
| W0156 | 1.010 | 0.070 | 0.210 | 0.010 | 0.050 | 0.000 |
| W0159 | 1.050 | 0.050 | 0.200 | 0.020 | 0.050 | 0.000 |
| W0160 | 1.090 | 0.010 | 0.160 | 0.020 | 0.040 | 0.000 |
| W0184 | 1.130 | 0.040 | 0.220 | 0.020 | 0.060 | 0.010 |
| W0219 | 1.210 | 0.010 | 0.180 | 0.010 | 0.050 | 0.000 |
| W0282 | 1.150 | 0.080 | 0.160 | 0.010 | 0.050 | 0.000 |
| W0318 | 0.930 | 0.030 | 0.140 | 0.000 | 0.030 | 0.000 |
| W0325 | 1.110 | 0.020 | 0.190 | 0.030 | 0.050 | 0.010 |
| W0355 | 1.200 | 0.030 | 0.190 | 0.010 | 0.060 | 0.000 |
| W0363 | 1.070 | 0.010 | 0.180 | 0.010 | 0.050 | 0.000 |
| W0417 | 1.060 | 0.030 | 0.230 | 0.030 | 0.060 | 0.010 |
| W0425 | 1.100 | 0.020 | 0.190 | 0.020 | 0.050 | 0.010 |
| W0428 | 0.960 | 0.040 | 0.180 | 0.000 | 0.040 | 0.000 |
| W0436 | 1.090 | 0.020 | 0.160 | 0.020 | 0.040 | 0.010 |
| W0484 | 1.050 | 0.050 | 0.220 | 0.020 | 0.060 | 0.000 |
| W0489 | 0.780 | 0.010 | 0.260 | 0.000 | 0.050 | 0.000 |
| W0523 | 1.110 | 0.060 | 0.180 | 0.030 | 0.050 | 0.010 |
| W0526 | 1.100 | 0.040 | 0.160 | 0.020 | 0.040 | 0.010 |
| W0546 | 1.050 | 0.030 | 0.180 | 0.020 | 0.050 | 0.000 |

TABLE 14

| | TAP media-Low light (70 μE), High CO₂ | | | | | |
|---|---|---|---|---|---|---|
| | K mean | STDEV | r mean | STDEV | Kr/4 mean | STDEV |
| WT | 0.890 | 0.020 | 0.180 | 0.020 | 0.040 | 0.000 |
| W0085 | 0.320 | 0.080 | 0.180 | 0.050 | 0.010 | 0.000 |
| W0109 | 0.890 | 0.050 | 0.170 | 0.010 | 0.040 | 0.000 |
| W0127 | 0.740 | 0.100 | 0.200 | 0.010 | 0.040 | 0.000 |
| W0149 | 0.830 | 0.060 | 0.160 | 0.010 | 0.030 | 0.000 |
| W0156 | 0.770 | 0.080 | 0.180 | 0.010 | 0.030 | 0.000 |
| W0159 | 0.870 | 0.040 | 0.130 | 0.010 | 0.030 | 0.000 |
| W0160 | 0.880 | 0.020 | 0.100 | 0.010 | 0.020 | 0.000 |
| W0184 | 0.880 | 0.040 | 0.170 | 0.020 | 0.040 | 0.000 |
| W0219 | 1.070 | 0.010 | 0.090 | 0.000 | 0.020 | 0.000 |
| W0282 | 0.840 | 0.060 | 0.140 | 0.000 | 0.030 | 0.000 |
| W0318 | 0.650 | 0.070 | 0.120 | 0.000 | 0.020 | 0.000 |
| W0325 | 0.860 | 0.030 | 0.160 | 0.020 | 0.030 | 0.000 |
| W0355 | 1.050 | 0.040 | 0.090 | 0.010 | 0.020 | 0.000 |
| W0363 | 0.840 | 0.030 | 0.130 | 0.020 | 0.030 | 0.000 |
| W0417 | 0.810 | 0.070 | 0.180 | 0.030 | 0.040 | 0.000 |
| W0425 | 0.850 | 0.030 | 0.170 | 0.030 | 0.040 | 0.010 |
| W0428 | 0.680 | 0.030 | 0.140 | 0.000 | 0.020 | 0.000 |
| W0436 | 0.840 | 0.050 | 0.160 | 0.010 | 0.030 | 0.000 |
| W0484 | 0.920 | 0.050 | 0.190 | 0.010 | 0.040 | 0.000 |
| W0489 | 0.670 | 0.040 | 0.220 | 0.000 | 0.040 | 0.000 |
| W0523 | 0.920 | 0.060 | 0.150 | 0.020 | 0.030 | 0.000 |
| W0526 | 0.790 | 0.070 | 0.170 | 0.030 | 0.030 | 0.000 |
| W0546 | 0.750 | 0.020 | 0.170 | 0.010 | 0.030 | 0.000 |

TABLE 15

| | MASM media-High light (130 μE), Low CO₂ | | | | | |
|---|---|---|---|---|---|---|
| | K mean | STDEV | r mean | STDEV | Kr/4 mean | STDEV |
| SE50 | 0.887 | 0.052 | 0.112 | 0.007 | 0.025 | 0.002 |
| W0085 | 0.621 | 0.026 | 0.093 | 0.012 | 0.015 | 0.002 |
| W0109 | 1.092 | 0.079 | 0.062 | 0.004 | 0.017 | 0.001 |
| W0127 | 0.588 | 0.042 | 0.203 | 0.024 | 0.030 | 0.003 |
| W0149 | 0.738 | 0.052 | 0.138 | 0.033 | 0.026 | 0.007 |
| W0156 | 0.579 | 0.010 | 0.151 | 0.028 | 0.022 | 0.004 |
| W0159 | 1.204 | 0.013 | 0.071 | 0.006 | 0.021 | 0.002 |
| W0160 | 0.569 | 0.062 | 0.097 | 0.011 | 0.014 | 0.001 |
| W0184 | 0.825 | 0.028 | 0.100 | 0.004 | 0.021 | 0.001 |
| W0219 | 1.239 | 0.010 | 0.075 | 0.003 | 0.023 | 0.001 |
| W0282 | 0.701 | 0.057 | 0.117 | 0.025 | 0.020 | 0.003 |
| W0318 | 0.625 | 0.045 | 0.121 | 0.017 | 0.019 | 0.003 |
| W0325 | 0.655 | 0.025 | 0.131 | 0.011 | 0.021 | 0.003 |
| W0355 | 1.165 | 0.017 | 0.071 | 0.003 | 0.021 | 0.001 |
| W0363 | 0.592 | 0.031 | 0.128 | 0.012 | 0.019 | 0.001 |
| W0417 | 0.676 | 0.059 | 0.095 | 0.017 | 0.016 | 0.002 |
| W0425 | 0.594 | 0.028 | 0.180 | 0.019 | 0.027 | 0.003 |
| W0428 | 0.687 | 0.016 | 0.114 | 0.011 | 0.020 | 0.002 |
| W0436 | 0.931 | 0.037 | 0.066 | 0.001 | 0.015 | 0.001 |
| W0484 | 0.536 | 0.022 | 0.168 | 0.018 | 0.022 | 0.002 |
| W0489 | 0.912 | 0.156 | 0.116 | 0.061 | 0.025 | 0.008 |
| W0523 | 1.229 | 0.014 | 0.058 | 0.004 | 0.018 | 0.001 |
| W0526 | 1.055 | 0.024 | 0.071 | 0.003 | 0.019 | 0.001 |
| W0546 | 0.924 | 0.125 | 0.074 | 0.004 | 0.017 | 0.002 |

TABLE 16

| | MASM media-High light (130 μE), High CO₂ | | | | | |
|---|---|---|---|---|---|---|
| | K mean | STDEV | r mean | STDEV | Kr/4 mean | STDEV |
| WT | 1.029 | 0.038 | 0.071 | 0.013 | 0.018 | 0.003 |
| W0085 | 0.602 | 0.009 | 0.106 | 0.015 | 0.016 | 0.002 |
| W0109 | 1.058 | 0.062 | 0.085 | 0.018 | 0.022 | 0.004 |
| W0127 | 0.886 | 0.037 | 0.104 | 0.022 | 0.023 | 0.005 |
| W0149 | 0.980 | 0.048 | 0.106 | 0.008 | 0.026 | 0.002 |
| W0156 | 0.685 | 0.058 | 0.092 | 0.007 | 0.016 | 0.002 |
| W0159 | 1.195 | 0.008 | 0.081 | 0.007 | 0.024 | 0.002 |
| W0160 | 0.639 | 0.046 | 0.146 | 0.006 | 0.023 | 0.002 |
| W0184 | 1.015 | 0.062 | 0.084 | 0.007 | 0.021 | 0.002 |
| W0219 | 1.226 | 0.023 | 0.077 | 0.005 | 0.023 | 0.002 |
| W0282 | 0.908 | 0.058 | 0.088 | 0.024 | 0.020 | 0.004 |
| W0318 | 0.685 | 0.032 | 0.135 | 0.024 | 0.023 | 0.004 |
| W0325 | 0.921 | 0.067 | 0.095 | 0.008 | 0.022 | 0.002 |
| W0355 | 1.178 | 0.016 | 0.071 | 0.002 | 0.021 | 0.001 |
| W0363 | 0.668 | 0.011 | 0.129 | 0.024 | 0.021 | 0.004 |
| W0417 | 1.007 | 0.176 | 0.082 | 0.014 | 0.020 | 0.002 |
| W0425 | 0.920 | 0.072 | 0.123 | 0.016 | 0.028 | 0.002 |
| W0428 | 0.846 | 0.033 | 0.128 | 0.005 | 0.027 | 0.001 |
| W0436 | 1.109 | 0.017 | 0.075 | 0.004 | 0.021 | 0.001 |
| W0484 | 0.808 | 0.026 | 0.121 | 0.017 | 0.024 | 0.003 |
| W0489 | 0.951 | 0.066 | 0.090 | 0.007 | 0.021 | 0.002 |
| W0523 | 1.208 | 0.028 | 0.067 | 0.006 | 0.020 | 0.002 |
| W0526 | 1.082 | 0.038 | 0.083 | 0.013 | 0.022 | 0.003 |
| W0546 | 1.090 | 0.033 | 0.069 | 0.011 | 0.019 | 0.003 |

TABLE 17

| | MASM media-Low light (70 μE), High CO₂ | | | | | |
|---|---|---|---|---|---|---|
| | K mean | STDEV | r mean | STDEV | Kr/4 mean | STDEV |
| WT | 0.649 | 0.032 | 0.061 | 0.014 | 0.010 | 0.002 |
| W0085 | 0.191 | 0.052 | 0.079 | 0.023 | 0.004 | 0.001 |
| W0109 | 0.796 | 0.077 | 0.072 | 0.054 | 0.014 | 0.009 |
| W0127 | 0.493 | 0.046 | 0.137 | 0.010 | 0.017 | 0.002 |
| W0149 | 0.610 | 0.057 | 0.095 | 0.045 | 0.014 | 0.006 |
| W0156 | 0.335 | 0.066 | 0.077 | 0.029 | 0.006 | 0.002 |
| W0159 | 0.920 | 0.072 | 0.042 | 0.002 | 0.010 | 0.001 |
| W0160 | 0.341 | 0.012 | 0.081 | 0.017 | 0.007 | 0.001 |
| W0184 | 0.674 | 0.020 | 0.086 | 0.024 | 0.014 | 0.004 |
| W0219 | 1.113 | 0.042 | 0.047 | 0.000 | 0.013 | 0.001 |
| W0282 | 0.471 | 0.051 | 0.097 | 0.036 | 0.011 | 0.005 |
| W0318 | 0.434 | 0.057 | 0.064 | 0.029 | 0.007 | 0.003 |
| W0325 | 0.599 | 0.038 | 0.106 | 0.069 | 0.015 | 0.009 |
| W0355 | 0.675 | 0.033 | 0.050 | 0.004 | 0.008 | 0.001 |
| W0363 | 0.389 | 0.041 | 0.106 | 0.013 | 0.010 | 0.002 |
| W0417 | 0.387 | 0.030 | 0.089 | 0.010 | 0.009 | 0.001 |
| W0425 | 0.482 | 0.022 | 0.115 | 0.042 | 0.014 | 0.006 |
| W0428 | 0.475 | 0.052 | 0.085 | 0.028 | 0.010 | 0.003 |
| W0436 | 0.731 | 0.049 | 0.060 | 0.022 | 0.011 | 0.003 |
| W0484 | 0.377 | 0.007 | 0.138 | 0.019 | 0.013 | 0.002 |
| W0489 | 0.608 | 0.135 | 0.063 | 0.013 | 0.009 | 0.001 |
| W0523 | 0.831 | 0.164 | 0.071 | 0.033 | 0.014 | 0.005 |
| W0526 | 0.794 | 0.085 | 0.083 | 0.043 | 0.016 | 0.008 |
| W0546 | 0.708 | 0.036 | 0.083 | 0.029 | 0.015 | 0.005 |

All selected genes were screened for photosynthetic yield by MINI-PAM analysis. All strains were tested in both MASM and HSM media. Of the lines tested, none showed a significant increase in photosynthetic yield. This might reflect that MINI-PAM analysis is not sensitive enough to measure the photosynthetic yield difference between transgenic lines and WT. Alternative means may allow for measuring differences between WT and transgenic lines.

TABLE 18

| Photosynthetic | HSM Media | | MASM Media | |
|---|---|---|---|---|
| Yield (PY) | PY mean | STDEV | PY mean | STDEV |
| WT | 0.798 | 0.013 | 0.597 | 0.147 |
| W0006 | 0.782 | 0.031 | 0.764 | 0.030 |
| W0012 | 0.832 | 0.014 | 0.555 | 0.009 |
| W0013 | | | 0.563 | 0.033 |
| W0018 | | | 0.667 | 0.013 |
| W0024 | | | 0.589 | 0.033 |
| W0027 | 0.736 | 0.056 | 0.697 | 0.011 |
| W0032 | 0.316 | 0.253 | 0.595 | 0.032 |
| W0033 | 0.710 | 0.038 | 0.717 | 0.012 |
| W0038 | | | 0.685 | 0.056 |
| W0040 | 0.818 | 0.037 | 0.694 | 0.016 |
| W0046 | 0.000 | 0.000 | 0.305 | 0.288 |
| W0048 | | | 0.676 | 0.008 |
| W0049 | 0.724 | 0.069 | 0.677 | 0.010 |
| W0054 | 0.697 | 0.061 | 0.559 | 0.157 |
| W0057 | 0.716 | 0.066 | 0.502 | 0.016 |
| W0058 | 0.108 | 0.191 | 0.669 | 0.005 |
| W0062 | 0.693 | 0.054 | 0.651 | 0.016 |
| W0065 | 0.662 | 0.072 | 0.688 | 0.014 |
| W0074 | 0.719 | 0.040 | | |
| W0085 | 0.182 | 0.266 | 0.480 | 0.180 |
| W0087 | 0.409 | 0.037 | 0.569 | 0.009 |
| W0091 | | | 0.543 | 0.015 |
| W0104 | 0.830 | 0.019 | 0.705 | 0.003 |
| W0106 | 0.625 | 0.079 | 0.616 | 0.032 |
| W0109 | 0.564 | 0.199 | 0.693 | 0.011 |
| W0110 | 0.700 | 0.037 | 0.709 | 0.022 |
| W0127 | 0.633 | 0.101 | 0.540 | 0.023 |
| W0136 | 0.693 | 0.064 | | |
| W0138 | 0.666 | 0.087 | 0.650 | 0.050 |
| W0139 | 0.814 | 0.016 | 0.491 | 0.052 |
| W0143 | | | 0.405 | 0.333 |
| W0149 | 0.703 | 0.055 | 0.681 | 0.028 |
| W0150 | 0.623 | 0.116 | 0.707 | 0.021 |
| W0156 | 0.692 | 0.064 | 0.547 | 0.046 |
| W0159 | 0.521 | 0.191 | 0.621 | 0.102 |
| W0160 | 0.719 | 0.045 | 0.459 | 0.054 |
| W0162 | 0.564 | 0.120 | 0.271 | 0.262 |
| W0163 | 0.728 | 0.029 | 0.707 | 0.021 |
| W0165 | | | 0.674 | 0.019 |
| W0167 | 0.708 | 0.036 | 0.536 | 0.023 |
| W0177 | | | 0.576 | 0.006 |
| W0184 | 0.845 | 0.016 | 0.732 | 0.045 |
| W0190 | 0.340 | 0.244 | 0.617 | 0.066 |
| W0193 | | | 0.569 | 0.008 |
| W0201 | 0.596 | 0.141 | 0.610 | 0.019 |
| W0210 | 0.710 | 0.055 | 0.616 | 0.011 |
| W0211 | 0.516 | 0.231 | 0.647 | 0.004 |
| W0212 | 0.591 | 0.068 | 0.634 | 0.038 |
| W0215 | 0.663 | 0.089 | | |
| W0219 | 0.554 | 0.103 | 0.678 | 0.025 |
| W0227 | 0.418 | 0.292 | 0.628 | 0.118 |
| W0242 | 0.759 | 0.044 | 0.644 | 0.106 |
| W0255 | 0.580 | 0.158 | 0.429 | 0.369 |
| W0267 | 0.416 | 0.206 | 0.690 | 0.029 |
| W0268 | 0.715 | 0.033 | 0.501 | 0.014 |
| W0273 | 0.677 | 0.062 | 0.665 | 0.031 |
| W0280 | 0.286 | 0.242 | 0.740 | 0.019 |
| W0282 | 0.590 | 0.106 | 0.687 | 0.016 |
| W0288 | 0.844 | 0.036 | | |
| W0293 | 0.000 | 0.000 | 0.636 | 0.017 |
| W0297 | 0.832 | 0.012 | | |
| W0312 | 0.500 | 0.080 | 0.648 | 0.013 |
| W0318 | 0.343 | 0.161 | 0.633 | 0.01 |
| W0319 | 0.170 | 0.331 | 0.608 | 0.138 |
| W0320 | 0.668 | 0.057 | | |
| W0322 | 0.779 | 0.040 | 0.729 | 0.028 |
| W0323 | 0.726 | 0.063 | 0.672 | 0.008 |
| W0325 | 0.565 | 0.143 | 0.528 | 0.015 |
| W0331 | 0.750 | 0.052 | 0.523 | 0.137 |
| W0335 | 0.685 | 0.107 | 0.699 | 0.008 |
| W0339 | 0.714 | 0.017 | 0.648 | 0.016 |
| W0343 | 0.676 | 0.091 | 0.520 | 0.245 |
| W0351 | 0.816 | 0.030 | 0.633 | 0.052 |
| W0354 | 0.595 | 0.054 | 0.695 | 0.005 |
| W0355 | 0.436 | 0.150 | 0.495 | 0.359 |
| W0363 | 0.709 | 0.053 | 0.499 | 0.014 |
| W0365 | 0.556 | 0.143 | 0.492 | 0.016 |
| W0371 | 0.176 | 0.284 | 0.699 | 0.018 |
| W0417 | 0.653 | 0.078 | 0.684 | 0.013 |
| W0422 | 0.543 | 0.129 | 0.641 | 0.011 |
| w0425 | 0.669 | 0.023 | 0.573 | 0.009 |
| W0428 | 0.584 | 0.123 | 0.604 | 0.012 |
| W0430 | 0.676 | 0.061 | | |
| W0436 | 0.581 | 0.106 | 0.717 | 0.027 |
| W0445 | 0.691 | 0.010 | 0.671 | 0.031 |
| W0461 | 0.636 | 0.126 | 0.733 | 0.023 |
| W0462 | 0.840 | 0.019 | 0.679 | 0.006 |
| W0463 | 0.252 | 0.194 | 0.411 | 0.046 |
| W0475 | | | 0.606 | 0.077 |
| W0481 | 0.627 | 0.070 | 0.588 | 0.011 |
| W0484 | 0.712 | 0.048 | 0.385 | 0.051 |
| W0488 | 0.051 | 0.115 | 0.546 | 0.101 |
| W0489 | 0.824 | 0.025 | 0.576 | 0.029 |
| W0490 | 0.111 | 0.248 | 0.551 | 0.002 |
| W0496 | 0.808 | 0.008 | 0.638 | 0.073 |
| W0502 | 0.384 | 0.257 | 0.663 | 0.008 |
| W0512 | 0.236 | 0.246 | 0.665 | 0.045 |
| W0521 | 0.517 | 0.152 | 0.736 | 0.029 |
| W0523 | 0.703 | 0.082 | 0.716 | 0.029 |
| W0526 | 0.834 | 0.022 | 0.693 | 0.010 |
| W0532 | 0.630 | 0.044 | 0.682 | 0.023 |
| W0535 | 0.669 | 0.093 | | |
| W0546 | 0.654 | 0.086 | 0.363 | 0.012 |

Selected genes were screened using a lipid dye staining. Lipid dye staining is a high throughput method to find candidate strains that contain high lipid (and potentially high oil) content. In conjunction with lipid dye staining, all selected genes were processed for FT-IR analysis and HPLC analysis (MTBE extraction). A subset of selected genes from HPLC analysis were also processed for q-TOF analysis to get a more detailed look at how compound composition was altered with respect to WT samples. Several samples showed increased dye staining when stained with Nile Red and LipidTox Green. These samples, when cultured and extracted for HPLC analysis, also showed higher lipid content when compared to WT (wild type, SE50). Below is a comprehensive table that contains all of the Selected Genes, media conditions, and dye stains for this set of experiments. Numerical data indicates fold fluorescence over WT samples. Statistical significance was not calculated with this dataset because only one replicate of each sample was run.

TABLE 19

| | MASM | | | TAP | | | HSM | | |
|---|---|---|---|---|---|---|---|---|---|
| | Bodipy | Nile Red | LipidTox | Bodipy | Nile Red | LipidTox | Bodipy | Nile Red | LipidTox |
| WT | 1.00 | 1.00 | 1.00 | 1.00 | 1.00 | 1.00 | 1.00 | 1.00 | 1.00 |
| W0006 | 0.36 | 1.12 | 0.75 | 0.38 | 0.29 | 0.32 | 1.24 | 0.88 | 1.28 |
| W0012 | 0.40 | 2.17 | 1.25 | 1.61 | 1.25 | 1.10 | 0.54 | 0.19 | 0.63 |
| W0013 | 0.60 | 3.84 | 3.31 | 0.68 | 0.77 | 0.68 | 0.70 | 0.20 | 0.89 |
| W0018 | 0.38 | 1.13 | 0.86 | 1.11 | 0.84 | 0.82 | 0.58 | 0.17 | 0.81 |
| W0024 | 0.45 | 2.81 | 1.40 | 1.21 | 1.33 | 1.16 | 0.43 | 0.14 | 0.44 |
| W0027 | 1.24 | 1.35 | 1.17 | 0.55 | 0.52 | 0.60 | 0.60 | 0.15 | 0.56 |
| W0032 | 0.61 | 5.34 | 2.71 | 1.91 | 2.04 | 2.47 | 0.82 | 0.19 | 0.98 |
| W0033 | 0.63 | 4.54 | 2.40 | 1.28 | 1.77 | 0.99 | 0.70 | 0.14 | 1.08 |
| W0038 | 0.56 | 2.86 | 2.05 | 0.59 | 0.58 | 0.61 | 0.45 | 0.43 | 0.81 |
| W0040 | 0.28 | 0.94 | 1.21 | 0.90 | 0.85 | 0.91 | 0.56 | 0.17 | 0.74 |
| W0046 | 0.58 | 4.10 | 2.63 | 1.04 | 1.39 | 1.34 | 0.39 | 0.12 | 1.16 |
| W0048 | 0.42 | 2.73 | 1.56 | 1.49 | 1.68 | 1.64 | 0.42 | 0.14 | 0.44 |
| W0049 | 1.80 | 1.10 | 1.24 | 0.41 | 0.32 | 0.26 | 0.77 | 0.20 | 1.04 |
| W0054 | 1.48 | 0.79 | 0.89 | 2.65 | 3.00 | 2.60 | 0.80 | 0.34 | 1.31 |
| W0057 | 0.49 | 2.57 | 2.15 | 0.73 | 0.65 | 0.62 | 0.56 | 0.28 | 0.62 |
| W0058 | 0.43 | 2.12 | 1.21 | 0.67 | 0.47 | 0.70 | 0.81 | 0.20 | 0.88 |
| W0062 | 0.31 | 1.85 | 0.97 | 0.81 | 0.83 | 0.93 | 0.45 | 0.29 | 0.69 |
| W0065 | 0.47 | 2.36 | 1.13 | 0.89 | 0.80 | 0.70 | 0.47 | 0.12 | 0.51 |
| W0085 | 0.34 | 0.96 | 1.18 | 0.39 | 0.48 | 0.18 | 0.60 | 0.29 | 0.81 |
| W0087 | 0.35 | 1.84 | 0.75 | 1.32 | 1.08 | 0.93 | 0.87 | | 0.83 |
| W0091 | 0.40 | 2.90 | 1.62 | 0.85 | 0.84 | 1.01 | 0.48 | 0.16 | 0.45 |
| W0104 | 0.26 | 1.31 | 0.71 | 0.70 | 0.68 | 0.77 | 0.33 | 0.12 | 0.35 |
| W0106 | 0.41 | 2.92 | 1.51 | 0.67 | 0.75 | 0.78 | 0.38 | 0.11 | 0.73 |
| W0109 | 1.09 | 1.29 | 1.59 | 0.89 | 0.48 | 0.41 | 1.16 | 0.80 | 1.18 |
| W0110 | 1.56 | 1.23 | 1.10 | 0.63 | 0.71 | 0.68 | 0.39 | 0.14 | 1.85 |
| W0127 | 0.30 | 1.19 | 0.90 | 0.90 | 0.89 | 0.82 | 1.07 | 1.00 | 1.06 |
| W0138 | 2.46 | 1.02 | 1.02 | 0.75 | 0.73 | 0.91 | 0.89 | | 1.01 |
| W0139 | 0.32 | 2.01 | 1.07 | 1.01 | 0.95 | 0.89 | 0.62 | 0.22 | 0.75 |
| W0143 | 1.75 | 0.89 | 1.01 | 1.00 | 1.32 | 1.04 | 0.62 | 0.21 | 0.74 |
| W0149 | 1.08 | 1.01 | 1.52 | 0.75 | 0.76 | 0.83 | 1.11 | 0.91 | 1.12 |
| W0150 | 0.65 | 1.56 | 1.18 | 0.81 | 0.87 | 0.91 | 1.23 | 0.95 | 1.39 |
| W0156 | 0.35 | 1.43 | 0.68 | 0.90 | 0.90 | 0.85 | 0.73 | 0.20 | 0.74 |
| W0159 | 1.81 | 0.58 | 0.88 | 2.94 | 1.93 | 1.67 | 1.81 | 1.06 | 1.99 |
| W0160 | 0.64 | 4.36 | 3.94 | 1.05 | 1.10 | 1.10 | 0.40 | 0.31 | 0.78 |
| W0162 | 0.24 | 0.69 | 1.54 | 2.06 | 2.53 | 1.55 | 0.95 | 0.56 | 1.17 |
| W0163 | 1.77 | 1.20 | 1.17 | 1.00 | 0.87 | 0.80 | 0.41 | 0.15 | 0.86 |
| W0165 | 0.66 | 1.11 | 0.45 | 0.70 | 0.80 | 1.01 | 0.56 | 0.17 | 0.57 |
| W0167 | 0.51 | 3.55 | 2.03 | 1.25 | 1.22 | 1.25 | 0.90 | 0.24 | 1.22 |
| W0177 | 0.41 | 2.37 | 1.14 | 1.10 | 0.72 | 0.67 | 0.71 | 0.33 | 0.98 |
| W0184 | 0.46 | 1.84 | 0.92 | 0.81 | 0.58 | 0.30 | 1.50 | 1.06 | 1.78 |
| W0190 | 0.66 | 1.52 | 0.75 | 1.97 | 1.10 | 0.96 | 0.39 | 0.45 | 0.55 |
| W0193 | 0.45 | 0.86 | 1.09 | 0.63 | 0.59 | 0.66 | 1.04 | 0.39 | 1.11 |
| W0201 | 0.29 | 1.90 | 0.81 | 0.90 | 0.82 | 0.75 | 0.50 | 0.12 | 0.69 |
| W0210 | 0.51 | 3.20 | 2.40 | 0.95 | 0.80 | 0.65 | 0.41 | 0.14 | 0.59 |
| W0211 | 0.55 | 1.35 | 0.88 | 0.99 | 0.76 | 0.87 | 0.32 | 0.13 | 0.39 |
| W0212 | 0.45 | 2.66 | 1.46 | 1.21 | 1.32 | 1.28 | 0.72 | 0.18 | 0.86 |
| W0219 | 1.37 | 0.64 | 0.71 | 1.29 | 1.19 | 1.23 | 1.56 | 0.63 | 1.56 |
| W0227 | 0.36 | 1.21 | 0.85 | 1.02 | 0.96 | 1.02 | 0.38 | 0.14 | 0.44 |
| W0242 | 0.54 | 1.16 | 1.10 | 0.78 | 0.84 | 0.76 | 0.47 | 0.13 | 1.03 |
| W0255 | 0.23 | 0.77 | 0.74 | 0.80 | 0.68 | 0.71 | 1.29 | 0.37 | 1.13 |
| W0267 | 0.68 | 2.87 | 1.70 | 3.52 | 0.56 | 0.55 | 1.19 | 0.36 | 1.50 |
| W0268 | 0.45 | 2.39 | 1.58 | 0.95 | 0.99 | 0.97 | 0.33 | 0.14 | 0.57 |
| W0273 | 1.98 | 1.24 | 1.54 | 0.71 | 0.68 | 0.77 | 0.62 | | 1.03 |
| W0280 | 0.25 | 1.29 | 0.75 | 0.42 | 0.32 | 0.36 | 0.81 | 0.50 | 0.97 |
| W0282 | 0.47 | 2.76 | 2.09 | 1.54 | 1.18 | 0.74 | 0.76 | 0.26 | 0.63 |
| W0293 | 0.47 | 0.27 | 0.20 | 1.02 | 2.18 | 1.71 | 0.46 | 0.13 | 0.37 |
| W0312 | 1.45 | 0.47 | 0.56 | 0.68 | 0.57 | 0.58 | 0.69 | 0.22 | 0.98 |
| W0318 | 0.38 | 2.21 | 1.45 | 1.73 | 1.06 | 0.76 | 0.61 | 0.23 | 0.61 |
| W0319 | 1.12 | 1.03 | 1.04 | 1.91 | 1.22 | 0.10 | 1.54 | 0.34 | 1.12 |
| W0322 | 1.39 | 0.69 | 0.82 | 3.25 | 2.33 | 2.11 | 1.51 | | 2.87 |
| W0323 | 1.81 | 1.04 | 1.26 | 2.90 | 2.43 | 1.85 | 0.94 | 0.67 | 0.99 |
| W0325 | 0.59 | 2.63 | 1.54 | 0.99 | 0.96 | 1.14 | 0.72 | 0.22 | 0.84 |
| W0331 | 1.72 | 0.48 | 0.54 | 1.51 | 1.64 | 1.28 | 0.96 | 0.32 | 0.99 |
| W0335 | 0.53 | 1.07 | 0.62 | 0.79 | 0.83 | 1.00 | 0.44 | 0.12 | 0.74 |
| W0339 | 0.81 | 0.45 | 0.38 | 0.81 | 0.82 | 0.94 | 0.38 | 0.14 | 0.38 |
| W0343 | 0.20 | 1.72 | 1.07 | 1.23 | 1.10 | 1.02 | 0.47 | 0.16 | 1.13 |
| W0351 | 0.36 | 0.97 | 0.53 | 0.95 | 0.90 | 0.83 | 0.34 | 0.12 | 0.90 |
| W0354 | 1.14 | 1.17 | 0.87 | 0.83 | 0.24 | 0.36 | 0.45 | 0.16 | 0.60 |
| W0355 | 0.73 | 0.72 | 0.69 | 1.27 | 1.09 | 1.10 | 1.57 | 0.58 | 1.41 |
| W0363 | 0.55 | 3.14 | 2.19 | 1.32 | 1.11 | 1.05 | 0.73 | 0.28 | 0.80 |
| W0365 | 0.39 | 2.59 | 2.38 | 1.19 | 1.19 | 0.93 | 0.48 | 0.24 | 0.78 |
| W0371 | 0.36 | 2.76 | 1.62 | 1.25 | 1.29 | 1.07 | 0.67 | 0.39 | 0.72 |
| W0417 | 0.54 | 0.52 | 0.58 | 0.66 | 0.80 | 0.88 | 0.66 | 0.20 | 0.69 |
| W0422 | 0.39 | 2.40 | 1.77 | 1.59 | 0.91 | 0.79 | 0.72 | 0.41 | 0.90 |
| W0425 | 0.31 | 2.02 | 0.78 | 0.81 | 0.87 | 0.76 | 0.56 | 0.25 | 0.90 |
| W0428 | 0.34 | 2.39 | 1.94 | 0.79 | 0.70 | 0.57 | 0.96 | 0.78 | 1.07 |
| W0436 | 0.45 | 2.49 | 1.41 | 0.46 | 0.47 | 0.44 | 1.20 | 0.89 | 1.16 |
| W0445 | 0.95 | 0.57 | 0.55 | 0.84 | 1.40 | 1.20 | 0.59 | 0.18 | 1.05 |
| W0461 | 0.27 | 1.54 | 0.67 | 0.81 | 0.55 | 0.42 | 0.58 | 0.32 | 0.57 |
| W0462 | 0.34 | 1.89 | 0.78 | 1.11 | 0.80 | 0.83 | 0.49 | 0.13 | 0.50 |
| W0463 | 0.06 | 0.75 | 0.24 | 0.63 | 0.68 | 0.27 | 0.59 | 0.23 | 0.72 |
| W0475 | 2.00 | 0.80 | 1.17 | 0.78 | 0.86 | 1.05 | 1.35 | 1.05 | 1.62 |
| W0481 | 0.61 | 3.88 | 2.80 | 1.38 | 1.28 | 1.28 | 0.77 | 0.24 | 1.10 |
| W0484 | 0.36 | 1.91 | 1.75 | 0.62 | 0.57 | 0.76 | 0.99 | 0.36 | 1.11 |
| W0488 | 0.40 | 3.11 | 1.85 | 1.56 | 1.94 | 2.03 | 0.78 | 0.17 | 0.85 |
| W0489 | 2.31 | 12.13 | 11.31 | 2.70 | 1.64 | 1.89 | 0.19 | 0.13 | 0.76 |
| W0490 | 0.52 | 2.79 | 1.58 | 0.95 | 0.67 | 0.55 | 0.48 | 0.17 | 0.58 |
| W0496 | 0.28 | 1.12 | 0.49 | 1.98 | 1.64 | 1.34 | 0.73 | 0.25 | 0.69 |
| W0502 | 0.40 | 1.62 | 0.90 | 0.70 | 0.80 | 0.92 | 0.43 | 0.12 | 0.46 |
| W0512 | 0.41 | 2.27 | 1.18 | 0.67 | 0.59 | 0.64 | 0.59 | 0.25 | 0.71 |
| W0521 | 2.75 | 1.53 | 1.50 | 0.52 | 0.43 | 0.35 | 1.25 | 1.05 | 1.27 |
| W0523 | 1.35 | 1.41 | 1.10 | 0.56 | 0.44 | 0.49 | 0.68 | 0.21 | 0.71 |
| W0526 | 1.10 | 0.72 | 0.79 | 0.74 | 0.85 | 0.67 | 0.56 | 0.16 | 0.69 |
| W0532 | 2.79 | 1.39 | 1.57 | 2.60 | 1.98 | 1.68 | 1.36 | 0.91 | 1.34 |
| W0546 | 0.36 | 2.04 | 1.05 | 0.88 | 0.90 | 1.13 | 0.46 | 0.16 | 0.43 |

All selected genes were grown and processed for FT-IR analysis. It was hypothesized that an increase in lipid (and potentially oil) content would alter fatty acid methyl ester (FAME) content of the cell, which can be measured by IR spectroscopy. Below is a table that lists all of the predicted lipid content percentages for each strain when grown in HSM or MASM media. After running all of the selected genes through this high throughput screening method, no significant difference between WT samples and the selected genes was recorded. There are a couple of likely reasons why there were no significant differences: 1) There were no changes in lipid content or 2) small changes in lipid content are hard to distinguish using this method. That is, the current FT-IR model can predict between 14-18% lipids in Chlamydomonas reinhardtii. Due to the narrow range and the crudeness of the model, there is significant error associated with prediction (it is estimated that all values are +/−2%).

TABLE 20

| | MASM | | HSM | |
|---|---|---|---|---|
| | Lipid % | STDEV | Lipid % | STDEV |
| WT | 17.756 | 0.054 | 13.758 | 1.293 |
| W0006 | 15.814 | 0.162 | 13.846 | 1.661 |
| W0012 | 16.716 | 0.093 | 13.307 | 1.245 |
| W0013 | 17.133 | 0.131 | | |
| W0018 | 18.498 | 0.202 | | |
| W0024 | 16.949 | 0.117 | | |
| W0027 | 17.169 | 0.141 | 12.881 | 1.209 |
| W0032 | 15.576 | 0.045 | 12.380 | 0.773 |
| W0033 | 14.839 | 0.199 | 12.175 | 0.653 |
| W0038 | 16.245 | 0.471 | | |
| W0040 | 15.112 | 0.037 | 13.885 | 1.894 |
| W0046 | 17.125 | 0.141 | 11.188 | 1.409 |
| W0048 | 16.987 | 0.064 | | |
| W0049 | 14.764 | 0.049 | 12.372 | 0.635 |
| W0054 | 15.169 | 0.276 | 12.277 | 0.656 |
| W0057 | 15.859 | 0.358 | 12.711 | 1.391 |
| W0058 | 17.700 | 1.085 | 13.473 | 2.083 |
| W0062 | 18.053 | 0.354 | 13.576 | 0.505 |
| W0065 | 16.865 | 0.267 | 13.617 | 2.342 |
| W0074 | | | 12.880 | 1.453 |
| W0085 | 14.604 | 0.154 | 11.636 | 0.646 |
| W0087 | 17.737 | 0.699 | 15.034 | 2.089 |
| W0091 | 15.587 | 0.023 | | |
| W0104 | 17.993 | 0.065 | 13.523 | 1.059 |
| W0106 | 17.134 | 0.379 | 13.715 | 0.736 |
| W0109 | 18.016 | 0.230 | 13.441 | 1.469 |
| W0110 | 17.895 | 0.040 | 14.875 | 1.142 |
| W0127 | 16.693 | 0.374 | 14.320 | 1.538 |
| W0136 | | | 13.231 | 0.178 |
| W0138 | 17.909 | 0.139 | 12.390 | 1.144 |
| W0139 | 17.145 | 0.375 | 16.406 | 0.949 |
| W0143 | 15.791 | 0.494 | | |
| W0149 | 16.000 | 0.668 | 13.065 | 1.069 |
| W0150 | 17.162 | 0.304 | 13.472 | 0.953 |
| W0156 | 17.256 | 0.531 | 14.079 | 1.685 |
| W0159 | 15.935 | 0.241 | 12.061 | 0.497 |
| W0160 | 17.149 | 0.320 | 12.268 | 0.370 |
| W0162 | 13.168 | 0.746 | 12.362 | 0.510 |
| W0163 | 14.845 | 0.571 | 15.148 | 1.435 |
| W0167 | 15.795 | 0.117 | | |
| W0167 | 17.136 | 0.327 | 13.712 | 0.503 |
| W0177 | 16.990 | 0.242 | | |
| W0184 | 17.682 | 0.302 | 13.674 | 0.764 |
| W0190 | 17.462 | 0.626 | 11.563 | 1.137 |
| W0193 | 18.085 | 0.129 | | |
| W0201 | 16.773 | 0.062 | 13.662 | 1.216 |
| W0210 | 16.961 | 0.186 | 12.893 | 1.501 |
| W0211 | 17.036 | 0.171 | 13.262 | 1.488 |
| W0212 | 17.180 | 0.004 | 16.211 | 0.628 |
| W0215 | | | 13.003 | 1.388 |
| W0219 | 15.655 | 0.065 | 12.683 | 0.870 |
| W0227 | 16.896 | 0.292 | 12.654 | 0.980 |
| W0242 | 15.273 | 0.074 | 12.612 | 0.403 |
| W0255 | 13.465 | 0.032 | 12.678 | 1.060 |
| W0267 | 16.645 | 0.298 | 12.965 | 1.339 |
| W0268 | 17.308 | 0.073 | 12.784 | 0.678 |
| W0273 | | | 14.828 | 1.564 |
| W0280 | 18.033 | 0.227 | 13.247 | 1.040 |
| W0282 | 16.280 | 0.073 | 14.038 | 0.865 |
| W0288 | | | 14.092 | 1.787 |
| W0293 | 18.081 | 0.052 | 12.507 | 0.847 |
| W0297 | | | 13.427 | 1.231 |
| W0312 | 17.497 | 0.107 | 14.592 | 1.307 |
| W0318 | 16.428 | 0.127 | 13.028 | 0.062 |
| W0319 | 15.482 | 0.272 | 12.282 | 1.664 |
| W0320 | | | 12.071 | 1.064 |
| W0322 | 14.772 | 0.042 | 11.280 | 0.399 |
| W0323 | 15.010 | 0.154 | 12.631 | 0.261 |
| W0325 | 17.593 | 0.157 | 12.713 | 0.314 |
| W0331 | 14.556 | 0.421 | 14.013 | 1.023 |
| W0335 | 17.346 | 0.877 | 13.063 | 1.060 |
| W0339 | 17.178 | 0.056 | 15.889 | 0.612 |
| W0343 | 14.047 | 0.602 | 14.223 | 0.776 |
| W0351 | 16.970 | 0.240 | 12.964 | 1.455 |
| W0354 | 16.035 | 0.617 | 13.397 | 1.738 |
| W0355 | 15.110 | 0.249 | 11.540 | 0.759 |
| W0363 | 17.057 | 0.210 | 12.902 | 0.990 |
| W0365 | 17.621 | 0.293 | 12.208 | 0.785 |
| W0371 | 16.008 | 0.051 | 11.276 | 0.212 |
| W0417 | 18.275 | 0.240 | 13.139 | 1.798 |
| W0422 | 17.372 | 0.234 | 11.799 | 0.299 |
| W0425 | 16.945 | 0.293 | 14.804 | 0.326 |
| W0428 | 15.303 | 0.076 | 11.598 | 0.134 |
| W0430 | | | 12.206 | 1.399 |
| W0436 | 16.942 | 0.482 | 12.245 | 1.142 |
| W0445 | 16.427 | 0.083 | 12.659 | 0.950 |
| W0461 | 16.766 | 0.244 | 13.142 | 1.290 |
| W0462 | 18.006 | 0.742 | 15.633 | 1.582 |
| W0463 | 12.473 | 0.244 | 12.013 | 0.800 |
| W0475 | 17.740 | 0.171 | | |
| W0481 | 15.463 | 0.013 | 12.163 | 0.521 |
| W0484 | 17.244 | 0.195 | 14.846 | 1.987 |
| W0488 | 14.568 | 0.464 | 12.672 | 0.369 |
| W0489 | 20.062 | 0.445 | 14.291 | 1.632 |
| W0490 | 16.881 | 0.392 | 11.891 | 0.523 |
| W0496 | 18.514 | 0.421 | 11.994 | 0.256 |
| W0502 | 17.491 | 0.631 | 14.226 | 1.775 |
| W0512 | 17.030 | 0.190 | 13.009 | 1.115 |
| W0521 | 17.721 | 0.111 | 13.972 | 1.167 |
| W0523 | 18.652 | 0.020 | 12.082 | 1.071 |
| W0526 | 15.206 | 0.287 | 13.940 | 1.431 |
| W0532 | 14.055 | 0.051 | 12.617 | 0.489 |
| W0535 | | | 12.652 | 0.430 |
| W0546 | 15.318 | 0.256 | 15.523 | 0.822 |

All selected genes were processed for HPLC analysis to examine lipid and pigment content. The table below contains data regarding the lipid content of each strain. “Total lipid content” is further broken down into MAGs, DAGs, and TAGs. Several of these lines had increased lipid content when compared to WT. Most of these lines correlated well with lipid staining. For example, lines W0065, W0087, W0139, W0167, W0339, W0490, and W0512, which had increased lipid staining also showed significant increases in total lipid content, thereby buttressing the validity of lipid dye staining as a predictor of increased lipid content by extraction. As before, values significantly higher than wild type (ANOVA with Dunnett's post test, p<0.05) are highlighted in bold text while those that are lower are highlighted in underlined text.

Given that many of these lines had been characterized as having a high selection coefficient, it was expected that some of these lines may have altered chlorophyll/pigment content. Also shown below is the break down of pigment content into: Xanthophyll, Chlorophyll and B-carotene. Data from this table indicates that 33 lines had significant increases in chlorophyll content.

TABLE 21
Relative content of the major lipid chemical compounds

| | Total | | | | | | | |
|---|---|---|---|---|---|---|---|---|
| | Lipids | STDEV | MAGs | STDEV | DAGs | STDEV | TAGs | STDEV |
| WT | 14.0668 | 1.87438 | 35.3521 | 6.14369 | 47.2748 | 5.47671 | 3.2032 | 0.96339 |
| W0006 | 14.9412 | 1.76109 | 36.6529 | 3.33617 | 39.5773 | 3.02415 | 6.2592 | 0.84212 |
| W0012 | 14.5768 | 1.48370 | 12.8832 | 0.44303 | 62.8307 | 0.29186 | 5.9734 | 0.53367 |
| W0013 | 15.2304 | 0.43108 | 12.5856 | 0.29106 | 64.3807 | 0.60354 | 7.6057 | 0.22237 |
| W0018 | 14.0509 | 0.31203 | 23.9317 | 0.64480 | 56.9955 | 0.30242 | 4.7929 | 0.17745 |
| W0024 | 15.6671 | 1.15553 | 10.7191 | 0.03682 | 65.8086 | 0.34888 | 6.4611 | 0.11635 |
| W0027 | 11.8526 | 0.32832 | 25.5771 | 0.30668 | 53.6720 | 0.78836 | 4.6033 | 0.18302 |
| W0032 | 9.1783 | 0.41301 | 29.4381 | 0.26514 | 43.6607 | 0.64954 | 7.3220 | 0.49276 |
| W0033 | 8.4351 | 0.79699 | 30.5384 | 0.32750 | 43.5182 | 0.19556 | 5.5677 | 0.46447 |
| W0038 | 14.0738 | 0.04521 | 10.4433 | 0.27205 | 63.3074 | 0.45351 | 6.8167 | 0.34652 |
| W0040 | 13.5841 | 1.18475 | 23.4720 | 0.57020 | 55.8491 | 1.00937 | 5.2707 | 0.33597 |
| W0046 | 16.0166 | 1.60622 | 13.0477 | 0.24915 | 64.8447 | 0.60556 | 6.0110 | 0.22163 |
| W0048 | 15.3060 | 1.27396 | 13.5979 | 0.25911 | 64.7296 | 0.58973 | 5.9541 | 0.60985 |
| W0049 | 7.8232 | 0.79740 | 37.3827 | 0.36259 | 36.6566 | 0.28094 | 5.3081 | 0.22370 |
| W0054 | 8.7535 | 0.70698 | 35.9078 | 0.21569 | 39.4852 | 0.40894 | 4.2062 | 0.16592 |
| W0057 | 14.5512 | 1.32948 | 9.3047 | 0.21781 | 64.0636 | 0.38635 | 9.7581 | 0.06296 |
| W0058 | 13.2428 | 0.50621 | 11.7145 | 0.11476 | 65.2095 | 0.26001 | 5.5068 | 0.03661 |
| W0062 | 16.1153 | 2.84915 | 10.5330 | 0.44305 | 66.1459 | 1.90486 | 5.9928 | 0.01535 |
| W0065 | 17.8109 | 0.20860 | 15.5383 | 0.26437 | 65.2593 | 0.55324 | 4.3599 | 0.28604 |
| W0074 | 8.0190 | 0.22834 | 40.1337 | 2.23240 | 38.5518 | 1.64407 | 4.1382 | 0.33491 |
| W0085 | 8.9841 | 0.18944 | 39.5298 | 0.51049 | 37.9835 | 0.74207 | 3.4664 | 0.31075 |
| W0087 | 20.6224 | 0.68759 | 11.5162 | 0.33472 | 69.1157 | 0.76832 | 3.9676 | 0.25158 |
| W0091 | 13.9956 | 1.28455 | 13.8043 | 6.79271 | 57.9738 | 8.49498 | 7.0969 | 0.34369 |
| W0104 | 14.4232 | 1.44995 | 24.9969 | 0.32974 | 56.0248 | 1.98099 | 2.9527 | 0.32936 |
| W0106 | 16.1296 | 0.46967 | 12.2538 | 0.12536 | 63.5079 | 0.57866 | 5.7977 | 0.03388 |
| W0109 | 13.8242 | 1.06218 | 28.6629 | 0.31185 | 52.6824 | 1.45672 | 2.6491 | 0.31322 |
| W0110 | 12.0508 | 0.35260 | 29.8829 | 0.73860 | 49.0015 | 0.91187 | 2.4547 | 0.17479 |
| W0127 | 15.8568 | 0.15807 | 11.3813 | 0.15571 | 64.4764 | 0.06666 | 5.3612 | 0.26353 |
| W0136 | 8.5377 | 0.65426 | 37.0265 | 0.68425 | 41.6863 | 1.43932 | 5.0039 | 0.16514 |
| W0138 | 13.4268 | 1.26397 | 27.0602 | 0.43261 | 51.4283 | 1.74242 | 5.0443 | 0.44412 |
| W0139 | 18.3521 | 0.11907 | 10.4560 | 0.18860 | 64.7848 | 0.26345 | 7.3545 | 0.00525 |
| W0143 | 9.5965 | 0.88008 | 31.7656 | 0.28678 | 39.1909 | 2.09172 | 9.8702 | 0.20915 |
| W0149 | 8.8644 | 0.57703 | 27.3534 | 0.59987 | 54.7646 | 0.76383 | 2.9641 | 0.21941 |
| W0150 | 9.3274 | 0.89613 | 34.7431 | 0.40452 | 41.2667 | 1.37119 | 3.8506 | 0.69821 |
| W0156 | 8.9092 | 0.63970 | 10.3860 | 0.26455 | 54.4953 | 1.40445 | 3.7433 | 1.06266 |
| W0159 | 8.0476 | 1.48306 | 27.4111 | 1.20779 | 44.6004 | 3.90338 | 3.1695 | 1.27523 |
| W0160 | 9.6787 | 1.06193 | 14.6970 | 0.51404 | 60.3975 | 2.52832 | 2.5925 | 0.77695 |
| W0162 | 5.3325 | 0.67693 | 35.3124 | 1.43461 | 33.8900 | 2.10121 | 7.8125 | 0.66562 |
| W0163 | 12.1584 | 0.48449 | 35.1546 | 1.55797 | 47.6831 | 0.66982 | 4.2663 | 1.82962 |
| W0165 | 14.8779 | 0.52096 | 24.7560 | 0.62398 | 55.9041 | 0.55103 | 5.1504 | 0.56555 |
| W0167 | 18.0311 | 0.64597 | 9.6545 | 0.18621 | 67.6832 | 0.71718 | 6.8635 | 0.52491 |
| W0177 | 14.0110 | 0.13819 | 28.2532 | 0.26450 | 53.9001 | 0.82517 | 4.1856 | 0.57625 |
| W0184 | 14.5953 | 0.87420 | 20.4652 | 0.29418 | 60.2563 | 0.62992 | 3.9677 | 0.42441 |
| W0190 | 10.5859 | 0.33098 | 25.1210 | 0.36394 | 49.5684 | 0.93960 | 7.2633 | 0.45523 |
| W0193 | 12.6424 | 0.54629 | 26.9377 | 0.27717 | 53.5332 | 1.29345 | 2.5930 | 0.09531 |
| W0201 | 15.9826 | 1.81146 | 12.7725 | 0.28762 | 67.2860 | 0.64630 | 4.3768 | 0.35010 |
| W0210 | 15.8741 | 1.46951 | 11.9711 | 0.30188 | 66.0346 | 1.49659 | 5.6532 | 0.55084 |
| W0211 | 10.4020 | 1.00708 | 29.4012 | 0.48741 | 48.7633 | 0.76482 | 3.0383 | 0.22933 |
| W0212 | 15.5880 | 0.74772 | 16.0351 | 0.18581 | 66.4705 | 1.36871 | 2.3600 | 0.46089 |
| W0215 | 11.8392 | 1.08148 | 32.4606 | 1.57214 | 49.2662 | 1.54955 | 0.7885 | 1.10100 |
| W0219 | 9.2015 | 0.48258 | 31.0778 | 0.14555 | 44.5790 | 0.59936 | 1.6742 | 1.59053 |
| W0227 | 14.2224 | 0.70881 | 13.5858 | 0.02200 | 64.7563 | 0.90864 | 5.4265 | 0.24699 |
| W0242 | 7.7816 | 0.89039 | 36.1712 | 0.82446 | 37.8107 | 1.07240 | 4.2628 | 0.90345 |
| W0255 | 11.0396 | 0.68905 | 34.3873 | 0.42749 | 44.9121 | 1.09622 | 4.2183 | 0.33643 |
| W0267 | 12.2541 | 0.38516 | 9.9577 | 0.34865 | 61.6201 | 1.49057 | 10.2496 | 0.03842 |
| W0268 | 14.0828 | 1.43021 | 10.9787 | 0.64362 | 63.3817 | 1.72109 | 5.7263 | 1.11834 |
| W0273 | 16.0819 | 0.65552 | 28.0614 | 0.21869 | 57.0351 | 0.87182 | 1.7431 | 0.38095 |
| W0280 | 15.3632 | 1.34452 | 25.0263 | 0.37600 | 59.3697 | 0.62018 | 2.4323 | 0.50523 |
| W0282 | 11.8160 | 0.58660 | 12.1980 | 0.60756 | 58.3511 | 0.17159 | 6.6185 | 0.10576 |
| W0288 | 8.6583 | 1.35353 | 41.8530 | 2.58689 | 27.3949 | 6.61308 | 8.0554 | 2.39581 |
| W0293 | 16.4795 | 1.12524 | 32.9949 | 0.58895 | 53.1502 | 0.42131 | 2.1950 | 0.48646 |
| W0297 | 10.8481 | 0.47382 | 34.2134 | 0.71827 | 44.2262 | 0.46419 | 4.1053 | 0.49545 |
| W0312 | 13.9754 | 0.30996 | 31.3344 | 0.88765 | 48.6981 | 0.50382 | 4.2747 | 0.43840 |
| W0318 | 10.0693 | 0.30063 | 18.5304 | 0.28161 | 57.3665 | 0.87668 | 0.5573 | 0.24702 |
| W0319 | 9.3110 | 0.48897 | 36.1105 | 0.95367 | 41.7563 | 121566 | 4.4352 | 0.38371 |
| W0320 | 7.1164 | 1.09911 | 42.3098 | 0.73216 | 29.4522 | 5.34175 | 5.8180 | 1.56541 |
| W0322 | 10.6858 | 0.16995 | 36.0528 | 0.13289 | 44.1538 | 0.27471 | 4.5863 | 0.38604 |
| W0323 | 8.5497 | 0.47648 | 38.2402 | 0.71442 | 37.0480 | 2.26614 | 5.1276 | 0.26565 |
| W0325 | 6.7821 | 0.99476 | 43.8716 | 0.59254 | 30.9662 | 3.52290 | 6.1339 | 0.50505 |
| W0331 | 11.7440 | 0.99191 | 17.9899 | 0.38680 | 53.4240 | 2.02409 | 5.6382 | 1.13280 |
| W0335 | 15.7167 | 1.40347 | 38.6285 | 0.59211 | 48.5996 | 0.63248 | 1.2976 | 0.74006 |
| W0339 | 17.3021 | 1.34822 | 13.3088 | 3.31940 | 64.8985 | 4.43583 | 4.9574 | 0.21779 |
| W0343 | 8.8396 | 0.48528 | 43.5364 | 2.09646 | 31.3568 | 5.68481 | 8.3743 | 4.23207 |
| W0351 | 16.3621 | 0.78063 | 15.8478 | 0.60046 | 63.7643 | 0.81461 | 6.2619 | 0.58782 |
| W0354 | 9.9670 | 1.52106 | 39.5679 | 0.37463 | 38.3430 | 2.60585 | 3.4881 | 0.29023 |
| W0355 | 8.4155 | 0.61472 | 39.2374 | 0.53511 | 36.7073 | 1.94076 | 5.0095 | 0.18583 |
| W0363 | 15.4875 | 3.16681 | 11.4438 | 0.67130 | 62.5358 | 1.12777 | 4.7937 | 2.78714 |
| W0365 | 9.1880 | 0.52207 | 39.4986 | 0.20691 | 38.1327 | 0.83370 | 4.5510 | 0.43855 |
| W0371 | 13.8593 | 0.67312 | 10.9116 | 0.73550 | 63.1736 | 1.40801 | 8.6149 | 0.31956 |
| W0417 | 12.5242 | 0.25454 | 18.6777 | 2.33700 | 57.2538 | 0.95223 | 6.9027 | 0.59841 |
| W0422 | 13.3333 | 1.29709 | 17.7544 | 0.53735 | 63.3936 | 2.34725 | 0.7780 | 0.65137 |
| W0425 | 17.1600 | 0.11263 | 14.4218 | 0.08430 | 63.3560 | 0.09919 | 5.3455 | 0.15474 |
| W0428 | 7.3023 | 0.85982 | 40.0326 | 0.65972 | 34.0193 | 2.22621 | 6.9687 | 0.43065 |
| W0430 | 9.2451 | 1.24244 | 15.7794 | 0.66845 | 58.4513 | 2.33629 | 6.1112 | 0.38531 |
| W0436 | 11.0616 | 0.94498 | 38.8846 | 1.27324 | 41.2525 | 2.59901 | 4.8889 | 0.64353 |
| W0445 | 8.5912 | 0.81512 | 37.2786 | 1.72446 | 37.5036 | 2.83375 | 4.8347 | 1.31521 |
| W0461 | 8.9452 | 1.04624 | 32.0502 | 0.56459 | 42.8082 | 2.76812 | 6.4246 | 1.59027 |
| W0462 | 13.0373 | 0.10681 | 34.0823 | 1.03391 | 46.3737 | 0.15910 | 4.1773 | 0.01850 |
| W0463 | 7.0190 | 2.17268 | 46.6188 | 5.20783 | 33.0280 | 4.48569 | 5.4797 | 1.25752 |
| W0475 | 10.9812 | 1.27381 | 36.6389 | 0.65806 | 43.6302 | 2.34522 | 3.4538 | 0.14783 |
| W0481 | 13.7156 | 0.12473 | 10.5912 | 0.06288 | 62.5577 | 0.29226 | 5.8273 | 0.12062 |
| W0488 | 12.6890 | 1.82488 | 12.3419 | 0.43704 | 60.7599 | 2.24388 | 7.6021 | 0.11721 |
| W0489 | 11.7977 | 0.73582 | 34.5743 | 0.92317 | 42.5219 | 1.22913 | 5.3044 | 0.59664 |
| W0490 | 17.8934 | 0.57928 | 12.9184 | 0.40142 | 65.3581 | 0.98861 | 5.6642 | 0.14855 |
| W0496 | 13.2748 | 1.39055 | 11.9268 | 6.27517 | 59.4092 | 7.74401 | 9.9866 | 0.89293 |
| W0502 | 13.6335 | 0.57357 | 39.2635 | 0.99197 | 44.7743 | 0.65615 | 2.7786 | 0.88865 |
| W0512 | 18.1685 | 0.72033 | 22.5393 | 0.56866 | 61.2325 | 0.54287 | 3.3834 | 0.37733 |
| W0518 | 14.8088 | 0.98328 | 39.7176 | 0.54067 | 45.6999 | 0.70049 | 2.9921 | 0.83273 |
| W0521 | 12.1721 | 0.78373 | 33.8545 | 0.64898 | 48.8069 | 1.15336 | 1.8380 | 0.59009 |
| W0523 | 8.2477 | 0.98224 | 37.1357 | 0.59349 | 36.9537 | 2.30520 | 8.0061 | 0.45534 |
| W0526 | 10.5213 | 0.56077 | 41.1093 | 0.48452 | 41.2698 | 0.56407 | 3.2519 | 0.14304 |
| W0532 | 8 4291 | 0.47277 | 38.2866 | 0.83141 | 37.4207 | 0.51099 | 5.6867 | 0.52194 |
| W0535 | 9.5018 | 0.49099 | 39.9680 | 1.09993 | 38.3882 | 2.00995 | 3.9191 | 0.09265 |
| W0546 | 15.6667 | 0.85279 | 12.9912 | 0.73292 | 64.9536 | 1.41591 | 4.0931 | 0.31179 |

TABLE 22
Relative content of the major pigment chemical compounds

| | Xantho- | | Chloro- | | b- | |
|---|---|---|---|---|---|---|
| | phyll | STDEV | phyll | STDEV | carotene | STDEV |
| WT | 4.4834 | 0.99026 | 9.1254 | 1.22105 | 0.56111 | 0.508461 |
| W0006 | 6.4183 | 0.28539 | 9.6376 | 0.64530 | 1.45478 | 0.338925 |
| W0012 | 6.0348 | 0.09214 | 9.2661 | 0.34172 | 3.01172 | 0.247482 |
| W0013 | 4.5809 | 0.13773 | 8.3016 | 0.45320 | 2.54540 | 0.121630 |
| W0018 | 5.1139 | 0.15816 | 7.8995 | 0.36191 | 1.26650 | 0.000247 |
| W0024 | 5.6601 | 0.06856 | 8.6901 | 0.41302 | 2.66098 | 0.020462 |
| W0027 | 5.4438 | 0.08782 | 9.2258 | 0.42016 | 1.47807 | 0.033668 |
| W0032 | 6.4894 | 0.27823 | 11.9857 | 0.51343 | 1.10415 | 0.057000 |
| W0033 | 6.4776 | 0.30642 | 13.0331 | 0.25828 | 0.86494 | 0.077180 |
| W0038 | 6.3490 | 0.00233 | 10.1302 | 0.16153 | 2.95334 | 0.001200 |
| W0040 | 5.0401 | 0.14725 | 8.8132 | 0.26179 | 1.55491 | 0.036080 |
| W0046 | 5.0687 | 0.15073 | 8.5311 | 0.39541 | 2.49673 | 0.031030 |
| W0048 | 4.9276 | 0.14176 | 8.3392 | 0.27355 | 2.45160 | 0.129604 |
| W0049 | 6.6794 | 0.27034 | 12.8807 | 0.33956 | 1.09246 | 0.116118 |
| W0054 | 6.7365 | 0.10810 | 12.7977 | 0.33744 | 0.86650 | 0.057941 |
| W0057 | 5.3961 | 0.15949 | 8.7494 | 0.22747 | 2.72821 | 0.015009 |
| W0058 | 5.6390 | 0.00676 | 9.1450 | 0.44869 | 2.78518 | 0.103769 |
| W0062 | 5.7136 | 0.15697 | 8.7415 | 1.29611 | 2.87323 | 0.024086 |
| W0065 | 4.6858 | 0.03273 | 8.1500 | 0.15299 | 2.00670 | 0.079953 |
| W0074 | 6.1902 | 0.22378 | 10.2051 | 0.57979 | 0.78112 | 0.155852 |
| W0085 | 6.0463 | 0.08608 | 12.8414 | 0.21845 | 0.13265 | 0.072048 |
| W0087 | 5.1441 | 0.14635 | 7.8422 | 0.34272 | 2.41412 | 0.136935 |
| W0091 | 6.9805 | 0.31385 | 11.3684 | 1.36200 | 2.77611 | 0.317274 |
| W0104 | 5.2314 | 0.52040 | 9.3740 | 1.30521 | 1.42018 | 0.145964 |
| W0106 | 5.9475 | 0.05524 | 9.5685 | 0.65565 | 2.92469 | 0.040755 |
| W0109 | 5.4590 | 0.43679 | 9.6807 | 1.11643 | 0.86597 | 0.092137 |
| W0110 | 6.0624 | 0.06245 | 11.5287 | 0.35161 | 1.06971 | 0.058895 |
| W0127 | 5.9325 | 0.06053 | 9.8593 | 0.01375 | 2.98936 | 0.100202 |
| W0136 | 5.4934 | 0.44769 | 10.1803 | 0.95697 | 0.60960 | 0.058810 |
| W0138 | 5.1861 | 0.55357 | 10.0770 | 1.11686 | 1.20420 | 0.080699 |
| W0139 | 5.6107 | 0.01470 | 9.0330 | 0.04989 | 2.76099 | 0.015504 |
| W0143 | 5.6591 | 0.49957 | 13.1737 | 1.52951 | 0.34058 | 0.060722 |
| W0149 | 4.7113 | 0.26596 | 9.2719 | 0.57241 | 0.93463 | 0.061972 |
| W0150 | 6.4578 | 0.37307 | 12.8740 | 1.13911 | 0.80780 | 0.146109 |
| W0156 | 10.6027 | 0.17559 | 15.3055 | 0.39147 | 5.46723 | 0.138647 |
| W0159 | 8.2149 | 1.45081 | 14.6343 | 2.26064 | 1.96971 | 0.462128 |
| W0160 | 7.2508 | 1.01294 | 12.1776 | 1.62069 | 2.88454 | 0.521530 |
| W0162 | 8.3342 | 0.46760 | 14.0176 | 0.66975 | 0.63334 | 0.135732 |
| W0163 | 5.0811 | 0.27058 | 6.9396 | 0.49954 | 0.87533 | 0.085252 |
| W0165 | 4.4824 | 0.21430 | 8.7173 | 0.42875 | 0.98989 | 0.038443 |
| W0167 | 5.3298 | 0.32250 | 7.9164 | 0.67037 | 2.55251 | 0.225080 |
| W0177 | 4.6197 | 0.13982 | 8.0288 | 0.30943 | 1.01270 | 0.069801 |
| W0184 | 4.8718 | 0.23456 | 8.7981 | 0.50076 | 1.64088 | 0.060715 |
| W0190 | 5.5786 | 0.17414 | 11.1810 | 0.67544 | 1.28771 | 0.051967 |
| W0193 | 5.7468 | 0.21719 | 10.0360 | 0.81181 | 1.15329 | 0.042471 |
| W0201 | 5.2061 | 0.35432 | 8.0764 | 0.54319 | 2.28219 | 0.116453 |
| W0210 | 5.6877 | 0.57817 | 8.2193 | 0.77796 | 2.43406 | 0.303453 |
| W0211 | 6.6925 | 0.23330 | 10.8801 | 0.46648 | 1.22465 | 0.040991 |
| W0212 | 4.7776 | 0.26415 | 8.6105 | 0.59690 | 1.74626 | 0.050194 |
| W0215 | 6.4543 | 0.28228 | 9.7163 | 0.92354 | 1.31416 | 0.081570 |
| W0219 | 7.6415 | 0.49557 | 13.6582 | 0.14433 | 1.36925 | 0.205715 |
| W0227 | 5.0879 | 0.29582 | 9.1528 | 0.39961 | 1.99076 | 0.011784 |
| W0242 | 7.1477 | 0.54484 | 14.1151 | 0.90882 | 0.49252 | 0.268744 |
| W0255 | 5.4692 | 0.55220 | 10.9188 | 0.77451 | 0.09431 | 0.070701 |
| W0267 | 5.4767 | 0.13210 | 10.1184 | 0.84009 | 2.57758 | 0.131302 |
| W0268 | 6.8802 | 0.66506 | 10.2804 | 0.87328 | 2.75253 | 0.271931 |
| W0273 | 3.9545 | 0.16778 | 8.5006 | 0.42538 | 0.70532 | 0.055855 |
| W0280 | 4.2491 | 0.41003 | 7.8187 | 0.67246 | 1.10397 | 0.148327 |
| W0282 | 7.9142 | 0.45021 | 12.5621 | 0.18801 | 2.35609 | 0.246688 |
| W0288 | 5.9281 | 0.81425 | 16.7687 | 2.53045 | 0.00000 | 0.000000 |
| W0293 | 3.5821 | 0.22948 | 7.5584 | 0.36417 | 0.51943 | 0.054308 |
| W0297 | 5.6209 | 0.11932 | 11.5506 | 0.71465 | 0.28355 | 0.045845 |
| W0312 | 5.2510 | 0.25976 | 9.8202 | 0.43688 | 0.62153 | 0.071673 |
| W0318 | 7.6595 | 0.22006 | 12.5192 | 0.48258 | 3.36707 | 0.085534 |
| W0319 | 5.8702 | 0.06287 | 11.5305 | 0.31301 | 0.29728 | 0.097245 |
| W0320 | 5.5478 | 1.10265 | 16.8723 | 2.96486 | 0.00000 | 0.000000 |
| W0322 | 5.0919 | 0.19249 | 9.5728 | 0.18203 | 0.54244 | 0.076493 |
| W0323 | 6.0259 | 0.47554 | 13.2183 | 1.09644 | 0.34002 | 0.071106 |
| W0325 | 4.8874 | 0.82375 | 14.1408 | 1.77338 | 0.00000 | 0.000000 |
| W0331 | 7.7447 | 0.48433 | 12.3480 | 0.61912 | 2.85511 | 0.174636 |
| W0335 | 3.5869 | 0.16401 | 7.4731 | 0.47811 | 0.41427 | 0.069514 |
| W0339 | 5.3791 | 0.29393 | 9.5871 | 1.11763 | 1.86914 | 0.300811 |
| W0343 | 4.2488 | 0.36727 | 12.4836 | 0.82411 | 0.00000 | 0.000000 |
| W0351 | 4.6872 | 0.23851 | 8.1972 | 0.38144 | 1.24167 | 0.079029 |
| W0354 | 5.7277 | 0.80044 | 12.2924 | 1.80113 | 0.58093 | 0.060658 |
| W0355 | 5.9332 | 0.44434 | 12.8346 | 1.08825 | 0.27800 | 0.138877 |
| W0363 | 7.1404 | 1.45609 | 10.7676 | 2.40895 | 3.31869 | 0.721162 |
| W0365 | 5.8038 | 0.38965 | 11.5117 | 0.66120 | 0.50219 | 0.014491 |
| W0371 | 5.4405 | 0.23551 | 9.2595 | 0.67296 | 2.59985 | 0.144000 |
| W0417 | 5.9191 | 0.03854 | 8.3859 | 0.50783 | 2.86086 | 0.317068 |
| W0422 | 5.7085 | 0.66804 | 9.8845 | 1.07942 | 2.48105 | 0.239693 |
| W0425 | 6.0483 | 0.00879 | 8.6310 | 0.02771 | 2.19737 | 0.009832 |
| W0428 | 4.9273 | 0.44324 | 14.0521 | 1.57618 | 0.00000 | 0.000000 |
| W0430 | 6.5219 | 0.91318 | 11.0076 | 1.66661 | 2.12863 | 0.303785 |
| W0436 | 4.7427 | 0.24676 | 10.1918 | 0.90471 | 0.03953 | 0.024216 |
| W0445 | 5.8015 | 0.57920 | 14.5816 | 1.55197 | 0.00000 | 0.000000 |
| W0461 | 5.4403 | 0.64365 | 12.5188 | 1.46918 | 0.75796 | 0.187086 |
| W0462 | 5.0813 | 0.19420 | 9.6716 | 0.91666 | 0.61386 | 0.063651 |
| W0463 | 5.5471 | 0.64006 | 9.2161 | 0.00497 | 0.11039 | 0.099719 |
| W0475 | 5.4801 | 0.57221 | 10.2450 | 1.33771 | 0.55183 | 0.070496 |
| W0481 | 6.8483 | 0.17724 | 10.9246 | 0.19019 | 3.25088 | 0.017428 |
| W0488 | 6.4416 | 0.90455 | 10.1149 | 1.55335 | 2.73959 | 0.340222 |
| W0489 | 5.9823 | 0.26606 | 11.1075 | 0.66913 | 0.50971 | 0.044426 |
| W0490 | 5.5490 | 0.26039 | 8.2172 | 0.42331 | 2.29312 | 0.114783 |
| W0496 | 5.9241 | 0.54378 | 10.4420 | 1.42781 | 2.31131 | 0.614617 |
| W0502 | 4.2481 | 0.12159 | 8.5919 | 0.33680 | 0.34366 | 0.043426 |
| W0512 | 4.3462 | 0.17017 | 7.2528 | 0.22272 | 1.24588 | 0.062427 |
| W0518 | 3.8090 | 0.22592 | 7.4899 | 0.23174 | 0.29157 | 0.063057 |
| W0521 | 4.5845 | 0.19686 | 10.4304 | 0.69586 | 0.48568 | 0.039267 |
| W0523 | 5.2303 | 0.55840 | 12.2971 | 1.52600 | 0.37706 | 0.111870 |
| W0526 | 4.4768 | 0.30650 | 9.6030 | 0.52959 | 0.28907 | 0.059982 |
| W0532 | 5.5267 | 0.27063 | 12.8380 | 0.31051 | 0.24131 | 0.106993 |
| W0535 | 5.3597 | 0.26322 | 12.1795 | 0.68329 | 0.18547 | 0.025639 |
| W0546 | 6.2002 | 0.39649 | 9.6320 | 0.51023 | 2.12995 | 0.120424 |

After data from the HPLC was obtained, there were several lines that warranted further, detailed analysis on the constituent compounds within the lines. To this end, the same extractions from the HPLC were run through the LC-Q-TOF. Lines were selected by having significant differences from WT. The first set of samples that were analyzed were samples that contained high total extractable lipid contents. These lines were: W0087, W0139, W0512, W0167, W0490, W0339, W0162 (negative), and W0325 (negative). Samples that had high chlorophyll content were also analyzed by LC-Q-TOF analysis. High chlorophyll samples that were selected were: W0156, W0159, W0288, W0320, W0445, and W0163 (negative). Data is summarized in tables below, where values indicate percentage of total area under the curve(s) for each category. Note: each category (MAG, TAG, etc) is comprised of several constituent compounds. For brevity, these compounds were summed to give the values in the table.

TABLE 23

| | MAG | DAG | DGTS | DGDG | TAG | Ceramide | LPC | ester |
|---|---|---|---|---|---|---|---|---|
| WT | 0.000 | 10.610 | 34.770 | 1.290 | 26.750 | 5.280 | 0.000 | 0.550 |
| W0087 | 0.980 | 18.160 | 21.230 | 2.550 | 30.470 | 0.000 | 6.880 | 1.230 |
| W0139 | 0.460 | 17.520 | 24.050 | 2.370 | 33.920 | 0.000 | 7.630 | 1.100 |
| W0156 | 1.160 | 14.870 | 16.990 | 1.220 | 31.430 | 0.000 | 2.380 | 1.390 |
| W0159 | 0.000 | 0.940 | 29.780 | 0.940 | 25.810 | 0.000 | 0.000 | 0.820 |
| W0163 | 0.000 | 0.000 | 34.660 | 0.000 | 33.750 | 1.270 | 0.000 | 0.000 |
| W0167 | 0.000 | 14.780 | 21.230 | 1.170 | 33.410 | 0.000 | 0.000 | 1.350 |
| W0288 | 0.000 | 1.160 | 14.620 | 0.000 | 14.240 | 0.000 | 1.160 | 1.690 |
| W0320 | 0.000 | 0.000 | 7.660 | 0.000 | 20.150 | 0.000 | 0.000 | 1.840 |
| W0325 | 0.000 | 0.000 | 5.650 | 0.000 | 48.940 | 0.000 | 0.000 | 0.000 |
| W0339 | 0.000 | 21.530 | 17.790 | 2.480 | 31.950 | 0.000 | 0.000 | 1.150 |
| W0445 | 0.000 | 0.000 | 3.370 | 0.000 | 13.290 | 0.000 | 0.000 | 0.000 |
| W0489 | 0.000 | 0.000 | 9.890 | 0.000 | 18.800 | 0.000 | 6.310 | 0.680 |
| W0490 | 0.000 | 22.250 | 22.230 | 2.900 | 34.290 | 0.000 | 0.000 | 0.800 |
| W0512 | 0.000 | 2.280 | 27.130 | 2.280 | 17.370 | 0.000 | 8.550 | 1.290 |

TABLE 24

| | Chlorophyll | Chlorophyll | Hydroxy- | Methyl | Pheophorbide | Pheophytin | |
|---|---|---|---|---|---|---|---|
| | a | b | chlorophyl a | Pheophorbide a | a | a | Unknown |
| WT | 7.459 | 0.000 | 0.000 | 0.000 | 0.594 | 4.294 | 0.929 |
| W0087 | 9.524 | 0.000 | 0.000 | 0.000 | 0.382 | 1.306 | 0.000 |
| W0139 | 6.413 | 0.000 | 0.000 | 0.239 | 0.000 | 1.651 | 0.000 |
| W0156 | 8.687 | 0.000 | 0.000 | 0.000 | 0.387 | 2.136 | 0.627 |
| W0159 | 18.651 | 3.929 | 2.763 | 0.000 | 0.413 | 3.197 | 0.000 |
| W0163 | 5.848 | 0.000 | 0.000 | 0.000 | 0.386 | 4.978 | 0.000 |
| W0167 | 16.085 | 0.000 | 0.000 | 0.000 | 0.000 | 1.277 | 0.000 |
| W0288 | 22.203 | 8.967 | 0.000 | 5.439 | 2.617 | 12.179 | 0.000 |
| W0320 | 15.331 | 8.309 | 3.561 | 11.671 | 2.732 | 11.401 | 0.000 |
| W0325 | 12.509 | 6.719 | 0.000 | 0.000 | 0.452 | 6.073 | 0.000 |
| W0339 | 12.762 | 4.740 | 0.000 | 0.000 | 0.000 | 1.229 | 0.000 |
| W0445 | 15.349 | 3.249 | 17.940 | 2.347 | 4.946 | 14.450 | 0.000 |
| W0489 | 19.500 | 5.649 | 0.000 | 0.000 | 0.000 | 15.738 | 0.000 |
| W0490 | 8.658 | 0.000 | 0.000 | 0.000 | 0.013 | 1.288 | 0.000 |
| W0512 | 12.512 | 0.000 | 0.000 | 0.000 | 0.176 | 2.783 | 0.000 |

### SUMMARY

Based on the process of wild type competition and regeneration of transgenic lines, 34 of 90 selected genes were validated as having a competitive growth advantage due to overexpression of the gene. These genes are listed in the table below.

TABLE 25

| Gene | | | Description (best arabidopsis | | |
|---|---|---|---|---|---|
| # | Winner | Locus ID | TAIR10 hit defline) | % CDS | Class |
| 2 | W0318 | Cre01.g000850 | | 100 | 3 |
| 6 | W0091 | Cre01.g059600 | Transport protein particle (TRAPP) | 75 | 3 |
| | | | component | | |
| 8 | W0422 | Cre02.g091100 | Ribosomal protein L23/L15e family | 100 | 3 |
| | | | protein | | |
| 9 | W0033 | Cre02.g106600 | Ribosomal protein S19e family | 100 | 1 |
| | | | protein | | |
| 10 | W0106 | Cre02.g114600 | 2-cysteine peroxiredoxin B | 56 | 3 |
| 11 | W0057 | Cre02.g120150 | ribulose bisphosphate carboxylase | 52 | 3 |
| | | | small chain 1A | | |
| 11 | W0255 | Cre02.g120150 | ribulose bisphosphate carboxylase | 100 | 1 |
| | | | small chain 1A | | |
| 13 | W0065 | Cre05.g234550 | fructose-bisphosphate aldolase 2 | 92 | 2 |
| 13 | W0335 | Cre05.g234550 | fructose-bisphosphate aldolase 2 | 100 | 1 |
| 14 | W0162 | Cre06g298650 | eukaryotic translation initiation | 95 | 2 |
| | | | factor 4A1 | | |
| 24 | W0018 | Cre13.g581650 | ribosomal protein L12-A | 67 | 3 |
| 25 | W0363 | Cre13.g590500 | fatty acid desaturase 6 | 100 | 5 |
| 25 | W0371 | Cre13.g590500 | fatty acid desaturase 6 | 57 | 3 |
| 26 | W0038 | Cre14.g621550 | thioredoxin M-type 4 | 11 | 2 |
| 32 | W0134 | Cre01.g010900 | glyceraldehyde-3-phosphate | 100 | 1 |
| | | | dehydrogenase B subunit | | |
| 32 | W0268 | Cre01.g010900 | glyceraldehyde-3-phosphate | 11 | 4 |
| | | | dehydrogenase B subunit | | |
| 34 | W0049 | Cre01.g043350 | Pheophorbide a oxygenase family | 0 | 3 |
| | | | protein with Rieske [2Fe—2S] domain | | |
| 35 | W0062 | Cre01.g050308 | Ribosomal protein L3 family protein | 70 | 1 |
| 36 | W0430 | Cre01.g072350 | SPFH/Band 7/PHB domain-containing | 100 | 2 |
| | | | membrane-associated protein family | | |
| 37 | W0190 | Cre02.g075700 | Ribosomal protein L19e family | 98 | 2 |
| | | | protein | | |
| 37 | W0462 | Cre02.g075700 | Ribosomal protein L19e family | 100 | 3 |
| | | | protein | | |
| 45 | W0058 | Cre03.g198000 | Protein phosphatase 2C family | 84 | 1 |
| | | | protein | | |
| 46 | W0149 | Cre03.g204250 | S-adenosyl-L-homocysteine hydrolase | 9 | 2 |
| 51 | W0325 | Cre09.g416500 | zinc finger (C2H2 type) family protein | 97 | 3 |
| 53 | W0167 | Cre10.g447950 | | 100 | 2 |
| 59 | W0024 | Cre12.g551451 | | 0 | 3 |
| 60 | W0150 | Cre13.g572300 | | 23 | 1 |
| 62 | W0445 | Cre14.g611150 | Small nuclear ribonucleoprotein | 10 | 2 |
| | | | family protein | | |
| 63 | W0282 | Cre14.g612800 | | 100 | 1 |
| 64 | W0351 | Cre14.g624000 | F-box/RNI-like superfamily protein | 100 | 2 |
| 66 | W0048 | Cre17.g722200 | mitochondrial ribosomal protein L11 | 100 | 2 |
| 68 | W0481 | Cre23.g766250 | photosystem II light harvesting | 12 | 2 |
| | | | complex gene 2.2 | | |
| 73 | W0172 | Cre02.g134700 | Ribosomal protein L4/L1 family | 36 | 3 |
| 74 | W0490 | Cre02.g139950 | | 100 | 3 |
| 75 | W0227 | Cre03.g210050 | Ribosomal protein L35 | 71 | 2 |
| 75 | W0343 | Cre03.g210050 | Ribosomal protein L35 | 100 | 5 |
| 82 | W0194 | Cre09.g386650 | ADP/ATP carrier 3 | 29 | 2 |
| 82 | W0475 | Cre09.g386650 | ADP/ATP carrier 3 | 100 | only primary |
| | | | | | data |
| 83 | W0087 | Cre10.g417700 | ribosomal protein 1 | 100 | 5 |
| 83 | W0355 | Cre10.g417700 | ribosomal protein 1 | 99 | 3 |
| 86 | W0489 | Cre12.g528750 | Ribosomal protein L11 family protein | 96 | 3 |
| 88 | W0201 | Cre17.g700750 | | 24 | 1 |
| 88 | W0211 | Cre17.g700750 | | 0 | 3 |
| 88 | W0496 | Cre17.g700750 | | 100 | 5 |

S. dimorphus
Transgenic S. dimorphus Lines Entering Validation Process

Eight of the 94 selected genes were represented by multiple winning transgenic lines containing different lengths of the CDS. These lines were considered to be non-identical and a representative winning line containing each fractional CDS was included in the validation process. Winning lines W0770 and W0771, despite different scaffold coordinates, have the same gene sequence and were thus consolidated as a single selected gene for regeneration. Two winners, W0687 and W1171, did not have viable original lines and were not included in the original line 1:1 competitions, but were regenerated by cloning the gene out of the cDNA library. Lastly, W0925 contained two independent insertion events of two different genes (g5205 and g5307). Each gene was considered selected and was individually regenerated, denoted by W09255 and W0925 L respectively, and included in 1:1 competitions. In all, 102 winner lines representing 94 selected genes entered the validation process.

Turbidostat Competitions with Original Lines

Starter cultures (5 ml) of each algae line were grown in TAP media to saturation in deep-well blocks. The cultures were then acclimated to HSM media by diluting back 1:10 in deep-well blocks. Cultures were grown two days in HSM media prior to inoculation in turbidostats. The wild type strain was treated in the same manner though at larger scale. For inoculation into turbidostats, OD₇₅₀ readings of wild type and selected gene cultures were taken and used to generate a mixed culture containing wild type and the transgenic line at a ratio of 9:1 with a final OD₇₅₀ of approximately 0.2. 10 ml of this mixture was used to inoculate turbidostats with a final volume of 30 ml. Four replicate turbidostats were inoculated from each winner line. The turbidostats were filled with HSM media and the gating density was set to an OD₇₅₀ of approximately 0.3 to maintain the culture at early- to mid-logarithmic growth. Constant light of ^{˜}150 μEinstein (μE) was provided, with a constant stream of 0.2% CO₂ bubbling into the culture.

A sample of the mixture used for turbidostat inoculation (time=0) was sorted using fluorescent-activated cell sorting (FACS) into 96-well microplates containing TAP media (four 96-well plates per sample). After ten days of turbidostat growth, a sample was taken and used for the same sorting procedure.

After approximately five days of growth, sorted plates were replicated onto solid TAP media containing 10 μg/ml hygromycin and 10 μg/ml paromomycin (to select for the transgenic line). Green wells in the sorted plates were counted to represent the total number of wild type and transgenic lines growing in permissive media and colonies on the replicated selective TAP plates were counted to represent the total number of transgenic lines. These numbers can then be used to calculate a selection coefficient as described previously for C. reinhardtii.

For en masse experiments, selected gene lines were grown to saturation in 5 ml cultures in TAP media. The cultures were then acclimated to HSM media by diluting back 1:10 in deep-well blocks. Cultures were grown two days in HSM media prior to inoculation in turbidostats. Cultures were normalized by OD₇₅₀ and pooled. This pooled mixture was sorted by FACS into 96-well microplates containing TAP media for a baseline reading of the distribution of genes. Twelve plates were sorted for baseline analysis at the time of turbidostat inoculation. Twelve replicate turbidostats were inoculated from this pool and cultured as before in HSM for two weeks. After two weeks, samples were taken from turbidostats and sorted into liquid cultures (four 96-well plates per turbidostat). After approximately five days of growth in 96-well plates, cultures were amplified by PCR and submitted for sequencing. Sanger reads were processed using CLC bio's Genomics Workbench software and a custom plugin. The plugin imports the data into the Genomic Workbench, trimming each sequence for quality and vector. The sequences are then compared to the Scenedesmus dimorphus genome using blastn. The gene locus for the top hit is determined and the relation of the BLAST hit and gene CDS was determined. A final result table was generated containing primarily the gene locus and how many times it was hit by a sequence within the dataset. These were compared to the gene loci identified in primary screening and winner numbers were assigned. The distribution of these genes can be compared between the baseline and the two week time point.

For en masse experiments, Selected Gene lines were grown to saturation in 5 ml cultures in TAP media. The cultures were then acclimated to HSM media by diluting back 1:10 in deep-well blocks. Cultures were grown two days in HSM media prior to inoculation in turbidostats. Cultures were normalized by OD₇₅₀ and pooled. This pooled mixture was sorted by FACS into 96-well microplates containing TAP media for a baseline reading of the distribution of genes. Twelve plates were sorted for baseline analysis at the time of turbidostat inoculation. Twelve replicate turbidostats were inoculated from this pool and cultured as before in HSM for two weeks. After two weeks, samples were taken from turbidostats and sorted into liquid cultures (four 96-well plates per turbidostat). After approximately five days of growth in 96-well plates, cultures were amplified by PCR and submitted for sequencing. Sanger reads were processed using CLC bio's Genomics Workbench software and a custom plugin developed specifically for this project. The plugin imports the data into the Genomic Workbench, trimming each sequence for quality and vector. The sequences are then compared to the Scenedesmus dimorphus genome using blastn (genome previously sequenced by Sapphire). The gene locus for the top hit is determined and the relation of the BLAST hit and gene CDS is determined. A final result table is generated containing primarily the gene locus and how many times it was hit by a sequence within the dataset. These were compared to the gene loci identified in primary screening and winner numbers were assigned. The distribution of these genes can be compared between the baseline and the two week time point.

### Regeneration of Lines

Cold Fusion technology (System Biosciences Inc, USA) was used to re-clone all the selected lines. This method allows cloning of PCR fragments via homology regions at each end of the PCR product and the linearized destination vector. The screening primers used earlier in the project for detection of cloned cDNA were used for this purpose. A vector was built that contains all the regions of the cDNA expression vector except the region between the sites homologous to the screening primers. This region was replaced with the restriction sites NdeI and SpeI (see FIG. 3). A further modification was also made to the expression vector by the addition of 1-CeuI sites flanking the entire cassette. These homing endonuclease sites facilitate linearization for transformation and since the recognition site is 29 base pairs in length it is unlikely to be found in any cDNA fragment cloned into the library.

Cell lysate of the original selected lines was used as PCR template for cloning. The cDNA shuttle vector was digested with NdeI and SpeI and purified by gel extraction. PCR product and linearized vector were used for the Cold Fusion reaction as per the manufacturer's guidelines. Cloning in this manner creates an expression cassette identical to the one found in the original lines. In the two cases where the original line was no longer available (W0687 and W1171), the cDNA insert was PCR amplified from the plasmid cDNA library originally used for primary screening and cloned into the cDNA overexpression vector (shown above). Cloned constructs were confirmed by DNA sequencing.

Re-cloned genes were transformed into Chlamydomonas reinhardtii CC-1690 and selected for resistance to both hygromycin and paromomycin (each at 10 μg/ml). For each gene, 36 transgenic lines were PCR screened and sequenced. Twelve sequence confirmed lines per gene were selected to enter turbidostats in competition with wild type. In six cases (W0677, W0934, W0936, W0950, W0967, and W0984), 11 lines were sequence confirmed and advanced.

Turbidostat Competitions with Regenerated Lines

Regenerated lines were grown in TAP media (1 ml) to saturation in 96-well deep-well blocks. The cultures were then acclimated to HSM media by diluting back 1:10 in 96-well deep-well blocks. Cultures were grown two days in HSM media prior to inoculation in turbidostats. The wild type strain was treated in the same manner though at larger scale. The twelve regenerated lines were normalized by OD₇₅₀ and pooled. The pooled mixture was then mixed at a ratio of 1:9 with the wild type strain at a final OD₇₅₀ of approximately 0.2. 10 ml of this mixture was used to inoculate turbidostats with a final volume of 30 ml. Four replicate turbidostats were inoculated from each regenerated winner. The turbidostats were filled with HSM media and set to an OD₇₅₀ of approximately 0.3, which represents an early- to mid-log growth phase. Constant light of ^{˜}150 μEinstein (μE) was provided, with a constant stream of 0.2% CO₂ bubbling into the culture.

A sample of each turbidostat at day 2 was sorted using FACS into 96-well microplates containing TAP media (four 96-well plates per sample). After fourteen days of turbidostat growth, a sample was taken and used for the same sorting procedure.

After approximately five days of growth, sorted plates were replicated onto solid TAP media containing 10 μg/ml hygromycin and 10 μg/ml paromomycin (to select for the transgenic line). Green wells in the sorted plates were counted to represent the total number of wild type and transgenic lines growing in permissive media and colonies on the replicated selective TAP plates were counted to represent the total number of transgenic lines. Selection coefficients were calculated as described above.

An additional en masse experiment using regenerated lines was completed. Regenerated lines were grown in TAP media (1 ml) to saturation in 96-well deep-well blocks. The cultures were then acclimated to HSM media by diluting back 1:10 in 96-well deep-well blocks. Cultures were grown two days in HSM media prior to inoculation in turbidostats. Cultures were normalized by OD₇₅₀ and pooled. This pooled mixture was sorted by FACS into 96-well liquid cultures for a baseline reading of the distribution of genes. Twelve plates were sorted for baseline analysis prior to entering turbidostats. Twelve replicate turbidostats were inoculated from this pool and cultured as before in HSM for two weeks. After samples were taken from turbidostats and sorted into 96-well liquid cultures (four plates per turbidostat). After approximately five days of growth in 96-well plates, cultures were amplified by PCR and submitted for sequencing. Analysis proceeded as described above.

### Growth and Photosynthesis Assays

Winner lines that advanced to the regeneration phase were analyzed by a high-throughput 96-well plate-based assay. Briefly, cultures were grown to stationary phase in TAP, MASM-NH₄Cl, or HSM media. Cultures were diluted to OD₇₅₀=0.2 and grown overnight. Overnight growth was followed by a second dilution to OD₇₅₀=0.05. These initial culture densities put the cells in lag or early log phase. At this point, 200 μl of each culture was added to a 96-well microtiter plate in randomized replicates. 96-well microtiter plates used in this assay contain opaque sides and a transparent base so that light exposure is equal across the entire plate. Plates were sealed using a PDMS lid in order to allow for gas exchange but minimize culture volume loss to evaporation. Sealed plates were then set onto a shaker within a growth chamber supplied with 5% CO₂. Intermittent shaking was set to occur for 15 s/min at 1700 rpm. Light incidence upon each plate lid was 125-130 μE. OD₇₅₀ was read every 6 hours for a maximum of 160 hours (until the cultures clearly enter stationary phase as evidenced by the leveling of the curve). The resulting OD₇₅₀ readings, which reflect culture growth, were plotted vs. time.

Selected Genes that advanced to the regeneration phase were also assessed for photosynthetic quantum yield using an IMAGING-PAM photosynthesis yield analyzer (Walz, Germany). The IMAGING-PAM works by pulsing cultures with saturating light, which briefly suppresses photochemical yield and induces maximal fluorescence yield. The Photosynthesis Yield Analyzer IMAGING-PAM specializes in the quick and reliable assessment of the effective quantum yield of photochemical energy conversion in photosynthesis. The fluorescence yield (F) and the maximal yield (Fₘ) are measured and the photosynthesis yield (Y=ΔF/Fₘ) is calculated. Samples were grown to mid-log phase in a 96-well deep-well block in either HSM or MASM-NH₄Cl and subsequently replicated on solid HSM or MASM-NH₄Cl media. Plates were incubated in a CO₂ controlled growth box under constant light of 80-100 [symbol] E for five days. Plates were analyzed with the MAXI IMAGING-PAM and ImageWin software.

Flow cytometry was used to determine cell size differences relative to wild type for all selected gene lines that advanced to the regeneration phase. The magnitude of the forward scatter is roughly proportional to the cell size. Therefore, the data can be used to distinguish which lines differ from wild type. Samples were grown to mid-log phase in HSM media under constant light of 80-100 μE in a CO₂ controlled growth box. Data was acquired using the BD Biosciences Influx cell sorter.

### Biochemical Assays

Selected genes that advanced to the regeneration phase were analyzed for increased lipid content by lipid dye staining. Briefly, cultures were grown to mid-log phase in MASM, TAP, or HSM media. 10 μl of culture was diluted in 200 μl of media and was stained with two dyes: Nile Red and Bodipy 493/503 (both of which stain neutral lipids). Stained samples were incubated at room temperature for 30 minutes and then processed by the Guava EasyCyte for fluorescent characteristics. Median fluorescence of each sample was used in calculations to determine fold change fluorescence in comparison to wild-type cultures.

### S Dimorphus Validation Results

### Original Line Competitions

Of the 102 selected lines, 100 were successfully competed against wild type in turbidostats. The calculated s values for one week of growth competition are shown in the graphs below. The majority of lines have an average positive s value in this experiment (85 lines). A one-sample, one-sided t-test was employed by calculating a 95% confidence interval (CI, α=0.025) from the standard deviation followed by comparison of this CI to the average. Any s measurements with a CI less than the average were determined to be statistically greater than zero. 20 lines passed this statistical test. 13 lines showed an s value of 0 or below for all replicates and are considered to have failed validation (W0610, W0673, W0729, W0800, W0819, W0827, W0873, W0923, W1010, W1076, W1084, W1094, W1202). Two other filters were applied to classify additional lines. Any line with only one replicate having a positive s value that is less than 0.01 did not advance (W0713, W1058, W1124). Any line with a replicate s value greater than zero obtained from five or fewer colonies must have had an additional replicate with a positive s value to advance. This rule was applied to eliminate any line advancing on data that may be considered noise (W1209). While these lines would normally not be carried forward to additional experiments, W1094 was regenerated and data shown where available. A few lines had negative mean s values but had individual replicates with positive values—these were advanced to the next stage of validation. In all, 17 lines representing 16 selected genes are considered to have failed validation following original line turbidostat competitions.

The original lines representing the selected genes were also run in an en masse competition experiment. All lines were combined in approximately equal amounts and allowed to grow and compete in replicate turbidostats for two weeks. Twenty lines showed a level of competitive advantage (relative to the population of all transgenic lines) in at least one of the replicates in the en masse pools. 3 of these lines are validated genes (W0667, W0785, W0979).

### Regenerated Line Competitions

Regenerated lines for all of the original winner lines representing 94 selected genes were created. 16 lines were regenerated but not screened due to poor performance in the competition of the original line with wild type (W0610, W0673, W0713, W0729, W0800, W0819, W0827, W0873, W0923, W1010, W1058, W1076, W1084, W1124, W1202, W1209). W0771 was regenerated and despite different scaffold coordinates, it is the same gene sequence as W0770 and did not proceed any further. All other regenerated lines entered into competitions with wild type in turbidostats.

The samples that entered turbidostat competition contained a pool of 12 transgenic lines unless noted previously. It is likely that only some of these lines are expressing the selected gene to a level sufficient to cause the phenotype of increased selection coefficient. The other lines within the pool could thus have no selective advantage over wild type in turbidostat growth or could be at a disadvantage. For this reason the competition was continued for fourteen days.

The table below incorporates the selection coefficients calculated from the original lines (mean and standard deviation) as well as the s calculations (mean and standard deviation) from the regenerated lines. Missing data represents original lines that were not available for screening or those lines that did not advance to the regenerated line competition phase.

TABLE 26

| | Original | | Regenerated | |
|---|---|---|---|---|
| | day 0-day 10 | | day 2-day 14 | |
| Line | s_{avg} | stdev | s_{avg} | stdev |
| W0601 | 0.1860 | 0.2371 | −0.0186 | 0.0365 |
| W0607 | 0.9255 | 0.0271 | −0.0146 | 0.0224 |
| W0610 | −0.0557 | 0.0497 | | |
| W0629 | 0.2387 | 0.1006 | −0.0061 | 0.0451 |
| W0647 | 0.6547 | 0.3511 | −0.0420 | 0.0341 |
| W0663 | 0.2710 | 0.1141 | −0.0773 | 0.1112 |
| W0667 | 0.4874 | 0.3940 | −0.0155 | 0.0911 |
| W0670 | −0.1246 | 0.1356 | −0.0578 | 0.0328 |
| W0673 | −0.2018 | 0.1055 | | |
| W0674 | 0.3515 | 0.2701 | −0.0532 | 0.0597 |
| W0675 | 0.2283 | 0.0781 | −0.0291 | 0.0306 |
| W0677 | 0.1880 | 0.4192 | −0.0440 | 0.0269 |
| W0687 | | | 0.0116 | 0.0410 |
| W0702 | 0.1619 | 0.1323 | −0.0742 | 0.0226 |
| W0709 | 0.4420 | 0.2625 | −0.0651 | 0.1281 |
| W0713 | −0.1005 | 0.0809 | | |
| W0729 | −0.2557 | 0.0265 | | |
| W0752 | 0.0472 | 0.0296 | −0.0271 | 0.0301 |
| W0757 | −0.0006 | 0.0542 | 0.0670 | 0.1431 |
| W0758 | 0.1593 | 0.0738 | −0.0787 | 0.0704 |
| W0770 | 0.5818 | 0.2188 | 0.0703 | 0.1759 |
| W0771 | 0.1614 | 0.4611 | | |
| W0774 | 0.2539 | 0.3491 | −0.0025 | 0.0552 |
| W0775 | 0.4824 | 0.4818 | −0.0093 | 0.0412 |
| W0776 | 0.3438 | 0.3225 | 0.0514 | 0.0377 |
| W0785 | 0.2839 | 0.0918 | −0.0084 | 0.0511 |
| W0793 | 0.2812 | 0.4884 | −0.0096 | 0.0288 |
| W0798 | 0.3122 | 0.2593 | −0.0705 | 0.0851 |
| W0800 | −0.2448 | 0.0734 | | |
| W0801 | −0.0648 | 0.0786 | −0.0132 | 0.0244 |
| W0802 | 0.3771 | 0.3932 | −0.0164 | 0.1142 |
| W0819 | −0.1102 | 0.0570 | | |
| W0823 | 0.1577 | 0.0602 | −0.0394 | 0.0527 |
| W0825 | 0.0195 | 0.0692 | −0.0387 | 0.0131 |
| W0827 | −0.1960 | 0.0509 | | |
| W0828 | 0.3890 | 0.1722 | −0.0220 | 0.0114 |
| W0829 | 0.2811 | 0.2320 | −0.0184 | 0.0522 |
| W0832 | 0.3439 | 0.1895 | −0.0285 | 0.0094 |
| W0841 | 0.1662 | 0.0849 | −0.0145 | 0.0524 |
| W0846 | −0.1099 | 0.0959 | −0.0512 | 0.0357 |
| W0857 | 0.5765 | 0.5118 | −0.0672 | 0.0316 |
| W0871 | −0.0028 | 0.2900 | 0.1707 | 0.2106 |
| W0873 | −0.2854 | 0.1754 | | |
| W0883 | 0.2734 | 0.2583 | 0.2741 | 0.0229 |
| W0894 | 0.0052 | 0.1110 | −0.0355 | 0.0567 |
| W0905 | 0.0603 | 0.2935 | −0.0189 | 0.0216 |
| W0913 | 0.0574 | 0.2810 | −0.0855 | 0.0866 |
| W0923 | −0.3923 | 0.0335 | | |
| W0925 | 0.2285 | 0.2757 | | |
| W09255 | | | −0.0615 | 0.0894 |
| W0925L | | | −0.0191 | 0.0700 |
| W0929 | −0.0379 | 0.2062 | −0.0172 | 0.0250 |
| W0931 | −0.0897 | 0.0863 | −0.0401 | 0.0224 |
| W0934 | 0.0875 | 0.0691 | 0.0886 | 0.0248 |
| W0936 | −0.1019 | 0.1286 | −0.0330 | 0.0455 |
| W0942 | 0.0701 | 0.1542 | −0.0102 | 0.0389 |
| W0949 | 0.5089 | 0.1335 | 0.0476 | 0.0316 |
| W0950 | 0.0896 | 0.3179 | 0.0151 | 0.0336 |
| W0956 | 0.2239 | 0.0502 | 0.0075 | 0.0648 |
| W0965 | 0.3735 | 0.3698 | −0.0084 | 0.0271 |
| W0967 | 0.1122 | 0.2423 | −0.0861 | 0.0212 |
| W0968 | 0.1666 | 0.0554 | −0.0323 | 0.0147 |
| W0977 | −0.1210 | 0.1679 | −0.0102 | 0.0523 |
| W0979 | 0.2584 | 0.3285 | 0.0336 | 0.0285 |
| W0980 | 0.2657 | 0.0966 | −0.0382 | 0.0273 |
| W0981 | 0.4276 | 0.3828 | −0.0284 | 0.0204 |
| W0982 | 0.2176 | 0.1275 | −0.0498 | 0.0216 |
| W0983 | 0.1179 | 0.0874 | −0.0539 | 0.0605 |
| W0984 | 0.4459 | 0.0976 | −0.0554 | 0.0056 |
| W0994 | 0.0833 | 0.0961 | −0.0699 | 0.0394 |
| W1002 | 0.2353 | 0.3068 | −0.0322 | 0.0243 |
| W1004 | 0.3746 | 0.1777 | −0.0027 | 0.0403 |
| W1010 | −0.2136 | 0.1107 | | |
| W1036 | 0.0529 | 0.1483 | 0.0350 | 0.0493 |
| W1039 | 0.0066 | 0.1259 | −0.0162 | 0.1088 |
| W1040 | 0.2049 | 0.0303 | −0.0579 | 0.0066 |
| W1058 | −0.0216 | 0.0340 | | |
| W1064 | 0.0806 | 0.0731 | −0.0282 | 0.0185 |
| W1071 | 0.0099 | 0.0334 | −0.0405 | 0.0181 |
| W1076 | −0.1045 | 0.0645 | | |
| W1083 | 0.0725 | 0.2307 | −0.0222 | 0.0580 |
| W1084 | −0.1472 | 0.0460 | | |
| W1092 | 0.1009 | 0.2290 | 0.0021 | 0.0307 |
| W1094 | −0.2178 | 0.0515 | −0.0571 | 0.0553 |
| W1097 | 0.0817 | 0.1888 | −0.0496 | 0.0467 |
| W1104 | 0.4774 | 0.2000 | −0.0350 | 0.0418 |
| W1117 | 0.1495 | 0.0736 | −0.0227 | 0.0253 |
| W1118 | −0.0305 | 0.0930 | −0.0286 | 0.0410 |
| W1123 | 0.1170 | 0.1880 | 0.1178 | 0.0346 |
| W1124 | −0.0889 | 0.0776 | | |
| W1137 | 0.3100 | 0.1679 | −0.0758 | 0.0896 |
| W1146 | 0.0608 | 0.0438 | 0.0302 | 0.0369 |
| W1171 | | | −0.0401 | 0.0235 |
| W1182 | 0.0072 | 0.0366 | 0.0355 | 0.0367 |
| W1187 | 0.0459 | 0.0977 | −0.0186 | 0.0254 |
| W1192 | 0.0011 | 0.0423 | −0.0665 | 0.0686 |
| W1197 | 0.4619 | 0.3591 | −0.1122 | 0.0957 |
| W1202 | −0.2160 | 0.0992 | | |
| W1203 | 0.5441 | 0.1586 | 0.0007 | 0.0394 |
| W1208 | 0.1246 | 0.2636 | −0.0058 | 0.0324 |
| W1209 | 0.0133 | 0.0345 | | |
| W1210 | 0.3206 | 0.0834 | −0.0116 | 0.0242 |
| W1227 | 0.3757 | 0.3110 | −0.0299 | 0.0176 |
| W1233 | 0.0618 | 0.1370 | 0.1134 | 0.0642 |
| W1235 | −0.0362 | 0.0968 | −0.0560 | 0.0067 |

The regenerated lines were also run in an en masse competition experiment. All lines were combined in approximately equal amounts and allowed to grow and compete in replicate turbidostats. Samples were taken two weeks after setup. 13 lines showed a consistent level of competitive advantage (relative to the population of all transgenic lines) across all the replicates in the en masse pools. Nine of these lines were considered validated genes (W0883, W0934, W1004, W1036, W1083, W1104, W1123, W1210, W1233).

### Validated Genes

The data for the selection coefficients divided the winner lines into five classes. In general, the s value from the original line is a better representation of the selective advantage of a gene. Regenerated line data, because it results from the combined phenotype of 12 independent clones, is less representative of absolute selective advantage and is more of a binary test to confirm that the original line data is due solely to selected gene expression. Class 1 includes those lines that had original lines that were significantly greater than 0 (95% confidence interval as described previously) and regenerated lines that had positive s average values. This class contains 3 lines (W0770, W0949, W1203) representing 3 selected genes that are considered validated with very high confidence.

Class 2 includes lines that had original lines that were significantly greater than 0 and at least one regenerated line replicate with a positive s value. This class contains 10 lines (W0607, W0629, W0675, W0785, W0823, W0956, W0980, W1004, W1104, W1210). These Selected Genes represented by Class 2 are considered validated with a high degree of confidence.

Class 3 includes lines that had average s values greater than 0.05 for both the original and regenerated lines. This class contains 5 lines (W0776, W0883, W0934, W1123, W1233), one of which is represented in Class 1. Class 4 includes those lines with average s values greater than 0.05 for the original lines and average s values greater than 0 for the regenerated line. This class contains 5 lines (W0950, W0979, W1036, W1092, W1146). Finally, Class 5 includes lines with average s values greater than 0.05 for the original lines and a minimum of one regenerated line replicate with a s value greater than 0.05. This class contains 6 lines (W0667, W0774, W0802, W0829, W0841, W1083), one of which is represented by a Selected Gene in Class 2. In all, 27 genes are considered validated.

11 validated genes were represented by more than one winner from the primary screen. Furthermore, 4 of these 11 genes have winning lines that contain predicted coding sequences of different lengths. Locus ID g9576 (W1004, W1083) has lines of 100% and 19% CDS and both were validated in Class 2 and Class 5 respectively. Similarly, locus ID g13997 (W0934, W1203) has lines of 93% and 100% CDS that were also validated. The third gene, locus ID g17628, has lines of 100% and 58% CDS. The line containing 58% CDS (W0950) has been validated in Class 4. However, the line containing 100% CDS (W0923) had s values that were less than zero for all four replicates in the original line turbidostat competitions and did not advance any further in the validation process. This example suggests a truncated form of the protein or some gene regulatory mechanism may be responsible for the observed phenotype. Locus ID g14780 (W0677, W0776) is similar to the preceding example such that it has lines of 100% and 46% CDS, but only the shorter gene was validated.

During the primary screen, a winning line (W0925) was identified that contains two individual genes. PCR amplification of a pooled turbidostat competition resulted in a doublet when visualized by agarose gel electrophoresis. Several winning lines were successively plated on solid media to isolate single colonies. Repeated amplification of the doublet and sequence identification of both bands suggested that two independent integration events occurred in the same cell. The original winning line derived from the primary screen was treated as a single selected gene, but each gene was considered selected and regenerated separately. The regenerated lines were referred to as W0925S (locus ID g5205) and W0925 L (locus ID g5307) to represent the small and large gene sizes observed from PCR amplification. When competed against wild type, the original line had an average s value of 0.2284, but was not statistically different than 0 due to its large standard deviation. Neither regenerated line had data to suggest it was the dominant gene of the two. All four replicate s values of W0925 L were less than zero and W0925S had a negative average s value. This Selected Gene was not considered validated.

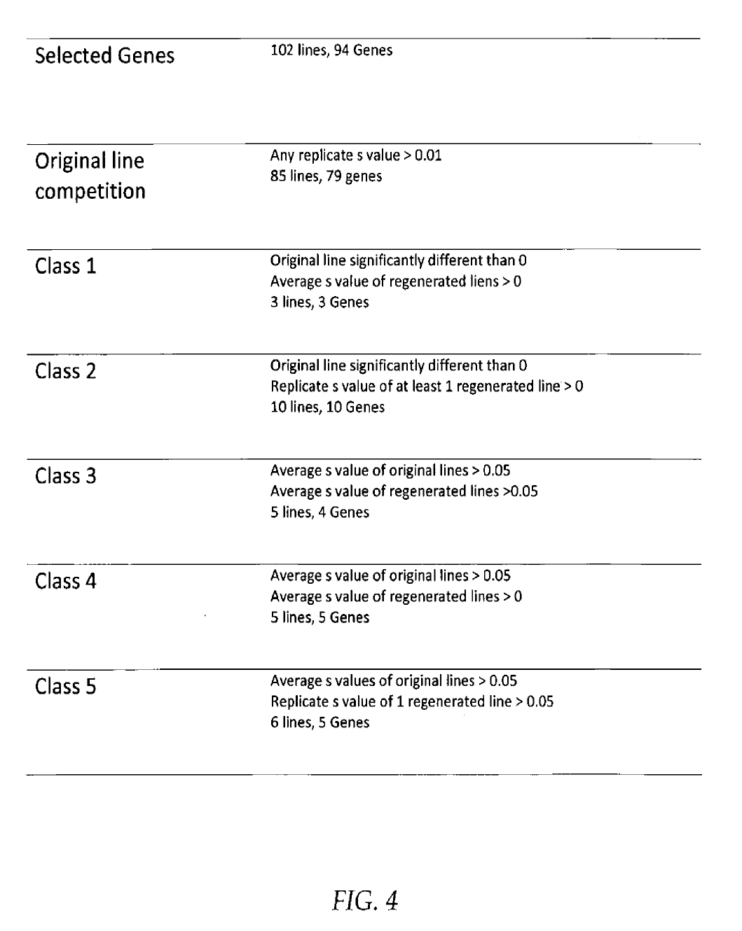

The validation process for S. dimorphus genes is reflected in FIG. 4. The table below lists all 94 selected genes and the winner lines representing them, along with the Class to which they are assigned. Winner lines that contain the same gene are listed together. 27 of these selected genes are considered validated, and are indicated by bold text in the Locus ID column.

TABLE 27

| Gene | Winner | Locus ID | C. reinhardtii Description | % CDS | Class |
|---|---|---|---|---|---|
| 1 | W1210 | g16071 | | 100 | 2 |
| 2 | W0729 | g17973 | ribosomal protein L5 B | 25 | |
| 3 | W1058 | g18243 | Nucleoside diphosphate kinase family protein; | 100 | |
| | | | Tetratricopeptide repeat (TPR)-like superfamily | | |
| | | | protein | | |
| 4 | W0929 | g195 | | 95 | |
| 5 | W1137 | g2549 | Ribosomal protein L19 family protein | 83 | |
| 6 | W1076 | g4589 | | 100 | |
| 7 | W1080 | g5150 | 2Fe—2S ferredoxin-like superfamily protein | 100 | |
| 7 | W1097 | g5150 | 2Fe—2S ferredoxin-like superfamily protein | 100 | |
| 7 | W1140 | g5150 | 2Fe—2S ferredoxin-like superfamily protein | 100 | |
| 8 | W0801 | g6846 | | 81 | |
| 9 | W0674 | g764 | NADH dehydrogenase subunit 9 | 52 | |
| 10 | W0828 | g8032 | PS II oxygen-evolving complex 1 | 39 | |
| 11 | W0931 | g9484 | Mechanosensitive ion channel protein; Protein | 58 | |
| | | | kinase superfamily protein; Outward rectifying | | |
| | | | potassium channel protein; LEUNIG_homolog; | | |
| | | | DERLIN-1 | | |
| 12 | W1071 | scaffold10: 905619-906481 | | | |
| 13 | W0829 | scaffold110: 302109-303275 | | | 5 |
| 13 | W1155 | scaffold110: 302109-303275 | | | |
| 13 | W1170 | scaffold110: 302109-303275 | | | |
| 13 | W1176 | scaffold110: 302109-303275 | | | |
| 14 | W0967 | scaffold131: 485473-486287 | | | |
| 15 | W1084 | scaffold152: 341659-342590 | | | |
| 16 | W1227 | scaffold178: 604743-605443 | | | |
| 16 | W1215 | scaffold178: 604743-605443 | | | |
| 17 | W1010 | scaffold18: 836026-836584 | | | |
| 18 | W0610 | scaffold185: 45139-46581 | | | |
| 19 | W0774 | scaffold42: 463800-464650 | | | 5 |
| 20 | W1183 | scaffold43: 818145-818878 | | | |
| 20 | W1208 | scaffold43: 818145-818878 | | | |
| 21 | W1209 | scaffold48: 103563-104365 | | | |
| 22 | W0977 | scaffold56: 1559519-1560130 | | | |
| 23 | W1002 | scaffold70: 617462-618203 | | | |
| 24 | W0994 | scaffold82: 654412-655260 | | | |
| 25 | W0713 | scaffold9: 1148396-1149053 | | | |
| 26 | W0647 | scaffold9: 1498620-1499365 | | | |
| 27 | W1094 | g11979 | GRIM-19 protein | 100 | |
| 28 | W0785 | g12290 | | 100 | 2 |
| 28 | W1169 | g12290 | | 100 | |
| 29 | W0601 | g13638 | senescence-associated gene 29 | 2 | |
| 30 | W0611 | g14780 | ribulose bisphosphate carboxylase small chain | 100 | |
| | | | 1A; Cyclin family protein | | |
| 30 | W0677 | g14780 | ribulose bisphosphate carboxylase small chain | 100 | |
| | | | 1A; Cyclin family protein | | |
| 30 | W0723 | g14780 | ribulose bisphosphate carboxylase small chain | 100 | |
| | | | 1A; Cyclin family protein | | |
| 30 | W0776 | g14780 | ribulose bisphosphate carboxylase small chain | 46 | 3 |
| | | | 1A; Cyclin family protein | | |
| 30 | W0805 | g14780 | ribulose bisphosphate carboxylase small chain | 100 | |
| | | | 1A; Cyclin family protein | | |
| 30 | W0912 | g14780 | ribulose bisphosphate carboxylase small chain | 100 | |
| | | | 1A; Cyclin family protein | | |
| 30 | W0951 | g14780 | ribulose bisphosphate carboxylase small chain | 100 | |
| | | | 1A; Cyclin family protein | | |
| 31 | W1123 | g1509 | Protein kinase superfamily protein with | 100 | 3 |
| | | | octicosapeptide/Phox/Bem1p domain | | |
| 32 | W0894 | g17352 | | 100 | |
| 33 | W0956 | g18330 | Protein kinase superfamily protein | 42 | 2 |
| 34 | W0857 | g2142 | | 100 | |
| 35 | W0798 | g2798 | | 13 | |
| 36 | W0687 | g2831 | | 38 | |
| 36 | W0974 | g2831 | | 100 | |
| 36 | W0981 | g2831 | | 100 | |
| 37 | W0757 | g3360 | | 4 | |
| 38 | W0936 | g3478 | FKBP-like peptidyl-prolyl cis-trans isomerase | 100 | |
| | | | family protein | | |
| 39 | W0607 | g3921 | ubiquitin-associated (UBA)/TS-N domain- | 100 | 2 |
| | | | containing protein | | |
| 39 | W0626 | g3921 | ubiquitin-associated (UBA)/TS-N domain- | 100 | |
| | | | containing protein | | |
| 40 | W0825 | g409 | | 100 | |
| 41 | W0871 | g4764 | | 100 | |
| 42 | W0925S | g5205 | mRNA capping enzyme family protein | 26 | |
| 43 | W0925L | g5307 | Aha1 domain-containing protein | 100 | |
| 44 | W0979 | g664 | Nucleic acid-binding, OB-fold-like protein | 100 | 4 |
| 45 | W1233 | g7387 | demeter-like 2 | 100 | 3 |
| 46 | W0913 | g7755 | Chlorophyll A-B binding family protein | 80 | |
| 47 | W1100 | g884 | | 100 | |
| 47 | W1104 | g884 | | 100 | 2 |
| 48 | W1004 | g9576 | photosystem II subunit Q-2 | 97 | 2 |
| 48 | W1083 | g9576 | photosystem II subunit Q-2 | 19 | 5 |
| 48 | W0932 | g9576 | photosystem II subunit Q-2 | 97 | |
| 48 | W1098 | g9576 | photosystem II subunit Q-2 | 19 | |
| 49 | W0832 | scaffold107: 31016-31748 | | | |
| 50 | W0965 | scaffold108: 15239-16070 | | | |
| 51 | W1182 | scaffold110: 1538332-1539144 | | | |
| 52 | W0971 | scaffold119: 1014531-1015301 | | | |
| 52 | W0975 | scaffold119: 1014531-1015301 | | | |
| 52 | W0982 | scaffold119: 1014531-1015301 | | | |
| 52 | W0988 | scaffold119: 1014531-1015301 | | | |
| 53 | W0667 | scaffold126: 355759-356343 | | | 5 |
| 54 | W0770 | scaffold18: 1489301-1489559 | | | 1 |
| 54 | W0771 | scaffold18: 1494447-1495555 | | | |
| 55 | W1197 | scaffold187: 101177-101934 | | | |
| 56 | W0673 | scaffold239: 234823-235585 | | | |
| 57 | W0802 | scaffold33: 535965-537528 | | | 5 |
| 58 | W0758 | scaffold419: 37021-37461 | | | |
| 59 | W1124 | scaffold48: 1027034-1027677 | | | |
| 60 | W1092 | scaffold64: 287639-288387 | | | 4 |
| 61 | W0968 | scaffold70: 188310-189043 | | | |
| 62 | W0827 | scaffold99: 550309-551108 | | | |
| 63 | W0800 | g13463 | Zincin-like metalloproteases family protein | 11 | |
| 64 | W0675 | g14907 | | 100 | 2 |
| 65 | W0949 | g14943 | ATP synthase delta-subunit gene | 100 | 1 |
| 66 | W0635 | g16080 | Ribosomal L28e protein family | 100 | |
| 66 | W0650 | g16080 | Ribosomal L28e protein family | 100 | |
| 66 | W0702 | g16080 | Ribosomal L28e protein family | 100 | |
| 67 | W0883 | g18194 | gamma carbonic anhydrase like 1 | 100 | 3 |
| 68 | W1202 | g2708 | Ribosomal protein L10 family protein | 39 | |
| 69 | W0905 | g8071 | LYR family of Fe/S cluster biogenesis protein | 100 | |
| 70 | W0752 | g9102 | subtilisin-like serine protease 3; high | 100 | |
| | | | chlorophyll fluorescence phenotype 173 | | |
| 71 | W0873 | scaffold145: 369643-370825 | | | |
| 72 | W0980 | scaffold240: 19496-20329 | | 2 | |
| 73 | W0983 | scaffold292: 8940-9640 | | | |
| 74 | W0793 | scaffold54: 373084-373489 | | | |
| 74 | W1154 | scaffold54: 373084-373489 | | | |
| 74 | W1179 | scaffold54: 373084-373489 | | | |
| 75 | W0686 | g10777 | | 100 | |
| 75 | W0714 | g10777 | | 100 | |
| 75 | W1192 | g10777 | | 100 | |
| 76 | W1187 | g11681 | | 100 | |
| 76 | W0838 | g11681 | | 100 | |
| 76 | W0844 | g11681 | | 100 | |
| 77 | W0728 | g12727 | FK506- and rapamycin-binding protein 15 kD-2 | 6 | |
| 77 | W0753 | g12727 | FK506- and rapamycin-binding protein 15 kD-2 | 6 | |
| 77 | W0755 | g12727 | FK506- and rapamycin-binding protein 15 kD-2 | 6 | |
| 77 | W1118 | g12727 | FK506- and rapamycin-binding protein 15 kD-2 | 100 | |
| 78 | W1036 | g13214 | | 3 | 4 |
| 79 | W0709 | g15296 | Ribosomal protein L13 family protein | 100 | |
| 79 | W1014 | g15296 | Ribosomal protein L13 family protein | 100 | |
| 79 | W1074 | g15296 | Ribosomal protein L13 family protein | 100 | |
| 80 | W0923 | g17628 | receptor for activated C kinase 1C | 100 | |
| 80 | W0950 | g17628 | receptor for activated C kinase 1C | 58 | 4 |
| 81 | W0819 | g2176 | NagB/RpiA/CoA transferase-like superfamily | 100 | |
| | | | protein | | |
| 82 | W0841 | g4280 | | 100 | 5 |
| 83 | W0775 | g7811 | Leucine-rich repeat transmembrane protein | 4 | |
| | | | kinase | | |
| 84 | W1146 | g8264 | | 26 | 4 |
| 85 | W0823 | scaffold67: 222004-223125 | | | 2 |
| 85 | W0916 | scaffold67: 222004-223125 | | | |
| 86 | W0670 | scaffold99: 669053-669536 | | | |
| 87 | W0937 | g10479 | photosystem II light harvesting complex gene | 100 | |
| | | | 2.2 | | |
| 87 | W0942 | g10479 | photosystem II light harvesting complex gene | 36 | |
| | | | 2.2 | | |
| 87 | W0984 | g10479 | photosystem II light harvesting complex gene | 100 | |
| | | | 2.2 | | |
| 88 | W0846 | g13646 | acyl carrier protein 1 | 97 | |
| 88 | W0848 | g13646 | acyl carrier protein 1 | 97 | |
| 88 | W0973 | g13646 | acyl carrier protein 1 | 97 | |
| 88 | W1039 | g13646 | acyl carrier protein 1 | 100 | |
| 88 | W1047 | g13646 | acyl carrier protein 1 | 100 | |
| 89 | W0659 | g13997 | aldehyde dehydrogenase 2C4 | 100 | |
| 89 | W0796 | g13997 | aldehyde dehydrogenase 2C4 | 100 | |
| 89 | W0934 | g13997 | aldehyde dehydrogenase 2C4 | 93 | 3 |
| 89 | W1203 | g13997 | aldehyde dehydrogenase 2C4 | 100 | 1 |
| 90 | W1064 | g14035 | | 100 | |
| 91 | W0629 | g2506 | photosystem II subunit X | 100 | 2 |
| 91 | W0924 | g2506 | photosystem II subunit X | 100 | |
| 91 | W1028 | g2506 | photosystem II subunit X | 100 | |
| 91 | W1115 | g2506 | photosystem II subunit X | 100 | |
| 92 | W1117 | g3574 | ribosomal protein L4 | 21 | |
| 92 | W1156 | g3574 | ribosomal protein L4 | 63 | |
| 92 | W1171 | g3574 | ribosomal protein L4 | 63 | |
| 92 | W1173 | g3574 | ribosomal protein L4 | 63 | |
| 93 | W0663 | g4729 | Ribosomal protein L31e family protein | 100 | |
| 93 | W0969 | g4729 | Ribosomal protein L31e family protein | 100 | |
| 93 | W0987 | g4729 | Ribosomal protein L31e family protein | 100 | |
| 94 | W0966 | g5891 | Ribosomal protein L6 family protein | 100 | |
| 94 | W0978 | g5891 | Ribosomal protein L6 family protein | 100 | |
| 94 | W1040 | g5891 | Ribosomal protein L6 family protein | 100 | |
| 94 | W1134 | g5891 | Ribosomal protein L6 family protein | 100 | |
| 94 | W1139 | g5891 | Ribosomal protein L6 family protein | 100 | |
| 95 | W1151 | scaffold176: 330612-331330 | | | |
| 95 | W1221 | scaffold176: 330612-331330 | | | |
| 95 | W1235 | scaffold176: 330612-331330 | | | |

In order to further rank and distinguish winner lines and selected genes from each other, an ANOVA with Tukey-Kramer HSD test was completed on each set of selection coefficient data. This test is a single-step multiple comparison procedure and statistical test to find which means are significantly different from one another. The test compares the means of every sample to the means of every other sample; that is, it applies simultaneously to the set of all pairwise comparisons and identifies where the difference between two means is greater than the standard error would be expected to allow.

### Growth and Biochemical Characteristics

Selected genes that were carried forward after initial turbidostat competitions (84 lines) were tested in microtiter plate growth assays using three different media: HSM, MASM, and TAP. HSM and MASM are both minimal medias with different nitrogen sources (NH₄ for HSM, NO₃ for MASM) while TAP contains an organic carbon source (acetate) and supports mixotrophic growth.

The OD₇₅₀ versus time data were not suitable for logistic curve fitting for all wells. Therefore, an exponential analysis was performed in order to calculate growth rates. With this type of analysis, the OD₇₅₀ data were natural log transformed, and plotted with time. Then, the linear region of these data was selected to define the log phase growth region of the curve. The most difficult part of this type of analysis was to determine which data represent the linear region. This experiment studied clones having different growth profiles; therefore a subjective time range to analyze was not suitable. In order to overcome this challenge, an algorithm for selecting the linear region of the In (OD₇₅₀) versus time data was developed and programmed into MS Excel VBA to analyze the data.

The linear selection algorithm uses a two phase process. Phase one of the algorithm steps through all the transformed data using all possible starting points and between 4 and 7 consecutive points to calculate the Slope, R², and the t value of the slope. Any slopes failing the t-test were rejected, α=0.05 confidence level (Kachigan. Multivariate Statistical Analysis, 2^{nd} Ed. (1991) ISBN 0-942154-91-6; p178). Of the slopes which had a significant value by the t-test, the one having the maximum product of Slope*R² was selected as representing the linear region. The slope of this linear region was used to score the growth rates of the clone. Growth rate for each well was determined independently. These resulting growth rates were then analyzed using JMP® software (SAS Institute, Inc., Cary, N.C.).

Below is a summary table for the microtiter plate experiments. An ANOVA with Dunnett's statistic test (p<0.05) was applied to the samples to determine which were significantly different than wild type. Those lines that are statistically different than wild type are highlighted in bold text below. W1210 is not included in this analysis due to low density of the starter culture.

Table 28

| | HSM | | MASM | | TAP | |
|---|---|---|---|---|---|---|
| Winner | Mean | Stdev | Mean | stdev | Mean | stdev |
| W0601 | 0.1073 | 0.0122 | 0.1053 | 0.0251 | 0.1112 | 0.0043 |
| W0607 | 0.1145 | 0.0152 | 0.0721 | 0.0296 | 0.1376 | 0.0133 |
| W0629 | 0.1236 | 0.0167 | 0.1139 | 0.0042 | 0.1453 | 0.0141 |
| W0647 | 0.1148 | 0.0063 | 0.0876 | 0.0186 | 0.1368 | 0.0046 |
| W0663 | 0.1196 | 0.0230 | 0.1187 | 0.0038 | 0.2033 | 0.0448 |
| W0667 | 0.1234 | 0.0190 | 0.1104 | 0.0065 | 0.1679 | 0.0108 |
| W0670 | 0.1041 | 0.0044 | 0.0479 | 0.0075 | 0.1332 | 0.0018 |
| W0674 | 0.0939 | 0.0098 | 0.0885 | 0.0167 | 0.1072 | 0.0164 |
| W0675 | 0.1154 | 0.0107 | 0.1203 | 0.0067 | 0.1592 | 0.0092 |
| W0677 | 0.0978 | 0.0050 | 0.1142 | 0.0029 | 0.1295 | 0.0067 |
| W0702 | 0.1261 | 0.0123 | 0.1251 | 0.0103 | 0.1380 | 0.0110 |
| W0709 | 0.1174 | 0.0026 | 0.0772 | 0.0239 | 0.1286 | 0.0183 |
| W0752 | 0.1148 | 0.0229 | 0.1039 | 0.0159 | 0.1336 | 0.0093 |
| W0757 | 0.1252 | 0.0082 | 0.1169 | 0.0039 | 0.1349 | 0.0080 |
| W0758 | 0.1179 | 0.0052 | 0.1043 | 0.0050 | 0.1374 | 0.0092 |
| W0770 | 0.1141 | 0.0062 | 0.0974 | 0.0145 | 0.1224 | 0.0043 |
| W0774 | 0.1240 | 0.0050 | 0.1151 | 0.0080 | 0.1342 | 0.0176 |
| W0775 | 0.1126 | 0.0036 | 0.1019 | 0.0125 | 0.1230 | 0.0085 |
| W0776 | 0.1173 | 0.0048 | 0.1173 | 0.0054 | 0.1285 | 0.0083 |
| W0785 | 0.0953 | 0.0088 | 0.1089 | 0.0143 | 0.1283 | 0.0163 |
| W0793 | 0.1020 | 0.0066 | 0.0923 | 0.0153 | 0.1179 | 0.0115 |
| W0798 | 0.0908 | 0.0115 | 0.0939 | 0.0191 | 0.1272 | 0.0064 |
| W0801 | 0.1152 | 0.0058 | 0.1065 | 0.0097 | 0.1381 | 0.0063 |
| W0802 | 0.1063 | 0.0107 | 0.0752 | 0.0346 | 0.1221 | 0.0087 |
| W0823 | 0.1130 | 0.0091 | 0.1214 | 0.0045 | 0.1375 | 0.0161 |
| W0825 | 0.0827 | 0.0056 | 0.0974 | 0.0077 | 0.1509 | 0.0106 |
| W0828 | 0.0903 | 0.0137 | 0.0844 | 0.0139 | 0.1067 | 0.0108 |
| W0829 | 0.0747 | 0.0125 | 0.1195 | 0.0058 | 0.1115 | 0.0153 |
| W0832 | 0.1119 | 0.0041 | 0.1086 | 0.0046 | 0.1231 | 0.0140 |
| W0841 | 0.1698 | 0.0209 | 0.1335 | 0.0083 | 0.1815 | 0.0303 |
| W0846 | 0.0965 | 0.0088 | 0.1156 | 0.0152 | 0.1312 | 0.0088 |
| W0857 | 0.1034 | 0.0071 | 0.0765 | 0.0297 | 0.1234 | 0.0057 |
| W0871 | 0.1006 | 0.0039 | 0.1052 | 0.0076 | 0.1309 | 0.0062 |
| W0883 | 0.1230 | 0.0040 | 0.1128 | 0.0028 | 0.1506 | 0.0102 |
| W0894 | 0.1083 | 0.0114 | 0.1110 | 0.0037 | 0.1307 | 0.0110 |
| W0905 | 0.1115 | 0.0050 | 0.0885 | 0.0070 | 0.1533 | 0.0149 |
| W0913 | 0.0990 | 0.0168 | 0.1155 | 0.0084 | 0.1291 | 0.0206 |
| W0925 | 0.1103 | 0.0094 | 0.1185 | 0.0079 | 0.1477 | 0.0105 |
| W0929 | 0.1144 | 0.0075 | 0.1075 | 0.0132 | 0.1481 | 0.0069 |
| W0931 | 0.1341 | 0.0058 | 0.1193 | 0.0017 | 0.1585 | 0.0090 |
| W0934 | 0.1327 | 0.0256 | 0.1050 | 0.0050 | 0.1534 | 0.0135 |
| W0936 | 0.1195 | 0.0031 | 0.1193 | 0.0028 | 0.1427 | 0.0070 |
| W0942 | 0.1116 | 0.0075 | 0.1076 | 0.0041 | 0.1224 | 0.0018 |
| W0949 | 0.1052 | 0.0049 | 0.1018 | 0.0069 | 0.1174 | 0.0083 |
| W0950 | 0.1208 | 0.0050 | 0.1002 | 0.0250 | 0.1178 | 0.0179 |
| W0956 | 0.0987 | 0.0053 | 0.1017 | 0.0058 | 0.1270 | 0.0133 |
| W0965 | 0.1068 | 0.0085 | 0.0701 | 0.0230 | 0.1270 | 0.0090 |
| W0967 | 0.1017 | 0.0263 | 0.1162 | 0.0038 | 0.1263 | 0.0033 |
| W0968 | 0.1162 | 0.0097 | 0.1139 | 0.0024 | 0.1167 | 0.0090 |
| W0977 | 0.1159 | 0.0063 | 0.0987 | 0.0064 | 0.1338 | 0.0203 |
| W0979 | 0.1099 | 0.0028 | 0.0883 | 0.0199 | 0.1276 | 0.0094 |
| W0980 | 0.1264 | 0.0046 | 0.1135 | 0.0139 | 0.1312 | 0.0185 |
| W0981 | 0.1364 | 0.0040 | 0.1164 | 0.0112 | 0.1560 | 0.0051 |
| W0982 | 0.1454 | 0.0207 | 0.1242 | 0.0031 | 0.1634 | 0.0042 |
| W0983 | 0.1272 | 0.0054 | 0.1126 | 0.0153 | 0.1439 | 0.0071 |
| W0984 | 0.1165 | 0.0038 | 0.1141 | 0.0134 | 0.1476 | 0.0126 |
| W0994 | 0.0896 | 0.0137 | 0.0811 | 0.0205 | 0.1329 | 0.0071 |
| W1002 | 0.1135 | 0.0078 | 0.1083 | 0.0202 | 0.1410 | 0.0084 |
| W1004 | 0.1054 | 0.0054 | 0.1118 | 0.0153 | 0.1219 | 0.0065 |
| W1036 | 0.1095 | 0.0092 | 0.1052 | 0.0044 | 0.1366 | 0.0054 |
| W1039 | 0.1204 | 0.0153 | 0.1140 | 0.0142 | 0.1508 | 0.0093 |
| W1040 | 0.1330 | 0.0048 | 0.1202 | 0.0111 | 0.1651 | 0.0166 |
| W1064 | 0.1290 | 0.0103 | 0.1256 | 0.0076 | 0.1527 | 0.0070 |
| W1071 | 0.1063 | 0.0041 | 0.0989 | 0.0244 | 0.1310 | 0.0309 |
| W1083 | 0.1077 | 0.0080 | 0.1043 | 0.0237 | 0.1167 | 0.0061 |
| W1092 | 0.1045 | 0.0021 | 0.1084 | 0.0102 | 0.1171 | 0.0091 |
| W1094 | 0.1073 | 0.0086 | 0.0939 | 0.0228 | 0.1235 | 0.0120 |
| W1097 | 0.1211 | 0.0038 | 0.1223 | 0.0079 | 0.1378 | 0.0071 |
| W1104 | 0.0997 | 0.0040 | 0.0874 | 0.0129 | 0.1116 | 0.0078 |
| W1117 | 0.1188 | 0.0036 | 0.1325 | 0.0073 | 0.1404 | 0.0082 |
| W1118 | 0.1141 | 0.0032 | 0.1326 | 0.0054 | 0.1342 | 0.0043 |
| W1123 | 0.1197 | 0.0102 | 0.1033 | 0.0215 | 0.1428 | 0.0082 |
| W1137 | 0.1302 | 0.0068 | 0.1187 | 0.0085 | 0.1553 | 0.0006 |
| W1146 | 0.1172 | 0.0044 | 0.1198 | 0.0091 | 0.1488 | 0.0093 |
| W1182 | 0.1210 | 0.0084 | 0.1195 | 0.0113 | 0.1353 | 0.0090 |
| W1187 | 0.1034 | 0.0059 | 0.0889 | 0.0190 | 0.1105 | 0.0031 |
| W1192 | 0.1067 | 0.0150 | 0.1022 | 0.0169 | 0.1362 | 0.0128 |
| W1197 | 0.0943 | 0.0080 | 0.0803 | 0.0180 | 0.1140 | 0.0084 |
| W1203 | 0.1208 | 0.0050 | 0.1021 | 0.0160 | 0.1284 | 0.0056 |
| W1208 | 0.0970 | 0.0129 | 0.0966 | 0.0074 | 0.1335 | 0.0047 |
| W1227 | 0.1211 | 0.0039 | 0.1193 | 0.0079 | 0.1430 | 0.0030 |
| W1233 | 0.1198 | 0.0018 | 0.1264 | 0.0053 | 0.1543 | 0.0052 |
| W1235 | 0.1280 | 0.0124 | 0.1261 | 0.0072 | 0.1889 | 0.0101 |
| WT | 0.1301 | 0.0100 | 0.1249 | 0.0062 | 0.1961 | 0.0218 |

88 Winner lines were screened for photosynthetic yield by PAM analysis. All strains were tested in both HSM and MASM media. Statistical significance was not calculated with this dataset because only one replicate of each sample was analyzed. The results are provided in the table below.

TABLE 29

| Photosynthetic | | |
|---|---|---|
| Yield | Fᵥ/Fₘ | |
| Winner | HSM | MASM |
| WT | 0.705 | 0.732 |
| W0601 | 0.685 | 0.697 |
| W0607 | 0.679 | 0.694 |
| W0629 | 0.682 | 0.713 |
| W0647 | 0.685 | 0.699 |
| W0663 | 0.619 | 0.665 |
| W0667 | 0.693 | 0.726 |
| W0670 | 0.697 | 0.726 |
| W0674 | 0.680 | 0.706 |
| W0675 | 0.701 | 0.726 |
| W0677 | 0.726 | 0.711 |
| W0702 | 0.692 | 0.706 |
| W0709 | 0.707 | 0.726 |
| W0752 | 0.697 | 0.712 |
| W0757 | 0.688 | 0.692 |
| W0758 | 0.684 | 0.698 |
| W0770 | 0.686 | 0.700 |
| W0774 | 0.699 | 0.711 |
| W0775 | 0.706 | 0.710 |
| W0776 | 0.705 | 0.731 |
| W0785 | 0.691 | 0.696 |
| W0793 | 0.706 | 0.719 |
| W0798 | 0.717 | 0.712 |
| W0801 | 0.737 | 0.730 |
| W0802 | 0.678 | 0.682 |
| W0823 | 0.688 | 0.713 |
| W0825 | 0.676 | 0.704 |
| W0828 | 0.676 | 0.555 |
| W0829 | | 0.710 |
| W0832 | 0.681 | 0.688 |
| W0841 | 0.707 | 0.730 |
| W0846 | 0.699 | 0.721 |
| W0857 | 0.703 | 0.707 |
| W0871 | 0.700 | 0.721 |
| W0883 | 0.716 | 0.737 |
| W0894 | 0.733 | 0.735 |
| W0905 | 0.714 | 0.725 |
| W0913 | 0.710 | 0.706 |
| W0925 | 0.696 | 0.710 |
| W0929 | 0.697 | 0.719 |
| W0931 | 0.696 | 0.715 |
| W0934 | 0.694 | 0.732 |
| W0936 | 0.700 | 0.731 |
| W0942 | 0.691 | 0.729 |
| W0949 | 0.698 | 0.667 |
| W0950 | 0.717 | 0.737 |
| W0956 | 0.720 | 0.731 |
| W0965 | 0.685 | 0.695 |
| W0967 | 0.676 | 0.717 |
| W0968 | 0.685 | 0.715 |
| W0977 | 0.685 | 0.711 |
| W0979 | 0.682 | 0.697 |
| W0980 | 0.702 | 0.731 |
| W0981 | 0.698 | 0.735 |
| W0982 | 0.701 | 0.727 |
| W0983 | 0.699 | 0.728 |
| W0984 | 0.699 | 0.732 |
| W0994 | 0.694 | 0.704 |
| W1002 | 0.732 | 0.724 |
| W1004 | 0.698 | 0.689 |
| W1036 | 0.674 | 0.712 |
| W1039 | 0.693 | 0.719 |
| W1040 | 0.689 | 0.711 |
| W1064 | 0.698 | 0.713 |
| W1071 | 0.694 | 0.705 |
| W1083 | 0.700 | 0.707 |
| W1084 | 0.692 | |
| W1092 | 0.696 | 0.696 |
| W1094 | 0.695 | 0.726 |
| W1097 | 0.709 | 0.731 |
| W1104 | 0.710 | 0.702 |
| W1117 | 0.699 | 0.725 |
| W1118 | 0.693 | 0.720 |
| W1123 | 0.703 | 0.729 |
| W1124 | 0.679 | 0.721 |
| W1137 | 0.701 | 0.720 |
| W1146 | 0.672 | 0.719 |
| W1182 | 0.714 | 0.735 |
| W1187 | 0.699 | 0.702 |
| W1192 | 0.704 | 0.729 |
| W1197 | 0.698 | 0.696 |
| W1202 | 0.717 | 0.738 |
| W1203 | 0.699 | 0.723 |
| W1208 | 0.698 | 0.720 |
| W1209 | 0.702 | 0.720 |
| W1210 | 0.695 | 0.725 |
| W1227 | 0.700 | 0.727 |
| W1233 | 0.682 | 0.727 |
| W1235 | 0.702 | 0.732 |

Flow cytometry was used to determine cell size for all selected genes that advanced to the regeneration phase. Cell density for each sample was calculated using the Guava EasyCyte flow cytometer. Samples with densities below 200,000 cells/ml were excluded—these samples were 10% of the wild type density. Following subsequent data acquisition on the BD Influx cell sorter, the main population was gated for single cells and analyzed for the mean forward scatter. An ANOVA with Dunnett's statistic test (p<0.05) was performed on the summary data (Larson. Analysis of Variance with Just Summary Statistics as Input. American Statistician (1992) vol. 46 pp. 151-152) to determine which samples were significantly different than wild type. Most Selected Gene lines were larger than wild type, with only 3 lines being smaller. Data and statistical analysis are available in the table below.

TABLE 30

| | | | | Dunnett's Test | |
|---|---|---|---|---|---|
| Raw Data | | | | Abs(Diff)- | |
| Winner | Mean | stdev | N | LSD | p-Value |
| W0601 | 16291 | 4143.7 | 9579 | −114.87 | 0.9988 |
| W0607 | 17805 | 4264.5 | 7237 | 1383.28 | <.0001* |
| W0629 | 17530 | 4123.7 | 8579 | 1118.28 | <.0001* |
| W0647 | 18142 | 3361.7 | 9724 | 1736.89 | <.0001* |
| W0663 | 17675 | 3292.1 | 9685 | 1269.69 | <.0001* |
| W0667 | 18271 | 3721.3 | 9740 | 1865.97 | <.0001* |
| W0670 | 18205 | 4377.4 | 9784 | 1800.20 | <.0001* |
| W0674 | 20980 | 4349.5 | 9181 | 4571.93 | <.0001* |
| W0675 | 17494 | 3363.1 | 2863 | 991.66 | <.0001* |
| W0677 | 19382 | 3727.9 | 9644 | 2976.47 | <.0001* |
| W0702 | 16813 | 3580.4 | 5949 | 378.14 | <.0001* |
| W0709 | 21130 | 4832.4 | 9681 | 4724.67 | <.0001* |
| W0752 | 19089 | 4359.3 | 7517 | 2669.62 | <.0001* |
| W0757 | 19022 | 3829 | 7530 | 2602.72 | <.0001* |
| W0758 | 15916 | 3235.9 | 5193 | 44.93 | 0.0058* |
| W0770 | 18418 | 3628.6 | 9789 | 2013.22 | <.0001* |
| W0774 | 17285 | 4012.2 | 9746 | 880.00 | <.0001* |
| W0775 | 19448 | 3813.3 | 4712 | 2995.02 | <.0001* |
| W0776 | 17379 | 3258.2 | 5380 | 936.68 | <.0001* |
| W0785 | 18592 | 4792.3 | 9707 | 2186.80 | <.0001* |
| W0793 | 19299 | 3516 | 375 | 2355.68 | <.0001* |
| W0798 | 19135 | 3772.5 | 9747 | 2730.01 | <.0001* |
| W0801 | 23847 | 4919.4 | 7640 | 7428.60 | <.0001* |
| W0802 | 19264 | 4393.1 | 1596 | 2680.92 | <.0001* |
| W0823 | 17270 | 3586 | 7246 | 848.35 | <.0001* |
| W0825 | 27394 | 7096.4 | 9768 | 10989.12 | <.0001* |
| W0828 | 20461 | 4118.4 | 2185 | 3924.76 | <.0001* |
| W0829 | 21391 | 4579.9 | 3957 | 4922.48 | <.0001* |
| W0832 | 19236 | 4060.9 | 3927 | 2766.76 | <.0001* |
| W0841 | 17345 | 3122.7 | 7171 | 922.70 | <.0001* |
| W0846 | 18096 | 4400.1 | 9771 | 1691.13 | <.0001* |
| W0857 | 18398 | 3661.3 | 9577 | 1992.12 | <.0001* |
| W0871 | 26713 | 6703.7 | 9618 | 10307.34 | <.0001* |
| W0883 | 17920 | 3812.8 | 6987 | 1496.05 | <.0001* |
| W0894 | 24617 | 5064 | 9705 | 8211.79 | <.0001* |
| W0905 | 21225 | 4678.5 | 1586 | 4640.89 | <.0001* |
| W0913 | 21687 | 4230.3 | 8154 | 5272.42 | <.0001* |
| W0925 | 16879 | 3505.6 | 2597 | 365.06 | <.0001* |
| W0929 | 19181 | 4591.5 | 9789 | 2776.22 | <.0001* |
| W0931 | 16547 | 3273.3 | 9459 | 140.48 | <.0001* |
| W0934 | 17804 | 3308.5 | 9713 | 1398.83 | <.0001* |
| W0936 | 19998 | 3970.5 | 9772 | 3593.14 | <.0001* |
| W0942 | 19044 | 3114.6 | 5074 | 2597.09 | <.0001* |
| W0949 | 17706 | 4005.1 | 9744 | 1300.99 | <.0001* |
| W0950 | 21034 | 4161.4 | 9566 | 4628.06 | <.0001* |
| W0956 | 22300 | 4661.8 | 6243 | 5868.54 | <.0001* |
| W0965 | 20885 | 4896.8 | 1681 | 4310.26 | <.0001* |
| W0967 | 21322 | 5075.9 | 7755 | 4904.49 | <.0001* |
| W0968 | 18101 | 4037.9 | 7773 | 1683.63 | <.0001* |
| W0977 | 27710 | 5788.8 | 4579 | 11254.59 | <.0001* |
| W0979 | 20503 | 3623 | 2778 | 3997.15 | <.0001* |
| W0980 | 21094 | 4215.1 | 7627 | 4675.50 | <.0001* |
| W0981 | 18157 | 3214.1 | 5303 | 1713.56 | <.0001* |
| W0982 | 17088 | 3388 | 9728 | 682.91 | <.0001* |
| W0983 | 17183 | 2907.1 | 9752 | 778.03 | <.0001* |
| W0984 | 17005 | 3187 | 9710 | 599.82 | <.0001* |
| W0994 | 19580 | 4452.1 | 9772 | 3175.14 | <.0001* |
| W1002 | 22074 | 4503.5 | 1291 | 5454.17 | <.0001* |
| W1004 | 19687 | 4807.3 | 3338 | 3201.56 | <.0001* |
| W1036 | 16971 | 3806.5 | 6753 | 544.84 | <.0001* |
| W1039 | 17715 | 3158.5 | 9685 | 1309.69 | <.0001* |
| W1040 | 17854 | 3556.3 | 9782 | 1449.19 | <.0001* |
| W1064 | 17564 | 3512.7 | 9783 | 1159.19 | <.0001* |
| W1071 | 31584 | 6255.6 | 9807 | 15179.32 | <.0001* |
| W1083 | 18176 | 3667.5 | 1703 | 1603.31 | <.0001* |
| W1092 | 17047 | 3281.8 | 8708 | 636.10 | <.0001* |
| W1094 | 30892 | 6261.2 | 9722 | 14486.88 | <.0001* |
| W1097 | 16585 | 3349.2 | 1848 | 24.85 | 0.0236* |
| W1104 | 17119 | 4781 | 9737 | 713.96 | <.0001* |
| W1117 | 15287 | 3406.6 | 9445 | 712.41 | <.0001* |
| W1118 | 15736 | 3511.9 | 9751 | 265.03 | <.0001* |
| W1123 | 21475 | 4251.3 | 9756 | 5070.05 | <.0001* |
| W1137 | 17158 | 3234.1 | 4974 | 709.49 | <.0001* |
| W1146 | 16313 | 3291.6 | 9818 | −91.63 | 0.9312 |
| W1182 | 20574 | 4268.5 | 9718 | 4168.86 | <.0001* |
| W1187 | 19995 | 5600.3 | 7712 | 3577.16 | <.0001* |
| W1192 | 21773 | 5235.7 | 7260 | 5351.47 | <.0001* |
| W1197 | 16915 | 3793.2 | 7139 | 492.42 | <.0001* |
| W1203 | 18289 | 4617.9 | 9645 | 1883.48 | <.0001* |
| W1208 | 20668 | 4493.7 | 9173 | 4259.89 | <.0001* |
| W1210 | 17800 | 3306.3 | 3839 | 1328.60 | <.0001* |
| W1227 | 16534 | 3496.8 | 9833 | 129.45 | <.0001* |
| W1233 | 20348 | 5153.1 | 9768 | 3943.12 | <.0001* |
| W1235 | 17750 | 4682.9 | 4564 | 1294.31 | <.0001* |
| WT | 16203 | 3911 | 9649 | −202.50 | 1 |

Selected genes that advanced to the regeneration phase were stained with lipid dyes. Lipid dye staining is a high throughput method to find candidate strains that potentially contain high lipid (and potentially high oil) content. Each plate contained a positive control line that historically has high fluorescence when stained for neutral lipids (SN03). While most lines demonstrated varied levels of staining, there were two instances (W0802, W0968) in which the fold increase over wild type was consistent for both lipid dyes in each different media. A table of the fold difference over wild type for both lipid dyes in each different media can be found in the table below. Statistical significance was not calculated with this dataset because only one replicate of each sample was run.

TABLE 31

| | Nile Red | | | Bodipy 493/503 | | |
|---|---|---|---|---|---|---|
| Winner | TAP | HSM | MASM | TAP | HSM | MASM |
| W0601 | 3.853 | 4.045 | 10.435 | 0.754 | 3.684 | 7.895 |
| W0607 | 4.303 | 0.663 | 7.212 | 0.589 | 0.990 | 5.819 |
| W0629 | 1.406 | 0.767 | 5.616 | 0.599 | 0.574 | 5.331 |
| W0647 | 3.730 | 0.678 | 7.601 | 0.601 | 0.391 | 5.805 |
| W0663 | 1.239 | 1.154 | 6.590 | 0.347 | 0.723 | 8.593 |
| W0667 | 1.205 | 1.055 | 9.992 | 0.398 | 0.858 | 10.079 |
| W0670 | 5.131 | 2.369 | 2.285 | 6.281 | 1.994 | 1.798 |
| W0674 | 7.735 | 1.879 | 2.978 | 3.322 | 0.218 | 1.469 |
| W0675 | 1.664 | 0.765 | 20.225 | 0.786 | 0.502 | 7.534 |
| W0677 | 2.284 | 1.225 | 7.811 | 0.798 | 0.360 | 5.684 |
| W0702 | 2.300 | 1.278 | 37.270 | 2.722 | 0.811 | 9.782 |
| W0709 | 3.945 | 2.735 | 5.309 | 1.595 | 5.598 | 7.952 |
| W0752 | 3.606 | 4.587 | 9.321 | 0.923 | 3.845 | 9.560 |
| W0757 | 5.269 | 1.415 | 7.203 | 2.364 | 1.335 | 5.799 |
| W0758 | 2.652 | 0.865 | 1.762 | 2.385 | 0.962 | 1.656 |
| W0770 | 1.349 | 0.696 | 1.992 | 0.457 | 0.362 | 1.856 |
| W0774 | 7.725 | 1.949 | 5.760 | 1.973 | 3.395 | 3.691 |
| W0775 | 2.017 | 1.413 | 4.804 | 0.622 | 1.112 | 4.301 |
| W0776 | 0.959 | 1.304 | 8.918 | 0.655 | 0.778 | 7.820 |
| W0785 | 2.065 | 1.918 | 2.432 | 2.371 | 1.261 | 4.736 |
| W0793 | 1.860 | 1.029 | 5.082 | 1.757 | 0.616 | 1.538 |
| W0798 | 3.039 | 2.064 | 7.754 | 1.077 | 1.179 | 4.756 |
| W0801 | 2.906 | 1.572 | 3.971 | 1.173 | 0.582 | 3.239 |
| W0802 | 11.692 | 6.319 | 9.721 | 1.330 | 5.735 | 5.971 |
| W0823 | 2.203 | 2.484 | 4.643 | 0.466 | 2.172 | 4.953 |
| W0825 | 5.958 | 1.818 | 8.218 | 1.525 | 1.967 | 3.558 |
| W0828 | 15.459 | 1.316 | 4.025 | 5.892 | 0.738 | 1.353 |
| W0829 | 1.881 | 1.162 | 2.095 | 0.635 | 0.806 | 3.393 |
| W0832 | 1.763 | 0.736 | 7.476 | 0.245 | 0.641 | 4.587 |
| W0841 | 0.795 | 0.908 | 2.017 | 0.377 | 0.425 | 1.767 |
| W0846 | 1.412 | 1.013 | 2.581 | 1.545 | 0.515 | 1.864 |
| W0857 | 1.401 | 1.488 | 4.224 | 0.465 | 1.048 | 4.116 |
| W0871 | 1.614 | 3.974 | 9.288 | 0.646 | 1.532 | 6.593 |
| W0883 | 2.470 | 1.220 | 5.716 | 0.736 | 0.698 | 4.502 |
| W0894 | 1.293 | 6.199 | 3.477 | 0.833 | 2.489 | 1.120 |
| W0905 | 5.097 | 1.894 | 4.415 | 1.114 | 5.081 | 6.908 |
| W0913 | 5.881 | 3.602 | 3.049 | 0.534 | 4.677 | 2.932 |
| W0925 | 5.110 | 1.008 | 3.467 | 0.794 | 1.224 | 3.588 |
| W0929 | 2.543 | 4.021 | 2.197 | 0.870 | 5.087 | 2.749 |
| W0931 | 1.938 | 1.468 | 1.942 | 0.773 | 1.376 | 2.179 |
| W0934 | 0.834 | 0.964 | 2.222 | 0.547 | 0.404 | 1.538 |
| W0936 | 1.437 | 3.785 | 3.553 | 1.157 | 3.319 | 2.231 |
| W0942 | 0.794 | 1.334 | 1.817 | 0.419 | 0.734 | 1.526 |
| W0949 | 1.913 | 2.233 | 2.855 | 1.890 | 1.565 | 2.318 |
| W0950 | 1.218 | 1.641 | 2.021 | 0.698 | 1.052 | 2.182 |
| W0956 | 3.296 | 6.461 | 8.879 | 4.628 | 2.759 | 2.555 |
| W0965 | 11.649 | 4.120 | 1.820 | 1.465 | 5.111 | 1.065 |
| W0967 | 2.787 | 3.033 | 5.436 | 0.862 | 1.894 | 5.414 |
| W0968 | 7.993 | 6.252 | 7.342 | 2.779 | 5.066 | 3.207 |
| W0977 | 9.804 | 1.281 | 10.379 | 2.461 | 1.686 | 7.843 |
| W0979 | 3.085 | 1.031 | 7.152 | 0.408 | 1.512 | 4.771 |
| W0980 | 1.498 | 0.381 | 1.692 | 0.583 | 0.372 | 2.138 |
| W0981 | 1.058 | 1.547 | 2.272 | 0.867 | 1.055 | 2.325 |
| W0982 | 1.049 | 1.224 | 1.925 | 0.952 | 0.599 | 1.468 |
| W0983 | 0.935 | 1.398 | 2.174 | 0.829 | 0.935 | 2.201 |
| W0984 | 1.750 | 1.209 | 3.566 | 1.146 | 0.615 | 3.191 |
| W0994 | 13.754 | 1.362 | 3.976 | 4.497 | 1.273 | 4.557 |
| W1002 | 2.914 | 1.074 | 2.866 | 1.046 | 0.495 | 2.374 |
| W1004 | 10.534 | 3.508 | 6.932 | 1.349 | 5.496 | 5.336 |
| W1036 | 1.313 | 0.785 | 2.448 | 0.402 | 0.483 | 1.744 |
| W1039 | 1.749 | 0.964 | 3.047 | 0.357 | 1.051 | 3.271 |
| W1040 | 1.879 | 0.651 | 2.979 | 0.417 | 0.457 | 3.135 |
| W1064 | 1.617 | 1.098 | 2.204 | 0.393 | 0.665 | 2.272 |
| W1071 | 9.081 | 1.190 | 4.946 | 0.885 | 1.756 | 2.165 |
| W1071 | 1.846 | 7.330 | 5.120 | 1.118 | 4.361 | 4.285 |
| W1092 | 2.076 | 1.910 | 3.382 | 2.221 | 1.383 | 2.952 |
| W1094 | 1.857 | 2.343 | 1.957 | 2.656 | 1.666 | 0.936 |
| W1097 | 1.958 | 0.743 | 4.292 | 1.841 | 0.231 | 3.094 |
| W1104 | 2.026 | 5.441 | 2.179 | 0.827 | 4.038 | 1.025 |
| W1117 | 4.056 | 1.465 | 10.523 | 2.632 | 1.289 | 9.112 |
| W1118 | 1.437 | 3.198 | 3.139 | 0.835 | 3.320 | 3.268 |
| W1123 | 1.079 | 0.556 | 1.752 | 0.483 | 0.731 | 2.895 |
| W1137 | 1.517 | 1.124 | 1.896 | 0.651 | 1.353 | 2.205 |
| W1146 | 1.342 | 0.589 | 1.370 | 0.759 | 0.410 | 2.684 |
| W1182 | 1.339 | 1.816 | 2.116 | 0.676 | 1.395 | 2.459 |
| W1187 | 2.551 | 1.384 | 3.842 | 0.742 | 1.708 | 3.783 |
| W1192 | 0.814 | 2.084 | 1.931 | 0.648 | 2.040 | 2.412 |
| W1197 | 5.042 | 1.567 | 4.674 | 1.607 | 0.460 | 3.475 |
| W1203 | 5.179 | 0.579 | 9.705 | 2.210 | 0.819 | 10.642 |
| W1208 | 4.413 | 4.981 | 3.360 | 2.072 | 6.184 | 4.020 |
| W1227 | 4.376 | 0.999 | 4.107 | 2.315 | 2.411 | 4.402 |
| W1233 | 3.838 | 2.653 | 2.608 | 1.776 | 4.050 | 2.877 |
| W1235 | 0.811 | 1.487 | 3.263 | 0.676 | 1.221 | 3.777 |
| SN03+ | 10.492 | 6.249 | 12.071 | 8.015 | 4.405 | 7.369 |

Based on the process of wild type competition and regeneration of transgenic lines, 27 of 94 selected S. dimorphus genes were validated as having a competitive growth advantage due to overexpression of the gene. These genes are listed in the table below.

TABLE 32

| Gene | Winner | Locus ID | C. reinhardtii description | % CDS | Class |
|---|---|---|---|---|---|
| 1 | W1210 | g16071 | | 100 | 2 |
| 13 | W0829 | scaffold110: | | | 5 |
| | | 302109-303275 | | | |
| 13 | W1155 | scaffold110: | | | |
| | | 302109-303275 | | | |
| 13 | W1170 | scaffold110: | | | |
| | | 302109-303275 | | | |
| 13 | W1176 | scaffold110: | | | |
| | | 302109-303275 | | | |
| 19 | W0774 | scaffold42: | | | 5 |
| | | 463800-464650 | | | |
| 28 | W0785 | g12290 | | 100 | 2 |
| 28 | W1169 | g12290 | | 100 | |
| 30 | W0611 | g14780 | ribulose bisphosphate carboxylase | 100 | |
| | | | small chain 1A; Cyclin family protein | | |
| 30 | W0677 | g14780 | ribulose bisphosphate carboxylase | 100 | |
| | | | small chain 1A; Cyclin family protein | | |
| 30 | W0723 | g14780 | ribulose bisphosphate carboxylase | 100 | |
| | | | small chain 1A; Cyclin family protein | | |
| 30 | W0776 | g14780 | ribulose bisphosphate carboxylase | 46 | 3 |
| | | | small chain 1A; Cyclin family protein | | |
| 30 | W0805 | g14780 | ribulose bisphosphate carboxylase | 100 | |
| | | | small chain 1A; Cyclin family protein | | |
| 30 | W0912 | g14780 | ribulose bisphosphate carboxylase | 100 | |
| | | | small chain 1A; Cyclin family protein | | |
| 30 | W0951 | g14780 | ribulose bisphosphate carboxylase | 100 | |
| | | | small chain 1A; Cyclin family protein | | |
| 31 | W1123 | g1509 | Protein kinase superfamily protein with | 100 | 3 |
| | | | octicosapeptide/Phox/Bem1p domain | | |
| 33 | W0956 | g18330 | Protein kinase superfamily protein | 42 | 2 |
| 39 | W0607 | g3921 | ubiquitin-associated (UBA)/TS-N | 100 | 2 |
| | | | domain-containing protein | | |
| 39 | W0626 | g3921 | ubiquitin-associated (UBA)/TS-N | 100 | |
| | | | domain-containing protein | | |
| 44 | W0979 | g664 | Nucleic acid-binding, OB-fold-like | 100 | 4 |
| | | | protein 100 | | |
| 45 | W1233 | g7387 | demeter-like 2 | 100 | 3 |
| 47 | W1100 | g884 | | 100 | |
| 47 | W1104 | g884 | | 100 | 2 |
| 48 | W1004 | g9576 | photosystem II subunit Q-2 | 97 | 2 |
| 48 | W1083 | g9576 | photosystem II subunit Q-2 | 19 | 5 |
| 48 | W0932 | g9576 | photosystem II subunit Q-2 | 97 | |
| 48 | W1098 | g9576 | photosystem II subunit Q-2 | 19 | |
| 53 | W0667 | scaffold126: | | | 5 |
| | | 355759-356343 | | | |
| 54 | W0770 | scaffold18: | | | 1 |
| | | 1489301-1489559 | | | |
| 54 | W0771 | scaffold18: | | | |
| | | 1494447-1495555 | | | |
| 57 | W0802 | scaffold33: | | | 5 |
| | | 535965-537528 | | | |
| 60 | W1092 | scaffold64: | | | 4 |
| | | 287639-288387 | | | |
| 64 | W0675 | g14907 | | 100 | 2 |
| 65 | W0949 | g14943 | ATP synthase delta-subunit gene | 100 | 1 |
| 67 | W0883 | g18194 | gamma carbonic anhydrase like 1 | 100 | 3 |
| 72 | W0980 | scaffold240: | | | 2 |
| | | 19496-20329 | | | |
| 78 | W1036 | g13214 | | 3 | 4 |
| 80 | W0923 | g17628 | receptor for activated C kinase 1C | 100 | |
| 80 | W0950 | g17628 | receptor for activated C kinase 1C | 58 | 4 |
| 82 | W0841 | g4280 | | 100 | 5 |
| 84 | W1146 | g8264 | | 26 | 4 |
| 85 | W0823 | scaffold67 | | | 2 |
| | | :222004-223125 | | | |
| 85 | W0916 | scaffold67: | | | |
| | | 222004-223125 | | | |
| 89 | W0659 | g13997 | aldehyde dehydrogenase 2C4 | 100 | |
| 89 | W0796 | g13997 | aldehyde dehydrogenase 2C4 | 100 | |
| 89 | W0934 | g13997 | aldehyde dehydrogenase 2C4 | 93 | 3 |
| 89 | W1203 | g13997 | aldehyde dehydrogenase 2C4 | 100 | 1 |
| 91 | W0629 | g2506 | photosystem II subunit X | 100 | 2 |
| 91 | W0924 | g2506 | photosystem II subunit X | 100 | |
| 91 | W1028 | g2506 | photosystem II subunit X | 100 | |
| 91 | W1115 | g2506 | photosystem II subunit X | 100 | |

Desmodesmus Sp. Validation

Three of the Desmodesmus sp. 93 selected genes were represented by multiple winning transgenic lines containing different lengths of the cDNA. These lines were considered to be non-identical and a representative winning line containing each cDNA was included in the validation process. Locus ID g2004 did not have a viable original line (W1385, W1387, W1411) and was not included in the original line 1:1 turbidostat competitions, but was regenerated by cloning the gene out of the cDNA library. In all, 96 winning lines representing 93 selected genes entered the validation process.

Turbidostat Competitions with Original Lines

Selected gene original lines, wild type C. reinhardtii, and the YFP strain (see below) were grown in TAP media to saturation in 50 ml flasks. 3 ml of culture was acclimated in 50 ml HSM media and grown 2 days prior to turbidostat setup. Cultures were normalized to the lowest OD₇₅₀ value and mixed 1:1 with the YFP strain. 8 ml of mixture was inoculated in three replicate turbidostats and filled with HSM to a final volume of 35 ml. Turbidostats were grown under a constant stream of 0.2% CO₂ and a 16H/8H light-dark diurnal cycle. A light intensity of ^{˜}150 μE/m² was provided during the 16H phase of the cycle.

Starting on the day of setup (day 0), each turbidostat was sampled for FACS and the corresponding media bottle was weighed to track the number of generations. FACS was performed on the Guava easyCyte flow cytometer (EMD Millipore; Billerica, Mass.) to calculate the relative ratios of the Selected Gene and YFP strain in each turbidostat. Data were collected every other day through day 10.

The common competitor strain was generated by transforming C. reinhardtii CC-1690 with a plasmid containing nuclear-optimized YFP (Venus) linked to the bleomycin-resistance gene and FMDV 2A cleavage peptide, all under the control of the AR4 promoter. Since the YFP strain outperforms wild type, all Selected Genes and wild type were evaluated relative to its performance.

Using Guava CytoSoft software, gates were applied to each flow cytometry run to differentiate non-green fluorescent cells from the Venus strain (a YFP-expressing common competitor). The winner ratio was calculated for each sample as

$$r = \frac{M 1}{M 2}$$

where M1 is the number of non-fluorescent counts in gate M1 (red), and M2 is the number of fluorescent counts in gate M2 (blue). Note that both strains fluoresce in the red channel (y-axis) due to the presence of chlorophyll.

The selection coefficient equation, In(rₜ)=In(r₀)+st, is in the form of a line y=b+mx, where the selection coefficient (s) is equivalent to the slope (m) of the natural log of the ratio over time (generally days). While turbidostats maintain optical density within a relatively narrow range, slight variances in density can affect the growth rate of a turbidostat population, resulting in a variable number of generations for replicate turbidostats. In order to control for this effect, media consumption between Guava samplings was used to calculate the number of generations at each time point, and selection coefficients were calculated in units of generations⁻¹ by plotting In(rₜ) vs. the number of generations. The calculated selection coefficient (i.e. the slope) was then used to rank and select potential winning clones as Validated Genes.

For en masse experiments, selected gene lines were grown in 1 ml of TAP media to saturation in 96-well deep-well blocks. The cultures were then acclimated to HSM media by diluting back 1:10 in deep-well blocks. Cultures were grown two days in HSM media prior to inoculation in turbidostats. Cultures were normalized by OD₇₅₀ and pooled. This pooled mixture was sorted by FACS into 96-well microplates containing TAP media for a baseline reading of the distribution of genes. Eight plates were sorted for baseline analysis at the time of turbidostat inoculation. Twelve replicate turbidostats were inoculated from this pool and cultured as before in HSM for two weeks. After two weeks, samples were taken from turbidostats and sorted into liquid cultures (four 96-well plates per turbidostat). After approximately five days of growth in 96-well plates, cultures were amplified by PCR and submitted for sequencing.

Prior to the start of the en masse competition, selected genes derived from Arthrospira sp. (Spirulina) libraries were compared to the Desmodesmus sp. genome using blastn. These selected genes possess a unique locus identifier in the Desmodesmus sp. genome that makes it possible to compete the selected genes from both species together. Sanger reads were processed using CLC bio's Genomics Workbench software and a custom plugin described previously. The sequences are then compared to the Desmodesmus sp. genome using blastn. The gene locus for the top hit is determined and the relation of the BLAST hit and gene CDS is determined. A final result table is generated containing primarily the gene locus and how many times it was hit by a sequence within the dataset. Spirulina genes were then correlated back to the relevant CDS in that genome. The distribution of these genes can be compared between the baseline and the two week time point.

Hit counts and total sequences were used to calculate the ratio of each variant present in a given timepoint. These numbers were then used to calculate a selection coefficient using the formula described previously. The selection coefficients used in this analysis do not conform strictly to some of the assumptions upon which the formula is based, in that this is not a single clone compared against a uniform population. Each clone is compared to the rest of the pool, which itself is made up of many other clones. However, within the experiment, the calculated selection coefficients provide a valid way to compare and rank potentially winning clones.

### Regeneration of Lines

Cold Fusion technology (System Biosciences; Mountain View, Calif.) was used to re-clone all the selected lines. This method allows cloning of PCR fragments via homology regions at each end of the PCR product and the linearized destination vector. The screening primers used earlier in the project for detection of cloned cDNA were used for this purpose. A vector was built that contains all the regions of the cDNA expression vector except the region between the sites homologous to the screening primers. This region was replaced with the restriction sites NdeI and SpeI (see FIG. 3). A further modification was also made to the expression vector by the addition of 1-CeuI sites flanking the entire cassette. These homing endonuclease sites facilitate linearization for transformation and since the recognition site is 29 base pairs in length it is unlikely to be found in any cDNA fragment cloned into the library.

Cell lysate of the original selected lines was used as PCR template for cloning. The cDNA shuttle vector was digested with NdeI and SpeI and purified by gel extraction. PCR product and linearized vector were used for the Cold Fusion reaction as per the manufacturer's guidelines. Cloning in this manner creates an expression cassette identical to the one found in the original lines. In the case where the original line was no longer available (W1411), the cDNA insert was PCR amplified from the plasmid cDNA library originally used for primary screening and cloned into the cDNA overexpression vector. Cloned constructs were confirmed by DNA sequencing.

Re-cloned genes were transformed into Chlamydomonas reinhardtii CC-1690 and selected for resistance to both hygromycin and paromomycin (each at 10 μg/ml). For each gene, 24 transgenic lines were PCR screened and sequenced. Twelve sequence confirmed lines per gene were selected to enter turbidostats in competition with wild type via a common competitor.

Turbidostat Competitions with Regenerated Lines

Regenerated lines were grown in 1 ml of TAP media to saturation in 96-well deep-well blocks. The cultures were then acclimated to HSM media by diluting back 1:10 in 96-well deep-well blocks. Cultures were grown two days in HSM media prior to inoculation in turbidostats. The wild type and YFP strain were treated in the same manner though at larger scale. The twelve regenerated lines were normalized by OD₇₅₀ and pooled. The pooled mixture was then mixed at a ratio of 1:1 with the YFP strain and used for three replicate turbidostats. Each turbidostat was filled with HSM to a final volume of 35 ml. Cultures were grown under a constant stream of 0.2% CO₂ and a 16H/8H light-dark diurnal cycle. A light intensity of ^{˜}150 μE/m² was provided during the 16H phase of the cycle.

Starting on the day of setup (day 0), each turbidostat was sampled for FACS and the corresponding media bottle was weighed to approximate the number of generations. FACS was performed on the Guava easyCyte flow cytometer to calculate the relative ratios of the Selected Gene and YFP strain in each turbidostat. Data were collected every other day through day 14. Selection coefficients were calculated as described above for original line competitions.

### Growth and Photosynthesis Assays

Validated lines were analyzed by a high-throughput 96-well plate-based assay. Briefly, cultures were grown to stationary phase in TAP, HSM, modified HSM (mHSM), and MASM(F) media. Cultures were diluted to OD₇₅₀=0.2 and grown overnight. Overnight growth was followed by a second dilution to OD₇₅₀=0.05. These initial culture densities put the cells in lag or early log phase. At this point, 200 μl of each culture was added to a 96-well microtiter plate in randomized replicates. 96-well microtiter plates used in this assay contain opaque sides and a transparent base so that light exposure is equal across the entire plate. Plates were sealed using a PDMS lid in order to allow for gas exchange but minimize culture volume loss to evaporation. Sealed plates were then set onto a shaker within a growth chamber supplied with 5% CO₂. Intermittent shaking was set to occur for 15 s/min at 1700 rpm. Light incidence upon each plate lid was 140-150 μE. OD₇₅₀ was read at approximately 6 hour intervals for a maximum of 96 hours. The resulting OD₇₅₀ readings, which reflect culture growth, were plotted vs. time. A linear selection algorithm was used to determine the growth rate (see results).

Selected Genes were also assessed for photosynthetic quantum yield using the FluorCAM 800MF (Photon Systems Instruments; Brno, Czech Republic). The FluorCAM works by exposing cultures to pulses of saturating light, which briefly suppresses photochemical yield and induces maximal fluorescence yield. The FluorCAM specializes in the quick and reliable assessment of the effective quantum yield of photochemical energy conversion in photosynthesis. Samples were grown in TAP media to saturation in 96-well deep-well blocks. Cultures were acclimated in additional media—HSM, mHSM, and MASM(F)—by 1:10 dilution in deep-well blocks. Blocks were incubated in a CO₂ controlled growth box under constant light of 80-100 μE for two days prior to screening. Samples were screened in triplicate in 96-well clear-bottom, white microplates. Wild type C. reinhardtii was included as a control. Samples were dark adapted ten minutes prior to imaging. The minimum fluorescence signal (F₀) and the maximal yield (Fₘ) were measured and the photosynthesis yield (Y=F_{V}/Fₘ) was calculated. Analysis was performed with FluorCam7 software.

Individual cells from each Selected Gene were imaged and certain observable traits measured in an attempt to find correlations between easily quantifiable phenotypes and growth advantage over wild type. Analysis was performed with a Fluid Imaging Technologies FlowCAM instrument. The FlowCam gathers images of cells passing through a capillary in front of various microscope objectives. Sapphire uses the FlowCAM in crop protection, cultural integrity, and production applications to observe the distribution of stressed versus healthy cells, pest types and frequency, and for the quantification of invading algal weeds. The C. reinhardtii analysis discussed here utilized a 50 uM glass capillary and 20× microscope objective.

Each Selected Gene line was grown to saturation in liquid TAP media. Cultures were than split back into HSM media (100 ul culture to 4.9 ml media) and sampled for analysis during subsequent log-phase growth. Culture samples were diluted 9:1 in dH₂O and 3000 images captured for each line (example at right). A filter was developed based on image size, aspect ratio, circle-fit, and ratio of blue to green pixels to sort out non-algae particles (i.e. air bubbles and dead cells) and images containing multiple algae cells. Manual review of filter-selected images was performed for each line.

### Biochemical Assays

Selected genes were processed by Fourier transform infrared spectroscopy (FT-IR) to analyze fatty acid content. Briefly, cultures were grown to saturation in TAP media and subsequently acclimated in HSM media in a CO₂ controlled growth box. 50 ml flasks were inoculated with each line at an OD₇₅₀ of 0.05 and grown under ^{˜}350 μE/m² of constant light. Cultures were harvested by centrifugation in mid-log phase (OD₇₅₀=0.4-0.5). Cell pellets were washed once with distilled water and centrifuged a second time to remove any excess water. 35 μl of a thick paste (^{˜}5-10 mg) was spotted onto a 96-well diffuse reflectance IR plate, dried for 1 hr in a vacuum oven (80° C.), and cooled in a desiccator. All samples were spotted in triplicate and NIR (near-infrared) spectra were collected using a Nicolet iS50 FT-IR spectrometer equipped with a 96-well plate reader XY autosampler from PIKE Technologies. Total relative lipid content (TRLC) was predicted for each spectrum using a PLS (partial least squares) model created in TQ Analyst. The range of the model spans from 11%-32% lipid as measured by FAME (fatty acid methyl ester) analysis with an RMSEP (root mean square error of prediction) of 2.3%.

### Validation Results

### Original Line Competitions

Of the 96 selected lines, 95 were successfully competed against wild type in turbidostats. The majority of lines have an average positive Δs_{wt} value in this experiment (91 lines). A one-sample, one-sided t-test was employed by calculating a 95% confidence interval (CI, α=0.025) from the standard deviation followed by comparison of this CI to the average. Any s measurements with a CI less than the average were determined to be statistically greater than zero. 55 lines passed this statistical test. One line showed a Δs_{wt} value of 0 or below for all replicates and is considered to have failed validation (W1813). A few lines had negative mean s values but had individual replicates with positive values—these were advanced to the next stage of validation. The original lines representing the selected genes were also run in an en masse competition experiment. All lines were combined in approximately equal amounts and allowed to grow and compete in replicate turbidostats for two weeks.

### Regenerated Line Competitions

Regenerated lines for all of the original winning lines representing 93 selected genes were created. All regenerated lines entered into competitions with wild type via a common competitor in turbidostats. The samples that entered turbidostat competition contained a pool of 12 transgenic lines. It is likely that only some of these lines are expressing the selected gene to a level sufficient to cause the phenotype of increased selection coefficient. The other lines within the pool could thus have no selective advantage over wild type in turbidostat growth or could be at a disadvantage. Since this would result in a lower overall selection coefficient, the competition was continued for fourteen days.

The table below includes the selection coefficients calculated from the original lines (mean and standard deviation) as well as the s calculations (mean and standard deviation) from the regenerated lines. Missing data represents original lines that were not available for screening. One regenerated line (rW1813) entered the competition phase despite failing to pass the original line competition threshold.

TABLE 33

| | Original Lines | | Regenerated Lines | |
|---|---|---|---|---|
| Winner ID | ΔS_{avg}/gen | STDEV | ΔS_{avg}/gen | STDEV |
| W1313 | 0.1589 | 0.0192 | −0.0403 | 0.0553 |
| W1314 | 0.1371 | 0.0298 | −0.0305 | 0.026 |
| W1315 | 0.2938 | 0.0134 | −0.0639 | 0.0142 |
| W1316 | 0.3082 | 0.1022 | −0.0562 | 0.023 |
| W1317 | 0.1178 | 0.0127 | −0.0246 | 0.031 |
| W1318 | 0.2224 | 0.0243 | −0.0345 | 0.0222 |
| W1324 | 0.2113 | 0.0555 | −0.0181 | 0.0318 |
| W1335 | 0.1403 | 0.0879 | −0.0572 | 0.0121 |
| W1336 | 0.2226 | 0.0111 | −0.0192 | 0.0139 |
| W1342 | 0.178 | 0.0527 | −0.0622 | 0.0251 |
| W1343 | −0.0613 | 0.093 | −0.0506 | 0.0162 |
| W1350 | 0.3299 | 0.0324 | 0.0026 | 0.0279 |
| W1352 | 0.2277 | 0.0421 | −0.0666 | 0.028 |
| W1363 | 0.2357 | 0.061 | −0.0187 | 0.0317 |
| W1370 | 0.1087 | 0.0537 | −0.0032 | 0.0189 |
| W1381 | 0.0865 | 0.1323 | −0.0631 | 0.0082 |
| W1382 | 0.3334 | 0.0252 | 0.0106 | 0.0099 |
| W1386 | 0.39 | 0.0447 | −0.069 | 0.0154 |
| W1399 | 0.0764 | 0.1134 | −0.0872 | 0.0342 |
| W1400 | 0.3382 | 0.0272 | −0.0657 | 0.0088 |
| W1401 | 0.326 | 0.0169 | −0.0467 | 0.0171 |
| W1402 | 0.3742 | 0.0523 | −0.0099 | 0.0254 |
| W1411 | | | −0.0209 | 0.0588 |
| W1416 | 0.1939 | 0.0943 | −0.0021 | 0.0446 |
| W1418 | 0.3153 | 0.0252 | −0.0388 | 0.0326 |
| W1424 | 0.2886 | 0.0207 | −0.0614 | 0.0198 |
| W1429 | 0.2865 | 0.0314 | −0.0316 | 0.0385 |
| W1440 | 0.2475 | 0.0784 | −0.0389 | 0.0298 |
| W1446 | 0.2851 | 0.0429 | 0.1336 | 0.0695 |
| W1452 | 0.3061 | 0.0899 | −0.0488 | 0.0039 |
| W1456 | 0.3038 | 0.0872 | −0.0498 | 0.0636 |
| W1460 | 0.3091 | 0.0322 | −0.0333 | 0.0343 |
| W1463 | 0.3782 | 0.0859 | −0.0294 | 0.0302 |
| W1468 | 0.3637 | 0.063 | −0.0616 | 0.016 |
| W1476 | 0.2578 | 0.0127 | −0.0473 | 0.0171 |
| W1479 | 0.2243 | 0.0691 | 0.0141 | 0.0072 |
| W1480 | 0.3464 | 0.029 | −0.0124 | 0.0224 |
| W1488 | 0.3062 | 0.0467 | −0.0175 | 0.0125 |
| W1491 | 0.2902 | 0.0157 | 0.0044 | 0.0281 |
| W1492 | 0.2945 | 0.013 | 0.0406 | 0.0134 |
| W1493 | 0.2025 | 0.1525 | 0.0323 | 0.0197 |
| W1495 | 0.1173 | 0.2066 | −0.0563 | 0.0486 |
| W1508 | 0.3263 | 0.0251 | −0.0278 | 0.0251 |
| W1509 | 0.1998 | 0.0647 | −0.004 | 0.0235 |
| W1510 | 0.3509 | 0.0849 | −0.0023 | 0.0341 |
| W1511 | 0.2848 | 0.1293 | −0.0006 | 0.0773 |
| W1517 | 0.3427 | 0.0843 | 0.0434 | 0.0073 |
| W1524 | 0.1894 | 0.1186 | −0.0439 | 0.0337 |
| W1525 | 0.357 | 0.018 | −0.0403 | 0.0268 |
| W1529 | 0.3575 | 0.0567 | 0.0237 | 0.028 |
| W1536 | 0.4195 | 0.0215 | −0.0547 | 0.0348 |
| W1559 | 0.3473 | 0.0557 | 0.021 | 0.0532 |
| W1564 | 0.2546 | 0.0516 | −0.0068 | 0.0268 |
| W1580 | 0.2229 | 0.0309 | 0.0228 | 0.0351 |
| W1586 | 0.3395 | 0.1292 | −0.0134 | 0.0027 |
| W1602 | 0.2609 | 0.1305 | −0.0095 | 0.0456 |
| W1604 | 0.1971 | 0.136 | −0.0144 | 0.0143 |
| W1613 | 0.1916 | 0.098 | −0.0174 | 0.0279 |
| W1615 | 0.3894 | 0.0541 | −0.0143 | 0.0305 |
| W1624 | 0.243 | 0.0704 | −0.0009 | 0.0291 |
| W1627 | 0.3036 | 0.0841 | −0.0302 | 0.0215 |
| W1644 | 0.2225 | 0.1369 | −0.049 | 0.0299 |
| W1646 | 0.4715 | 0.0566 | −0.0071 | 0.0485 |
| W1649 | 0.3943 | 0.1019 | −0.0064 | 0.026 |
| W1660 | 0.2854 | 0.0829 | 0.0342 | 0.0209 |
| W1663 | 0.2368 | 0.0042 | −0.0046 | 0.0395 |
| W1665 | 0.2261 | 0.0155 | −0.0055 | 0.0062 |
| W1667 | 0.4025 | 0.0496 | −0.0388 | 0.0141 |
| W1671 | 0.2123 | 0.156 | −0.015 | 0.0115 |
| W1686 | 0.3175 | 0.0328 | −0.0017 | 0.0361 |
| W1688 | 0.2124 | 0.0928 | −0.0311 | 0.0199 |
| W1696 | 0.3397 | 0.033 | −0.0421 | 0.0488 |
| W1702 | 0.2287 | 0.1093 | −0.0504 | 0.0265 |
| W1705 | 0.345 | 0.1233 | 0.0085 | 0.0401 |
| W1712 | 0.3892 | 0.0567 | −0.0526 | 0.005 |
| W1724 | 0.4523 | 0.0216 | 0.0393 | 0.0252 |
| W1732 | 0.2368 | 0.0467 | −0.0026 | 0.014 |
| W1739 | 0.0908 | 0.0856 | −0.0155 | 0.0225 |
| W1740 | 0.3893 | 0.0543 | −0.0186 | 0.022 |
| W1743 | 0.1917 | 0.0502 | −0.0312 | 0.0669 |
| W1758 | 0.0764 | 0.1474 | 0.0337 | 0.0125 |
| W1779 | 0.1991 | 0.0521 | 0.0167 | 0.036 |
| W1780 | 0.1032 | 0.026 | −0.0531 | 0.0164 |
| W1786 | 0.1349 | 0.1061 | −0.0339 | 0.0278 |
| W1796 | 0.1688 | 0.0486 | −0.0321 | 0.011 |
| W1806 | −0.0122 | 0.0824 | −0.0226 | 0.0116 |
| W1811 | 0.0521 | 0.0257 | −0.0378 | 0.0793 |
| W1812 | 0.1862 | 0.0493 | −0.0035 | 0.0239 |
| W1813 | −0.0379 | 0.016 | −0.0024 | 0.0184 |
| W1818 | 0.1305 | 0.0438 | −0.0148 | 0.0313 |
| W1826 | 0.209 | 0.0514 | −0.0367 | 0.0122 |
| W1827 | 0.0966 | 0.0502 | −0.0266 | 0.0342 |
| W1834 | −0.0521 | 0.1014 | −0.0146 | 0.0291 |
| W1849 | 0.1258 | 0.0644 | 0.0363 | 0.0058 |
| W1853 | 0.1789 | 0.0171 | 0.0739 | 0.0202 |
| W1856 | 0.1822 | 0.061 | 0.0128 | 0.0811 |

### Valadated Genes

The data for the selection coefficients divides the winning lines into four classes. In general, the Δs value from the original line is a better representation of the selective advantage of a gene. Regenerated line data, because it results from the combined phenotype of 12 independent clones, is less representative of absolute selective advantage and is more of a binary test to confirm that the original line data is due solely to selected gene expression. Class 1 includes those lines that had original lines that were significantly greater than 0 (95% confidence interval as described previously) and regenerated lines that had positive Δs average values. This class contains 15 lines (W1313, W1317, W1350, W1382, W1402, W1446, W1491, W1492, W1517, W1529, W1559, W1580, W1724, W1779, W1853) representing 15 selected genes.

Class 2 includes lines that had original lines that were significantly greater than 0 and had two regenerated line replicates with a positive Δs value. This class contains 7 lines (W1510, W1646, W1649, W1663, W1686, W1732, W1812) representing 7 selected genes.

Class 3 includes lines that had average Δs values greater than 0.05 for the original with regenerated lines that had positive Δs average values. This class contains 7 lines (W1479, W1493, W1660, W1705, W1758, W1849, W1856), one of which is represented by a Selected Gene in Class 1 (W1479) and another which is represented in Class 2 (W1660).

Finally, Class 4 includes those lines with average Δs values greater than 0.05 for the original lines and had two regenerated line replicates with a positive Δs value. This class contains 1 line (W1739).

The strong performance of specific winning lines in the en masse competition warranted additional regenerated line turbidostat competitions. Any winning line with a selection coefficient greater than 0 in six or more replicates of the en masse yet only one positive Δs value with the regenerated line was repeated in regenerated line 1:1 competitions. W1313 and W1317 initially did not satisfy the criteria to fall into any of the four classes, but are now considered Class 1 Validated Genes.

In all, 28 Desmodesmus sp. genes, represented by 30 winning lines, were considered validated. The validation process is reflected in the table below.

TABLE 34

| Selected Genes | 96 lines, 93 genes |
|---|---|
| Original Line Competiton | A replicate s value >0.01 |
| | 94 lines, 91 genes |
| Class 1 | Original line significantly different from 0 |
| | Average Δs values of regenerated line >0 |
| | 15 lines, 15 genes |
| Class 2 | Original line significantly different from 0 |
| | Replicate Δs values of 2 regenerated lines >0 |
| | 7 lines, 7 genes |
| Class 3 | Average Δs value of original lines >0.05 |
| | Average Δs value of regenerated lines >0 |
| | 7 lines, 5 genes |
| Class 4 | Average Δs values of original lines >0.05 |
| | Replicate Δs value of 2 regenerated lines >0 |
| | 1 line, I gene |

The table below lists all 93 selected genes and the winning lines representing them, along with the Class to which they are assigned. Winning lines that contain the same gene are listed together. 28 of these selected genes are considered validated, and are indicated by bold text in the Locus ID column.

TABLE 35

| | Winner | | | |
|---|---|---|---|---|
| Gene | ID | Locus ID | BLASTp description | Class |
| 1 | W1317 | g3274 | aldo/keto reductase family | 1 |
| 2 | W1468 | g5170 | | |
| 2 | W1474 | g5170 | | |
| 2 | W1516 | g5170 | | |
| 3 | W1480 | g6237 | LL-diaminopimelate aminotransferase | |
| 4 | W1646 | g7118 | small protein associating with GAPDH and PRK | 2 |
| 4 | W1659 | g7118 | small protein associating with GAPDH and PRK | |
| 4 | W1670 | g7118 | small protein associating with GAPDH and PRK | |
| 4 | W1730 | g7118 | small protein associating with GAPDH and PRK | |
| 5 | W1495 | g111 | | |
| 6 | W1400 | g2616 | | |
| 7 | W1624 | g2754 | | |
| 7 | W1649 | g2754 | | 2 |
| 8 | W1476 | g3029 | | |
| 9 | W1602 | g3907 | | |
| 10 | W1452 | g4823 | thioredoxin-like protein | |
| 11 | W1313 | g4907 | | 1 |
| 12 | W1498 | g5535 | | |
| 12 | W1696 | g5535 | | |
| 13 | W1705 | g5656 | phospholipase/carboxylesterase | 3 |
| 14 | W1336 | g5721 | | |
| 15 | W1456 | g6298 | | |
| 16 | W1525 | g655 | | |
| 17 | W1370 | g6598 | | |
| 18 | W1740 | g6615 | | |
| 19 | W1446 | g6739 | | 1 |
| 20 | W1491 | g76 | | 1 |
| 21 | W1508 | g8033 | | |
| 22 | W1463 | scaffold145: | | |
| | | 367069-368161 | | |
| 23 | W1402 | scaffold223: | | 1 |
| | | 117584-119864 | | |
| 24 | W1311 | scaffold428: | | |
| | | 13750-16208 | | |
| 24 | W1342 | scaffold428: | | |
| | | 13750-16208 | | |
| 25 | W1314 | scaffold458: | TOR kinase binding protein | |
| | | 139916-142258 | | |
| 25 | W1566 | scaffold458: | TOR kinase binding protein | |
| | | 139916-142258 | | |
| 25 | W1326 | scaffold458: | TOR kinase binding protein | |
| | | 139916-142333 | | |
| 26 | W1712 | scaffold459: | | |
| | | 6959-7079 | | |
| 27 | W1667 | g11029 | psbP domain-containing protein | |
| 28 | W1424 | g4138 | NPL4-domain-containing protein | |
| 29 | W1343 | scaffold118: | | |
| | | 210748-213562 | | |
| 30 | W1363 | scaffold382: | | |
| | | 133727-134579 | | |
| 31 | W1335 | scaffold4: | | |
| | | 561494-561855 | | |
| 32 | W1418 | g1360 | | |
| 33 | W1475 | g1656 | | |
| 33 | W1493 | g1656 | | 3 |
| 34 | W1673 | g1790 | light-harvesting chlorophyll-a/b binding protein | |
| 34 | W1686 | g1790 | light-harvesting chlorophyll-a/b binding protein | 2 |
| 34 | W1726 | g1790 | light-harvesting chlorophyll-a/b binding protein | |
| 35 | W1580 | g2186 | cytochrome c oxidase subunit | 1 |
| 36 | W1688 | g2533 | | |
| 37 | W1702 | g2961 | | |
| 38 | W1315 | g3149 | | |
| 39 | W1429 | g3558 | | |
| 40 | W1586 | g430 | | |
| 41 | W1440 | g446 | | |
| 41 | W1682 | g446 | | |
| 42 | W1381 | g4573 | | |
| 43 | W1559 | g4732 | | 1 |
| 44 | W1510 | g5667 | | 2 |
| 44 | W1555 | g5667 | | |
| 45 | W1382 | g5980 | predicted protein [C. reinhardtii] | 1 |
| 46 | W1511 | g7052 | | |
| 47 | W1517 | g7085 | hypothetical protein [V. carteri f. nagariensis] | 1 |
| 48 | W1724 | g7161 | | 1 |
| 49 | W1627 | g7574 | ribosomal protein S9 | |
| 49 | W1701 | g7574 | ribosomal protein S9 | |
| 50 | W1386 | g8029 | GDP-D-mannose pyrophosphorylase | 1 |
| 51 | W1529 | g8172 | | |
| 52 | W1613 | g8516 | | |
| 53 | W1401 | g904 | | |
| 54 | W1488 | g9426 | DEAD-box ATP-dependent RNA helicase 2-like | |
| 55 | W1604 | g9868 | | |
| 56 | W1509 | scaffold116: | | |
| | | 110230-110988 | | |
| 57 | W1564 | scaffold14: | | |
| | | 157001-157683 | | |
| 58 | W1732 | scaffold150: | | 2 |
| | | 396278-396306 | | |
| 59 | W1615 | scaffold19: | | |
| | | 34476-35175 | | |
| 60 | W1310 | scaffold20: | | |
| | | 41777-42284 | | |
| 60 | W1399 | scaffold20: | | |
| | | 41777-42284 | | |
| 61 | W1352 | scaffold250: | | |
| | | 278860-279443 | | |
| 62 | W1460 | scaffold264: | | |
| | | 186217-187272 | | |
| 63 | W1739 | scaffold318: | hypothetical protein [C. variabilis] | 4 |
| | | 127147-127942 | | |
| 64 | W1536 | scaffold343: | | |
| | | 214404-215059 | | |
| 65 | W1524 | scaffold357: | | |
| | | 50700-51706 | | |
| 66 | W1671 | scaffold557: | endoxylanase II | |
| | | 3085-3109 | | |
| 67 | W1324 | scaffold584: | | |
| | | 141077-141746 | | |
| 68 | W1644 | scaffold70: | | |
| | | 98097-98851 | | |
| 69 | W1318 | scaffold732: | | |
| | | 18860-19706 | | |
| 70 | W1492 | scaffold79: | | 1 |
| | | 428425-428443 | | |
| 71 | W1416 | g1253 | | |
| 71 | W1648 | g1253 | | |
| 72 | W1385 | g2004 | | |
| 72 | W1387 | g2004 | | |
| 72 | W1411 | g2004 | | |
| 73 | W1660 | g2209 | light-harvesting chlorophyll-a/b binding protein | 3 |
| 73 | W1663 | g2209 | light-harvesting chlorophyll-a/b binding protein | 2 |
| 74 | W1365 | g5156 | | |
| 74 | W1665 | g5156 | | |
| 75 | W1316 | g5809 | hypothetical protein [C. reinhardtii] | |
| 75 | W1384 | g5809 | hypothetical protein [C. reinhardtii] | |
| 76 | W1350 | g623 | RuBisCO small subunit | 1 |
| 76 | W1479 | g623 | RuBisCO small subunit | 3 |
| 76 | W1567 | g623 | RuBisCO small subunit | |
| 77 | W1758 | AmaxDRAFT_1006 | alpha/beta hydrolase fold protein | 3 |
| 78 | W1834 | AmaxDRAFT_1040 | photosystem I reaction centre subunit XI PsaL | |
| 79 | W1780 | AmaxDRAFT_2566 | oxidoreductase domain protein | |
| 80 | W1818 | AmaxDRAFT_2699 | multi-sensor signal transduction histidine kinase | |
| 81 | W1853 | AmaxDRAFT_3755 | hypothetical protein | 1 |
| 82 | W1806 | AmaxDRAFT_0253 | lipolytic protein G-D-S-L family | |
| 83 | W1827 | AmaxDRAFT_0292 | GDP-mannose 4,6-dehydratase | |
| 84 | W1796 | AmaxDRAFT_0673 | hypothetical protein | |
| 85 | W1743 | AmaxDRAFT_1243 | anion-transporting ATPase | |
| 86 | W1786 | AmaxDRAFT_2858 | multi-sensor signal transduction histidine kinase | |
| 87 | W1856 | AmaxDRAFT_3426 | putative ATP-dependent DNA helicase DinG | 3 |
| 88 | W1779 | AmaxDRAFT_4116 | serine/threonine protein kinase with | 1 |
| | | | pentapeptide repeats | |
| 89 | W1813 | AmaxDRAFT_5119 | heat shock protein Dna [symbol] domain protein | |
| 90 | W1812 | AmaxDRAFT_0926 | isoleucyl-tRNA synthetase | 2 |
| 91 | W1826 | AmaxDRAFT_4072 | conserved hypothetical protein | |
| 92 | W1849 | NZ_ABYK01000001:479 | | 3 |
| | | 96-48113 | | |
| 94 | W1760 | AmaxDRAFT_3680 | NB-ARC domain protein | |
| 94 | W1811 | AmaxDRAFT_3680 | NB-ARC domain protein | |

In order to further rank and distinguish winning lines and selected genes from each other, an ANOVA with Tukey-Kramer HSD test was completed on each set of selection coefficient data. This test is a single-step multiple comparison procedure and statistical test to find which means are significantly different from one another. The test compares the means of every sample to the means of every other sample; that is, it applies simultaneously to the set of all pairwise comparisons and identifies where the difference between two means is greater than the standard error would be expected to allow.

### Growth and Biochemical Characteristics

Validated Genes (30 lines) were tested in microtiter plate growth assays using four different media: HSM, mHSM, MASM(F), and TAP. HSM, mHSM, and MASM(F) are minimal medias with different nitrogen sources (NH₄ for HSM, NO₃ for mHSM and MASM) while TAP contains an organic carbon source (acetate) and supports mixotrophic growth.

The OD₇₅₀ versus time data were not suitable for logistic curve fitting for all wells. Therefore, an exponential analysis was performed in order to calculate growth rates. With this type of analysis, the OD₇₅₀ data were plotted with time. Then, the linear region of these data was selected to define the log phase growth region of the curve. The most difficult part of this type of analysis was to determine which data represent “the linear region.” This experiment studied clones having different growth profiles; therefore a subjective time range to analyze was not suitable. In order to overcome this challenge, an algorithm for selecting the linear region of the OD₇₅₀ versus time data was developed and programmed into MS Excel VBA to analyze the data.

The linear selection algorithm uses a two phase process. Phase one of the algorithm steps through all the transformed data using all possible starting points and between 4 and 7 consecutive points to calculate the Slope, R², and the t value of the slope. Any slopes failing the t-test were rejected, α=0.05 confidence level (Kachigan. Multivariate Statistical Analysis, 2^{nd} Ed. (1991) ISBN 0-942154-91-6; p178). Of the slopes which had a significant value by the t-test, the one having the maximum product of Slope*R² was selected as representing the linear region. The slope of this linear region was used to score the growth rates of the clone. Growth rate for each well was determined independently. These resulting growth rates were then analyzed in JMP.

Below is a summary table for the microtiter plate growth rate experiments. An ANOVA with Dunnett's statistic test (p<0.05) was applied to the samples to determine which were significantly different than wild type. Those lines that are statistically greater than wild type are highlighted in bold text below.

TABLE 36

| | TAP | | HSM | | mHSM | | MASM(F) | |
|---|---|---|---|---|---|---|---|---|
| Winner ID | Mean | STDEV | Mean | STDEV | Mean | STDEV | Mean | STDEV |
| Wild Type | 0.0384 | 0.0033 | 0.0203 | 0.0022 | 0.0276 | 0.0030 | 0.0166 | 0.0021 |
| W1313 | 0.0373 | 0.0032 | 0.0162 | 0.0028 | 0.0291 | 0.0040 | 0.0105 | 0.0032 |
| W1317 | 0.0312 | 0.0022 | 0.0175 | 0.0030 | 0.0255 | 0.0041 | 0.0106 | 0.0007 |
| W1350 | 0.0386 | 0.0019 | 0.0162 | 0.0021 | 0.0310 | 0.0042 | 0.0094 | 0.0024 |
| W1382 | 0.0372 | 0.0017 | 0.0218 | 0.0010 | 0.0232 | 0.0016 | 0.0142 | 0.0011 |
| W1402 | 0.0345 | 0.0014 | 0.0082 | 0.0023 | 0.0255 | 0.0012 | 0.0101 | 0.0015 |
| W1446 | 0.0350 | 0.0032 | 0.0228 | 0.0017 | 0.0314 | 0.0030 | 0.0091 | 0.0012 |
| W1479 | 0.0342 | 0.0021 | 0.0218 | 0.0014 | 0.0253 | 0.0036 | 0.0092 | 0.0015 |
| W1491 | 0.0295 | 0.0012 | 0.0190 | 0.0008 | 0.0166 | 0.0020 | 0.0080 | 0.0011 |
| W1492 | 0.0311 | 0.0037 | 0.0203 | 0.0017 | 0.0182 | 0.0009 | 0.0113 | 0.0016 |
| W1493 | 0.0299 | 0.0022 | 0.0167 | 0.0008 | 0.0157 | 0.0011 | 0.0087 | 0.0010 |
| W1510 | 0.0367 | 0.0028 | 0.0160 | 0.0010 | 0.0333 | 0.0079 | 0.0103 | 0.0012 |
| W1517 | 0.0376 | 0.0031 | 0.0157 | 0.0022 | 0.0206 | 0.0022 | 0.0080 | 0.0011 |
| W1529 | 0.0396 | 0.0021 | 0.0189 | 0.0021 | 0.0319 | 0.0033 | 0.0088 | 0.0015 |
| W1559 | 0.0344 | 0.0022 | 0.0191 | 0.0011 | 0.0150 | 0.0012 | 0.0119 | 0.0008 |
| W1580 | 0.0239 | 0.0007 | 0.0191 | 0.0025 | 0.0137 | 0.0022 | 0.0115 | 0.0012 |
| W1646 | 0.0299 | 0.0015 | 0.0178 | 0.0031 | 0.0234 | 0.0018 | 0.0100 | 0.0024 |
| W1649 | 0.0333 | 0.0014 | 0.0159 | 0.0009 | 0.0282 | 0.0021 | 0.0099 | 0.0018 |
| W1660 | 0.0402 | 0.0038 | 0.0140 | 0.0024 | 0.0199 | 0.0013 | 0.0108 | 0.0019 |
| W1663 | 0.0329 | 0.0033 | 0.0196 | 0.0040 | 0.0306 | 0.0021 | 0.0167 | 0.0021 |
| W1686 | 0.0341 | 0.0029 | 0.0220 | 0.0014 | 0.0230 | 0.0009 | 0.0124 | 0.0026 |
| W1705 | 0.0345 | 0.0037 | 0.0144 | 0.0060 | 0.0247 | 0.0023 | 0.0137 | 0.0005 |
| W1724 | 0.0362 | 0.0044 | 0.0132 | 0.0022 | 0.0328 | 0.0036 | 0.0138 | 0.0020 |
| W1732 | 0.0344 | 0.0022 | 0.0179 | 0.0011 | 0.0193 | 0.0015 | 0.0093 | 0.0006 |
| W1739 | 0.0303 | 0.0025 | 0.0151 | 0.0025 | 0.0185 | 0.0019 | 0.0098 | 0.0008 |
| W1758 | 0.0299 | 0.0031 | 0.0179 | 0.0019 | 0.0223 | 0.0016 | 0.0069 | 0.0014 |
| W1779 | 0.0328 | 0.0035 | 0.0165 | 0.0022 | 0.0135 | 0.0032 | 0.0076 | 0.0014 |
| W1812 | 0.0347 | 0.0109 | 0.0140 | 0.0020 | 0.0333 | 0.0039 | 0.0081 | 0.0004 |
| W1849 | 0.0309 | 0.0056 | 0.0179 | 0.0011 | 0.0226 | 0.0014 | 0.0072 | 0.0019 |
| W1853 | 0.0341 | 0.0021 | 0.0174 | 0.0029 | 0.0250 | 0.0014 | 0.0103 | 0.0009 |
| W1856 | 0.0309 | 0.0033 | 0.0184 | 0.0024 | 0.0267 | 0.0045 | 0.0087 | 0.0017 |

96 Selected Genes were screened for photosynthetic yield using the FluorCAM. All strains were tested in both HSM, mHSM, MASM(F), and TAP media. Values for photosynthetic yield are listed in the table below. Analysis of these data result in lines that are statistically different than wild type, however all lines are considered to be photosynthetically healthy based on their Fᵥ/Fₘ values.

TABLE 37

| | HSM | | mHSM | | MASM(F) | | TAP | |
|---|---|---|---|---|---|---|---|---|
| Winner ID | FᵥFₘ | STDEV | FᵥFₘ | STDEV | FᵥFₘ | STDEV | FᵥFₘ | STDEV |
| Wild Type | 0.7575 | 0.0046 | 0.7488 | 0.0064 | 0.7575 | 0.0046 | 0.7200 | 0.0076 |
| W1313 | 0.7500 | 0.0100 | 0.7667 | 0.0058 | 0.7600 | 0.0000 | 0.7100 | 0.0000 |
| W1314 | 0.7500 | 0.0000 | 0.7400 | 0.0000 | 0.7600 | 0.0000 | 0.6833 | 0.0058 |
| W1315 | 0.7500 | 0.0000 | 0.7400 | 0.0000 | 0.7600 | 0.0000 | 0.7333 | 0.0058 |
| W1316 | 0.7533 | 0.0058 | 0.7500 | 0.0000 | 0.7500 | 0.0000 | 0.6900 | 0.0000 |
| W1317 | 0.7333 | 0.0058 | 0.7600 | 0.0000 | 0.7667 | 0.0058 | 0.7300 | 0.0000 |
| W1318 | 0.7200 | 0.0000 | 0.7400 | 0.0000 | 0.7500 | 0.0000 | 0.7200 | 0.0000 |
| W1324 | 0.7400 | 0.0000 | 0.7500 | 0.0000 | 0.7700 | 0.0000 | 0.7300 | 0.0000 |
| W1335 | 0.7600 | 0.0000 | 0.7600 | 0.0000 | 0.7700 | 0.0000 | 0.7300 | 0.0000 |
| W1336 | 0.7200 | 0.0000 | 0.7333 | 0.0058 | 0.7400 | 0.0000 | 0.7300 | 0.0000 |
| W1342 | 0.7267 | 0.0058 | 0.7500 | 0.0000 | 0.7400 | 0.0000 | 0.7000 | 0.0000 |
| W1343 | 0.7500 | 0.0000 | 0.7467 | 0.0058 | 0.7500 | 0.0000 | 0.7100 | 0.0000 |
| W1350 | 0.7500 | 0.0000 | 0.7600 | 0.0000 | 0.7633 | 0.0058 | 0.7100 | 0.0000 |
| W1352 | 0.7500 | 0.0000 | 0.7500 | 0.0000 | 0.7700 | 0.0000 | 0.7133 | 0.0058 |
| W1363 | 0.7667 | 0.0058 | 0.7600 | 0.0000 | 0.7600 | 0.0000 | 0.7400 | 0.0000 |
| W1370 | 0.7567 | 0.0058 | 0.7767 | 0.0058 | 0.7600 | 0.0000 | 0.7200 | 0.0000 |
| W1381 | 0.7467 | 0.0058 | 0.7700 | 0.0000 | 0.7700 | 0.0000 | 0.7500 | 0.0000 |
| W1382 | 0.7600 | 0.0000 | 0.7667 | 0.0058 | 0.7700 | 0.0000 | 0.7400 | 0.0000 |
| W1386 | 0.7433 | 0.0058 | 0.7500 | 0.0000 | 0.7500 | 0.0000 | 0.7300 | 0.0000 |
| W1399 | 0.7333 | 0.0058 | 0.7600 | 0.0000 | 0.7600 | 0.0000 | 0.7000 | 0.0000 |
| W1400 | 0.7300 | 0.0000 | 0.7300 | 0.0000 | 0.7200 | 0.0000 | 0.7200 | 0.0000 |
| W1401 | 0.7300 | 0.0000 | 0.7300 | 0.0000 | 0.7500 | 0.0000 | 0.7000 | 0.0000 |
| W1402 | 0.7600 | 0.0000 | 0.7667 | 0.0058 | 0.7600 | 0.0000 | 0.7500 | 0.0000 |
| W1416 | 0.7200 | 0.0000 | 0.7700 | 0.0000 | 0.7700 | 0.0000 | 0.7400 | 0.0000 |
| W1418 | 0.7600 | 0.0000 | 0.7800 | 0.0000 | 0.7700 | 0.0000 | 0.7400 | 0.0000 |
| W1424 | 0.7333 | 0.0058 | 0.7500 | 0.0000 | 0.7667 | 0.0058 | 0.6767 | 0.0058 |
| W1429 | 0.7133 | 0.0058 | 0.7400 | 0.0000 | 0.7567 | 0.0058 | 0.6300 | 0.0000 |
| W1440 | 0.7433 | 0.0058 | 0.7300 | 0.0000 | 0.7300 | 0.0000 | 0.7200 | 0.0000 |
| W1446 | 0.7400 | 0.0000 | 0.7400 | 0.0000 | 0.7500 | 0.0000 | 0.7200 | 0.0000 |
| W1452 | 0.7400 | 0.0000 | 0.7600 | 0.0000 | 0.7700 | 0.0000 | 0.7300 | 0.0000 |
| W1456 | 0.7567 | 0.0058 | 0.7800 | 0.0000 | 0.7700 | 0.0000 | 0.7433 | 0.0058 |
| W1460 | 0.7467 | 0.0058 | 0.7500 | 0.0000 | 0.7700 | 0.0000 | 0.7333 | 0.0058 |
| W1463 | 0.7433 | 0.0058 | 0.7600 | 0.0000 | 0.7700 | 0.0000 | 0.7500 | 0.0000 |
| W1468 | 0.7333 | 0.0058 | 0.7800 | 0.0000 | 0.7800 | 0.0000 | 0.7400 | 0.0000 |
| W1476 | 0.7300 | 0.0000 | 0.7367 | 0.0058 | 0.7600 | 0.0000 | 0.6800 | 0.0000 |
| W1479 | 0.7633 | 0.0058 | 0.7700 | 0.0000 | 0.7733 | 0.0058 | 0.7300 | 0.0000 |
| W1480 | 0.7233 | 0.0058 | 0.7333 | 0.0058 | 0.7500 | 0.0000 | 0.7333 | 0.0058 |
| W1488 | 0.7533 | 0.0058 | 0.7567 | 0.0058 | 0.7700 | 0.0000 | 0.7330 | 0.0000 |
| W1491 | 0.7467 | 0.0058 | 0.7500 | 0.0000 | 0.7533 | 0.0058 | 0.6967 | 0.0058 |
| W1492 | 0.7367 | 0.0058 | 0.7400 | 0.0000 | 0.7700 | 0.0000 | 0.7100 | 0.0000 |
| W1493 | 0.7500 | 0.0000 | 0.7767 | 0.0058 | 0.7800 | 0.0000 | 0.7400 | 0.0000 |
| W1495 | 0.7400 | 0.0000 | 0.7500 | 0.0000 | 0.7700 | 0.0000 | 0.7333 | 0.0058 |
| W1508 | 0.7400 | 0.0000 | 0.7600 | 0.0000 | 0.7600 | 0.0000 | 0.6700 | 0.0000 |
| W1509 | 0.7400 | 0.0000 | 0.7400 | 0.0000 | 0.7700 | 0.0000 | 0.7200 | 0.0000 |
| W1510 | 0.7500 | 0.0000 | 0.7600 | 0.0000 | 0.7700 | 0.0000 | 0.7367 | 0.0058 |
| W1511 | 0.7600 | 0.0000 | 0.7700 | 0.0000 | 0.7800 | 0.0000 | 0.7500 | 0.0000 |
| W1517 | 0.7600 | 0.0000 | 0.7600 | 0.0000 | 0.7700 | 0.0000 | 0.7300 | 0.0000 |
| W1524 | 0.6900 | 0.0000 | 0.7600 | 0.0000 | 0.7700 | 0.0000 | 0.7400 | 0.0000 |
| W1525 | 0.7300 | 0.0000 | 0.7400 | 0.0000 | 0.7600 | 0.0000 | 0.7300 | 0.0000 |
| W1529 | 0.7333 | 0.0058 | 0.7467 | 0.0058 | 0.7400 | 0.0000 | 0.7100 | 0.0000 |
| W1536 | 0.7500 | 0.0000 | 0.7500 | 0.0000 | 0.7700 | 0.0000 | 0.7300 | 0.0000 |
| W1559 | 0.7500 | 0.0000 | 0.7500 | 0.0000 | 0.7700 | 0.0000 | 0.7333 | 0.0058 |
| W1564 | 0.7800 | 0.0000 | 0.7800 | 0.0000 | 0.7800 | 0.0000 | 0.7333 | 0.0058 |
| W1580 | 0.7467 | 0.0058 | 0.7767 | 0.0058 | 0.7767 | 0.0058 | 0.7533 | 0.0058 |
| W1586 | 0.7533 | 0.0058 | 0.7800 | 0.0000 | 0.7633 | 0.0058 | 0.7033 | 0.0058 |
| W1602 | 0.7333 | 0.0058 | 0.7400 | 0.0000 | 0.7400 | 0.0000 | 0.7433 | 0.0058 |
| W1604 | 0.7400 | 0.0000 | 0.7500 | 0.0000 | 0.7600 | 0.0000 | 0.7467 | 0.0058 |
| W1613 | 0.7633 | 0.0058 | 0.7633 | 0.0058 | 0.7733 | 0.0058 | 0.7500 | 0.0000 |
| W1615 | 0.7600 | 0.0000 | 0.7700 | 0.0000 | 0.7633 | 0.0058 | 0.7733 | 0.0058 |
| W1624 | 0.7467 | 0.0058 | 0.7567 | 0.0058 | 0.7700 | 0.0000 | 0.7300 | 0.0000 |
| W1627 | 0.7567 | 0.0058 | 0.7600 | 0.0000 | 0.7700 | 0.0000 | 0.7200 | 0.0000 |
| W1644 | 0.7500 | 0.0000 | 0.7800 | 0.0000 | 0.7800 | 0.0000 | 0.7400 | 0.0000 |
| W1646 | 0.7700 | 0.0000 | 0.7633 | 0.0058 | 0.7633 | 0.0058 | 0.6833 | 0.0058 |
| W1649 | 0.7667 | 0.0058 | 0.7700 | 0.0000 | 0.7800 | 0.0000 | 0.7400 | 0.0000 |
| W1660 | 0.7700 | 0.0000 | 0.7700 | 0.0000 | 0.7700 | 0.0000 | 0.7467 | 0.0058 |
| W1663 | 0.7433 | 0.0058 | 0.7700 | 0.0000 | 0.7567 | 0.0058 | 0.7400 | 0.0000 |
| W1665 | 0.7600 | 0.0000 | 0.7500 | 0.0000 | 0.7700 | 0.0000 | 0.7500 | 0.0000 |
| W1667 | 0.7600 | 0.0000 | 0.7500 | 0.0000 | 0.7600 | 0.0000 | 0.7400 | 0.0000 |
| W1671 | 0.7600 | 0.0000 | 0.7600 | 0.0000 | 0.7700 | 0.0000 | 0.7400 | 0.0000 |
| W1686 | 0.7800 | 0.0000 | 0.7800 | 0.0000 | 0.7700 | 0.0000 | 0.7300 | 0.0000 |
| W1688 | 0.7500 | 0.0000 | 0.7533 | 0.0058 | 0.7700 | 0.0000 | 0.7400 | 0.0000 |
| W1696 | 0.7500 | 0.0000 | 0.7700 | 0.0000 | 0.7700 | 0.0000 | 0.7567 | 0.0058 |
| W1702 | 0.7533 | 0.0058 | 0.7500 | 0.0000 | 0.7700 | 0.0000 | 0.7100 | 0.0000 |
| W1705 | 0.7467 | 0.0058 | 0.7600 | 0.0000 | 0.7700 | 0.0000 | 0.7367 | 0.0058 |
| W1712 | 0.7533 | 0.0058 | 0.7500 | 0.0000 | 0.7700 | 0.0000 | 0.6700 | 0.0000 |
| W1724 | 0.7667 | 0.0058 | 0.7567 | 0.0058 | 0.7700 | 0.0000 | 0.7433 | 0.0058 |
| W1732 | 0.7600 | 0.0000 | 0.7600 | 0.0000 | 0.7767 | 0.0058 | 0.7300 | 0.0000 |
| W1739 | 0.7600 | 0.0000 | 0.7633 | 0.0058 | 0.7800 | 0.0000 | 0.7433 | 0.0058 |
| W1740 | 0.7300 | 0.0000 | 0.7400 | 0.0000 | 0.7500 | 0.0000 | 0.7133 | 0.0058 |
| W1743 | 0.7600 | 0.0000 | 0.7600 | 0.0000 | 0.7733 | 0.0058 | 0.7300 | 0.0000 |
| W1758 | 0.7633 | 0.0058 | 0.7500 | 0.0000 | 0.7600 | 0.0000 | 0.7100 | 0.0000 |
| W1779 | 0.7333 | 0.0058 | 0.7500 | 0.0000 | 0.7700 | 0.0000 | 0.7400 | 0.0000 |
| W1780 | 0.7667 | 0.0058 | 0.7700 | 0.0000 | 0.7767 | 0.0058 | 0.7400 | 0.0000 |
| W1786 | 0.7700 | 0.0000 | 0.7533 | 0.0058 | 0.7700 | 0.0000 | 0.7500 | 0.0000 |
| W1796 | 0.7567 | 0.0058 | 0.7500 | 0.0000 | 0.7700 | 0.0000 | 0.7600 | 0.0000 |
| W1806 | 0.7567 | 0.0058 | 0.7433 | 0.0058 | 0.7700 | 0.0000 | 0.7133 | 0.0058 |
| W1811 | 0.7567 | 0.0058 | 0.7500 | 0.0000 | 0.7733 | 0.0058 | 0.7300 | 0.0000 |
| W1812 | 0.7700 | 0.0000 | 0.7600 | 0.0000 | 0.7700 | 0.0000 | 0.7500 | 0.0000 |
| W1813 | 0.7767 | 0.0058 | 0.7633 | 0.0058 | 0.7700 | 0.0000 | 0.7333 | 0.0058 |
| W1818 | 0.7700 | 0.0000 | 0.7600 | 0.0000 | 0.7700 | 0.0000 | 0.7500 | 0.0000 |
| W1826 | 0.7667 | 0.0058 | 0.7600 | 0.0000 | 0.7700 | 0.0000 | 0.7233 | 0.0058 |
| W1827 | 0.7667 | 0.0058 | 0.7600 | 0.0000 | 0.7700 | 0.0000 | 0.7400 | 0.0000 |
| W1834 | 0.7700 | 0.0000 | 0.7500 | 0.0000 | 0.7600 | 0.0000 | 0.7500 | 0.0000 |
| W1849 | 0.7800 | 0.0000 | 0.7667 | 0.0058 | 0.7700 | 0.0000 | 0.7500 | 0.0000 |
| W1853 | 0.7433 | 0.0058 | 0.7500 | 0.0000 | 0.7667 | 0.0058 | 0.7500 | 0.0000 |
| W1856 | 0.7600 | 0.0000 | 0.7567 | 0.0058 | 0.7700 | 0.0000 | 0.7300 | 0.0000 |

Fluid Imaging software was used to measure approximately 30 size, shape, and color characteristics for each image. An ANOVA with Dunnett's statistic test (p<0.05) was performed on the summary data (Larson. Analysis of Variance with Just Summary Statistics as Input. American Statistician (1992) vol. 46 pp. 151-152.) to determine which samples were significantly different than wild type. Summary statistics and analysis are listed below.

TABLE 38

| Raw Data | | | | Dunnett's Test | |
|---|---|---|---|---|---|
| | Mean | | | Abs(Dif)- | |
| Level | ESD | STDEV | N | LSD | p-Value |
| W1416 | 522.66 | 254.33 | 1482 | 261.6007 | <.0001* |
| W1495 | 463.85 | 225.36 | 2650 | 205.3498 | <.0001* |
| W1446 | 443.02 | 207.46 | 1417 | 181.7159 | <.0001* |
| W1463 | 440.19 | 214.55 | 2308 | 181.1756 | <.0001* |
| W1849 | 417.86 | 231.35 | 2347 | 158.9108 | <.0001* |
| W1826 | 413.91 | 180.54 | 2417 | 155.0733 | <.0001* |
| W1667 | 409.61 | 229.33 | 2597 | 151.0379 | <.0001* |
| W1834 | 395.72 | 156.72 | 2517 | 137.0344 | <.0001* |
| W1386 | 391.87 | 224.37 | 1964 | 132.1844 | <.0001* |
| W1479 | 390.27 | 181.69 | 2260 | 131.1726 | <.0001* |
| W1363 | 388.41 | 215.78 | 2598 | 129.8393 | <.0001* |
| W1440 | 382.84 | 171.1 | 2098 | 123.4385 | <.0001* |
| W1418 | 379.02 | 191.29 | 2476 | 120.2737 | <.0001* |
| W1318 | 375.16 | 197.37 | 2404 | 116.3028 | <.0001* |
| W1665 | 370.23 | 205.18 | 1955 | 110.5241 | <.0001* |
| W1342 | 366.8 | 202.95 | 1278 | 104.9034 | <.0001* |
| W1780 | 364.03 | 199.78 | 2140 | 104.7111 | <.0001* |
| W1818 | 356.7 | 176.12 | 2568 | 98.0875 | <.0001* |
| W1401 | 350.97 | 209.35 | 730 | 85.0603 | <.0001* |
| W1786 | 349.93 | 162.66 | 2325 | 90.9442 | <.0001* |
| W1660 | 348.31 | 161.67 | 2147 | 89.0046 | <.0001* |
| W1511 | 344.7 | 205.16 | 2422 | 85.8711 | <.0001* |
| W1491 | 344.18 | 228.3 | 2028 | 84.6341 | <.0001* |
| W1460 | 333.63 | 176.43 | 2413 | 74.7870 | <.0001* |
| W1316 | 327.02 | 151.39 | 2059 | 67.5391 | <.0001* |
| W1324 | 324.22 | 183.84 | 2238 | 65.0836 | <.0001* |
| W1350 | 323.66 | 154.83 | 2125 | 64.3119 | <.0001* |
| W1812 | 318.15 | 128.08 | 2445 | 59.3567 | <.0001* |
| W1724 | 318.04 | 167.79 | 2118 | 58.6782 | <.0001* |
| W1381 | 317.88 | 199.22 | 1679 | 57.4632 | <.0001* |
| W1343 | 317.14 | 149.25 | 2721 | 58.7323 | <.0001* |
| W1336 | 314.25 | 169.47 | 1773 | 54.0965 | <.0001* |
| W1743 | 307.06 | 137.2 | 2410 | 48.2123 | <.0001* |
| W1314 | 307.05 | 175.29 | 2538 | 48.3948 | <.0001* |
| W1732 | 306.84 | 146.32 | 2515 | 48.1515 | <.0001* |
| W1627 | 302.36 | 189.54 | 2128 | 43.0178 | <.0001* |
| W1853 | 300.04 | 158.39 | 2131 | 40.7037 | <.0001* |
| W1399 | 295.51 | 162.34 | 1618 | 34.9085 | <.0001* |
| W1400 | 293.11 | 175.19 | 2168 | 33.8447 | <.0001* |
| W1468 | 291.98 | 151.65 | 2585 | 33.3913 | <.0001* |
| W1335 | 290.81 | 159.54 | 1209 | 28.5774 | <.0001* |
| W1758 | 285.34 | 155.23 | 1838 | 25.3551 | <.0001* |
| W1644 | 284.26 | 181.71 | 2363 | 25.3370 | <.0001* |
| W1493 | 282.28 | 147.53 | 2405 | 23.4244 | <.0001* |
| W1456 | 274.96 | 124.36 | 2553 | 16.3263 | <.0001* |
| W1686 | 273.65 | 102.28 | 2059 | 14.1691 | <.0001* |
| W1702 | 272.87 | 104.09 | 2249 | 13.7532 | <.0001* |
| W1510 | 270.73 | 148.95 | 1713 | 10.4113 | <.0001* |
| W1696 | 270.49 | 118.06 | 2380 | 11.5945 | <.0001* |
| W1525 | 269.84 | 168.54 | 1979 | 10.1878 | <.0001* |
| W1315 | 266.53 | 144.87 | 2428 | 7.7104 | <.0001* |
| W1856 | 259.72 | 172.74 | 2236 | 0.5800 | 0.0337* |
| W1827 | 258.18 | 102.11 | 2653 | −0.3162 | 0.0620 |
| W1671 | 257.26 | 95.8 | 2710 | −1.1618 | 0.1065 |
| W1712 | 255.29 | 137.77 | 1552 | −5.5252 | 0.5915 |
| W1480 | 255.01 | 157.35 | 1921 | −4.7739 | 0.5171 |
| W1806 | 251.2 | 120.38 | 2201 | −8.0037 | 0.9892 |
| W1424 | 251.06 | 157.5 | 1566 | −9.7086 | 0.9992 |
| W1492 | 248.01 | 115.2 | 1991 | −11.6157 | 1.0000 |
| W1705 | 247.05 | 132.97 | 2222 | −12.1153 | 1.0000 |
| W1602 | 246.4 | 151.64 | 1809 | −13.6588 | 1.0000 |
| W1476 | 245.21 | 117.13 | 2018 | −14.3572 | 1.0000 |
| W1352 | 245.06 | 147.82 | 1707 | −15.2758 | 1.0000 |
| W1313 | 243.89 | 160.46 | 2480 | −14.8503 | 1.0000 |
| SE0050 | 243.63 | 141.8 | 2387 | −14.7342 | 1.0000 |
| W1580 | 243 | 140.87 | 2146 | −14.5273 | 1.0000 |
| W1517 | 240.99 | 129.04 | 2580 | −11.8057 | 1.0000 |
| W1604 | 240.43 | 140.52 | 2213 | −11.8316 | 1.0000 |
| W1536 | 239.04 | 115.14 | 1803 | −11.3344 | 1.0000 |
| W1740 | 238.39 | 132.09 | 1550 | −11.4319 | 1.0000 |
| W1813 | 235.91 | 119.74 | 2090 | −7.5476 | 0.9636 |
| W1559 | 235.85 | 139.97 | 2293 | −7.1100 | 0.9435 |
| W1488 | 234.33 | 132.86 | 1394 | −7.9452 | 0.9197 |
| W1739 | 234.26 | 145.9 | 2388 | −5.3626 | 0.6827 |
| W1688 | 233.23 | 98.88 | 1797 | −5.5400 | 0.6368 |
| W1586 | 231.19 | 117.38 | 2021 | −2.9708 | 0.2569 |
| W1615 | 228.31 | 146.09 | 2019 | −0.0951 | 0.0531 |
| W1452 | 224.91 | 154.14 | 1875 | 2.9766 | 0.0060* |
| W1796 | 223.65 | 162.79 | 1175 | 1.7199 | 0.0184* |
| W1370 | 222.79 | 143.5 | 2072 | 5.5358 | 0.0006* |
| W1508 | 220.92 | 122.46 | 1722 | 6.5667 | 0.0003* |
| W1524 | 220.65 | 125.95 | 2060 | 7.6512 | <.0001* |
| W1624 | 218.83 | 101.08 | 2555 | 10.3191 | <.0001* |
| W1429 | 211.36 | 140.37 | 2048 | 16.9162 | <.0001* |
| W1509 | 210.14 | 123.64 | 2279 | 18.5758 | <.0001* |
| W1779 | 208.49 | 109.04 | 997 | 15.7901 | <.0001* |
| W1663 | 206.93 | 82.06 | 2527 | 22.1789 | <.0001* |
| W1646 | 204.34 | 114.18 | 1116 | 20.7006 | <.0001* |
| W1564 | 196.07 | 53.79 | 1069 | 28.6870 | <.0001* |
| W1649 | 195.41 | 120.29 | 2406 | 33.5160 | <.0001* |
| W1811 | 195.19 | 107.88 | 2116 | 33.2242 | <.0001* |
| W1613 | 173.91 | 112.48 | 1712 | 53.5485 | <.0001* |
| W1529 | 173.77 | 91.97 | 1869 | 54.1019 | <.0001* |
| W1317 | 172.32 | 110.1 | 1847 | 55.4976 | <.0001* |
| W1402 | 164.09 | 109.38 | 1912 | 63.8850 | <.0001* |
| W1382 | 163.91 | 103.52 | 1781 | 63.7378 | <.0001* |

All Selected Genes were grown and processed for FT-IR analysis. It was hypothesized that an increase in lipid (and potentially oil) content would alter fatty acid methyl ester (FAME) content of the cell, which can be measured by IR spectroscopy. Below is a table that lists all of the predicted lipid content percentages for each strain when grown in HSM under constant light. An ANOVA with Dunnett's statistic test (p<0.05) was applied to the samples to determine which were significantly different than wild type. While the majority of selected genes did not show a significant difference than wild type, 12 lines did have mean % FAME value that was statistically lower than wild type.

TABLE 39

| Winner ID | % FAME | STD | % RSD |
|---|---|---|---|
| W1313 | 13.12 | 0.9541 | 7.27% |
| W1314 | 12.38 | 0.3539 | 2.86% |
| W1315 | 11.92 | 1.4809 | 12.42% |
| W1316 | 11.40 | 0.5431 | 4.77% |
| W1317 | 12.36 | 0.5159 | 4.17% |
| W1318 | 13.16 | 0.7433 | 5.65% |
| W1324 | 10.66 | 0.7702 | 7.22% |
| W1335 | 11.99 | 0.6210 | 5.18% |
| W1336 | 11.63 | 1.1521 | 9.90% |
| W1342 | 9.49 | 0.9097 | 9.59% |
| W1343 | 10.23 | 0.8750 | 8.55% |
| W1350 | 12.53 | 0.6067 | 4.84% |
| W1352 | 12.28 | 1.8258 | 14.87% |
| W1363 | 11.73 | 0.5486 | 4.68% |
| W1370 | 11.93 | 0.4700 | 3.94% |
| W1381 | 12.19 | 0.6636 | 5.44% |
| W1382 | 10.62 | 0.6538 | 6.16% |
| W1386 | 12.49 | 0.3247 | 2.60% |
| W1399 | 10.83 | 0.7877 | 7.27% |
| W1400 | 11.53 | 1.6359 | 14.18% |
| W1401 | 11.32 | 0.3197 | 2.83% |
| W1402 | 10.20 | 0.1389 | 1.36% |
| W1416 | 13.32 | 0.5356 | 4.02% |
| W1418 | 12.75 | 0.1620 | 1.27% |
| W1424 | 11.37 | 0.7400 | 6.51% |
| W1429 | 11.20 | 1.9793 | 17.68% |
| W1440 | 12.29 | 0.5478 | 4.46% |
| W1446 | 11.76 | 0.1102 | 0.94% |
| W1452 | 11.58 | 0.2608 | 2.25% |
| W1456 | 12.44 | 1.0748 | 8.64% |
| W1460 | 13.12 | 0.8775 | 6.69% |
| W1463 | 11.40 | 0.5532 | 4.85% |
| W1468 | 10.67 | 0.2491 | 2.33% |
| W1476 | 11.71 | 0.4658 | 3.98% |
| W1479 | 13.13 | 0.5434 | 4.14% |
| W1480 | 12.78 | 0.1361 | 1.06% |
| W1488 | 13.00 | 1.2453 | 9.58% |
| W1491 | 12.56 | 0.7337 | 5.84% |
| W1492 | 12.07 | 0.6954 | 5.76% |
| W1493 | 14.31 | 0.0751 | 0.52% |
| W1495 | 13.72 | 0.7770 | 5.66% |
| W1508 | 12.01 | 0.7264 | 6.05% |
| W1509 | 11.37 | 0.0603 | 0.53% |
| W1510 | 12.14 | 1.0916 | 8.99% |
| W1511 | 11.20 | 0.5077 | 4.53% |
| W1517 | 10.98 | 0.3863 | 3.52% |
| W1524 | 11.80 | 0.8895 | 7.54% |
| W1525 | 14.00 | 0.3132 | 2.24% |
| W1529 | 13.70 | 0.4267 | 3.12% |
| W1536 | 13.23 | 0.3889 | 2.94% |
| W1559 | 11.39 | 0.9469 | 8.31% |
| W1564 | 12.07 | 0.3378 | 2.80% |
| W1580 | 12.87 | 0.7253 | 5.64% |
| W1586 | 11.05 | 0.6646 | 6.01% |
| W1602 | 12.25 | 0.1992 | 1.63% |
| W1604 | 13.05 | 0.5977 | 4.58% |
| W1613 | 13.01 | 0.5014 | 3.85% |
| W1615 | 11.63 | 0.7451 | 6.41% |
| W1624 | 10.94 | 0.4715 | 4.31% |
| W1627 | 11.50 | 0.3225 | 2.81% |
| W1644 | 10.43 | 0.6724 | 6.45% |
| W1646 | 11.30 | 1.6393 | 14.51% |
| W1649 | 13.04 | 0.4879 | 3.74% |
| W1660 | 12.65 | 0.0777 | 0.61% |
| W1663 | 9.95 | 0.3550 | 3.57% |
| W1665 | 12.93 | 0.5955 | 4.60% |
| W1667 | 11.63 | 0.6941 | 5.97% |
| W1671 | 12.59 | 0.4000 | 3.18% |
| W1686 | 10.38 | 0.4352 | 4.19% |
| W1688 | 13.11 | 0.5514 | 4.20% |
| W1696 | 10.53 | 0.6038 | 5.74% |
| W1702 | 10.77 | 0.6149 | 5.71% |
| W1705 | 8.82 | 0.3061 | 3.47% |
| W1712 | 11.37 | 1.8017 | 15.85% |
| W1724 | 7.37 | 0.0666 | 0.90% |
| W1732 | 11.48 | 0.3449 | 3.00% |
| W1739 | 9.91 | 1.0604 | 10.70% |
| W1740 | 11.60 | 0.9608 | 8.28% |
| W1743 | 9.48 | 0.8479 | 8.94% |
| W1758 | 10.90 | 0.1550 | 1.42% |
| W1779 | 9.23 | 1.0365 | 11.23% |
| W1780 | 11.90 | 0.8297 | 6.97% |
| W1786 | 10.32 | 0.2750 | 2.66% |
| W1796 | 9.41 | 0.6615 | 7.03% |
| W1806 | 10.13 | 1.3212 | 13.05% |
| W1811 | 9.59 | 0.9018 | 9.41% |
| W1812 | 9.32 | 1.0922 | 11.72% |
| W1813 | 8.73 | 1.3703 | 15.69% |
| W1818 | 8.30 | 0.4461 | 5.37% |
| W1826 | 10.23 | 1.0332 | 10.10% |
| W1827 | 11.82 | 0.2211 | 1.87% |
| W1834 | 12.25 | 1.9653 | 16.04% |
| W1849 | 12.76 | 0.5508 | 4.32% |
| W1853 | 11.62 | 0.4933 | 4.24% |
| W1856 | 10.27 | 0.3408 | 3.32% |
| WT | 12.31 | 1.5939 | 12.95% |

Based on the process of wild type competition and regeneration of transgenic lines, 28 of 93 selected genes were validated as having a competitive growth advantage due to overexpression of the gene. These genes are listed in the table below.

TABLE 40

| | Winner | | | |
|---|---|---|---|---|
| Gene | ID | Locus ID | BLASTp description | Class |
| 1 | W1317 | g3274 | aldo/keto reductase family | 1 |
| 4 | W1646 | g7118 | small protein associating with GAPDH and PRK | 2 |
| 4 | W1659 | g7118 | small protein associating with GAPDH and PRK | |
| 4 | W1670 | g7118 | small protein associating with GAPDH and PRK | |
| 4 | W1730 | g7118 | small protein associating with GAPDH and PRK | |
| 7 | W1624 | g2754 | | |
| 7 | W1649 | g2754 | | 2 |
| 11 | W1313 | g4907 | | 1 |
| 13 | W1705 | g5656 | phospholipase/carboxylesterase | 3 |
| 19 | W1446 | g6739 | | 1 |
| 20 | W1491 | g76 | | 1 |
| 23 | W1402 | scaffold223: | | 1 |
| | | 117584-119864 | | |
| 33 | W1475 | g1656 | | |
| 33 | W1493 | g1656 | | 3 |
| 34 | W1673 | g1790 | light-harvesting chlorophyll-a/b binding protein | |
| 34 | W1686 | g1790 | light-harvesting chlorophyll-a/b binding protein | 2 |
| 34 | W1726 | g1790 | light-harvesting chlorophyll-a/b binding protein | |
| 35 | W1580 | g2186 | cytochrome c oxidase subunit | 1 |
| 43 | W1559 | g4732 | | 1 |
| 44 | W1510 | g5667 | | 2 |
| 44 | W1555 | g5667 | | |
| 45 | W1382 | g5980 | predicted protein [C. reinhardtii] | 1 |
| 47 | W1517 | g7085 | hypothetical protein [V. carteri f. nagariensis] | 1 |
| 48 | W1724 | g7161 | | 1 |
| 51 | W1529 | g8172 | | 1 |
| 58 | W1732 | scaffold150: | | 2 |
| | | 396278-396306 | | |
| 63 | W1739 | scaffold318: | hypothetical protein [C. variabilis] | 4 |
| | | 127147-127942 | | |
| 70 | W1492 | scaffold79: | | 1 |
| | | 428425-428443 | | |
| 73 | W1660 | g2209 | light-harvesting chlorophyll-a/b binding protein | 3 |
| 73 | W1663 | g2209 | light-harvesting chlorophyll-a/b binding protein | 2 |
| 76 | W1350 | g623 | RuBisCO small subunit | 1 |
| 76 | W1479 | g623 | RuBisCO small subunit | 3 |
| 76 | W1567 | g623 | RuBisCO small subunit | |
| 77 | W1758 | AmaxDRAFT_1006 | alpha/beta hydrolase fold protein | 3 |
| 81 | W1853 | AmaxDRAFT_3755 | hypothetical protein | 1 |
| 87 | W1856 | AmaxDRAFT_3426 | putative ATP-dependent DNA helicase DinG | 3 |
| 88 | W1779 | AmaxDRAFT_4116 | serine/threonine protein kinase with pentapeptide | 1 |
| | | | repeats | |
| 90 | W1812 | AmaxDRAFT_0926 | isoleucyl-tRNA synthetase | 2 |
| 92 | W1849 | NZ_ABYK01000001: | | 3 |
| | | 4799 6-48113 | | |

### Overall Summary

The table below lists all of the validated genes for increased biomass production in photosynthetic organisms.

| Seq ID No | Winner | Locus ID | BLAST Description | % CDS | Class | Source |
|---|---|---|---|---|---|---|
| 1 & 100 | W0018 | Cre13 · g581650 | ribosomal protein L12-A | 67 | 3 | C. reinhardtii |
| 2 & 101 | W0024 | Cre12 · g551451 | | 0 | 3 | C. reinhardtii |
| 3 & 102 | W0033 | Cre02 · g106600 | Ribosomal protein S19e family protein | 100 | 1 | C. reinhardtii |
| 4 & 103 | W0038 | Cre14 · g621550 | thioredoxin M-type 4 | 11 | 2 | C. reinhardtii |
| 5 & 104 | W0048 | Cre17 · g722200 | mitochondrial ribosomal protein L11 | 100 | 2 | C. reinhardtii |
| 6 & 105 | W0049 | Cre01 · g043350 | Pheophorbide a oxygenase family | 0 | 3 | C. reinhardtii |
| | | | protein with Rieske [2Fe—2S] domain | | | |
| 20 | W0057 | Cre02 · g120150 | ribulose bisphosphate carboxylase | 52 | 3 | C. reinhardtii |
| | | | small chain 1A | | | |
| 7 & 106 | W0058 | Cre03 · g198000 | Protein phosphatase 2C family protein | 84 | 1 | C. reinhardtii |
| 8 107 | W0062 | Cre01 · g050308 | Ribosomal protein L3 family protein | 70 | 1 | C. reinhardtii |
| 24 | W0065 | Cre05 · g234550 | fructose-bisphosphate aldolase 2 | 92 | 2 | C. reinhardtii |
| 9 &108 | W0087 | Cre10 · g417700 | ribosomal protein 1 | 100 | 5 | C. reinhardtii |
| 10 &109 | W0091 | Cre01 · g059600 | Transport protein particle (TRAPP) | 75 | 3 | C. reinhardtii |
| | | | component | | | |
| 11 & 110 | W0104 | Cre12 · g529650 | Ribosomal protein L7Ae/L30e/S12e/ | 86 | 1 | C. reinhardtii |
| | | | Gadd45 family protein | | | |
| 12 & 111 | W0106 | Cre02 · g114600 | 2-cysteine peroxiredoxin B | 56 | 3 | C. reinhardtii |
| 13 & 112 | W0134 | Cre01 · g010900 | glyceraldehyde-3-phosphate | 100 | 1 | C. reinhardtii |
| | | | dehydrogenase B subunit | | | |
| 14 &113 | W0149 | Cre03 · g204250 | S-adenosyl-L-homocysteine hydrolase | 9 | 2 | C. reinhardtii |
| 15 & 114 | W0150 | Cre13 · g572300 | | 23 | 1 | C. reinhardtii |
| 16 & 115 | W0162 | Cre06 · g298650 | eukaryotic translation initiation factor | 95 | 2 | C. reinhardtii |
| | | | 4A1 | | | |
| 17 & 116 | W0167 | Cre10 · g447950 | | 100 | 2 | C. reinhardtii |
| 18 & 117 | W0172 | Cre02 · g134700 | Ribosomal protein L4/L1 family | 36 | 3 | C. reinhardtii |
| 31 | W0190 | Cre02 · g075700 | Ribosomal protein L19e family | 98 | 2 | C. reinhardtii |
| | | | protein | | | |
| 32 | W0194 | Cre09 · g386650 | ADP/ATP carrier 3 | 29 | 2 | C. reinhardtii |
| 36 | W0201 | Cre17 · g700750 | | 24 | 1 | C. reinhardtii |
| 36 | W0211 | Cre17 · g700750 | | 0 | 3 | C. reinhardtii |
| 25 | W0227 | Cre03 · g210050 | Ribosomal protein L35 | 71 | 2 | C. reinhardtii |
| 19 & 118 | W0240 | Cre12 · g529400 | Ribosomal protein S27 | 100 | 1 | C. reinhardtii |
| 20 & 255 | W0255 | Cre02 · g120150 | ribulose bisphosphate carboxylase | 100 | 1 | C. reinhardtii |
| | | | small chain 1A | | | |
| 13 | W0268 | Cre01 · g010900 | glyceraldehyde-3-phosphate | 11 | 4 | C. reinhardtii |
| | | | dehydrogenase B subunit | | | |
| 21 & 129 | W0282 | Cre14 · g612800 | | 100 | 1 | C. reinhardtii |
| 22 & 121 | W0318 | Cre01 · g000850 | | 100 | 3 | C. reinhardtii |
| 23 & 122 | W0325 | Cre09 · g416500 | zinc finger (C2H2 type) family protein | 97 | 3 | C. reinhardtii |
| 24 & 123 | W0335 | Cre05 · g234550 | fructose-bisphosphate aldolase 2 | 100 | 1 | C. reinhardtii |
| 25 &124 | W0343 | Cre03 · g210050 | Ribosomal protein L35 | 100 | 5 | C. reinhardtii |
| 26 & 125 | W0351 | Cre14 · g624000 | F-box/RNI-like superfamily protein | 100 | 2 | C. reinhardtii |
| 9 | W0355 | Cre10 · g417700 | ribosomal protein 1 | 99 | 3 | C. reinhardtii |
| 27 & 126 | W0363 | Cre13 · g590500 | fatty acid desaturase 6 | 100 | 5 | C. reinhardtii |
| 27 | W0371 | Cre13 · g590500 | fatty acid desaturase 6 | 57 | 3 | C. reinhardtii |
| 28 &127 | W0422 | Cre02 · g091100 | Ribosomal protein L23/L15e family | 100 | 3 | C. reinhardtii |
| | | | protein | | | |
| 29 & 128 | W0430 | Cre01 · g072350 | SPFH/Band 7/PHB domain-containing | 100 | 2 | C. reinhardtii |
| | | | membrane-associated protein family | | | |
| 30 & 129 | W0445 | Cre14 · g611150 | Small nuclear ribonucleoprotein | 10 | 2 | C. reinhardtii |
| | | | family protein | | | |
| 31 & 130 | W0462 | Cre02 · g075700 | Ribosomal protein L19e family protein | 100 | 3 | C. reinhardtii |
| 32 & 131 | W0475 | Cre09 · g386650 | ADP/ATP carrier 3 | 100 | 1 | C. reinhardtii |
| 32 & 131 | W0475 | Cre09 · g386650 | ADP/ATP carrier 3 | 100 | only primary | C. reinhardtii |
| | | | | | data | |
| 33 & 132 | W0481 | Cre23 · g766250 | photosystem II light harvesting | 12 | 2 | C. reinhardtii |
| | | | complex gene 2.2 | | | |
| 34 & 133 | W0489 | Cre12 · g528750 | Ribosomal protein L11 family protein | 96 | 3 | C. reinhardtii |
| 35 & 134 | W0490 | Cre02 · g139950 | | 100 | 3 | C. reinhardtii |
| 36 & 135 | W0496 | Cre17 · g700750 | | 100 | 5 | C. reinhardtii |
| 37 & 136 | W0607 | g3921 | ubiquitin-associated (UBA)/TS-N | 100 | 2 | S. obliquus |
| | | | domain-containing protein | | | |
| 41 | W0611 | g14780 | ribulose bisphosphate carboxylase | 100 | | S. obliquus |
| | | | small chain 1A; Cyclin family protein | | | |
| 37 | W0626 | g3921 | ubiquitin-associated (UBA)/TS-N | 100 | | S. obliquus |
| | | | domain-containing protein | | | |
| 38 & 137 | W0629 | g2506 | photosystem II subunit X | 100 | 2 | S. obliquus |
| 66 | W0659 | g13997 | aldehyde dehydrogenase 2C4 | 100 | | S. obliquus |
| 39 & 138 | W0667 | scaffold126: | | | 5 | S. obliquus |
| | | 355759-356343 | | | | |
| 40 & 139 | W0675 | g14907 | | 100 | 2 | S. obliquus |
| 41 & 140 | W0677 | g14780 | ribulose bisphosphate carboxylase | 100 | | S. obliquus |
| | | | small chain 1A; Cyclin family protein | | | |
| 41 & 140 | W0723 | g14780 | ribulose bisphosphate carboxylase | 100 | | S. obliquus |
| | | | small chain 1A; Cyclin family protein | | | |
| 42 | W0770 | scaffold18: | | | 1 | S. obliquus |
| | | 1489301-1489559 | | | | |
| 43 | W0771 | scaffold18: | | | | S. obliquus |
| | | 1494447-1495555 | | | | |
| 44 & 141 | W0774 | scaffold42: | | | 5 | S. obliquus |
| | | 463800-464650 | | | | |
| 45 & 142 | W0776 | g14780 | ribulose bisphosphate carboxylase | 46 | 3 | S. obliquus |
| | | | small chain 1A; Cyclin family protein | | | |
| 46 & 143 | W0785 | g12290 | | 100 | 2 | S. obliquus |
| 66 | W0796 | g13997 | aldehyde dehydrogenase 2C4 | 100 | | S. obliquus |
| 47 | W0802 | scaffold33: | | | 5 | S. obliquus |
| | | 535965-537528 | | | | |
| 41 | W0805 | g14780 | ribulose bisphosphate carboxylase | 100 | | S. obliquus |
| | | | small chain 1A; Cyclin family protein | | | |
| 48 | W0823 | scaffold67: | | | 2 | S. obliquus |
| | | 222004-223125 | | | | |
| 49 & 144 | W0829 | scaffold110: | | | 5 | S. obliquus |
| | | 302109-303275 | | | | |
| 50 & 145 | W0841 | g4280 | | 100 | 5 | S. obliquus |
| 51 & 146 | W0883 | g18194 | gamma carbonic anhydrase like 1 | 100 | 3 | S. obliquus |
| 41 | W0912 | g14780 | ribulose bisphosphate carboxylase | 100 | | S. obliquus |
| | | | small chain 1A; Cyclin family protein | | | |
| 48 | W0916 | scaffold67: | | | | S. obliquus |
| | | 222004-223125 | | | | |
| 52 &147 | W0923 | g17628 | receptor for activated C kinase 1C | 100 | | S. obliquus |
| 38 | W0924 | g2506 | photosystem II subunit X | 100 | | S. obliquus |
| 59 | W0932 | g9576 | photosystem II subunit Q-2 | 97 | | S. obliquus |
| 53 &148 | W0934 | g13997 | aldehyde dehydrogenase 2C4 | 93 | 3 | S. obliquus |
| 54 & 149 | W0949 | g14943 | ATP synthase delta-subunit gene | 100 | 1 | S. obliquus |
| 55 &150 | W0950 | g17628 | receptor for activated C kinase 1C | 58 | 4 | S. obliquus |
| 41 | W0951 | g14780 | ribulose bisphosphate carboxylase | 100 | | S. obliquus |
| | | | small chain 1A; Cyclin family protein | | | |
| 56 & 151 | W0956 | g18330 | Protein kinase superfamlly protein | 42 | 2 | S. obliquus |
| 57 & 152 | W0979 | g664 | Nucleic acid-binding, OB-fold-like | 100 | 4 | S. obliquus |
| | | | protein | | | |
| 58 | W0980 | scaffold240: | | | 2 | S. obliquus |
| | | 19496-20329 | | | | |
| 59 &153 | W1004 | g9576 | photosystem II subunit Q-2 | 97 | 2 | S. obliquus |
| 38 | W1028 | g2506 | photosystem II subunit X | 100 | | S. obliquus |
| 60 &154 | W1036 | g13214 | | 3 | 4 | S. obliquus |
| 61 & 155 | W1083 | g9576 | photosystem II subunit Q-2 | 19 | 5 | S. obliquus |
| 62 & 156 | W1092 | scaffold64: | | | 4 | S. obliquus |
| | | 287639-288387 | | | | |
| 61 | W1098 | g9576 | photosystem II subunit Q-2 | 19 | | S. obliquus |
| 63 | W1100 | g884 | | 100 | | S. obliquus |
| 63 & 157 | W1104 | g884 | | 100 | 2 | S. obliquus |
| 38 | W1115 | g2506 | photosystem II subunit X | 100 | | S. obliquus |
| 64 & 158 | W1123 | g1509 | Protein kinase superfamily protein | 100 | 3 | S. obliquus |
| | | | with octicosapeptide/Phox/Bem1p | | | |
| | | | domain | | | |
| 65 & 159 | W1146 | g8264 | | 26 | 4 | S. obliquus |
| 49 | W1155 | scaffold110: | | | | S. obliquus |
| | | 302109-303275 | | | | |
| 46 | W1169 | g12290 | | 100 | | S. obliquus |
| 49 | W1170 | scaffold110: | | | | S. obliquus |
| | | 302109-303275 | | | | |
| 49 | W1176 | scaffold110: | | | | S. obliquus |
| | | 302109-303275 | | | | |
| 66 & 160 | W1203 | g13997 | aldehyde dehydrogenase 2C4 | 100 | 1 | S. obliquus |
| 67 & 161 | W1210 | g16071 | | 100 | 2 | S. obliquus |
| 68 & 162 | W1233 | g7387 | demeter-like 2 | 100 | 3 | S. obliquus |
| 69 & 163 | W1313 | g4907 | | | 1 | Desmodesmus sp. |
| 70 & 164 | W1317 | g3274 | aldo/keto reductase family | | 1 | Desmodesmus sp. |
| 71 & 165 | W1350 | g623 | RuBisCO small subunit | | 1 | Desmodesmus sp. |
| 72 & 166 | W1382 | g5980 | predicted protein [C. reinhardtii] | | 1 | Desmodesmus sp. |
| 73 & 167 | W1402 | scaffold223: | | | 1 | Desmodesmus sp. |
| | | 117584-119864 | | | | |
| 74 | W1446 | g6739 | | | 1 | Desmodesmus sp. |
| 78 | W1475 | g1656 | | | | Desmodesmus sp. |
| 75 & 167 | W1479 | g623 | RuBisCO small subunit | | 3 | Desmodesmus sp. |
| 76 & 169 | W1491 | g76 | | | 1 | Desmodesmus sp. |
| 77 & 170 | W1492 | scaffold79: | | | 1 | Desmodesmus sp. |
| | | 428425-428443 | | | | |
| 78 & 171 | W1493 | g1656 | | | 3 | Desmodesmus sp. |
| 79 & 172 | W1510 | g5667 | | | 2 | Desmodesmus sp. |
| 80 & 173 | W1517 | g7085 | hypothetical protein [V. carteri | | 1 | Desmodesmus sp. |
| | | | f. nagariensis] | | | |
| 81 & 174 | W1529 | g8172 | | | 1 | Desmodesmus sp. |
| 79 | W1555 | g5667 | | | | Desmodesmus sp. |
| 82 & 175 | W1559 | g4732 | | | 1 | Desmodesmus sp. |
| 75 | W1567 | g623 | RuBisCO small subunit | | | Desmodesmus sp. |
| 83 & 176 | W1580 | g2186 | cytochrome c oxidase subunit | | 1 | Desmodesmus sp. |
| 84 & 177 | W1624 | g2754 | | | | Desmodesmus sp. |
| 85 & 178 | W1646 | g7118 | small protein associating with GAPDH | | 2 | Desmodesmus sp. |
| | | | and PRK | | | |
| 86 & 179 | W1649 | g2754 | | | 2 | Desmodesmus sp. |
| 85 | W1659 | g7118 | small protein associating with GAPDH | | | Desmodesmus sp. |
| | | | and PRK | | | |
| 87 & 180 | W1660 | g2209 | light-harvesting chlorophyll-a/b | | 3 | Desmodesmus sp. |
| | | | binding protein | | | |
| 88 & 181 | W1663 | g2209 | light-harvesting chlorophyll-a/b | | 2 | Desmodesmus sp. |
| | | | binding protein | | | |
| 85 | W1670 | g7118 | small protein associating with GAPDH | | | Desmodesmus sp. |
| | | | and PRK | | | |
| 89 | W1673 | g1790 | light-harvesting chlorophyll-a/b | | | Desmodesmus sp. |
| | | | binding protein | | | |
| 89 & 182 | W1686 | g1790 | light-harvesting chlorophyll-a/b | | 2 | Desmodesmus sp. |
| | | | binding protein | | | |
| 90 & 183 | W1705 | g5656 | phospholipase/carboxylesterase | | 3 | Desmodesmus sp. |
| 91 & 184 | W1724 | g7161 | | | 1 | Desmodesmus sp. |
| 89 | W1726 | g1790 | light-harvesting chlorophyll-a/b | | | Desmodesmus sp. |
| | | | binding protein | | | |
| 85 | W1730 | g7118 | small protein associating with GAPDH | | | Desmodesmus sp. |
| | | | and PRK | | | |
| 92 & 185 | W1732 | scaffold150: | | | 2 | Desmodesmus sp. |
| | | 396278-396306 | | | | |
| 93 & 186 | W1739 | scaffold318: | hypothetical protein [C. variabilis] | | 4 | Desmodesmus sp. |
| | | 127147-127942 | | | | |
| 94 | W1758 | AmaxDRAFT_1006 | alpha/beta hydrolase fold protein | | 3 | A. maxima |
| 95 & 187 | W1779 | AmaxDRAFT_4116 | serine/threonine protein kinase with | | 1 | A. maxima |
| | | | pentapeptide repeats | | | |
| 96 & 188 | W1812 | AmaxDRAFT_0926 | isoleucyl-tRNA synthetase | | 2 | A. maxima |
| 97 | W1849 | NZ_ABYK01000001: | | | 3 | A. maxima |
| | | 479 96-48113 | | | | |
| 98 & 189 | W1853 | AmaxDRAFT_3755 | hypothetical protein | | 1 | A. maxima |
| 99 | W1856 | AmaxDRAFT_3426 | putative ATP-dependent DNA helicase | | 3 | A. maxima |
| | | | DinG | | | |

What is claimed is:

1. A photosynthetic organism transformed with at least one polynucleotide comprising:
(a) a nucleic acid sequence of SEQ ID NO: 1 to 99 or
(b) a nucleotide sequence with at least 80%, at least 85%, at least 90%, at least 95%, at least 98%, or at least 99% sequence identity to the nucleic acid sequence of SEQ ID NO: 1 to 99;
wherein the transformed photosynthetic organism's biomass is increased as compared to a biomass of an untransformed photosynthetic organism of the same species.

2. The transformed photosynthetic organism of 1, wherein the increase is measured by a competition assay, growth rate, carrying capacity, productivity, cell proliferation, seed yield, organ growth, or polysome accumulation.

3. The transformed photosynthetic organism of 2, wherein the increase is measured by a competition assay.

4. The transformed photosynthetic organism of 3, wherein the competition assay is performed in a turbidostat.

5. The transformed photosynthetic organism of 1, wherein the increase is shown by the transformed photosynthetic organism having a positive selection coefficient as compared an untransformed photosynthetic organism of the same species.

6. The transformed photosynthetic organism of 5, wherein the selection coefficient is from 0.05 to 0.10, from 0.10 to 0.5, from 0.5 to 0.75, from 0.75 to 1.0, from 1.0 to 1.5, from 1.5 to 2.0, or 2.0 to 3.0.

7. The transformed photosynthetic organism of 1, wherein the increase is measured by growth rate.

8. The transformed photosynthetic organism of 7, wherein the transformed photosynthetic organism has an increase in growth rate as compared to an untransformed photosynthetic organism of the same species of from 5% to 10%, from 10% to 15%, from 15% to 25%, from 25% to 50%, from 50% to 75%, from 75% to 100%, from 100% to 150%, from 150% to 200%, from 200% to 300%, or from 300% to 400%.

9. The transformed photosynthetic organism of 1, wherein the increase is measured by an increase in carrying capacity.

10. The transformed photosynthetic organism of 9, wherein the units of carrying capacity are mass per unit of volume or area.

11. The transformed photosynthetic organism of 1, wherein the increase is measured by an increase in productivity.

12. The transformed photosynthetic organism of 11, wherein the units of productivity are grams per meter squared per day or mass per acre, mass per unit area such as tons per acre/hectare, or volume per unit area such as bushels per acre/hectare.

13. The transformed photosynthetic organism of 12, wherein the transformed photosynthetic organism has an increase in productivity as measured in grams per meter squared per day, as compared to an untransformed photosynthetic organism of the same species of from 5% to 10%, from 10% to 15%, from 15% to 25%, from 25% to 50%, from 50% to 75%, from 75% to 100%, from 100% to 150%, from 150% to 200%, from 200% to 300%, or from 300% to 400%.

14. The transformed photosynthetic organism of 1, wherein the transformed photosynthetic organism is grown in an aqueous environment.

15. The transformed photosynthetic organism of 1, wherein the transformed photosynthetic organism is a bacterium.

16. The transformed photosynthetic organism of 15, wherein the bacterium is a cyanobacterium.

17. The transformed photosynthetic organism of 1, wherein the transformed photosynthetic organism is an alga.

18. The transformed photosynthetic organism of 17, wherein the alga is a microalga.

19. The transformed photosynthetic organism of 18, wherein the microalga is at least one of a Chlamydomonas sp., Volvacales sp., Desmid sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmus sp.

20. The transformed photosynthetic organism of 18, wherein the microalga is at least one of Chlamydomonas reinhardtii, N. oceanica, N. salina, Dunaliella salina, H. pluvalis, S. dimorphus, Dunaliella viridis, N. oculata, Dunaliella tertiolecta, S. Maximus, or A. Fusiformus.

21. The transformed photosynthetic organism of 1, wherein the transformed photosynthetic organism is a vascular plant.

22. The transformed photosynthetic organism of 21, wherein the transformed photosynthetic organism is Brassica (e.g., Brassica nigra, Brassica napus, Brassica hirta, Brassica rapa, Brassica campestris, Brassica carinata, and Brassica juncea), soybean (Glycine max), castor bean (Ricinus communis), cotton, safflower (Carthamus tinctorius), sunflower (Helianthus annuus), flax (Linum usitatissimum), corn (Zea mays), coconut (Cocos nucifera), palm (Elaeis guineensis), oil nut trees such as olive (Olea europaea), sesame, and peanut (Arachis hypogaea), as well as Arabidopsis, tobacco, wheat, sugarcane, sugar beet, barley, oats, amaranth, potato, rice, tomato, legumes (e.g., peas, beans, lentils, alfalfa, etc.), grasses (e.g. Miscanthus, switchgrass, energy cane), vegetable crops and fruits.

23. A transformed photosynthetic organism comprising at least one exogenous polynucleotide encoding a polypeptide comprising:
(a) at least one amino acid sequence of SEQ ID NO: 100 to 189 or
(b) an amino acid sequence with at least 80%, at least 85%, at least 90%, at least 95%, at least 98%, or at least 99% sequence identity to at least one of SEQ ID NO: 100 to 189;
wherein the transformed photosynthetic organism expresses the at least one exogenous polynucleotide; and
wherein the transformed photosynthetic organism's biomass is increased as compared to a biomass of an untransformed photosynthetic organism of the same species.

24. The transformed photosynthetic organism of 23, wherein the increase is measured by a competition assay, growth rate, carrying capacity, productivity, cell proliferation, seed yield, organ growth, or polysome accumulation.

25. The transformed photosynthetic organism of 24, wherein the increase is measured by a competition assay.

26. The transformed photosynthetic organism of 25, wherein the competition assay is performed in a turbidostat.

27. The transformed photosynthetic organism of 23, wherein the increase is shown by the transformed photosynthetic organism having a positive selection coefficient as compared to an untransformed photosynthetic organism of the same species.

28. The transformed photosynthetic organism of 27, wherein the selection coefficient is from 0.05 to 0.10, from 0.10 to 0.5, from 0.5 to 0.75, from 0.75 to 1.0, from 1.0 to 1.5, from 1.5 to 2.0, or 2.0 to 3.0.

29. The transformed photosynthetic organism of 23, wherein the increase is measured by growth rate.

30. The transformed photosynthetic organism of 29, wherein the transformed photosynthetic organism has an increase in growth rate as compared to an untransformed photosynthetic organism of the same species of from 5% to 10%, from 10% to 15%, from 15% to 25%, from 25% to 50%, from 50% to 75%, from 75% to 100%, from 100% to 150%, from 150% to 200%, from 200% to 300%, or from 300% to 400%.

31. The transformed photosynthetic organism of 23, wherein the increase is measured by an increase in carrying capacity.

32. The transformed photosynthetic organism of 31, wherein the units of carrying capacity are mass per unit of volume or area.

33. The transformed photosynthetic organism of 23, wherein the increase is measured by an increase in productivity.

34. The transformed photosynthetic organism of 33, wherein the units of culture productivity are grams per meter squared per day or mass per acre, mass per unit area such as tons per acre/hectare, or volume per unit area such as bushels per acre/hectare.

35. The transformed photosynthetic organism of 34, wherein the transformed photosynthetic organism has an increase in productivity as measured in grams per meter squared per day, as compared to an untransformed photosynthetic organism of the same species of from 5% to 10%, from 10% to 15%, from 15% to 25%, from 25% to 50%, from 50% to 75%, from 75% to 100%, from 100% to 150%, from 150% to 200%, from 200% to 300%, or from 300% to 400%.

36. The transformed photosynthetic organism of 23, wherein the transformed photosynthetic organism is grown in an aqueous environment.

37. The transformed photosynthetic organism of 23, wherein the transformed photosynthetic organism is a bacterium.

38. The transformed photosynthetic organism of 37, wherein the bacterium is a cyanobacterium.

39. The transformed photosynthetic organism of 23, wherein the transformed photosynthetic organism is an alga.

40. The transformed photosynthetic organism of 39, wherein the alga is a microalga.

41. The transformed photosynthetic organism of 40, wherein the microalga is at least one of a Chlamydomonas sp., Volvacales sp., Desmid sp., Dunaliella sp., Scenedesmus sp., Chlorella sp., Hematococcus sp., Volvox sp., Nannochioropsis sp., Arthrospira sp., Sprirulina sp., Botryococcus sp., Haematococcus sp., or Desmodesmus sp.

42. The transformed photosynthetic organism of 40, wherein the microalga is at least one of Chlamydomonas reinhardtii, N. oceanica, N. salina, Dunaliella salina, H. pluvalis, S. dimorphus, Dunaliella viridis, N. oculata, Dunaliella tertiolecta, S. Maximus, or A. Fusiformus.

43. The transformed photosynthetic organism of 23, wherein the transformed photosynthetic organism is a vascular plant.

44. The transformed photosynthetic organism of 43, wherein the transformed photosynthetic organism is Brassica (e.g., Brassica nigra, Brassica napus, Brassica hirta, Brassica rapa, Brassica campestris, Brassica carinata, and Brassica juncea), soybean (Glycine max), castor bean (Ricinus communis), cotton, safflower (Carthamus tinctorius), sunflower (Helianthus annuus), flax (Linum usitatissimum), corn (Zea mays), coconut (Cocos nucifera), palm (Elaeis guineensis), oil nut trees such as olive (Olea europaea), sesame, and peanut (Arachis hypogaea), as well as Arabidopsis, tobacco, wheat, sugarcane, sugar beet, barley, oats, amaranth, potato, rice, tomato, legumes (e.g., peas, beans, lentils, alfalfa, etc.), grasses (e.g. Miscanthus, switchgrass, energy cane), vegetable crops and fruits.

45. A method of increasing biomass of a photosynthetic organism, comprising:
(a) transforming the photosynthetic organism with at least one polynucleotide to produce a transformed photosynthetic organism, wherein the polynucleotide comprises:
(i) a nucleic acid sequence of SEQ ID NO: 1 to 99; or
(ii) a nucleotide sequence with at least 80%, at least 85%, at least 90%, at least 95%, at least 98%, or at least 99% sequence identity to the nucleic acid sequence of SEQ ID NO: 1-99;
wherein the transformed photosynthetic organism expresses said polynucleotide; and
wherein the transformed photosynthetic organism produces an increase in biomass as compared to an untransformed photosynthetic organism of the same species.

46. The method of 45, wherein the increase is measured by a competition assay, growth rate, carrying capacity, productivity, cell proliferation, seed yield, organ growth, or polysome accumulation.

47. The method of 46, wherein the increase is measured by a competition assay.

48. The method of 47, wherein the competition assay is performed in a turbidostat.

49. The method of 45, wherein the increase is shown by the transformed photosynthetic organism having a positive selection coefficient as compared to an untransformed photosynthetic organism of the same species.

50. The method of 49, wherein the selection coefficient is from 0.05 to 0.10, from 0.10 to 0.5, from 0.5 to 0.75, from 0.75 to 1.0, from 1.0 to 1.5, from 1.5 to 2.0, or 2.0 to 3.0.

51. The method of 45, wherein the increase is measured by growth rate.

52. The method of 51, wherein the transformed photosynthetic organism has an increase in growth rate as compared to an untransformed photosynthetic organism of the same species of from 5% to 10%, from 10% to 15%, from 15% to 25%, from 25% to 50%, from 50% to 75%, from 75% to 100%, from 100% to 150%, from 150% to 200%, from 200% to 300%, or from 300% to 400%.

53. The method of 45, wherein the increase is measured by an increase in carrying capacity.

54. The method of 53, wherein the units of carrying capacity are mass per unit of volume or area.

55. The method of 45, wherein the increase is measured by an increase in culture productivity.

56. The method of 55, wherein the units of productivity are grams per meter squared per day, mass per unit area such as tons per acre/hectare, or volume per unit area such as bushels per acre/hectare.

57. The method of 45, wherein the transformed photosynthetic organism has an increase in productivity as measured in grams per meter squared per day, as compared to an untransformed photosynthetic organism of the same species of from 5% to 10%, from 10% to 15%, from 15% to 25%, from 25% to 50%, from 50% to 75%, from 75% to 100%, from 100% to 150%, from 150% to 200%, from 200% to 300%, or from 300% to 400%.

58. The method of 45, wherein the transformed photosynthetic organism is grown in an aqueous environment.

59. The method of 45, wherein the transformed photosynthetic organism is a bacterium.

60. The method of 59, wherein the bacterium is a cyanobacterium.

61. The method of 45, wherein the transformed photosynthetic organism is an alga.

62. The method of 61, wherein the alga is a microalga.

63. The method of 62, wherein the microalga is at least one of a Chlamydomonas sp., Volvacales sp., Desmid sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmus sp.

64. The method of 62, wherein the microalga is at least one of Chlamydomonas reinhardtii, N. oceanica, N. salina, Dunaliella salina, H. pluvalis, S. dimorphus, Dunaliella viridis, N. oculata, Dunaliella tertiolecta, S. Maximus, or A. Fusiformus.

65. The method of 45, wherein the transformed photosynthetic organism is a vascular plant.

66. The method of 65, wherein the transformed photosynthetic organism is Brassica (e.g., Brassica nigra, Brassica napus, Brassica hirta, Brassica rapa, Brassica campestris, Brassica carinata, and Brassica juncea), soybean (Glycine max), castor bean (Ricinus communis), cotton, safflower (Carthamus tinctorius), sunflower (Helianthus annuus), flax (Linum usitatissimum), corn (Zea mays), coconut (Cocos nucifera), palm (Elaeis guineensis), oil nut trees such as olive (Olea europaea), sesame, and peanut (Arachis hypogaea), as well as Arabidopsis, tobacco, wheat, sugarcane, sugar beet, barley, oats, amaranth, potato, rice, tomato, legumes (e.g., peas, beans, lentils, alfalfa, etc.), grasses (e.g. Miscanthus, switchgrass, energy cane), vegetable crops and fruits.

67. A method of increasing biomass of a photosynthetic organism, comprising:
(a) transforming the photosynthetic organism with at least one polynucleotide to produce a transformed photosynthetic organism, wherein the polynucleotide comprises:
(i) a nucleic acid sequence encodes a polypeptide with an amino acid sequence of SEQ ID NO: 100 to 189; or
(ii) a polypeptide with an amino acid sequence with at least 80%, at least 85%, at least 90%, at least 95%, at least 98%, or at least 99% sequence identity to the nucleic acid sequence of SEQ ID NO: 100 to 189;
wherein the transformed photosynthetic organism expresses the at least one polynucleotide to produce the polypeptide; and
wherein the transformed photosynthetic organism produces an increase in biomass as compared to an untransformed photosynthetic organism of the same species.

68. The method of 67, wherein the increase is measured by a competition assay, growth rate, carrying capacity, productivity, cell proliferation, seed yield, organ growth, or polysome accumulation.

69. The method of 68, wherein the increase is measured by a competition assay.

70. The method of 69, wherein the competition assay is performed in a turbidostat.

71. The method of 67, wherein the increase is shown by the transformed photosynthetic organism having a positive selection coefficient as compared to an untransformed photosynthetic organism of the same species.

72. The method of 71, wherein the selection coefficient is from 0.05 to 0.10, from 0.10 to 0.5, from 0.5 to 0.75, from 0.75 to 1.0, from 1.0 to 1.5, from 1.5 to 2.0, or 2.0 to 3.0.

73. The method of 67, wherein the increase is measured by growth rate.

74. The method of 73, wherein the transformed photosynthetic organism has an increase in growth rate as compared to an untransformed photosynthetic organism of from 5% to 10%, from 10% to 15%, from 15% to 25%, from 25% to 50%, from 50% to 75%, from 75% to 100%, from 100% to 150%, from 150% to 200%, from 200% to 300%, or from 300% to 400%.

75. The method of 67, wherein the increase is measured by an increase in carrying capacity.

76. The method of 75, wherein the units of carrying capacity are mass per unit of volume or area.

77. The method of 67, wherein the increase is measured by an increase in productivity.

78. The method of 77, wherein the units of productivity are grams per meter squared per day, mass per unit area such as tons per acre/hectare, or volume per unit area such as bushels per acre/hectare.

79. The method of 67, wherein the transformed photosynthetic organism has an increase in productivity as measured in grams per meter squared per day, as compared to an untransformed photosynthetic organism of from 5% to 10%, from 10% to 15%, from 15% to 25%, from 25% to 50%, from 50% to 75%, from 75% to 100%, from 100% to 150%, from 150% to 200%, from 200% to 300%, or from 300% to 400%.

80. The method of 67, wherein the transformed photosynthetic organism is grown in an aqueous environment.

81. The method of 67, wherein the transformed photosynthetic organism is a bacterium.

82. The method of 81, wherein the bacterium is a cyanobacterium.

83. The method of 67, wherein the transformed photosynthetic organism is an alga.

84. The method of 83, wherein the alga is a microalga.

85. The method of 84, wherein the microalga is at least one of a Chlamydomonas sp., Volvacales sp., Desmid sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmus sp.

86. The method of 85, wherein the microalga is at least one of Chlamydomonas reinhardtii, N. oceanica, N. salina, Dunaliella salina, H. pluvalis, S. dimorphus, Dunaliella viridis, N. oculata, Dunaliella tertiolecta, S. Maximus, or A. Fusiformus.

87. The method of 67, wherein the transformed photosynthetic organism is a vascular plant.

88. The method of 87, wherein the transformed photosynthetic organism is Brassica (e.g., Brassica nigra, Brassica napus, Brassica hirta, Brassica rapa, Brassica campestris, Brassica carinata, and Brassica juncea), soybean (Glycine max), castor bean (Ricinus communis), cotton, safflower (Carthamus tinctorius), sunflower (Helianthus annuus), flax (Linum usitatissimum), corn (Zea mays), coconut (Cocos nucifera), palm (Elaeis guineensis), oil nut trees such as olive (Olea europaea), sesame, and peanut (Arachis hypogaea), as well as Arabidopsis, tobacco, wheat, sugarcane, sugar beet, barley, oats, amaranth, potato, rice, tomato, legumes (e.g., peas, beans, lentils, alfalfa, etc.), grasses (e.g. Miscanthus, switchgrass, energy cane), vegetable crops and fruits.
